(19) United States
(12) Patent Application Publication
(10) Pub. No.: US 2004/0033549 A1
(43) Pub. Date: Feb. 19, 2004

(54) Quorum sensing signaling in bacteria

(76) Inventors: E. Peter Greenberg, Iowa City, IA (US); Martin Schuster, Iowa City, IA (US); Candi Lostroh, Grennell, IA (US)

Correspondence Address: LAHIVE & COCKFIELD, LLP., 28 STATE STREET, BOSTON, MA, 02109, US

(21) Appl. No.: 10/389,647

(22) Filed: Mar. 14, 2003

Related U.S. Application Data
(63) Continuation-in-part of application No. 09/653,730, filed on Sep. 1, 2000.
(60) Provisional application No. 60/153,022, filed on Sep. 3, 1999.

(51) Int. Cl. G01N 33/554; G01N 33/569

(52) U.S. Cl. 435/7.32

(57) ABSTRACT

The invention provides methods for identifying a modulator of quorum sensing signaling in bacteria, and for identifying a quorum sensing controlled gene in bacteria. In addition, the invention provides quorum sensing controlled genetic loci in Pseudomas aeruginosa. Novel indicator strains and vectors for engineering the strains for use in the method of the invention are also provided.

### RELATED APPLICATIONS

This application is a continuation-in-part of U.S. patent application Ser. No. 09/653,730, filed on Sep. 1, 2000, pending, which claims priority to U.S. Provisional Patent Application Serial No. 60/153,022 filed on Sep. 3, 1999. Each of the foregoing applications is incorporated herein in its entirety by reference.

### GOVERNMENT SUPPORT

This research was supported by grants and fellowships from the National Institutes of Health (GM59026), and the National Science Foundation (MCB9808308 and DBI9602247).

### BACKGROUND OF THE INVENTION

Many gram-negative bacteria have been shown to possess one or more quorum sensing systems (Fuqua, W. C. et al. (1996) Annu. Rev. Microbiol. 50:727-751; Salmond, G. P. C. et al. (1995) Mol. Microbiol. 16:615-624). These systems regulate a variety of physiological processes, including the activation of virulence genes and the formation of biofilms. The systems typically have acylated homoserine lactone ring autoinducers, in which the homoserine lactone ring is conserved. The acyl side chain, however, can vary in length and degree of substitution. In addition, it has been recently demonstrated that quorum sensing is involved in biofilm formation (Davies, D. G. et al. (1998) Science. 280(5361):295-8).

Pseudomonas aeruginosa has two quorum sensing systems, las and rhl, named for their role in the expression of elastase, and the RhlI/RhlR proteins, which were first described for their role in rhamnolipid biosynthesis. (Hanzelka, B. A. et al. (1996) J. Bacteriol. 178:5291-5294; Baldwin, T. O. et al. (1989) J. of Biolum. and Chemilum. 4:326-341; Passador, L., et al. (1993) Science 260:1127-1130; Pearson, J. P et al. (1994) PNAS 91:197-201; Pesci, E. C. et al.(1997) Trends in Microbiol. 5(4):132-135; Pesci, E. C. et al. (1997) J. Bacteriol. 179:3127-3132). The two systems have distinct autoinducer syntheses (lasI and rhlI), transcriptional regulators (lasR and rhlR), and autoinducers (N-(3-oxododecanoyl) homoserine lactone (HSL) and N-butyryl HSL) (Sitnikov, D. M. et al. (1995) Mol. Microbiol. 17:801-812). The rhl and las systems are involved in regulating the expression of a number of secreted virulence factors, biofilm development, and the stationary phase sigma factor (RpoS) (Davies, D. G. et al. (1998) Science 280:295-298; Latifi, A. et al. (1995) Mol. Microbiol. Rev. 17:333-344; Ochsner, U. A., et al. (1995) PNAS, 92:6424-6428; Pesci, E. C. et al.(1997) Trends in Microbiol. 5(4):132-135; Pesci, E. C. et al. (1997) J. Bacteriol. 179:3127-3132). Expression of the rhl system requires a functional las system, therefore the two systems in combination with RpoS constitute a regulatory cascade (Pesci, E. C. et al.(1997) Trends in Microbiol. 5(4):132-135; Pesci, E. C. et al. (1997) J. Bacteriol. 179:3127-3132, Seed et al. 1995).

The signal in the Las system is 3-oxo-dodecanoyl-HSL (3-oxo-C12-HSL) 2, while the signal used in the Rhl system is butanoyl-HSL (C4-HSL) 3. It has been shown that 3-oxo-C12-HSL increases expression of RhlR, indicating a hierarchy of regulation systems (Pesci, E. C. et al. (1997) Trends Microbiol. 5(4):132-4). The Las signal 3-oxo-C12-HSL is synthesized by LasI along with a small amount of N-(3-oxooctanoyl) HSL and N-(3-oxohexanoyl) HSL, while RhlI makes primarily the signal C4-HSL and a small amount of N-hexanoyl (Pearson, J. P. et al. (1997) J. Bacteriol. 179:5756-5757; Winson, M. K. et al. (1995) PNAS 92:9427-9431).

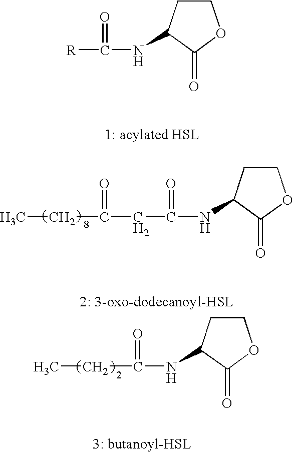

Bacterial signaling triggers the expression of a number of virulence factors in P. aeruginosa including two elastases, an alkaline protease and exotoxin A (Pesci, E. C. et al. (1997) Trends Microbiol. 5(4):132-4; Pesci, E. C. et al. (1997) J Bacteriol. 179(10):3127-32)—proteins that allow the organism to attack host tissue. Bacterial signaling also controls the expression of the antioxidant pyocyanin, a compound that allows the bacteria to neutralize one important host defense, the generation of superoxide radicals (Britigan, et al. (1999) Infect Immun. 67(3):1207-12, Hassan, H. M. et al. (1979) Arch Biochem Biophys. 196(2):385-95, Hassan, H. M. et al. 1980. J Bacteriol. 141(1):156-63). It has been shown in a neonatal mouse model that a defined mutant of P. aeruginosa which lacks the signal receptor protein (LasR) was significantly less virulent than the wild type PAO1, as measured by the ability to cause acute pneumonia, bacteremia and death (Tang, H. B. et al. (1996) Infect Immun. 64(1):37-43).

Biofilms are defined as an association of microorganisms, single or multiple species, that grow attached to a surface and produce a slime layer that provides a protective environment (Costerton, J. W. (1995) J Ind Microbiol. 15(3):137-40, Costerton, J. W. et al (1995) Annu Rev Microbiol. 49:711-45). Typically, biofilms produce large amounts of extracellular polysaccharides, responsible for the slimy appearance, and are characterized by an increased resistance to antibiotics (1000- to 1500-fold less susceptible). Several mechanisms are proposed to explain this biofilm resistance to antimicrobial agents (Costerton, J. W. et al (1999) Science. 284(5418):1318-22). One idea is that the extracellular matrix in which the bacterial cells are embedded provides a barrier toward penetration by the biocides. A further possibility is that a majority of the cells in a biofilm are in a slow-growing, nutrient-starved state, and therefore not as susceptible to the effects of anti-microbial agents. A third mechanism of resistance could be that the cells in a biofilm adopt a distinct and protected biofilm phenotype, e.g., by elevated expression of drug-efflux pumps.

In most natural settings, bacteria grow predominantly in biofilms. Biofilms of P. aeruginosa have been isolated from medical implants, such as indwelling urethral, venous or peritoneal catheters (Stickler, D. J. et al. (1998) Appl Environ Microbiol. 64(9):3486-90). Chronic P. aeruginosa infections in cystic fibrosis lungs are considered to be biofilms (Costerton, J. W. et al. (1999) Science. 284(5418):1318-22).

In industrial settings, the formation of biofilms is often referred to as ‘biofouling’. Biological fouling of surfaces is common and leads to material degradation, product contamination, mechanical blockage, and impedance of heat transfer in water-processing systems. Biofilms are also the primary cause of biological contamination of drinking water distribution systems, due to growth on filtration devices.

As noted earlier, many gram-negative bacteria have been shown to possess one or more quorum sensing systems that regulate a variety of physiological processes, including the activation of virulence genes and biofilm formation. One such gram negative bacterium is Pseudomonas aeruginosa.

P. aeruginosa is a soil and water bacterium that can infect animal hosts. Normally, the host defense system is adequate to prevent infection. However, in immunocompromised individuals (such as burn patients, patients with cystic fibrosis, or patients undergoing immunosuppressive therapy), P. aeruginosa is an opportunistic pathogen, and infection with P. aeruginosa can be fatal (Govan, J. R. et al. (1996) Microbiol Rev. 60(3):539-74; Van Delden, C. et al. (1998) Emerg Infect Dis. 4(4):551-60).

For example, Cystic fibrosis (CF), the most common inherited lethal disorder in Caucasian populations (˜1 out of 2,500 life births), is characterized by bacterial colonization and chronic infections of the lungs. The most prominent bacterium in these infections is P. aeruginosa—by their mid-twenties, over 80% of people with CF have P. aeruginosa in their lungs (Govan, J. R. et al. (1996) Microbiol Rev. 60(3):539-74). Although these infections can be controlled for many years by antibiotics, ultimately they “progress to mucoidy,” meaning that the P. aeruginosa forms a biofilm that is resistant to antibiotic treatment. At this point the prognosis is poor. The median survival age for people with CF is the late 20s, with P. aeruginosa being the leading cause of death (Govan, J. R. et al. (1996) Microbiol Rev. 60(3):539-74). According to the Cystic Fibrosis Foundation, treatment of CF cost more than $900 million in 1995 (Cystic Fibrosis Foundation,).

P. aeruginosa is also one of several opportunistic pathogens that infect people with AIDS, and is the main cause of bacteremia (bacterial infection of the blood) and pneumonitis in these patients (Rolston, K. V. et al. (1990) Cancer Detect Prev. 14(3):377-81; Witt, D. J. et al. (1987) Am J Med. 82(5):900-6). A recent study of 1635 AIDS patients admitted to a French hospital between 1991-1995 documented 41 cases of severe P. aeruginosa infection (Meynard, J. L. et al. (1999) J Infect. 38(3):176-81). Seventeen of these had bacteremia, which was lethal in 8 cases. Similar, numbers were obtained in a smaller study in a New York hospital, where the mortality rate for AIDS patients admitted with P. aeruginosa bacteremia was about 50% (Mendelson, M. H. et al. 1994. Clin Infect Dis. 18(6):886-95).

In addition, about two million Americans suffer serious burns each year, and 10,000-12,000 die from their injuries. The leading cause of death is infection (Lee, J. J. et al. (1990) J Burn Care Rehabil. 11 (6):575-80). P. aeruginosa bacteremia occurs in 10% of seriously burned patients, with a mortality rate of 80% (Mayhall, C. G. (1993) p. 614-664, Prevention and control of nosocomial infections. Williams & Wilkins, Baltimore; McManus, A. T et al. (1985) Eur J Clin microbiol. 4(2):219-23).

Such infections are often acquired in hospitals (“nosocomial infections”) when susceptible patients come into contact with other patients, hospital staff, or equipment. In 1995 there were approximately 2 million incidents of nosocomial infections in the U.S., resulting in 88,000 deaths and an estimated cost of $ 4.5 billion (Weinstein, R. A. (1998) Emerg Infect Dis. 4(3):416-20). Of the AIDS patients mentioned above who died of P. aeruginosa bacteremia, more than half acquired these infections in hospitals (Meynard, J. L. et al. (1999) J Infect. 38(3):176-81).

Nosocomial infections are especially common in patients in intensive care units as these people often have weakened immune systems and are frequently on ventilators and/or catheters. Catheter-associated urinary tract infections are the most common nosocomial infection (Richards, M. J. et al. (1999) Crit Care Med. 27(5):887-92) (31% of the total), and P. aeruginosa is highly associated with biofilm growth and catheter obstruction. While the catheter is in place, these infections are difficult to eliminate (Stickler, D. J. et al. (1998) Appl Environ Microbiol. 64(9):3486-90). The second most frequent nosocomial infection is pneumonia, with P. aeruginosa the cause of infection in 21% of the reported cases (Richards, M. J. et al. (1999) Crit Care Med. 27(5):887-92). The annual costs for diagnosing and treating nosocomial pneumonia has been estimated at greater than $2 billion (Craven, D. E. et al. (1991) Am J Med. 91(3B):44S-53S).

Treatment of these so-called nosocomial infections is complicated by the fact that bacteria encountered in hospital settings are often resistant to many antibiotics. In June 1998, the National Nosocomial Infections Surveillance (NNIS) System reported increases in resistance of P. aeruginosa isolates from intensive care units of 89% for quinolone resistance and 32% for imipenem resistance compared to the years 1993-1997 (Centers for Disease Control and Prevention). In fact, some strains of P. aeruginosa are resistant to over 100 antibiotics (Levy, S. (1998) Scientific American. March). There is a critical need to overcome the emergence of bacterial strains that are resistant to conventional antibiotics (Travis, J. (1994) Science. 264:360-362).

P. aeruginosa is also of great industrial concern (Bitton, G. (1994) Wastewater Microbiology. Wiley-Liss, New York, N.Y.; Steelhammer, J. C. et al. (1995) Indust. Water Treatm. :49-55). The organism grows in an aggregated state, the biofilm, which causes problems in many water processing plants. Of particular public health concern are food processing and water purification plants. Problems include corroded pipes, loss of efficiency in heat exchangers and cooling towers, plugged water injection jets leading to increased hydraulic pressure, and biological contamination of drinking water distribution systems (Bitton, G. (1994) Wastewater Microbiology. Wiley-Liss, New York, N.Y., 9). The elimination of biofilms in industrial equipment has so far been the province of biocides. Biocides, in contrast to antibiotics, are antimicrobials that do not possess high specificity for bacteria, so they are often toxic to humans as well. Biocide sales in the US run at about $ 1 billion per year (Peaff, G. (1994) Chem. Eng. Mews:15-23).

A particularly ironic connection between industrial water contamination and public health issues is an outbreak of P. aeruginosa peritonitis that was traced back to contaminated poloxamer-iodine solution, a disinfectant used to treat the peritoneal catheters. P. aeruginosa is commonly found to contaminate distribution pipes and water filters used in plants that manufacture iodine solutions. Once the organism has matured into a biofilm, it becomes protected against the biocidal activity of the iodophor solution. Hence, a common soil organism that is harmless to the healthy population, but causes mechanical problems in industrial settings, ultimately contaminated antibacterial solutions that were used to treat the very people most susceptible to infection.

Regulation of virulence genes by quorum sensing is well documented in P. aeruginosa. Recently, genes not directly involved in virulence including the stationary phase sigma factor rpoS and genes coding for components of the general secretory pathway (xcp) (Jamin, M. et al. (1991) Biochem J. 280(Pt 2):499-506) have been reported to be positively regulated by quorum sensing. Furthermore, the las quorum sensing system is required for maturation of P. aeruginosa biofilms (Chapon-Herve, V. et al. (1997) Mol. Microbiol. 24, 1169-1170; Davies, D. G., et al. (1998) Science 280, 295-298). Thus it seems clear that quorum sensing represents a global gene regulation system in P. aeruginosa. However, the number and types of genes controlled by quorum sensing have not been identified or studied extensively.

### SUMMARY OF THE INVENTION

In general, the invention pertains to the modulation of bacterial cell-to-cell signaling. The inhibition of quorum sensing signaling renders a bacterial population more susceptible to treatment, either directly through the host immune-response or in combination with traditional antibacterial agents and biocides. More particularly, the invention also pertains to a method for identifying modulators, e.g., inhibitors of cell-to-cell signaling in bacteria, and in particular one particular human pathogen, Pseudomonas aeruginosa.

Thus, in one aspect, the invention is a method for identifying a modulator of quorum sensing signaling in bacteria. The method comprises:

providing a cell comprising a quorum sensing controlled gene, wherein the cell is responsive to a quorum sensing signal molecule such that a detectable signal is generated;

contacting said cell with a quorum sensing signal molecule in the presence and absence of a test compound;

and detecting a change in the detectable signal to thereby identify the test compound as a modulator of quorum sensing signaling in bacteria.

In one embodiment the cell comprises a reporter gene operatively linked to a regulatory sequence of a quorum sensing controlled gene, such that the quorum sensing signal molecule modulates the transcription of the reporter gene, thereby providing a detectable signal.

Another aspect of the invention is a method for identifying a modulator of a quorum sensing signaling in Pseudomonas aeruginosa. The method comprises:

providing a wild type strain of Pseudomonas aeruginosa which produces a quorum sensing signal molecule;

providing a mutant strain of Pseudomonas aeruginosa which comprises a reporter gene operatively linked to a regulatory sequence of a quorum sensing controlled gene, wherein the mutant strain is responsive to the quorum sensing signal molecule produced by the wild type strain, such that a detectable signal is generated;

contacting the mutant strain with the quorum sensing signal molecule and a test compound; and

detecting a change in the detectable signal to thereby identify the test compound as a modulator of quorum sensing signaling in Pseudomonas aeruginosa.

In one embodiment, the endogenous lasI and rhlI quorum sensing systems are inactivated in the mutant strain of Pseudomonas aeruginosa. In another embodiment the mutant strain of Pseudomonas aeruginosa comprises a promoterless reporter gene inserted at a genetic locus in the chromosome, wherein the genetic locus comprises a nucleotide sequence selected from the group consisting of.

A further aspect of the invention is a mutant strain of Pseudomonas aeruginosa comprising a promoterless reporter gene inserted at a genetic locus in the chromosome, wherein the genetic locus comprises a nucleotide sequence selected from the group consisting of.

In one embodiment, the endogenous lasI and rhlI quorum sensing systems are inactivated in the mutant strain of Pseudomonas aeruginosa. In another embodiment the mutant strain of Pseudomonas aeruginosa is responsive to a quorum sensing signal molecule such that a detectable signal is generated by the reporter gene. In yet another embodiment, the reporter gene is contained in a transposable element.

Yet another aspect of the invention is a method for identifying a modulator of quorum sensing signaling in Pseudomonas aeruginosa. The method comprises:

providing a wild type strain of Pseudomonas aeruginosa which produces a quorum sensing signal molecule;

providing a mutant strain of Pseudomonas aeruginosa which comprises a promoterless reporter gene inserted at a genetic locus in the chromosome of said Pseudomonas aeruginosa, wherein the genetic locus comprises a nucleotide sequence selected from the group consisting of SEQ ID NOs:1-353; and wherein the mutant strain is responsive to the quorum sensing signal molecule produced by the wild type strain, such that a detectable signal is generated by the reporter gene;

contacting the mutant strain with the quorum sensing signal molecule and a test compound; and

detecting a change in the detectable signal to thereby identify the test compound as a modulator of quorum sensing signaling in Pseudomonas aeruginosa.

Another aspect of the invention is an isolated nucleic acid molecule comprising a nucleotide sequence which comprises:

a regulatory sequence derived from the genome of Pseudomonas aeruginosa, wherein the regulatory sequence regulates a quorum sensing controlled genetic locus of the Pseudomonas aeruginosa chromosome, and wherein the genetic locus comprises a nucleotide sequence selected from the group consisting of SEQ ID NOs:1-353; and

a reporter gene operatively linked to the regulatory sequence.

A further aspect of the invention provides an isolated nucleic acid molecule comprising a quorum sensing controlled genetic locus derived from the genome of Pseudomonas aeruginosa, wherein the genetic locus comprises a nucleotide sequence selected from the group consisting of SEQ ID NOs:1-353, operatively linked to a reporter gene.

In one embodiment, the invention is an isolated nucleic acid molecule comprising a polynucleotide having at least 80% identity to a quorum sensing controlled genetic locus derived from the genome of Pseudomonas aeruginosa, wherein the genetic locus comprises a nucleotide sequence selected from the group consisting of SEQ ID NOs:1-353, operatively linked to a reporter gene.

In another embodiment, the invention is an isolated nucleic acid molecule comprising a polynucleotide that hybridizes under stringent conditions to a quorum sensing controlled genetic locus derived from the genome of Pseudomonas aeruginosa, wherein the genetic locus comprises a nucleotide sequence selected from the group consisting of SEQ ID NOs:1-353, operatively linked to a reporter gene.

In one embodiment, an isolated nucleic acid molecule of the invention comprises a reporter gene contained in a transposable element.

Accordingly, a further aspect of the invention pertains to a vector comprising an isolated nucleic acid molecule of the invention. In another aspect, the invention provides cells containing an isolated nucleic acid molecule of the invention.

An additional aspect of the invention is a method for identifying a modulator of quorum sensing signaling in bacteria. The method comprises:

providing a cell containing an isolated nucleic acid molecule of the invention, wherein the cell is responsive to a quorum sensing signal molecule such that a detectable signal is generated;

contacting said cell with a quorum sensing signal molecule in the presence and absence of a test compound;

and detecting a change in the detectable signal to thereby identify the test compound as a modulator of quorum sensing signaling in bacteria.

Accordingly, in another aspect, the invention provides a compound identified by a method of the invention which modulates, e.g., inhibits, quorum sensing signaling in Pseudomonas aeruginosa. In one embodiment, the compound inhibits quorum sensing signaling in Pseudomonas aeruginosa by inhibiting an enzyme involved in the synthesis of a quorum sensing signal molecule, by interfering with quorum sensing signal reception, or by scavenging the quorum sensing signal molecule.

The invention also pertains to a method for identifying quorum sensing controlled genes in a cell, and specifically in one particular human pathogen, Pseudomonas aeruginosa. Thus, in one aspect, the invention provides a method for identifying a quorum sensing controlled gene in a cell, the method comprising:

providing a cell which is responsive to a quorum sensing signal molecule such that expression of a quorum sensing controlled gene is modulated, and wherein modulation of the expression of said quorum sensing controlled gene generates a detectable signal;

contacting said cell with a quorum sensing signal molecule;

and detecting a change in the detectable signal to thereby identify a quorum sensing signaling controlled gene.

In one embodiment the cell comprises a reporter gene operatively linked to a quorum sensing controlled gene or a regulatory sequence of a quorum sensing controlled gene, such that modulation of the expression of the quorum sensing controlled gene modulates the transcription of the reporter gene, thereby providing a detectable signal. In another embodiment the reporter gene is contained in a transposable element. In yet another embodiment, the quorum sensing signal molecule is produced by a second cell, e.g., a bacterial cell. In a further embodiment, the quorum sensing signal molecule is an autoinducer of said quorum sensing controlled gene, e.g., a homoserine lactone, or an analog thereof.

### BRIEF DESCRIPTION OF THE DRAWINGS

FIG. 1 depicts the paragdigm for quorum sensing signaling in the target bacterium, Pseudomonas aeruginosa.

FIG. 2 depicts patterns of β-galactosidase expression in representative qsc mutants and in a strain with a lasB::lacZ chromosomal fusion generated by site-specific mutation. Units of β-galactosidase are given as a function of culture density for cells grown without added signal molecules (∘), with added 3OC₁₂-HSL (●), with added C₄-HSL (▪), or with both signals added (□).

FIG. 3 depicts the nucleic acid sequence of the quorum sensing controlled locus on the P. aeruginosa chromosome mapped in the P. aeruginosa mutant strain qsc 102.

FIG. 4 depicts putative qsc operons. Open reading frames (ORFs) are indicated by the arrows. ORFs discovered in the qsc screen are indicated by their qsc number.

FIG. 5 depicts a growth curve of PAO1/pMW303G. Culture growth is monitored at 600 nm (closed circles) and β-galactosidase activity is measured with a chemiluminescent substrate analog in relative light units (RLU; open circles).

FIG. 6 is a map of the qsc insertions on the P. aeruginosa chromosome. Arrowheads indicate the direction of lacZ transcription. In addition to the qsc mutants, lasR and lasI, rhlR, and lasB are also mapped. The locations of las-boxes like elements are shown as black dots between the two DNA strands. The numbers indicate distance in megabases on the approximately 6 megabase chromosome.

FIG. 7 depicts putative las-type boxes in upstream DNA regions of qsc mutants. ORFs as described in Materials and Methods. Bases outlined in black represent residues conserved in all sequences and gray outlines are conserved in 8 of 10 sequences.

FIG. 8 depicts the principle of a bioassay for modulators of quorum sensing signaling. Strain PAO1 produces the signal 3-oxo-C12-HSL. Strain QSC102 responds by inducing lacZ.

FIG. 9 depicts the results of an assay performed using the test compound acetyl-butyrolactone, which is present in the wells at increasing concentration (mM, as indicated). There are two rows and two columns per concentration to show reproducibily of the assay.

FIG. 10A depicts the structure of a mobilizable plasmid for generating an indicator strain. Filled boxes represent chromosomal DNA derived from the P. aeruginosa locus where lacZ is inserted in strain QSC102.

FIG. 10B depicts induction of β-galactosidase as PAQ1 reaches high density. Cell growth is monitored at 600 nm (closed circles) and expression of β-galactosidase is measured in Miller units (open circles).

FIG. 11 depicts the reaction mechanism of the RhlI autoinducer synthase.

FIG. 12 depicts a continuous culture bioreactor.

FIG. 13 is a graph depicting growth of wild-type P. aeruginosa PAO1 (open squares), the receptor mutant PAO lasR rhlR (open triangles), and the signal generation mutant PAO-MW1 without added acyl-HSL (filled triangles), with 3OC12-HSL (open circles), and with C4-HSL and 3OC12-HSL (filled squares).

FIG. 14 depicts predicted quorum-regulated operons. Genes not listed in Tables 5 and 6 are depicted as black boxes. Arrows indicate direction of transcription. Black and white circles indicate putative las-rhl boxes with Heterology Index (HI) scores below 10 and below 13, respectively. Top, quorum-activated operons, and bottom, quorum-repressed operons.

FIG. 15 contains the nucleotide sequences corresponding to SEQ ID NOs:1-353. Each nucleotide sequence corresponds to a SEQ ID NO and a “PA” identification number. The nucleotide sequences as well as the corresponding polypeptide sequences can be accessed using the “PA” identification numbers via the Pseudomonas Genome Project (available on the internet at the Pseudomonas Genome Project website). The PA Identification numbers for each nucleotide and polypeptide sequence are also listed in Tables 5 and 6.

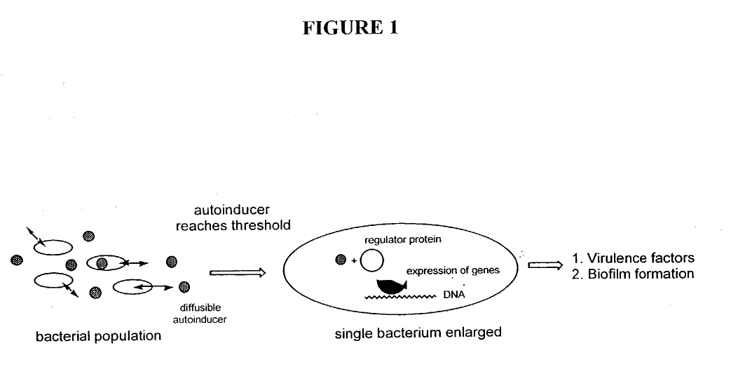

### DETAILED DESCRIPTION OF THE INVENTION

The instant invention is based, at least in part, on the identification of quorum sensing controlled genes (e.g., SEQ ID NOs:1-353) and polypeptides encoded by these quorum sensing controlled genes, referred to herein as quorum sensing controlled polypeptides (e.g., SEQ ID NOs:354-706) in bacteria, e.g., Pseudomonas aeruginosa. Furthermore, the invention is based on the discovery of new methods for the interruption of bacterial cell-to-cell signaling, i.e., quorum sensing signaling, in order to render a bacterial population more susceptible to treatment, either through the host immune-response or in combination with traditional antibacterial agents and biocides. Thus, the invention provides a bacterial indicator strain that allows for a high throughput screening assay for identifying compounds that modulate, e.g., inhibit bacterial cell-to-cell signaling. The compounds so identified will provide novel anti-pathogenics and anti-fouling agents. Accordingly, the invention also provides methods for identifying a compound capable of modulating biofilm formation. The present invention further provides methods for the identification and therapeutic use of compounds, e.g., modulators of biofilm formation, as treatments of biofilm-associated diseases or disorders. The present invention still further provides methods for modulating, e.g., inhibiting or preventing, biofilm formation, e.g., in a subject, and methods for modulating, e.g., inhibiting or preventing, biofouling.

In gram-negative bacteria, such as Pseudomonas aeruginosa, quorum sensing involves two proteins, the autoinducer synthase—the I protein—and the transcriptional activator or receptor protein—the “R protein.” The synthase produces an acylated homoserine lactone which can diffuse into the surrounding environment (Fuqua, C. et al (1998) Curr Opin Microbiol. 1(2):183-189; Fuqua, et al. 1994. J Bacteriol. 176(2):269-75). The autoinducer molecule is composed of an acyl chain in a peptide bond with the amino nitrogen of a homoserine lactone (HSL). For different quorum sensing systems, the side-chain may vary in length, degree of saturation, and oxidation state. As the density of bacteria increases, so does the concentration of this freely diffusible signal molecule. The signal molecule binds to the R-protein, which then activates transcription of numerous genes. Of particular interest are genes involved in pathogenicity and in biofilm formation, referred to herein as quorum sensing controlled genes. Once the concentration of the signal molecule reaches a defined threshold, it binds to the R-protein, which then activates transcription of numerous genes. It has been discovered that the trigger for activation or repression quorum sensing controlled genes is not signal accumulation alone. Rather, receptor levels (e.g., LasR and RhlR levels) may govern the onset of induction of quorum sensing controlled gene activation or repression of some quorum sensing controlled genes. Therefore, LasR and RhlR may control the precise timing of quorum-controlled gene transcription (e.g., transcription of SEQ ID NOs:1-353). However, activation or repression of any of the identified quorum sensing controlled genes requires sufficient signal. Signal can accumulate only when a critical population density has been reached. Therefore, although additional criteria must be met for transcriptional activation or repression of many genes, a quorum is nevertheless required.

Furthermore, it has been discovered that signal specificities of responses of quorum sensing controlled genes to 3OC12-HSL versus the signal specificities of responses of quorum sensing controlled genes to 3OC12-HSL and C4-HSL together showed great variability. Some of the quorum sensing controlled genes identified in Table 1 responded specifically to 3OC12-HSL, while others responded to 3OC12-HSL, but activation was boosted by addition of C4-HSL; still other quorum sensing controlled genes seemed to respond to C4-HSL, showing no response to 3OC12-HSL alone. Tables 5 and 6 illustrate maximum induction for each gene in the presence of 3OC12-HSL alone and in the presence of C4-HSL and 3OC12-HSL together. This data suggests that there is a continuum of specificity responses.

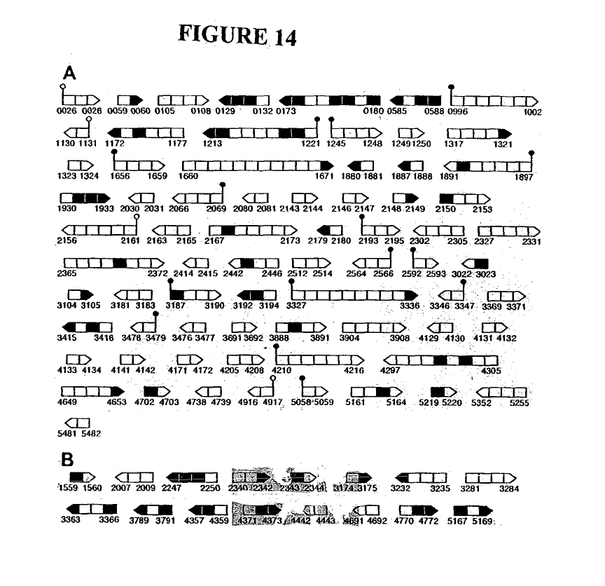

It has also been discovered that some of the quorum sensing genes identified herein contain regulatory sequences, e.g., las-rhl box sequences and/or are controlled by las-rhl box sequences as part of an operon containing a las-rhl box sequence. By using stringent criteria (a heterology index (HI) score of <10), 55 of all P. aeruginosa genes contain a box in their upstream regulatory region. Twenty-five (45%) of these genes are quorum controlled, and 15 represent the first gene in a predicted operon 185 of the P. aeriginosa genes contain a las-rhl box sequence in the upstream regulatory region. Of these, 48 (26%) are quorum controlled, and 19 represent the first gene in a predicted operon. The genes or operons containing a las-rhl box sequence are identified in FIG. 14 and in Tables 5 and 6. The quorum sensing controlled genes which contain las-rhl box sequences and/or are controlled by las-rhl box sequences are directly controlled by quorum sensing while those which do not contain a las-rhl box sequence or are not controlled by las-rhl box sequences may be indirectly controlled by quorum sensing. Accordinlgy, the genes which contain las-rhl box sequences and/or are controlled by las-rhl box sequences represent ideal targets for development of modulators of quorum sensing controlled genes. Accordingly, the invention also includes methods for identifying a modulator of quorum sensing signaling in bacteria, comprising providing a cell which comprises a quorum sensing controlled gene which contains a las-rhl box sequence and/or is controlled by a las-rhl box sequence, where the cell is responsive to a quorum sensing signal molecule such that a detectable signal is generated; contacting the cell with a quorum sensing signal molecule in the presence and absence of a test compound; and detecting a change in the detectable signal to thereby identify said test compound as a modulator of quorum sensing signaling in bacteria.

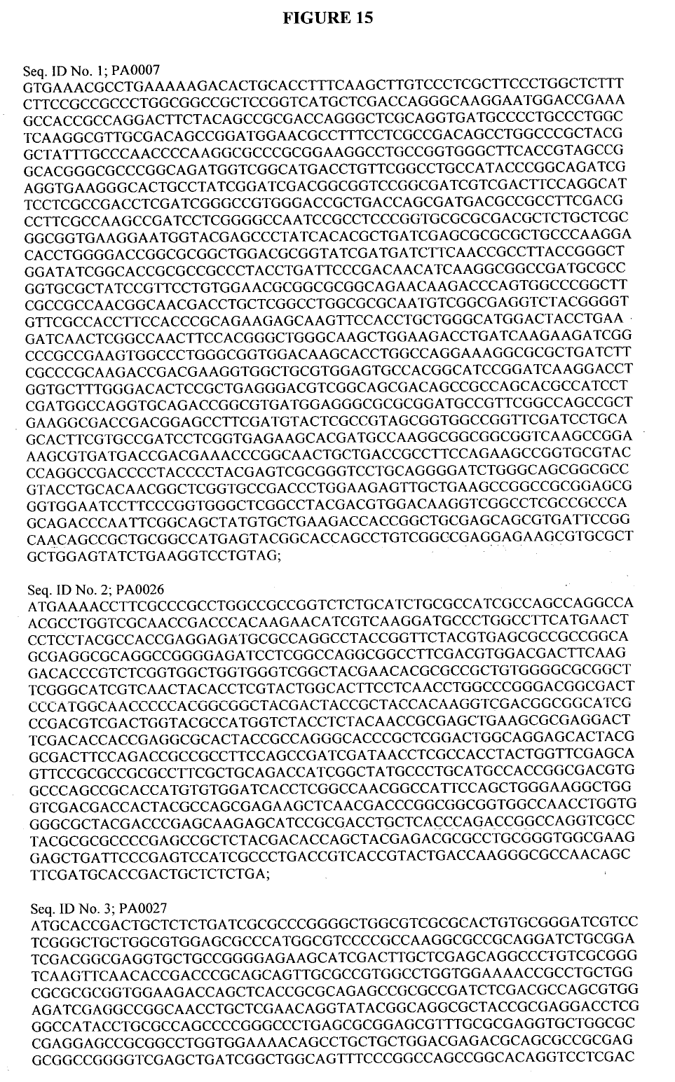

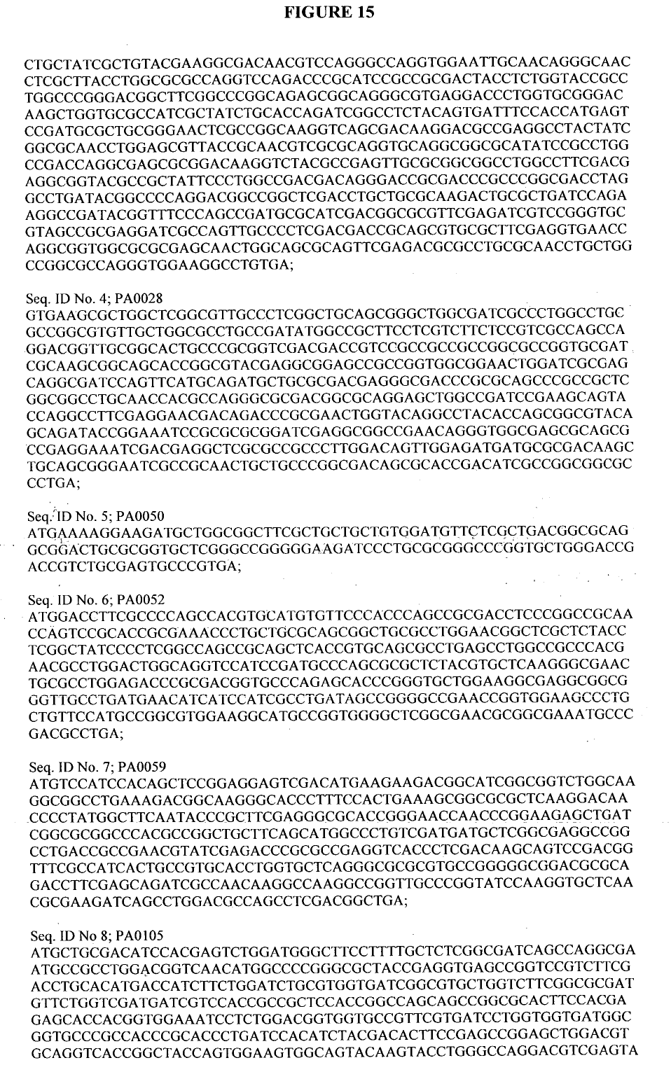

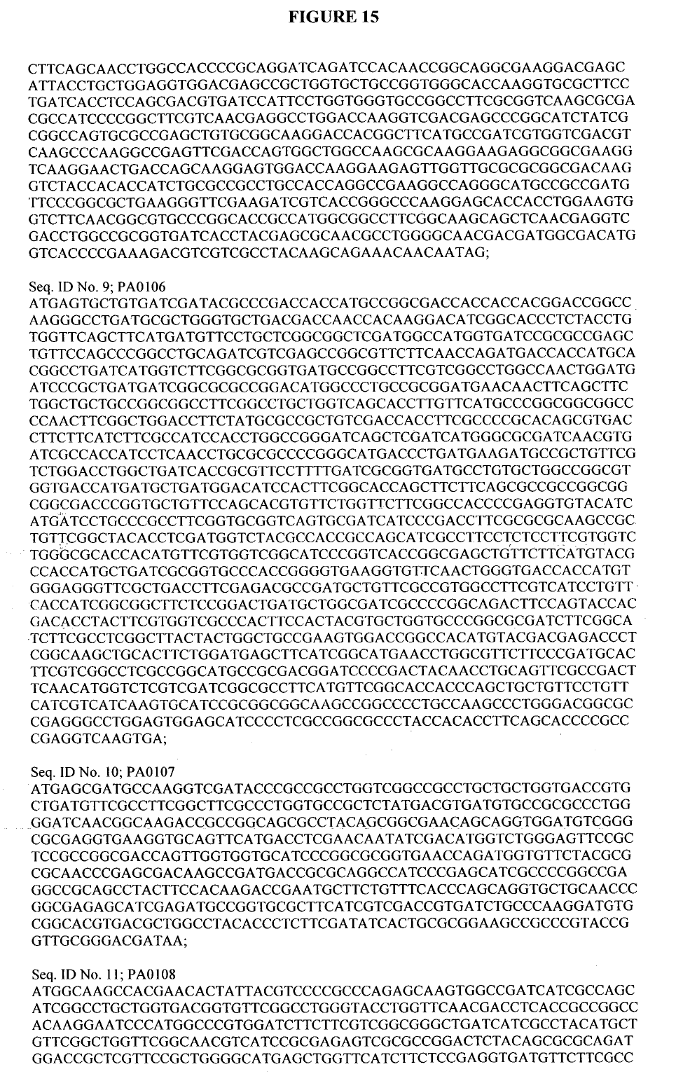

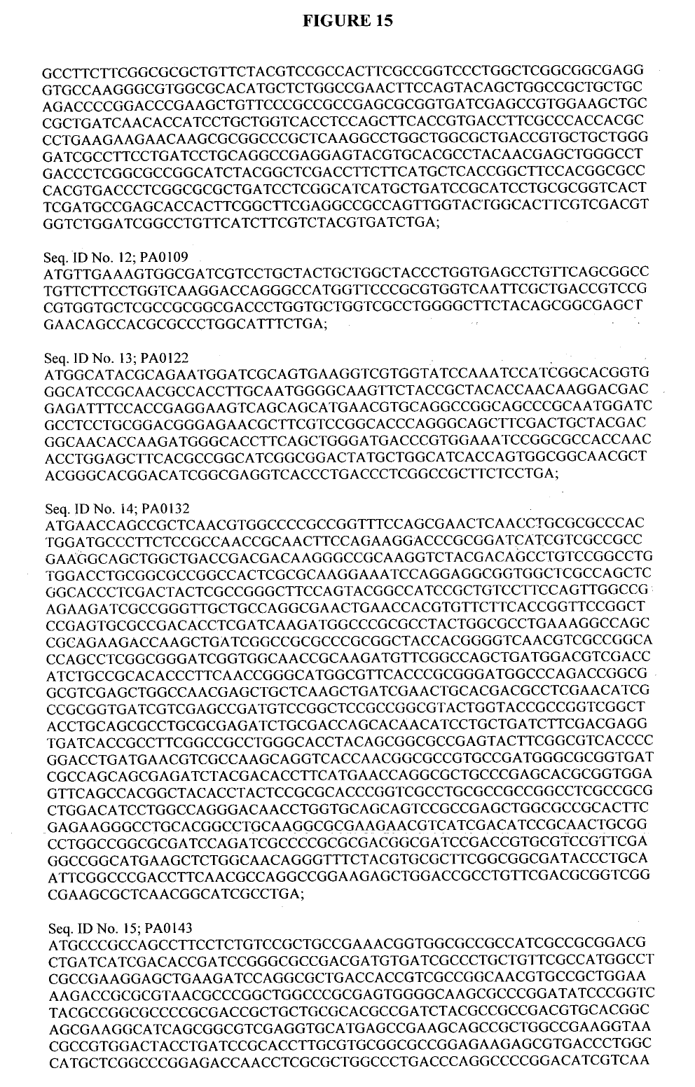

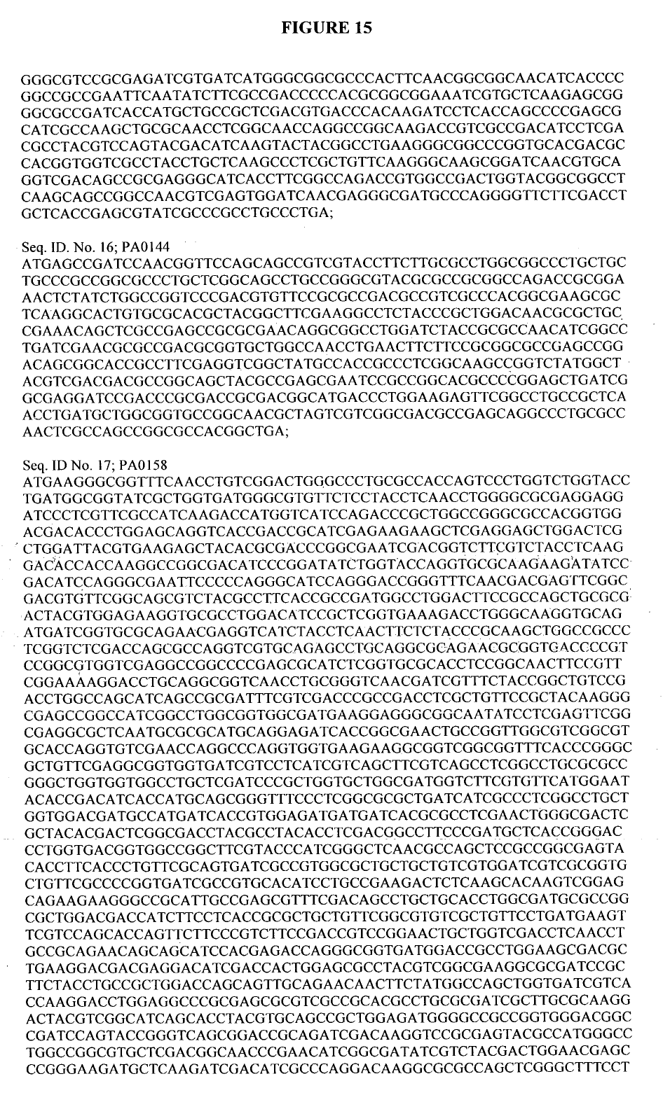

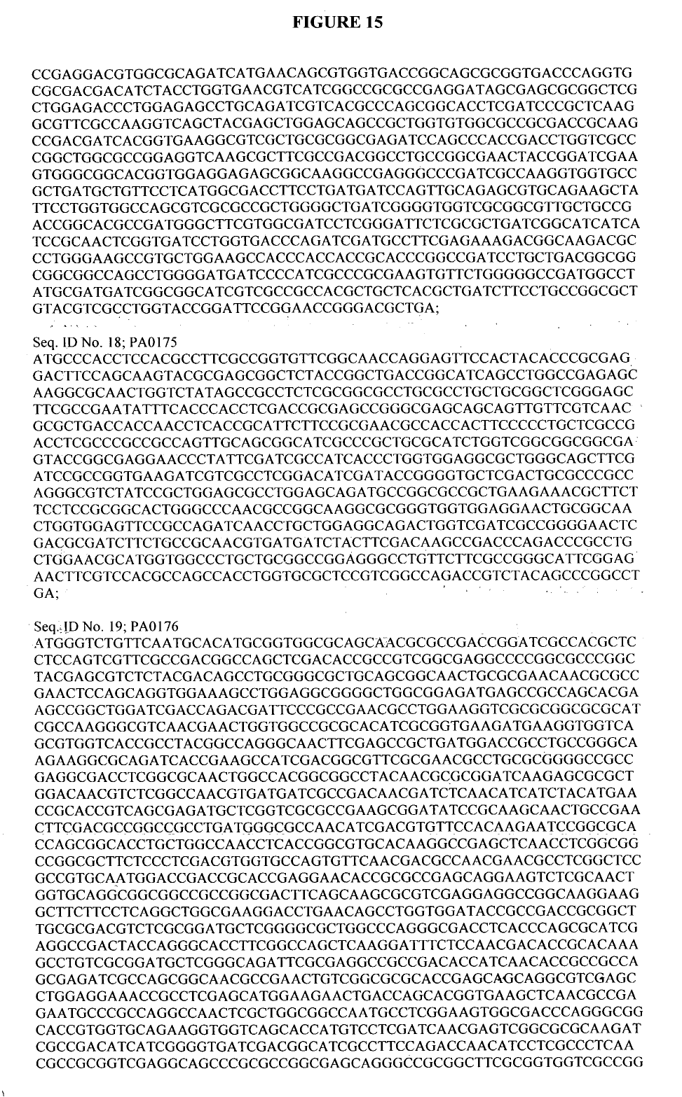

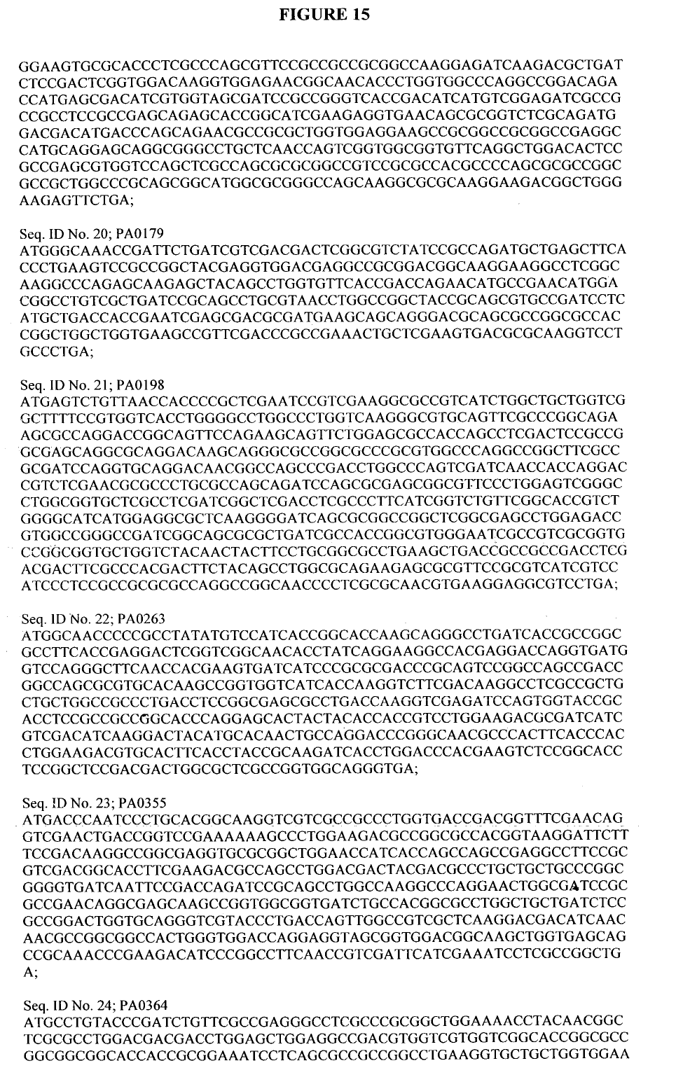

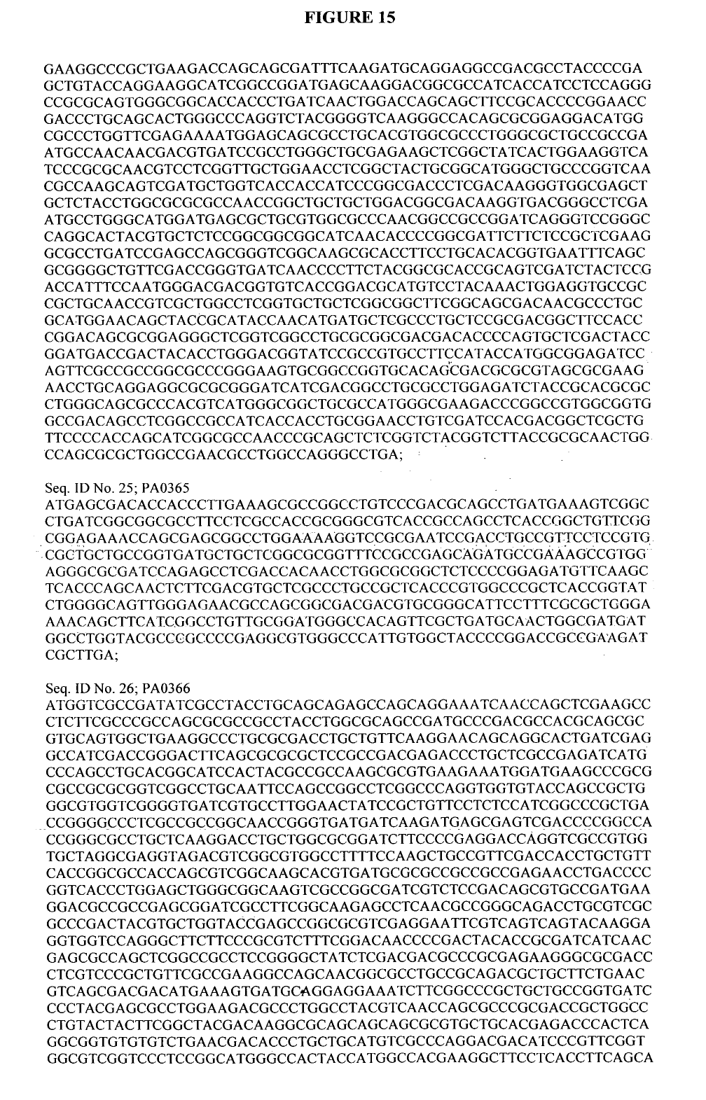

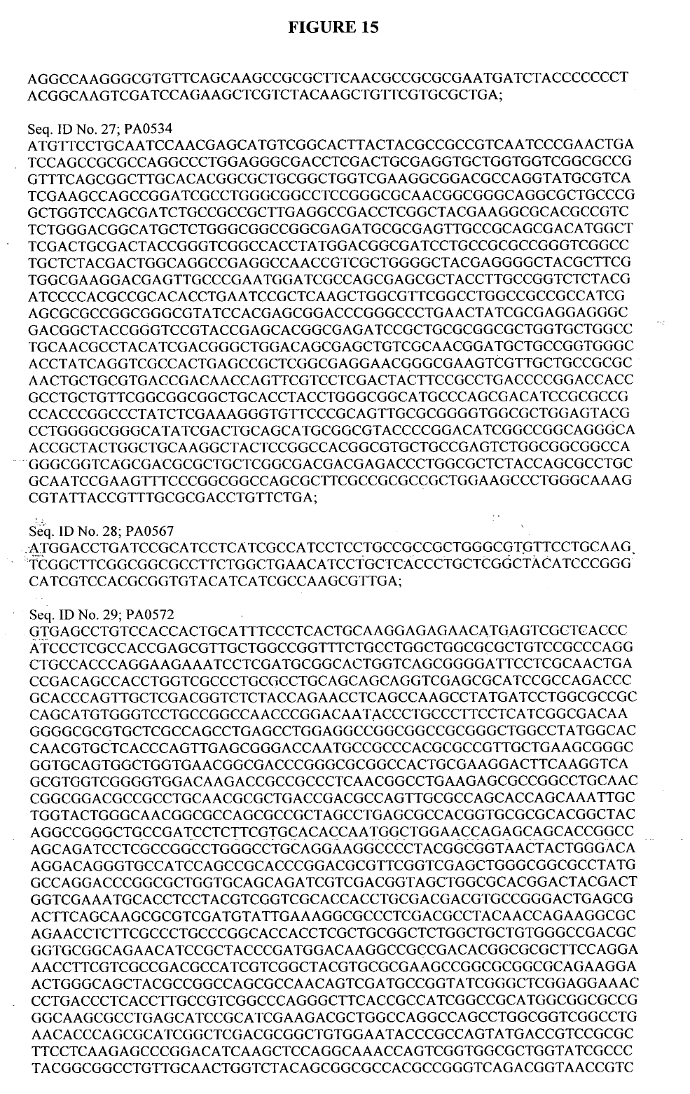

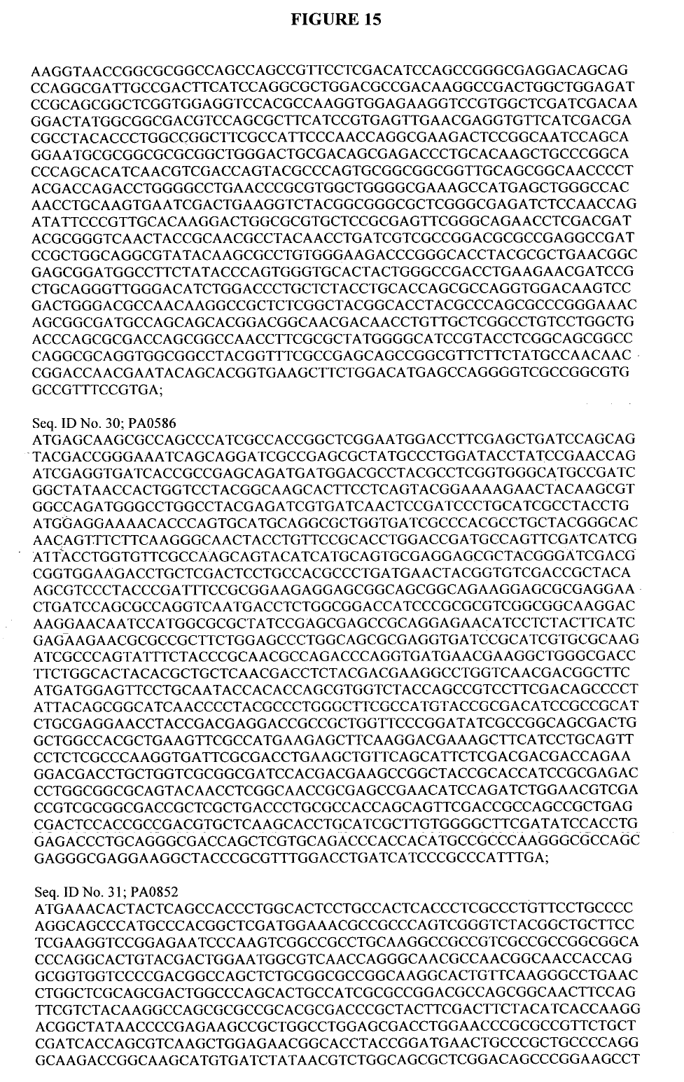

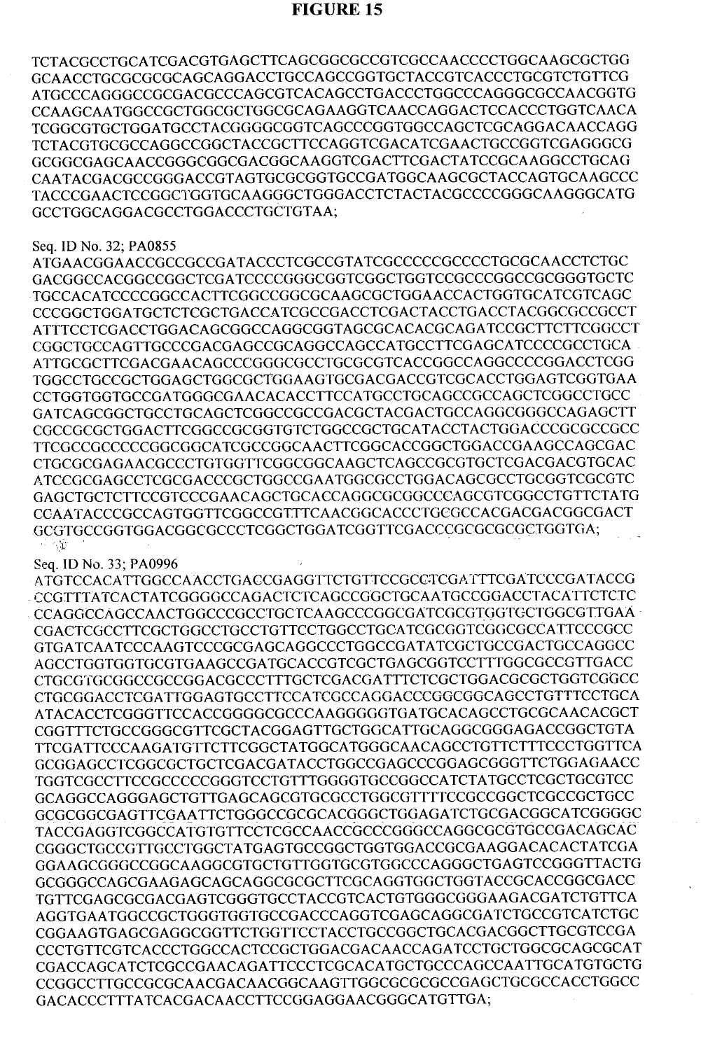

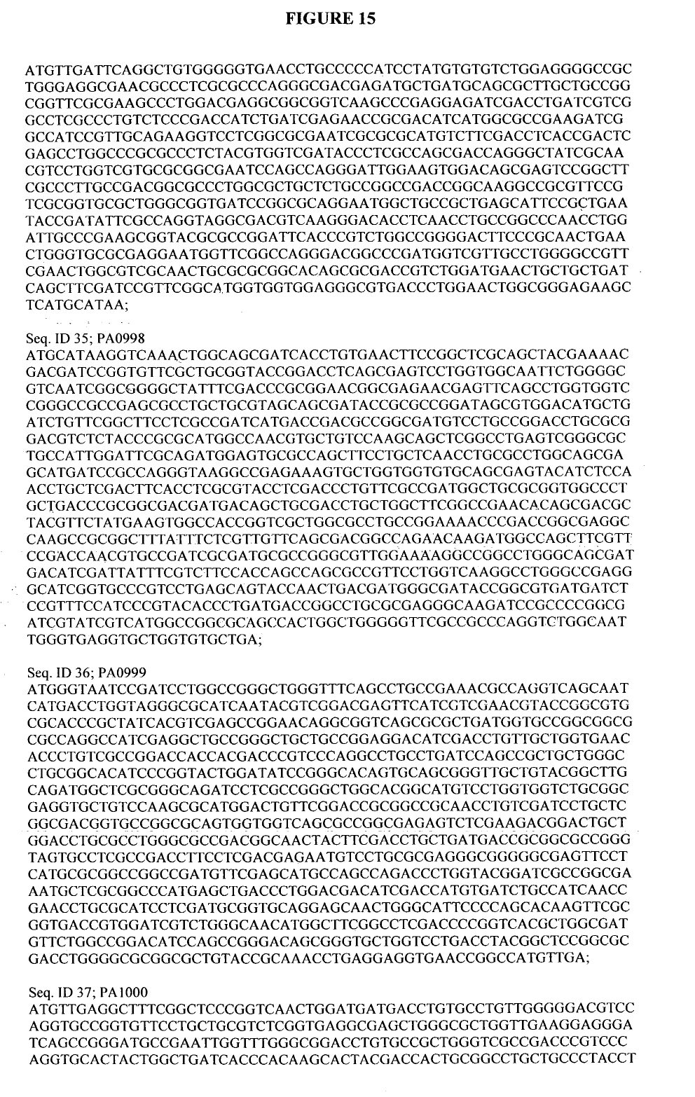

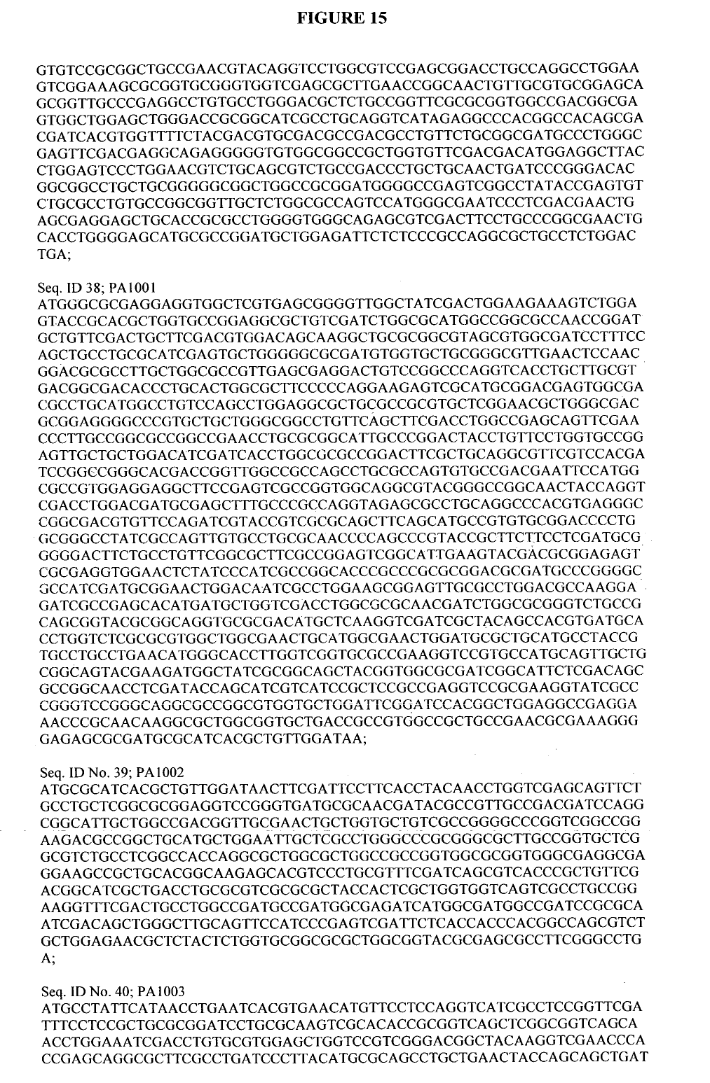

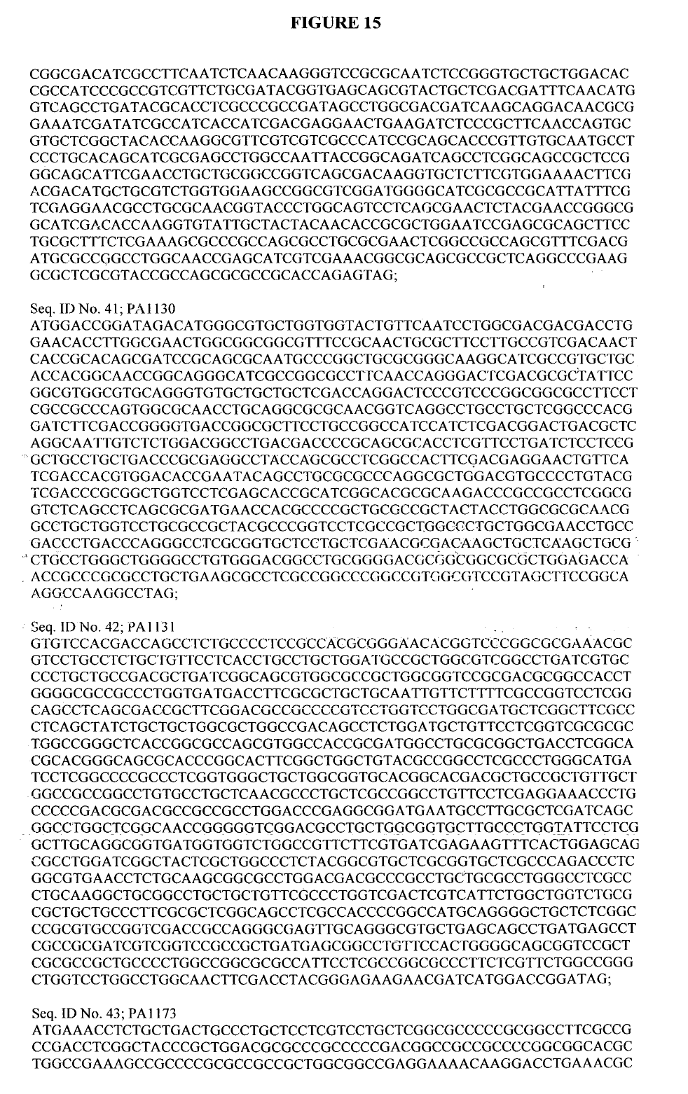

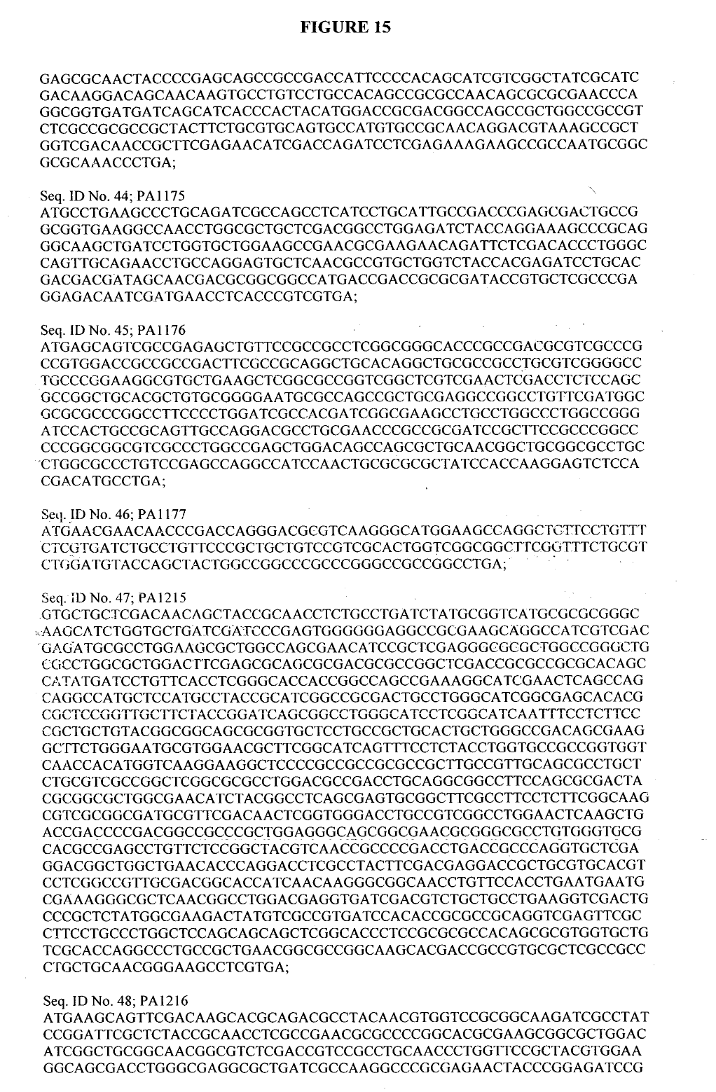

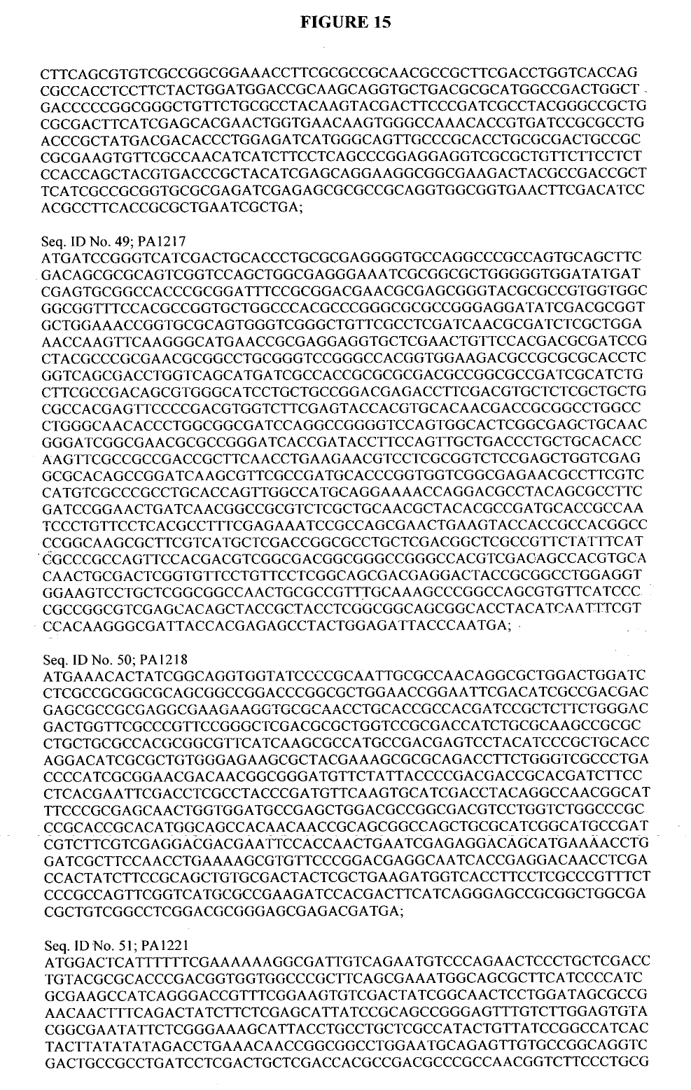

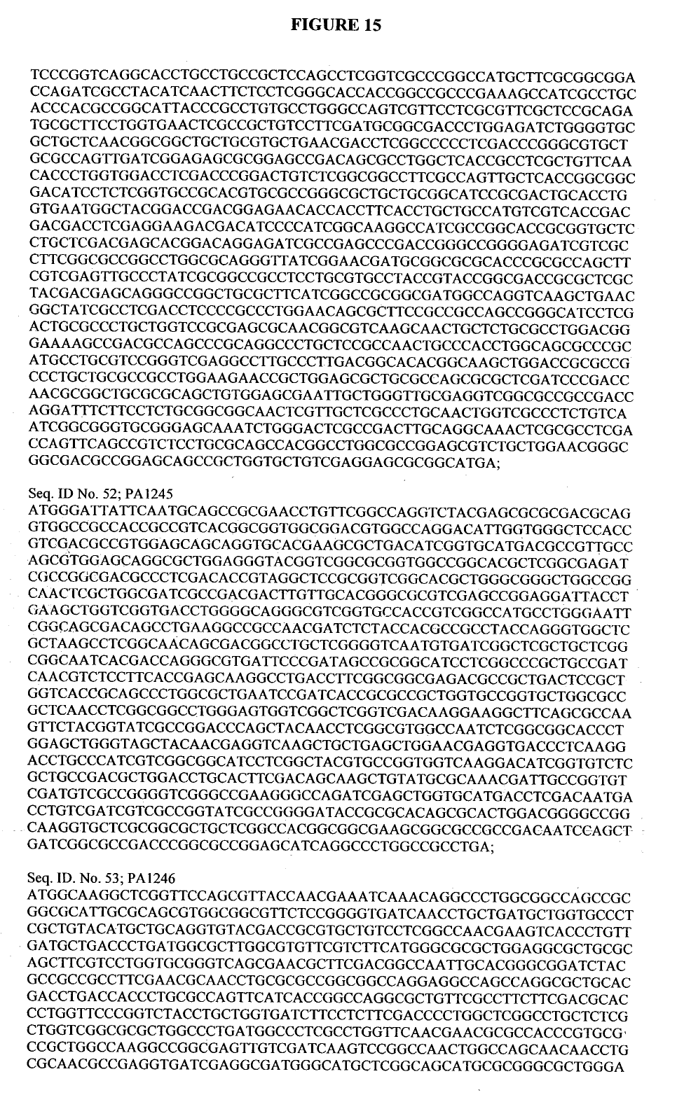

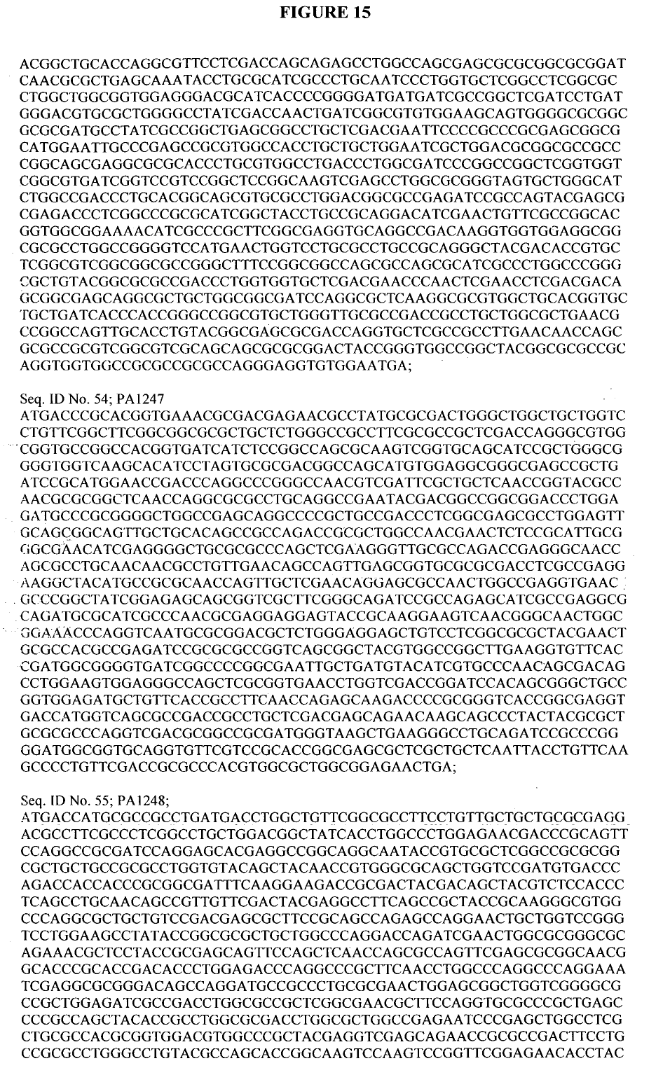

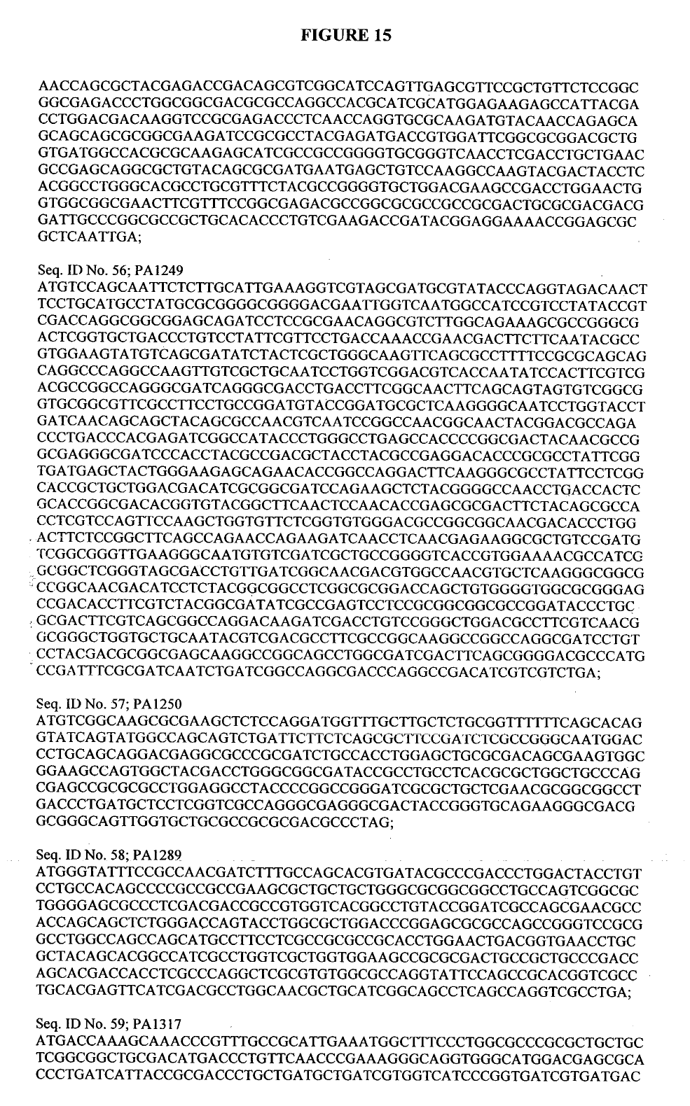

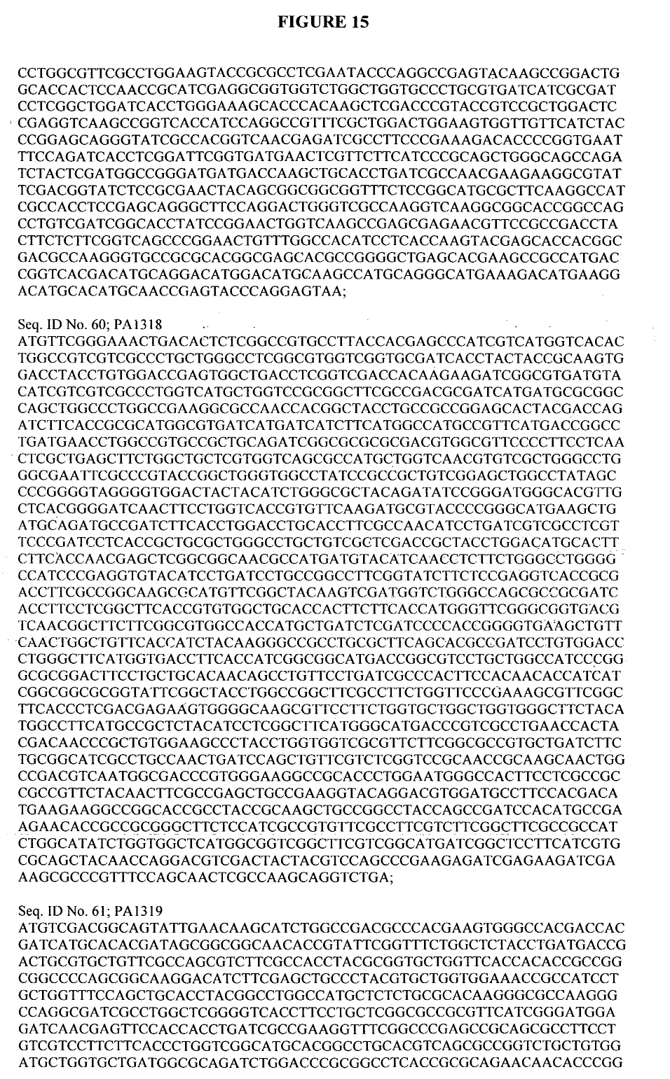

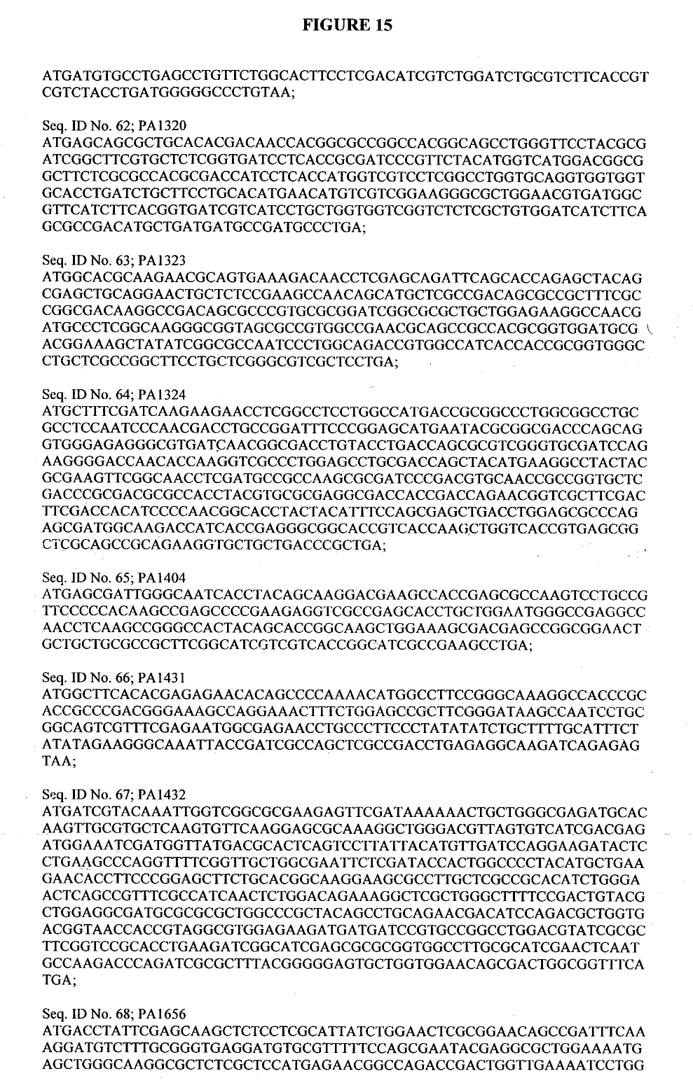

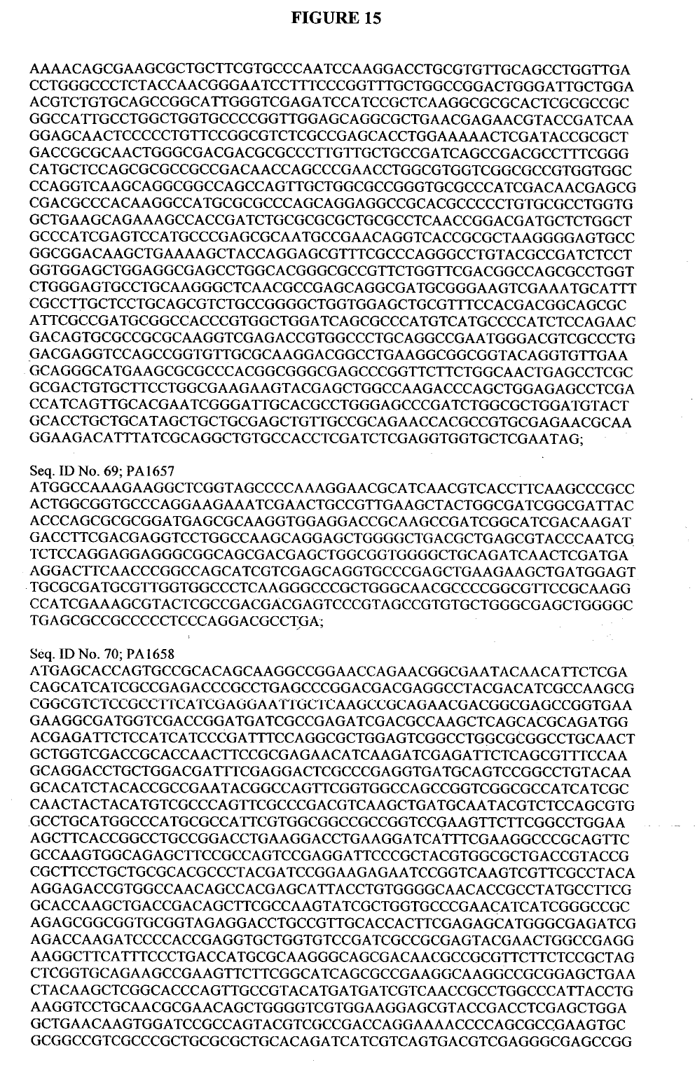

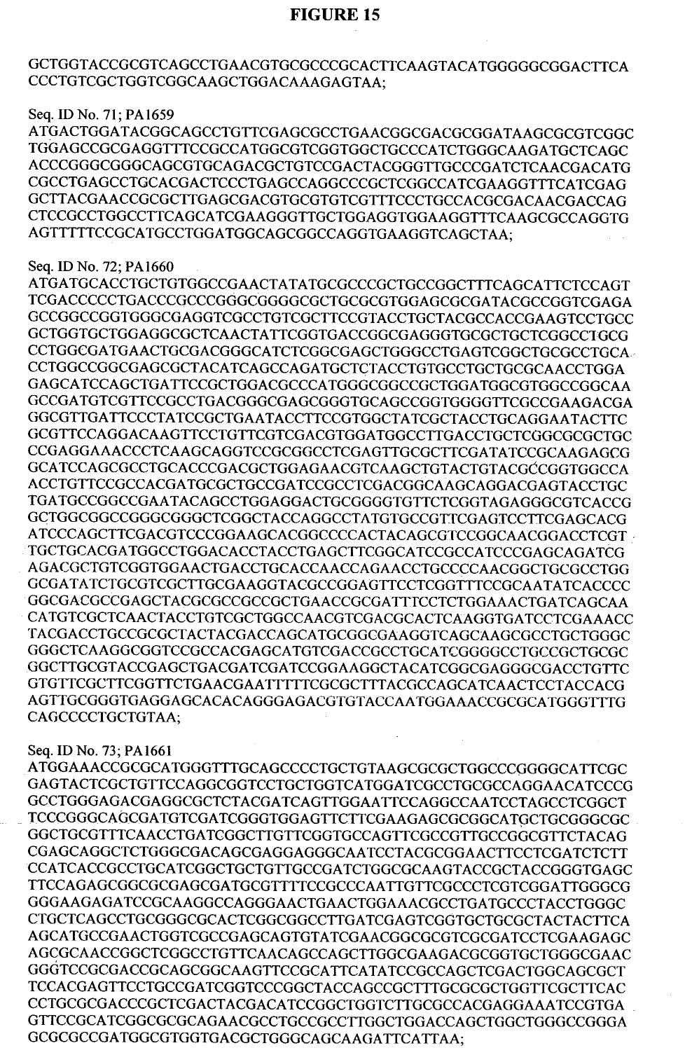

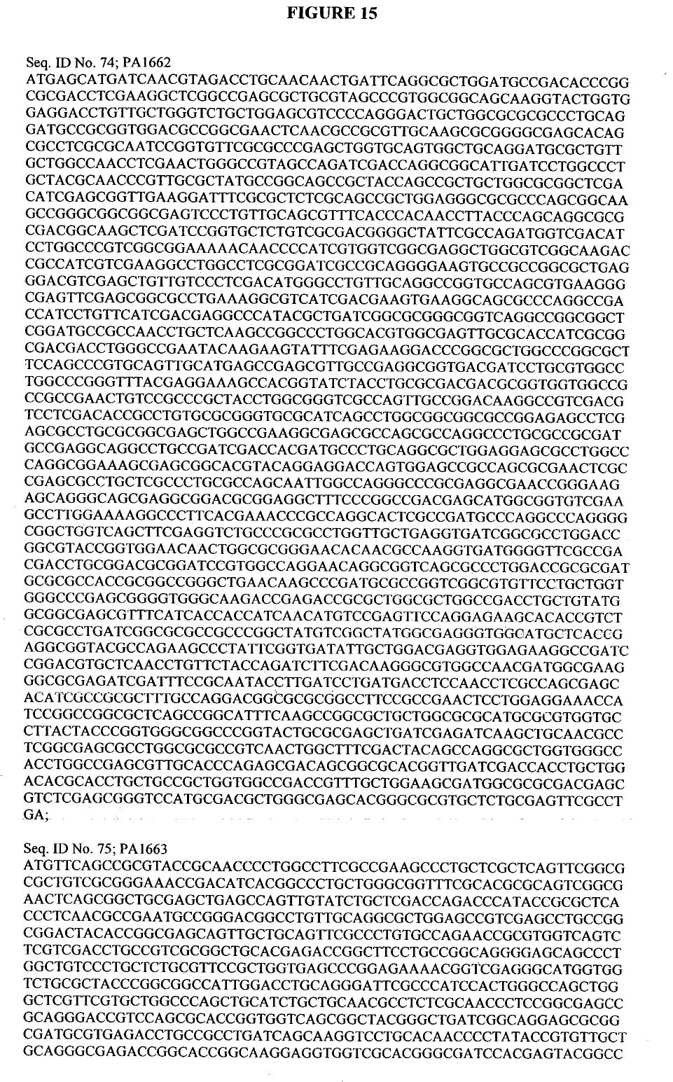

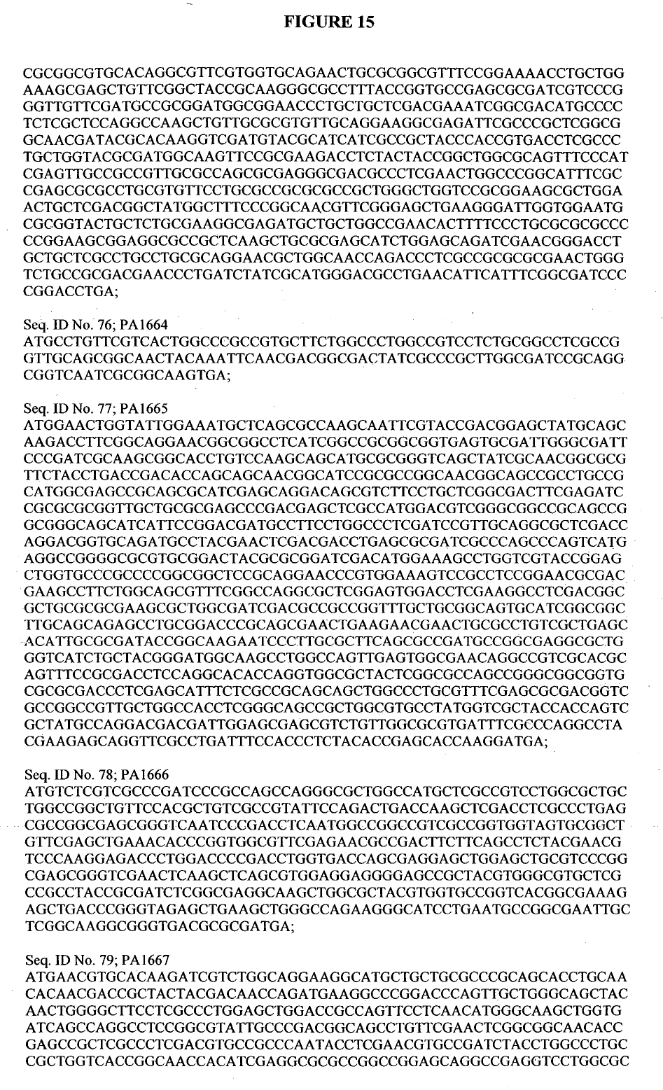

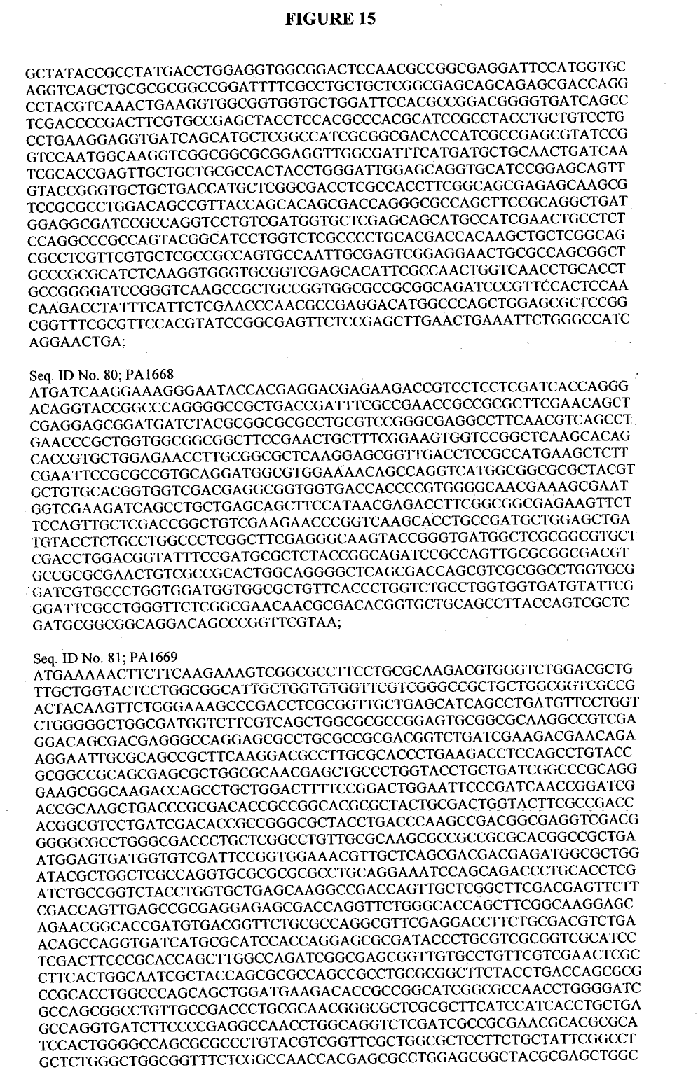

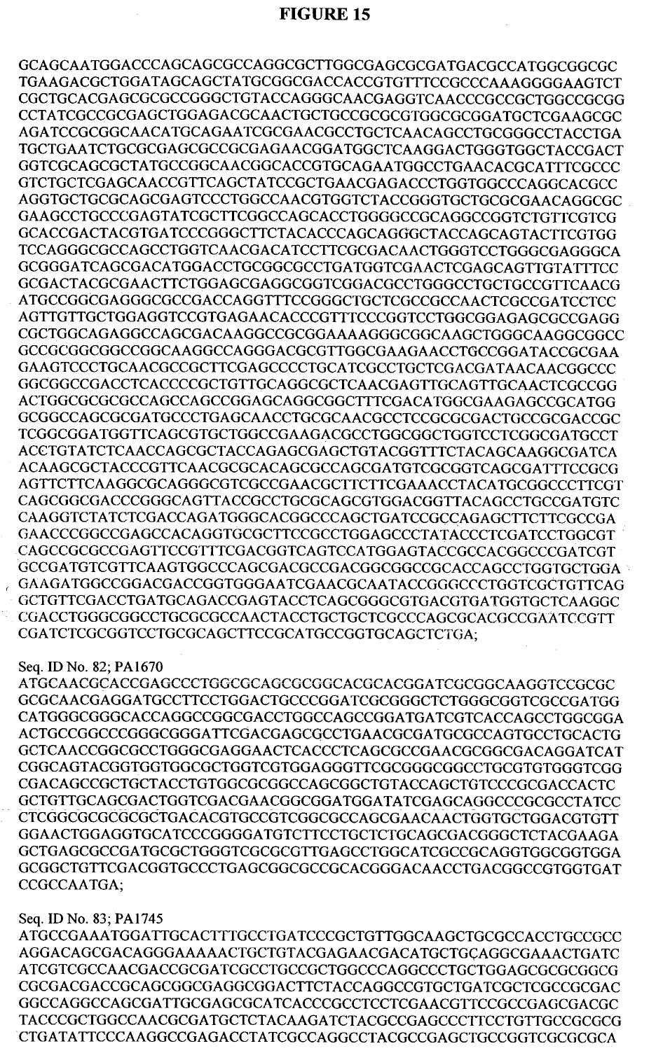

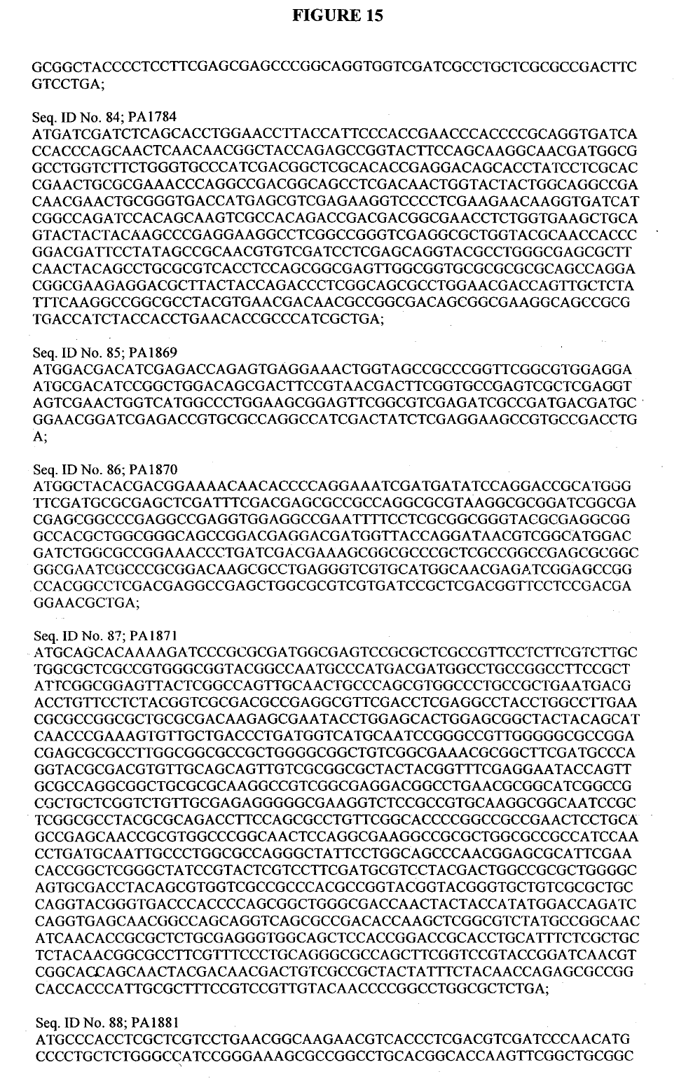

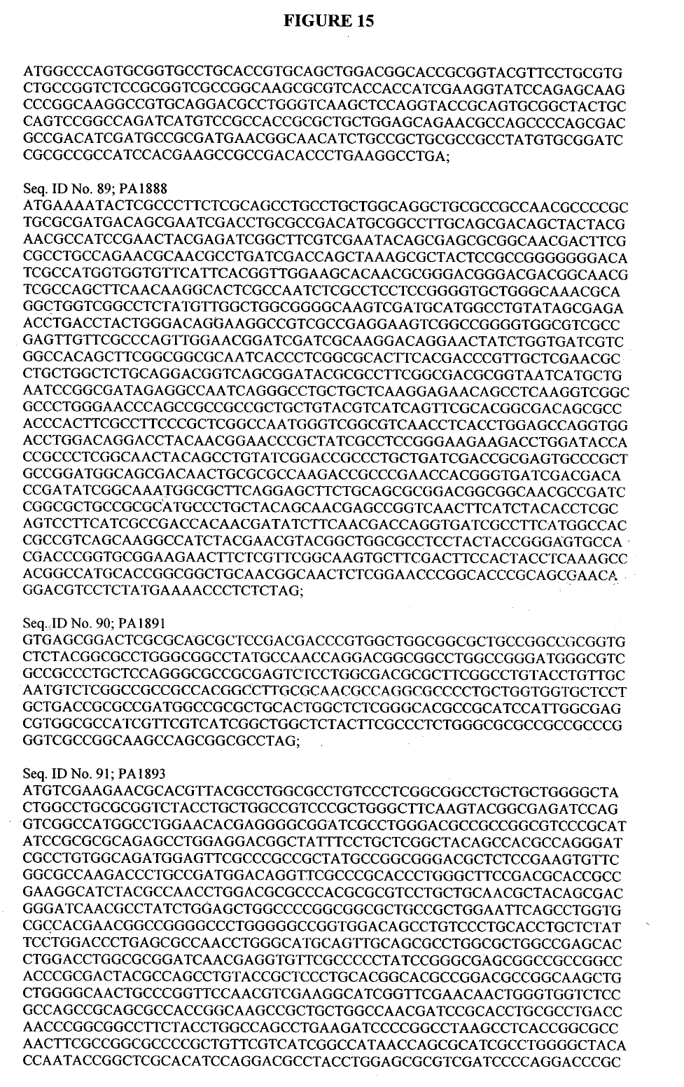

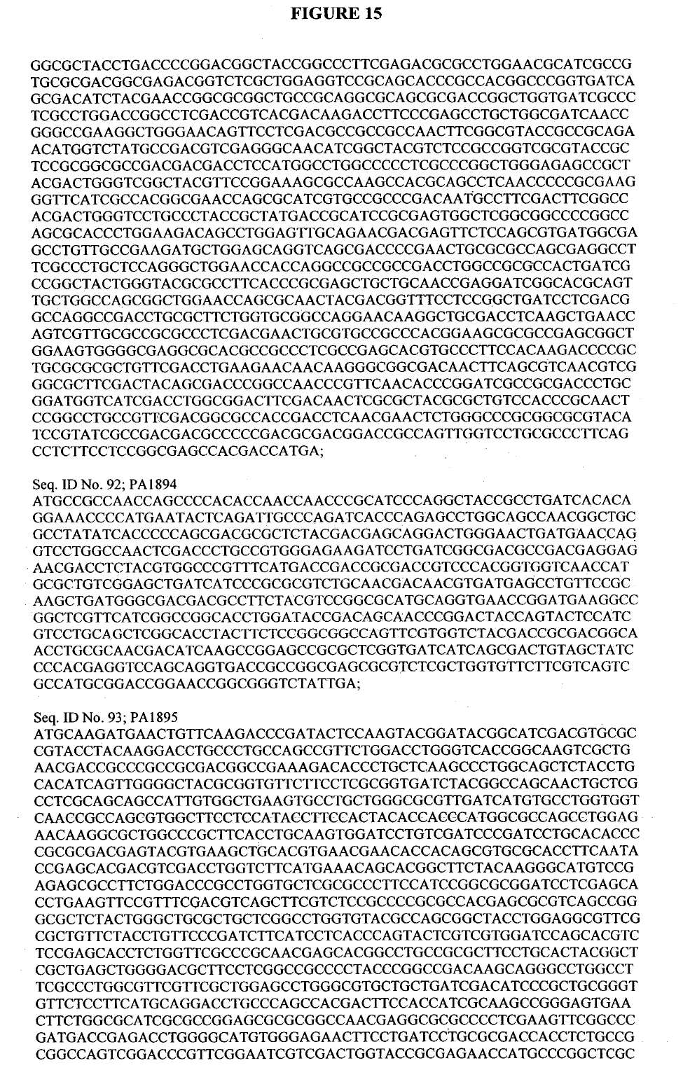

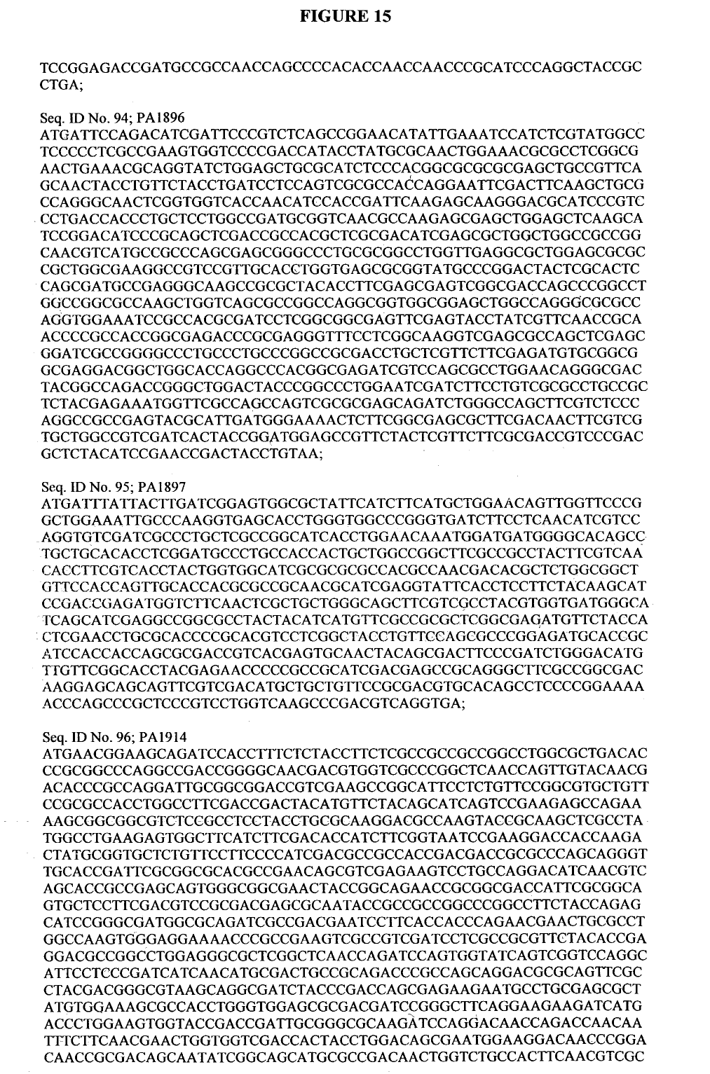

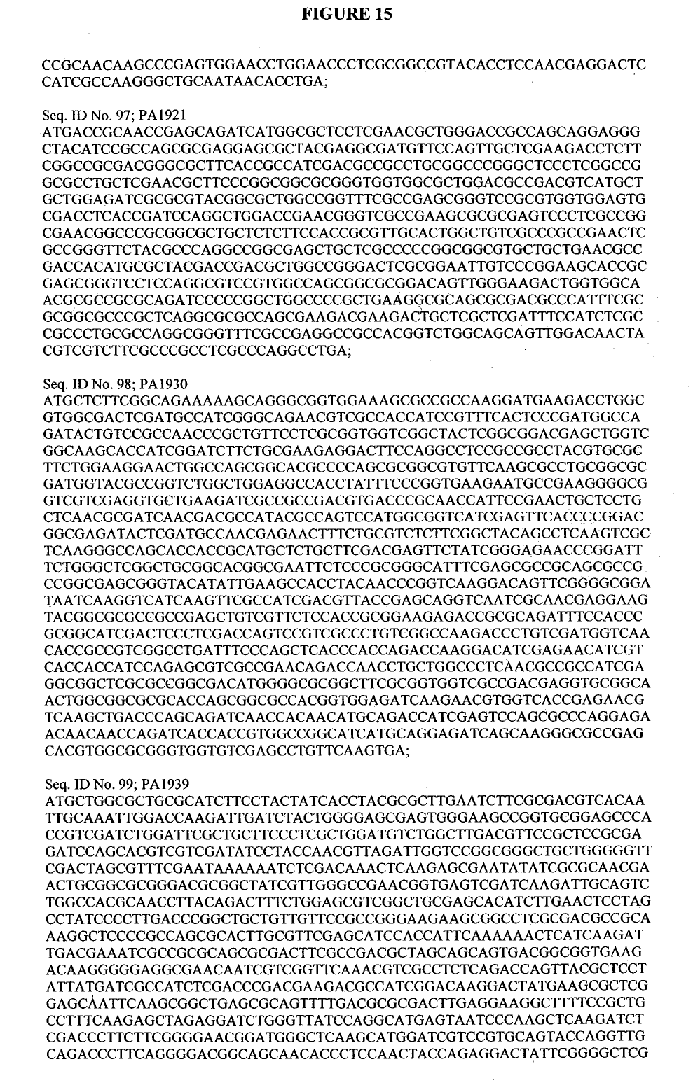

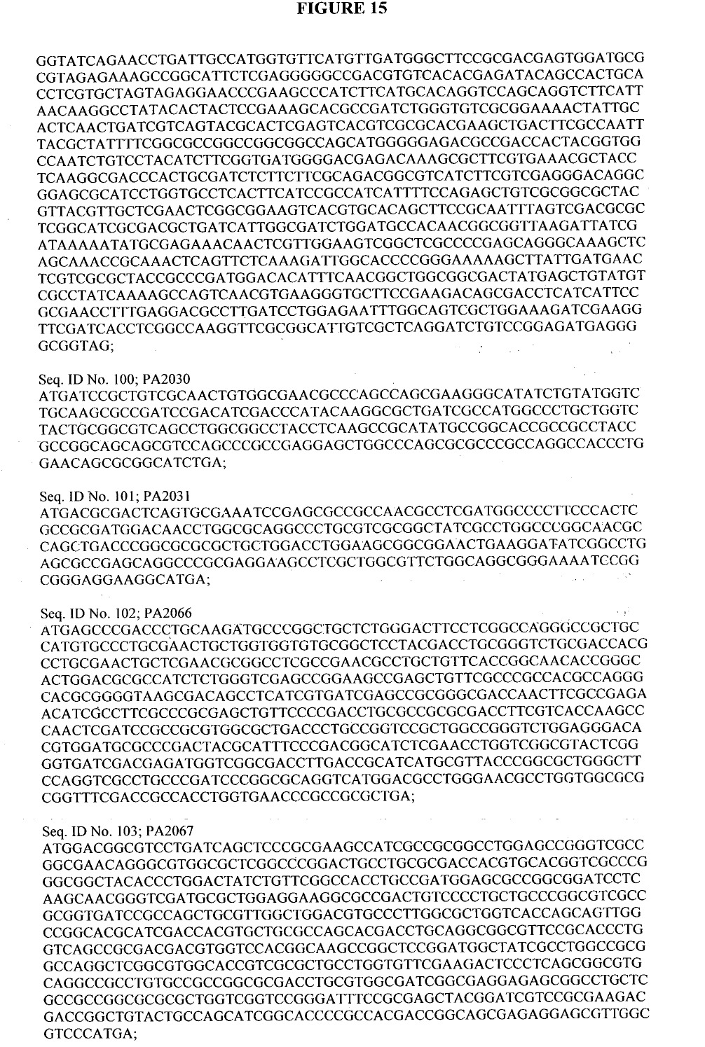

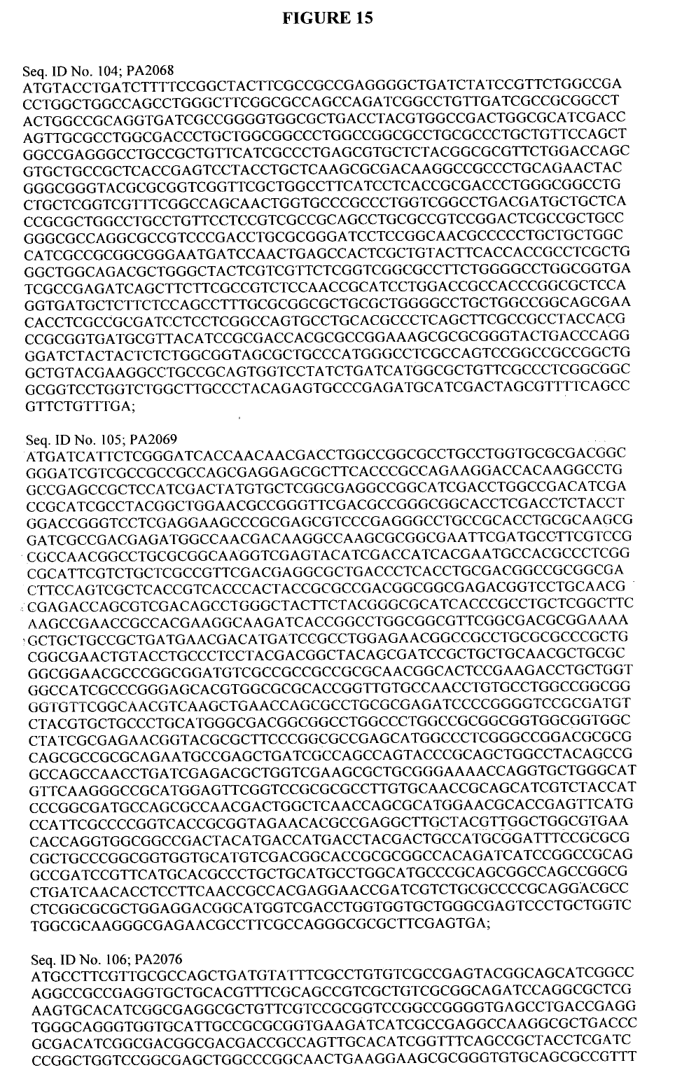

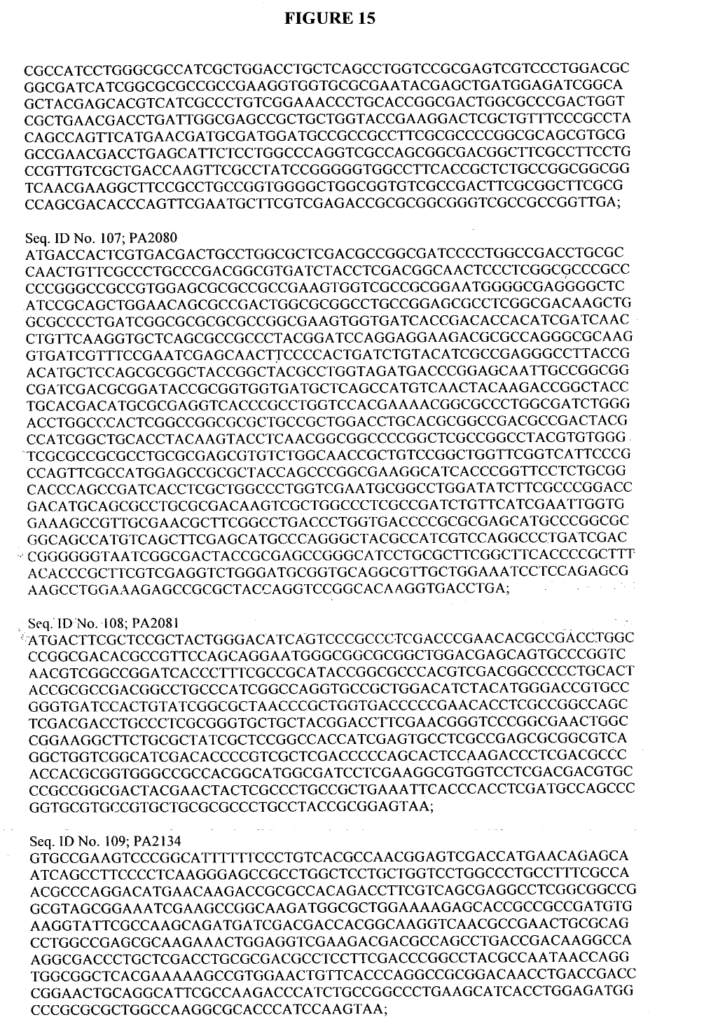

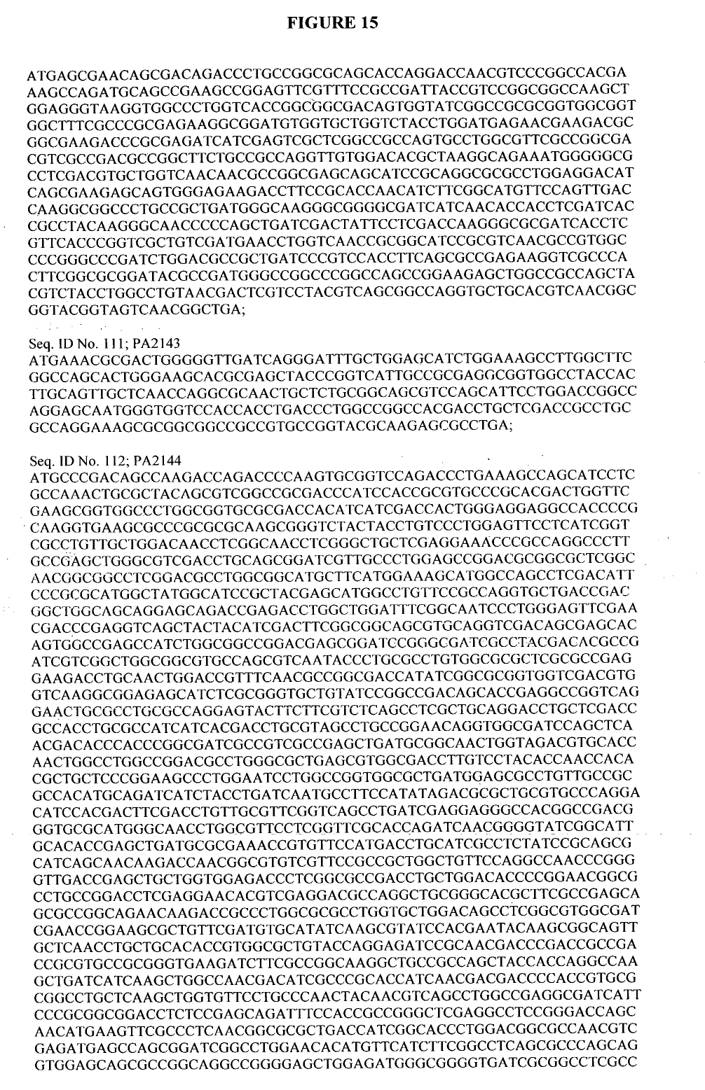

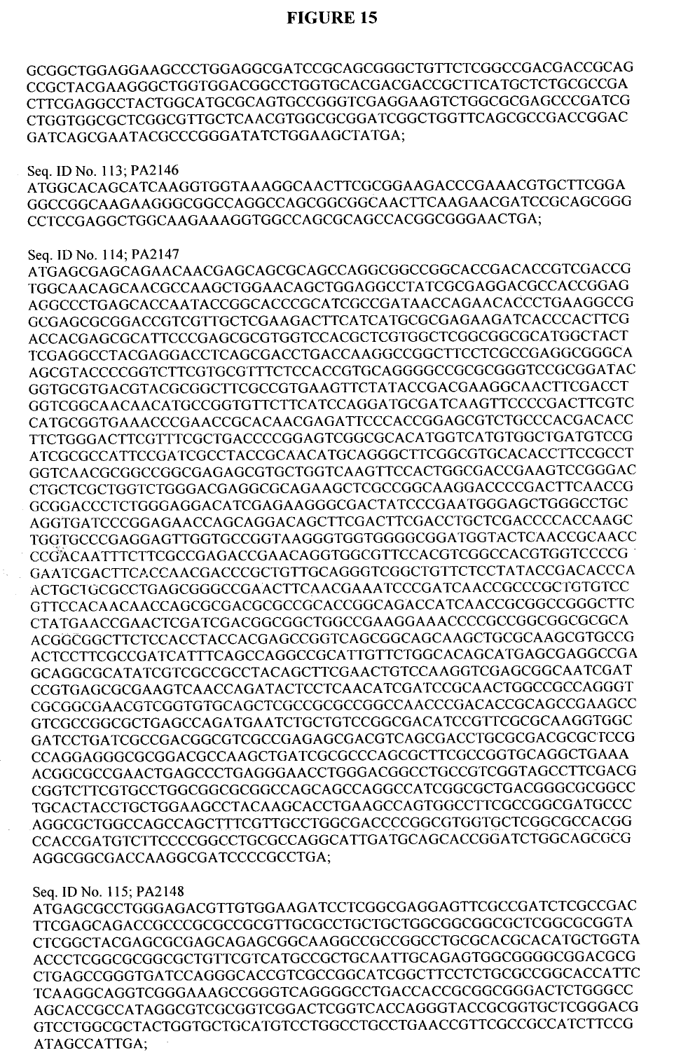

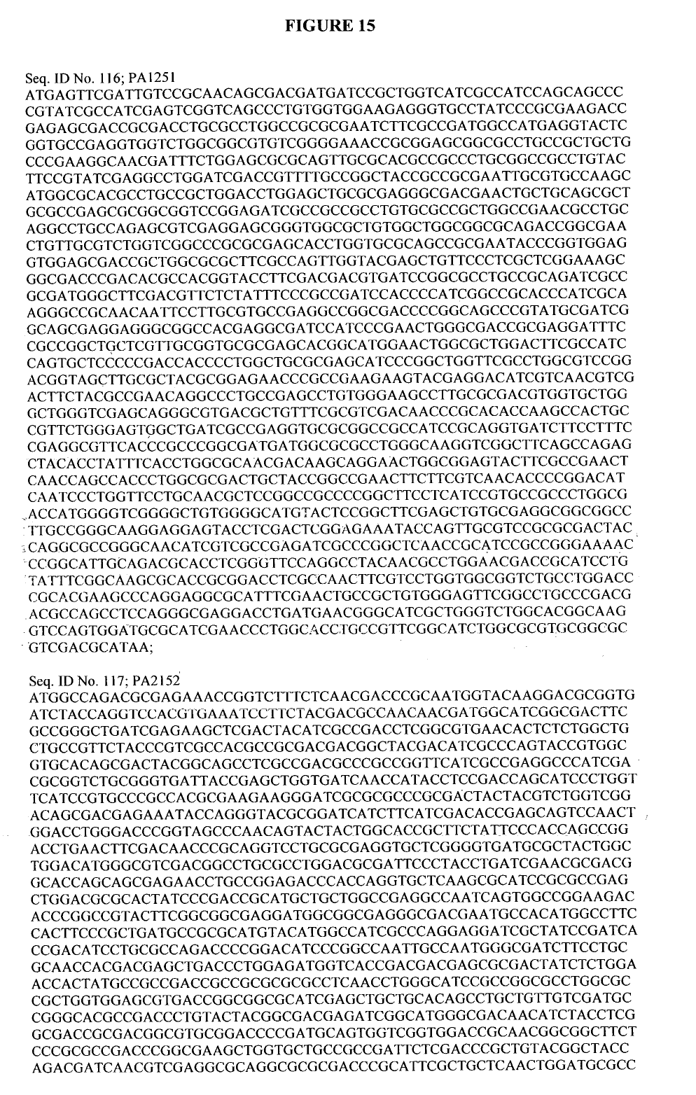

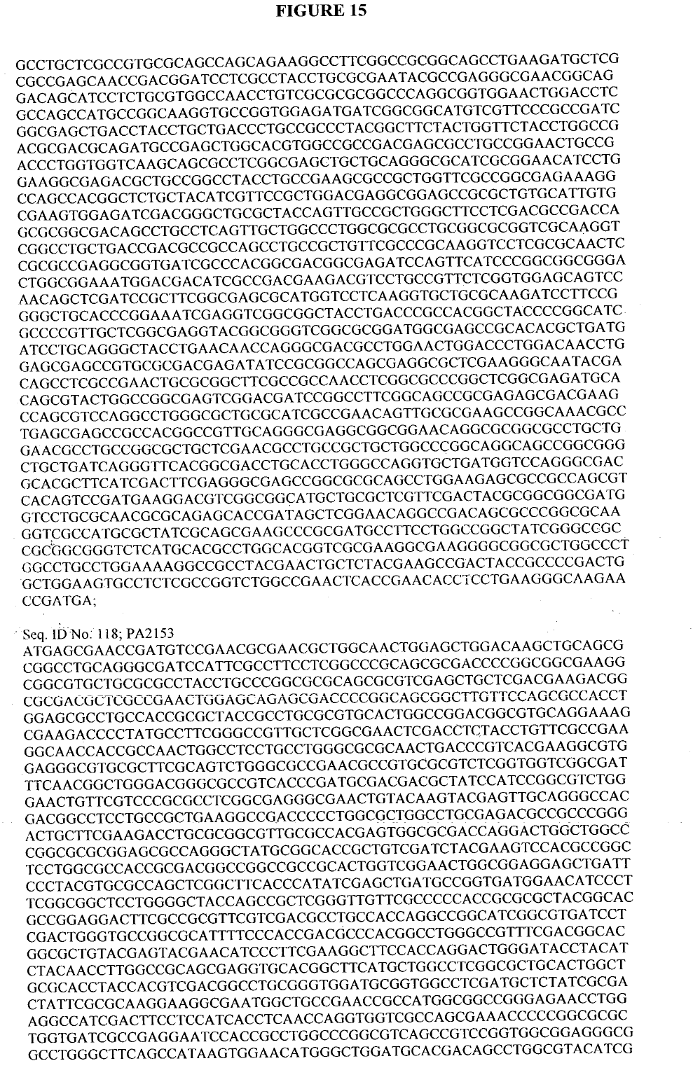

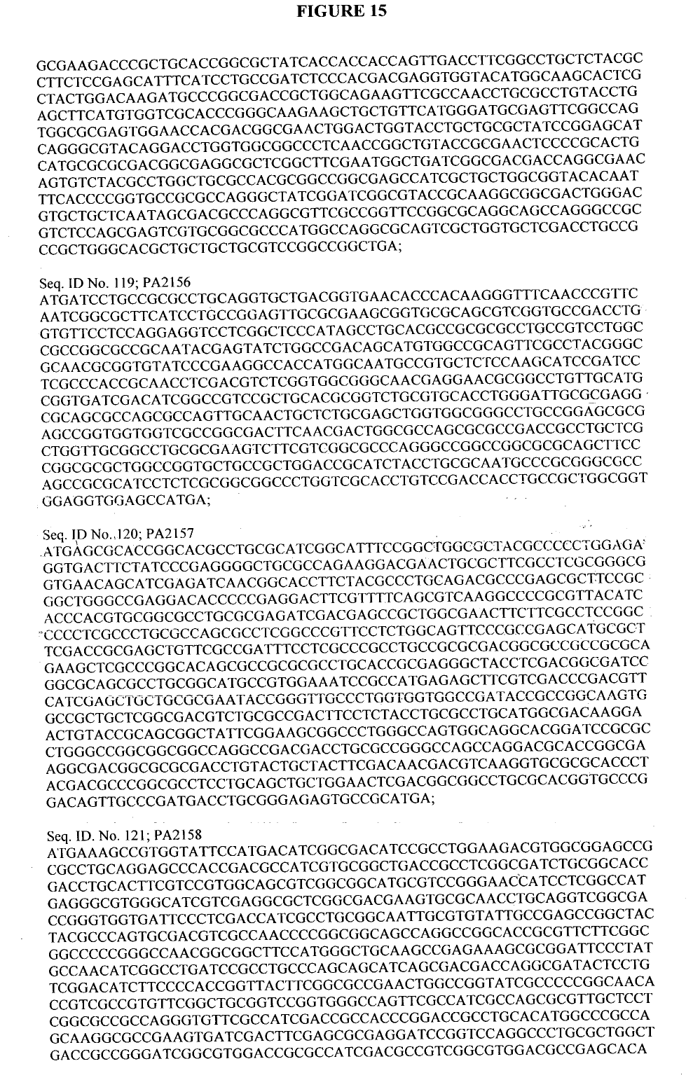

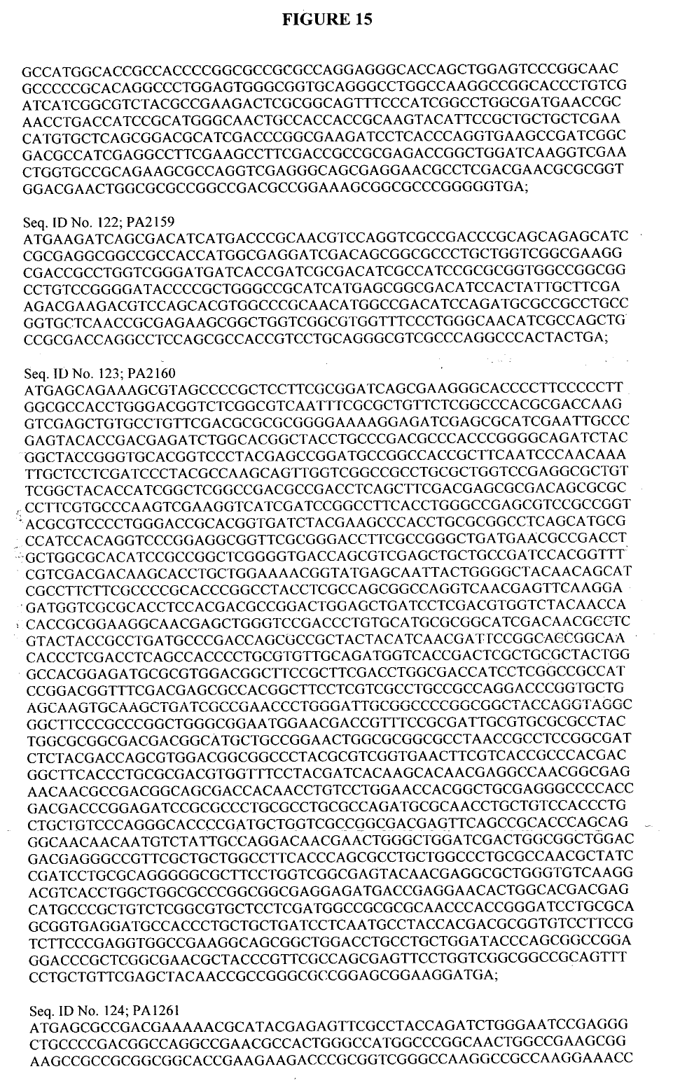

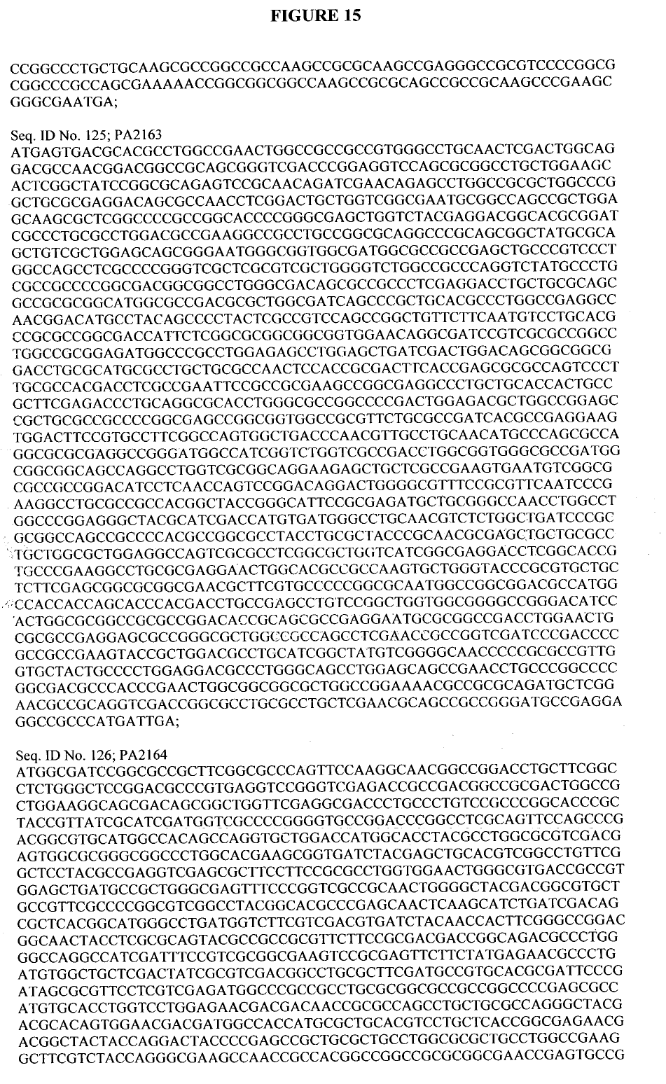

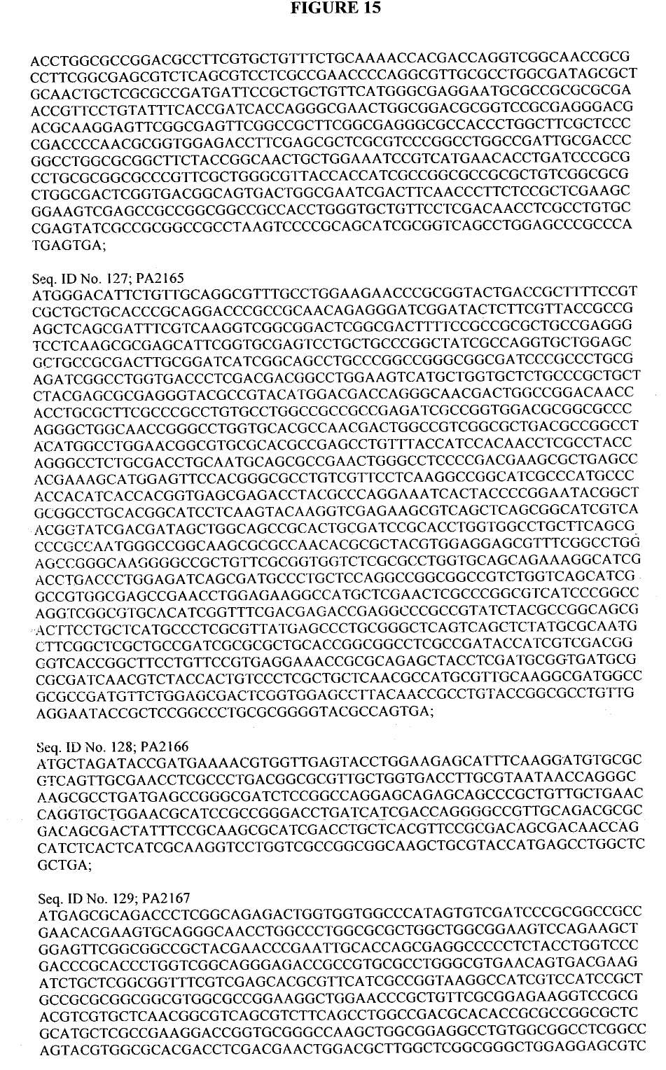

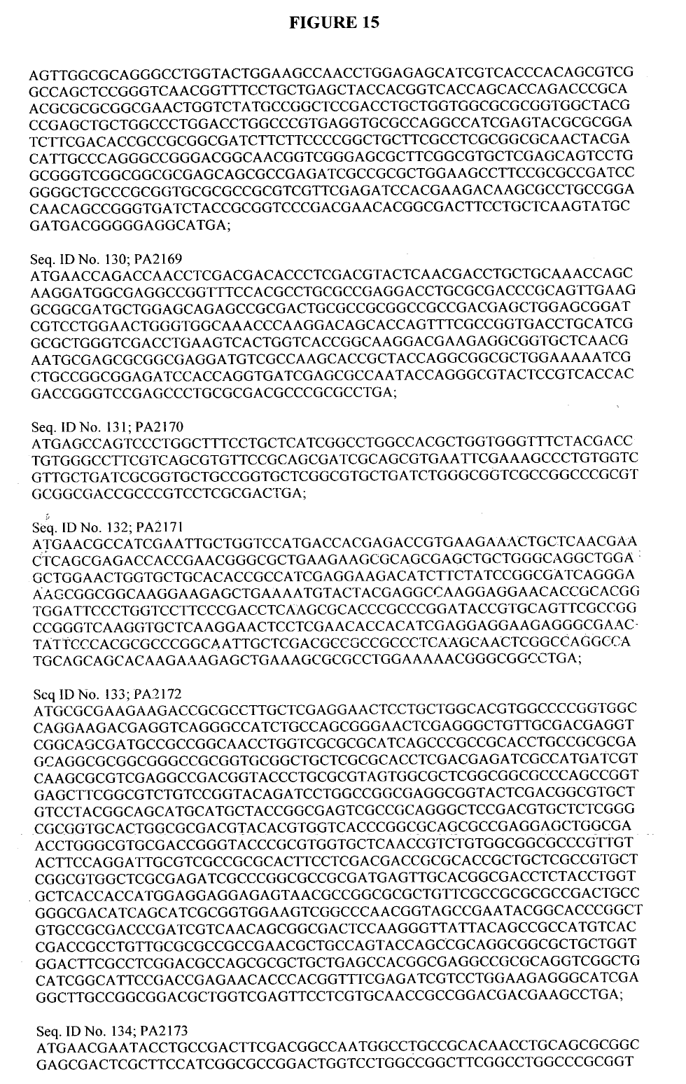

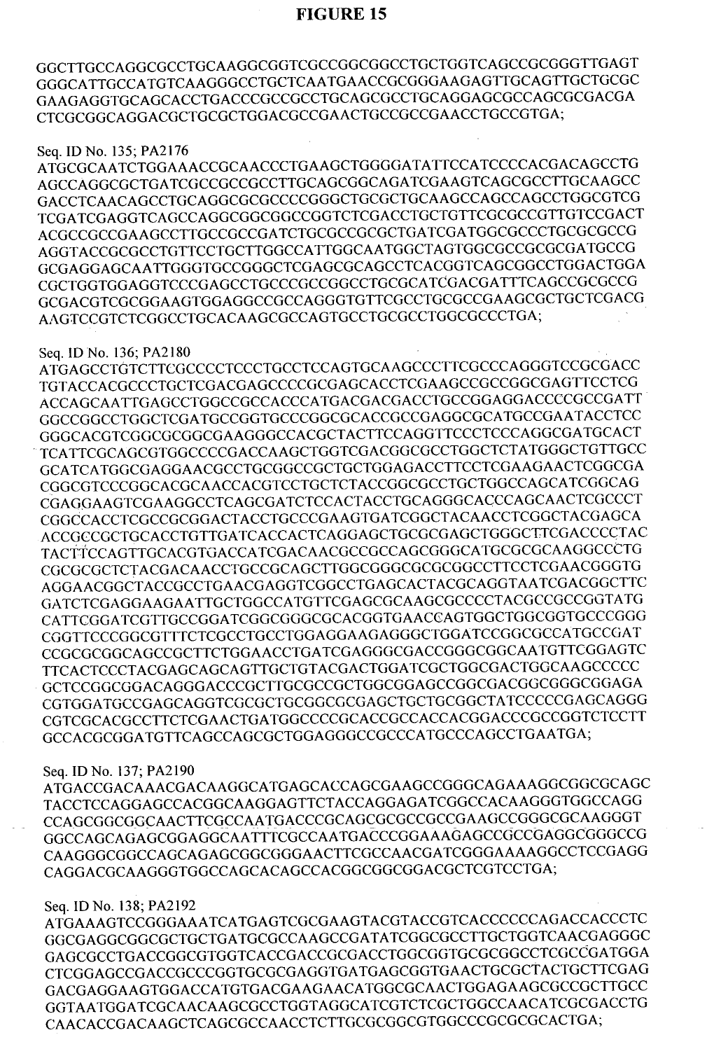

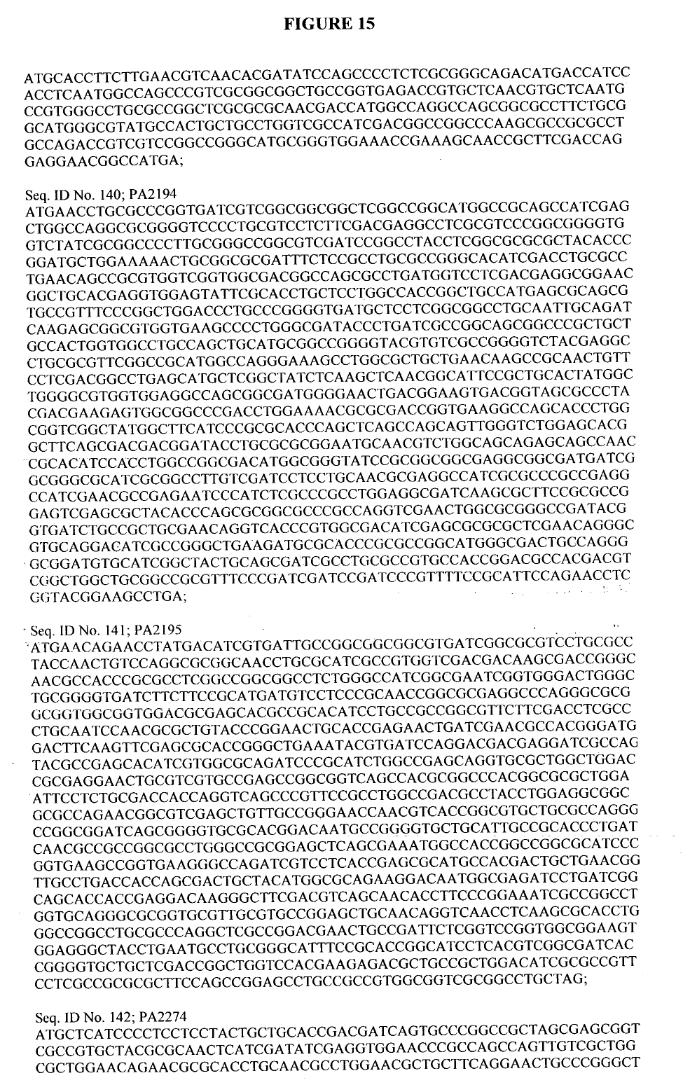

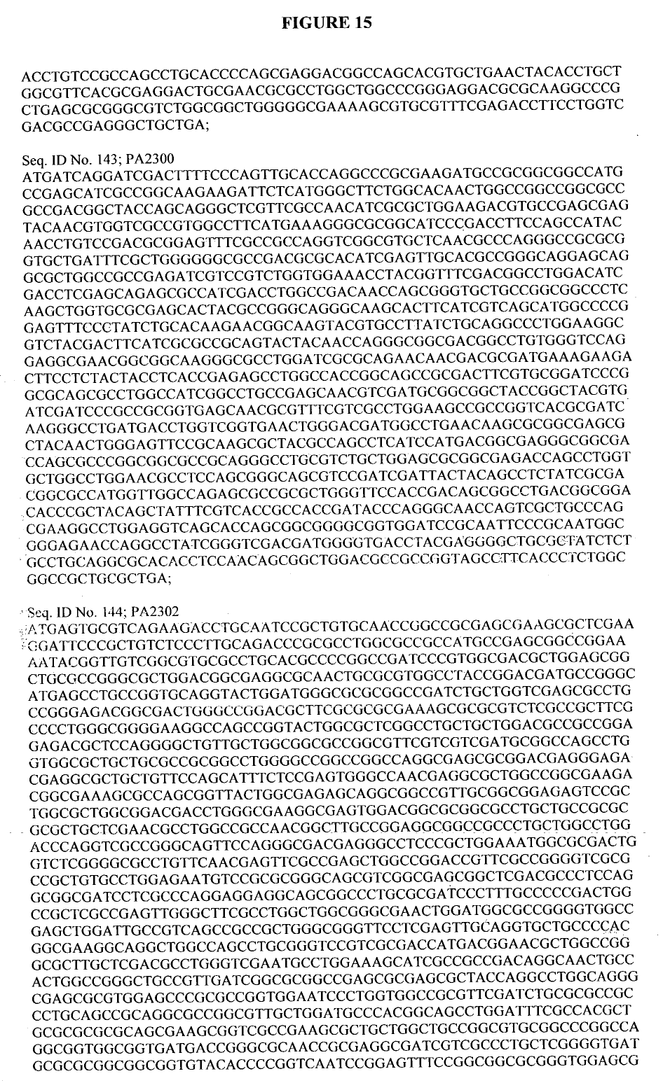

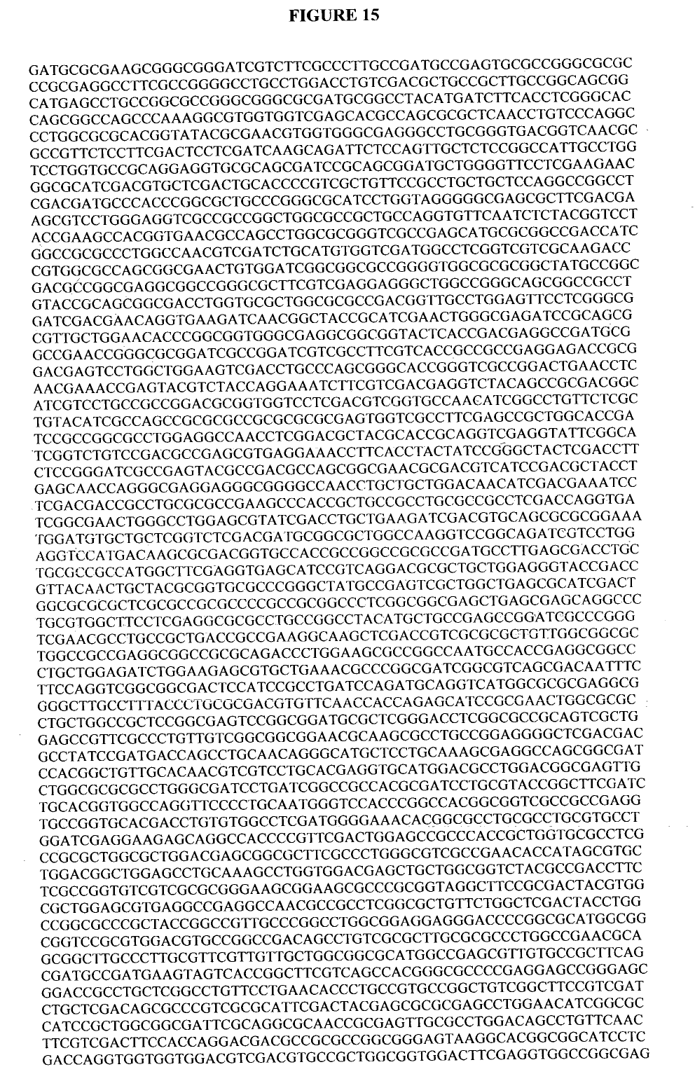

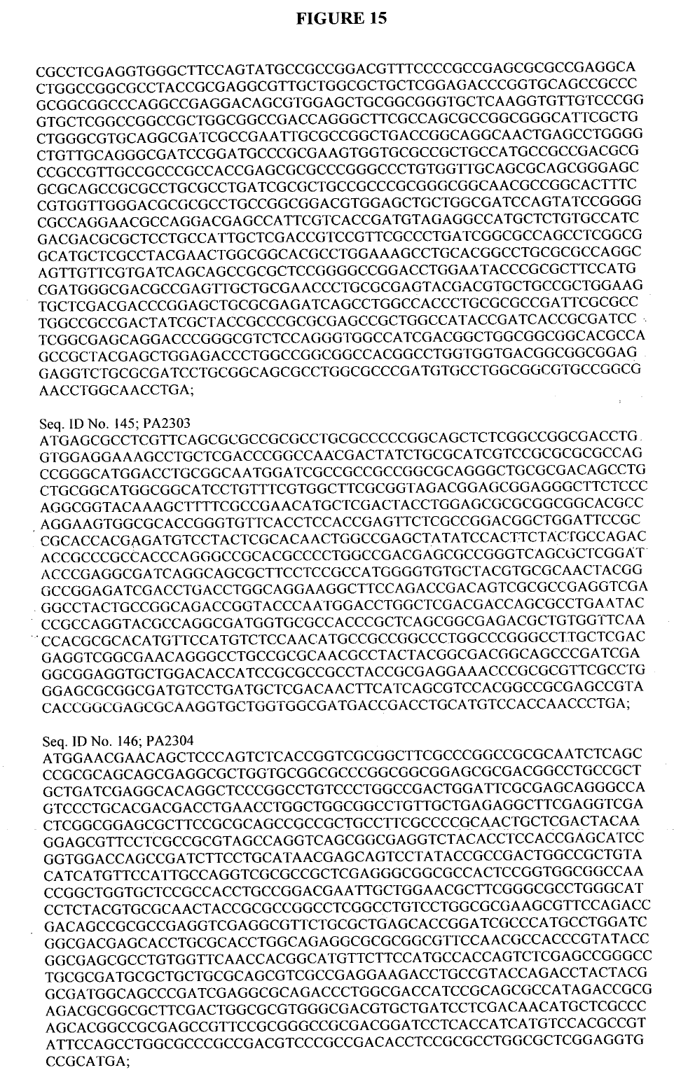

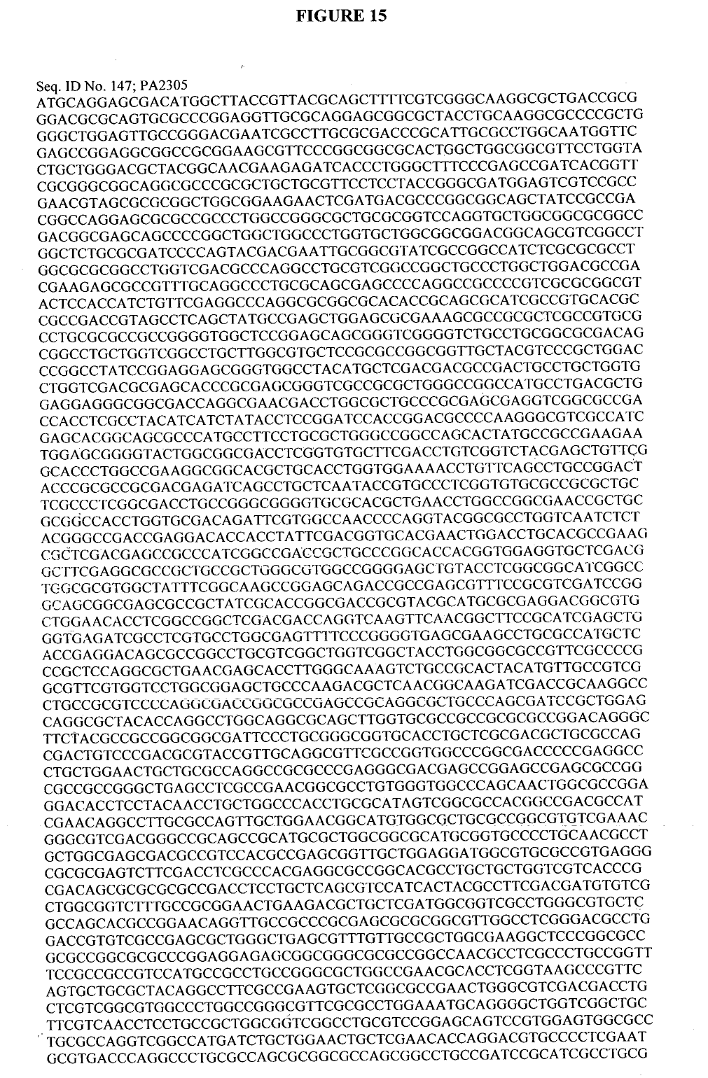

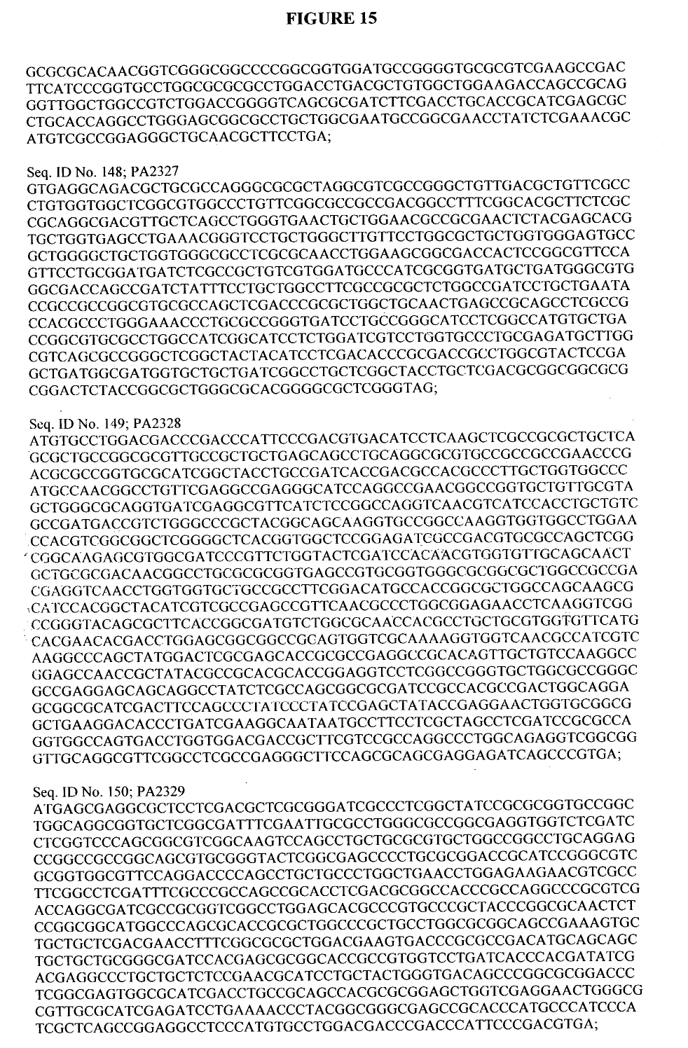

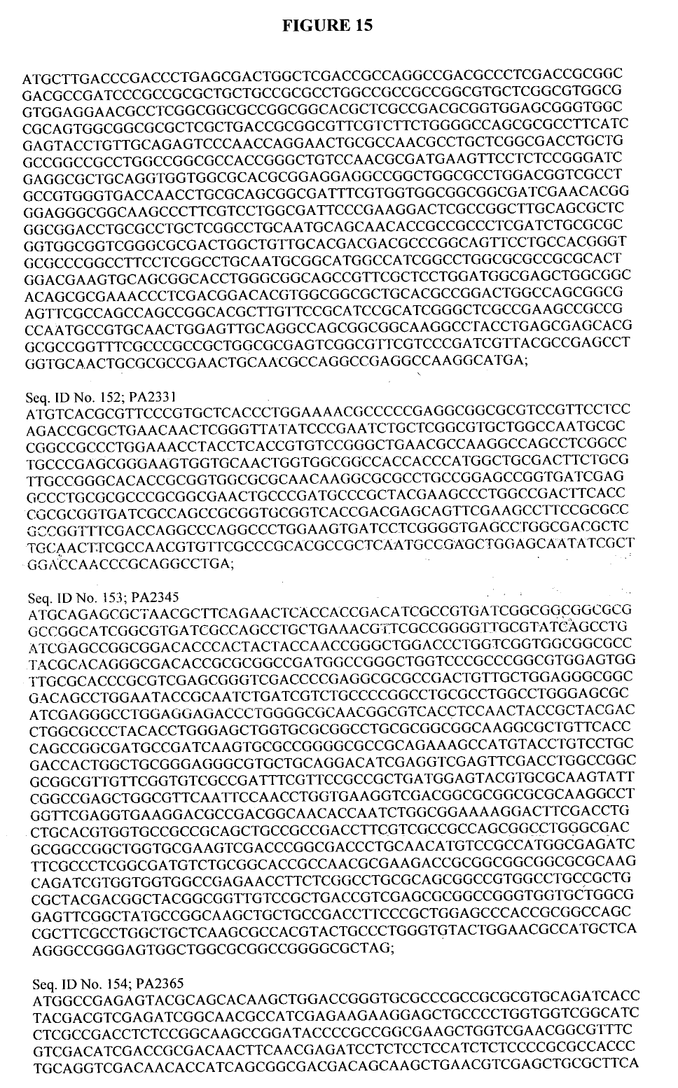

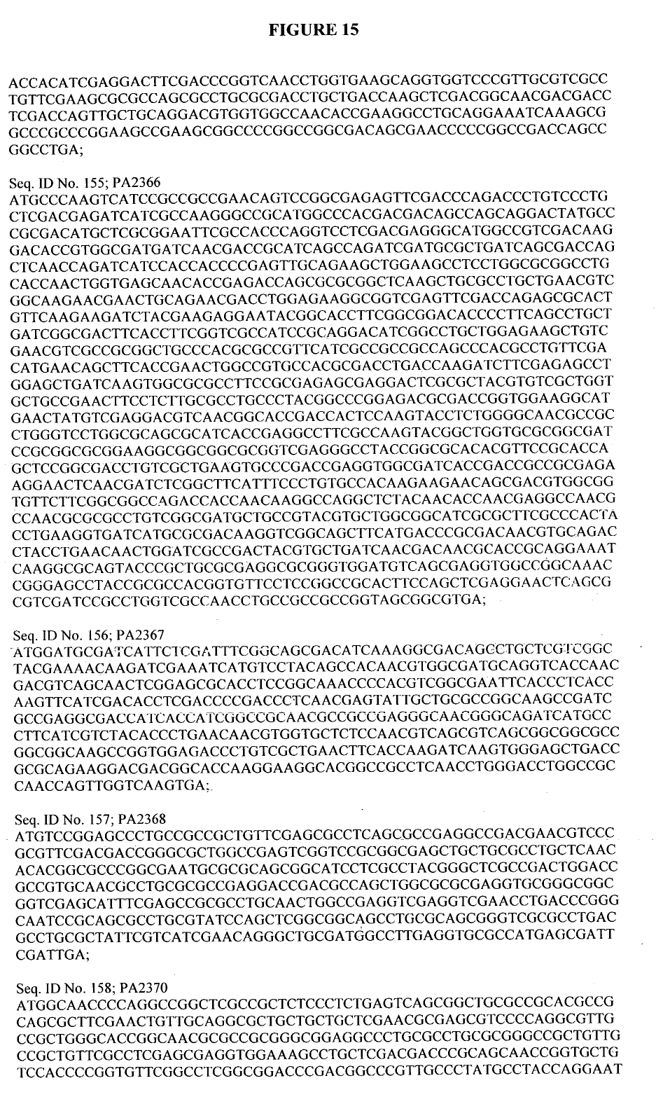

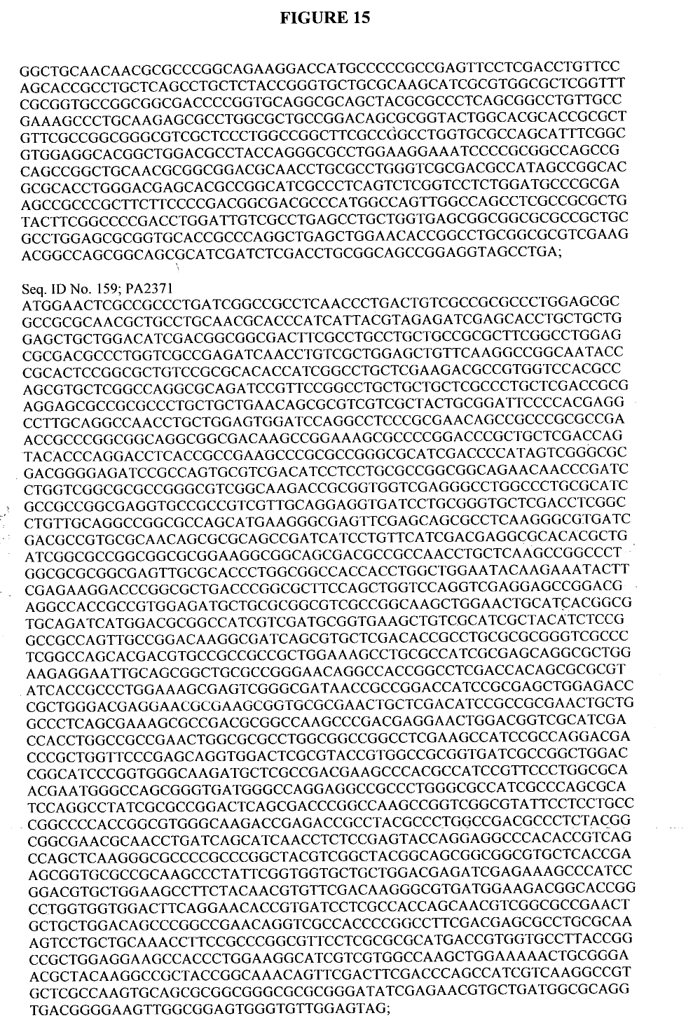

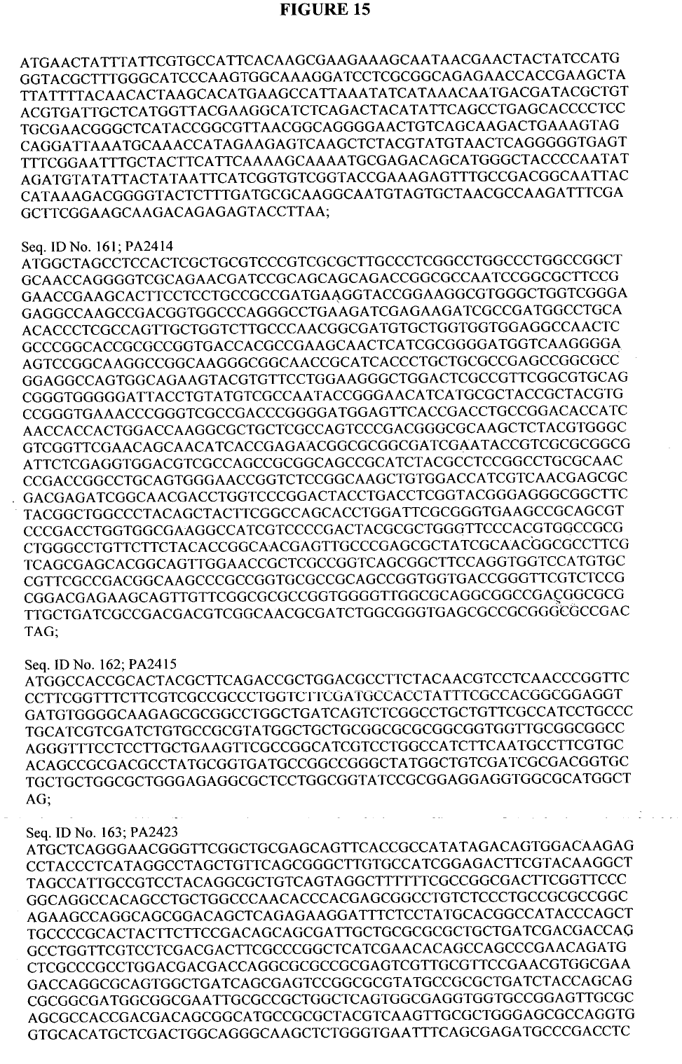

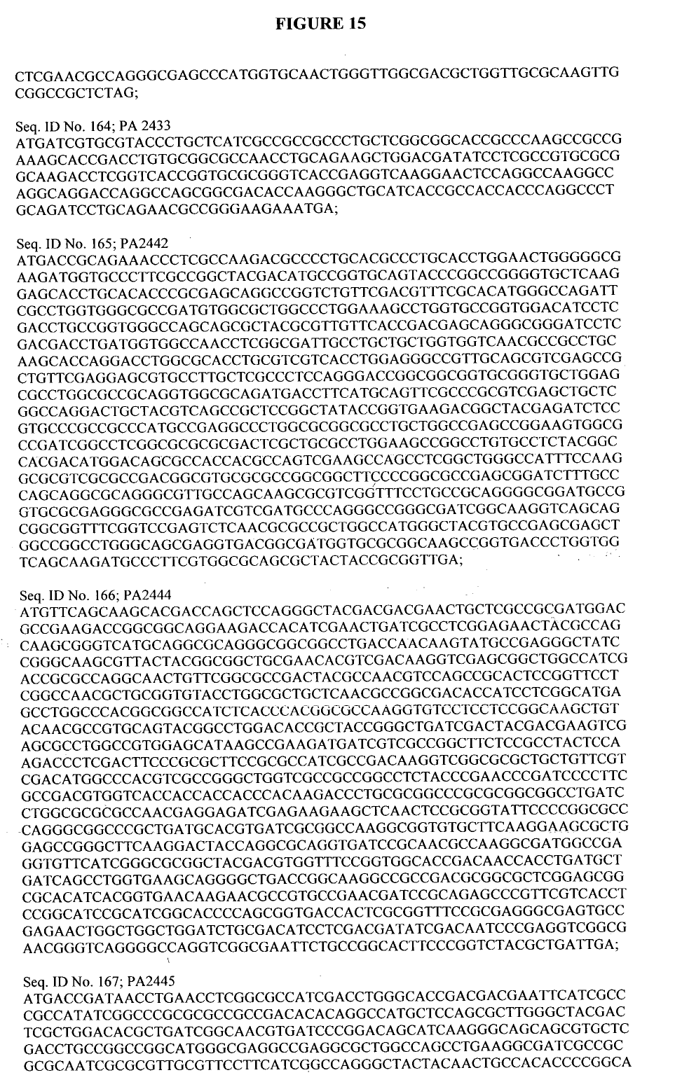

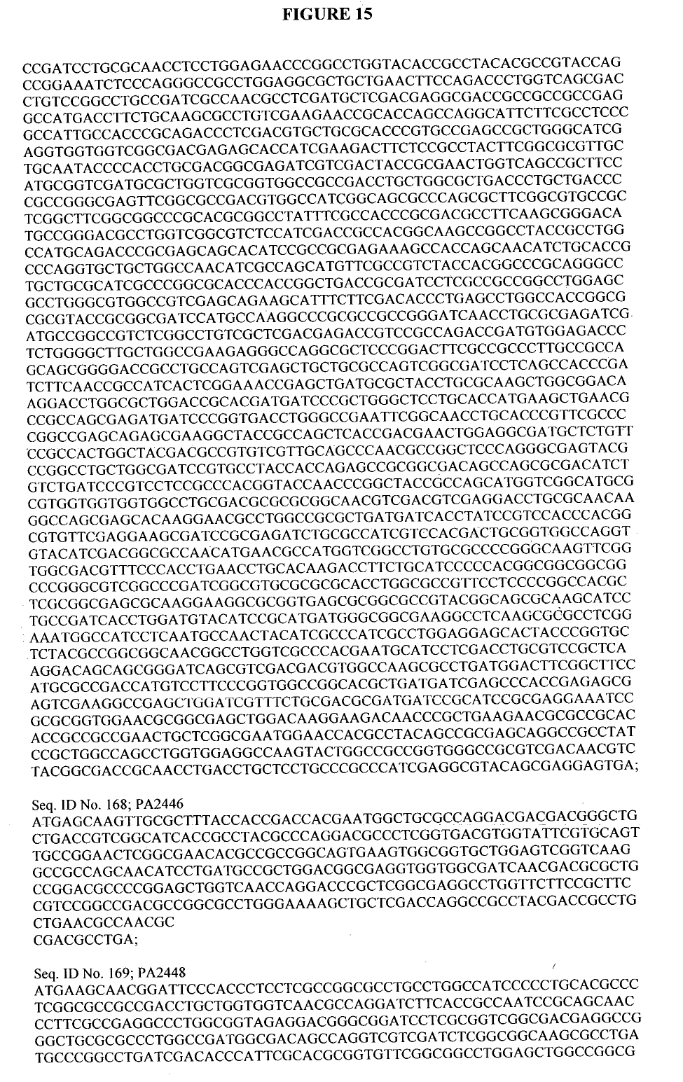

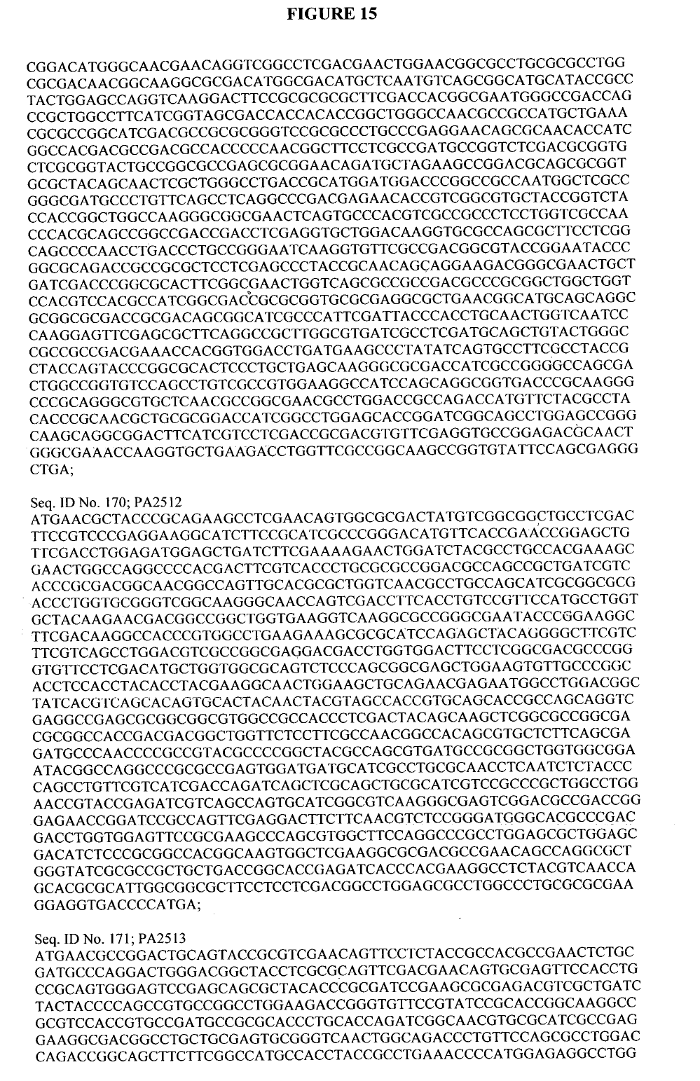

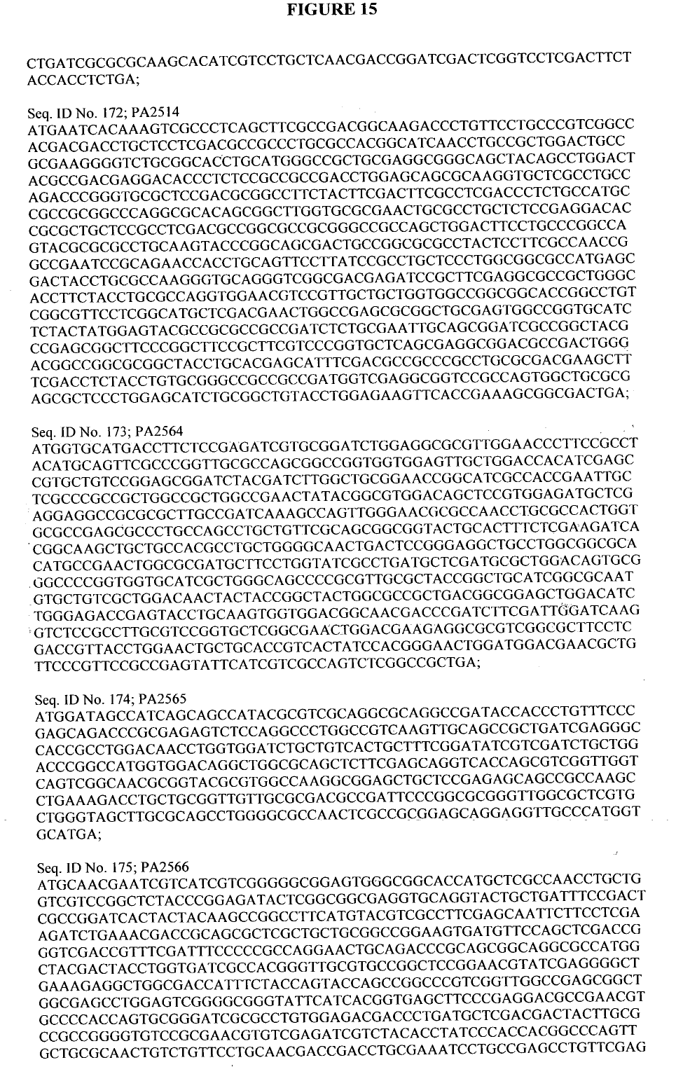

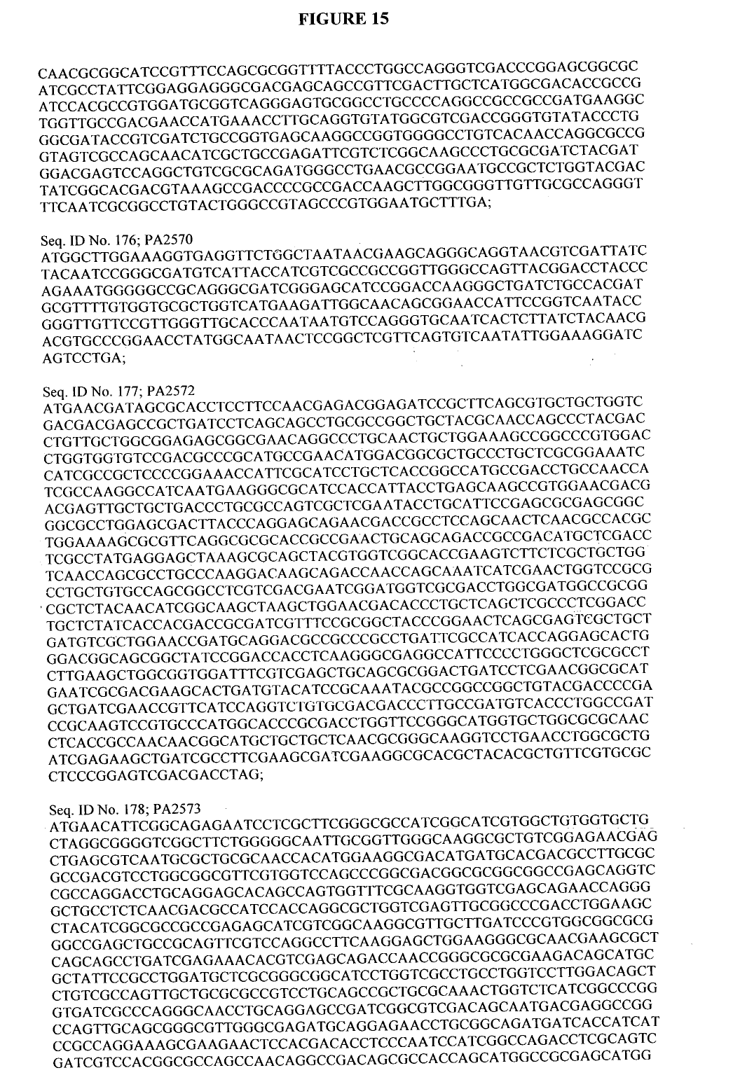

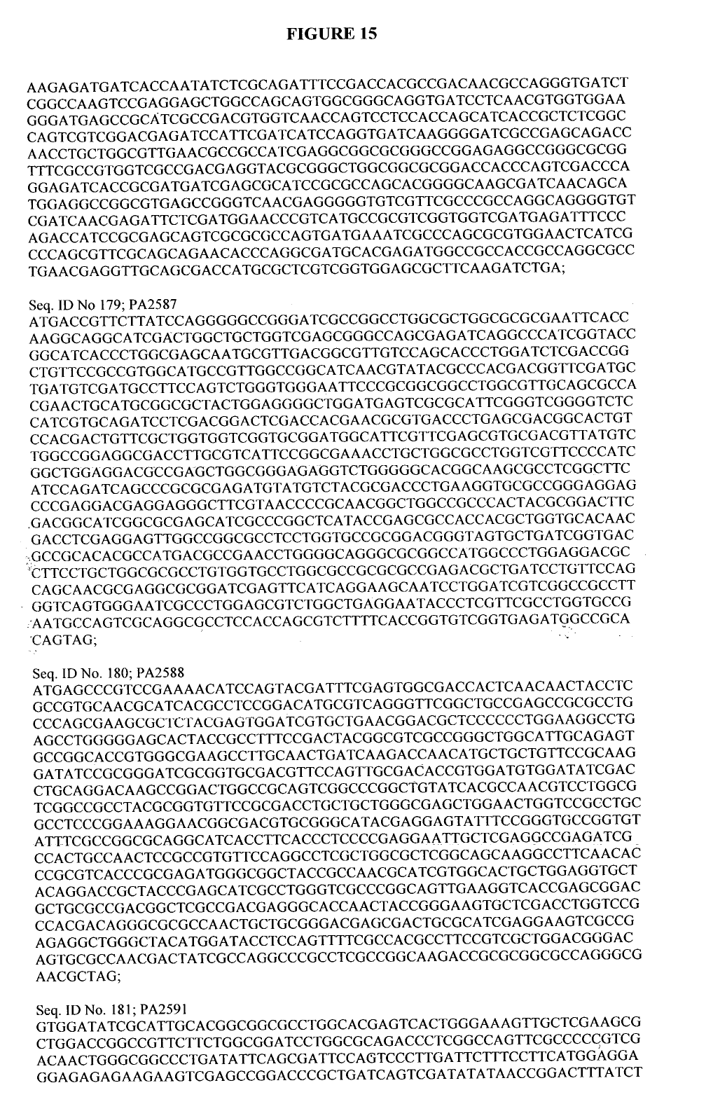

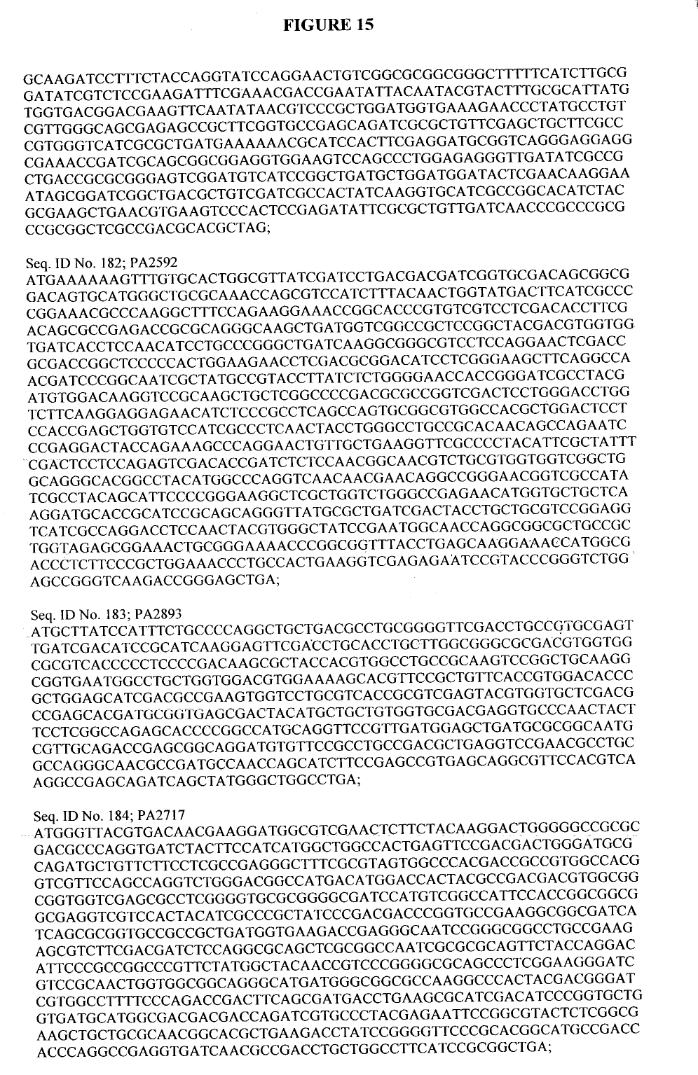

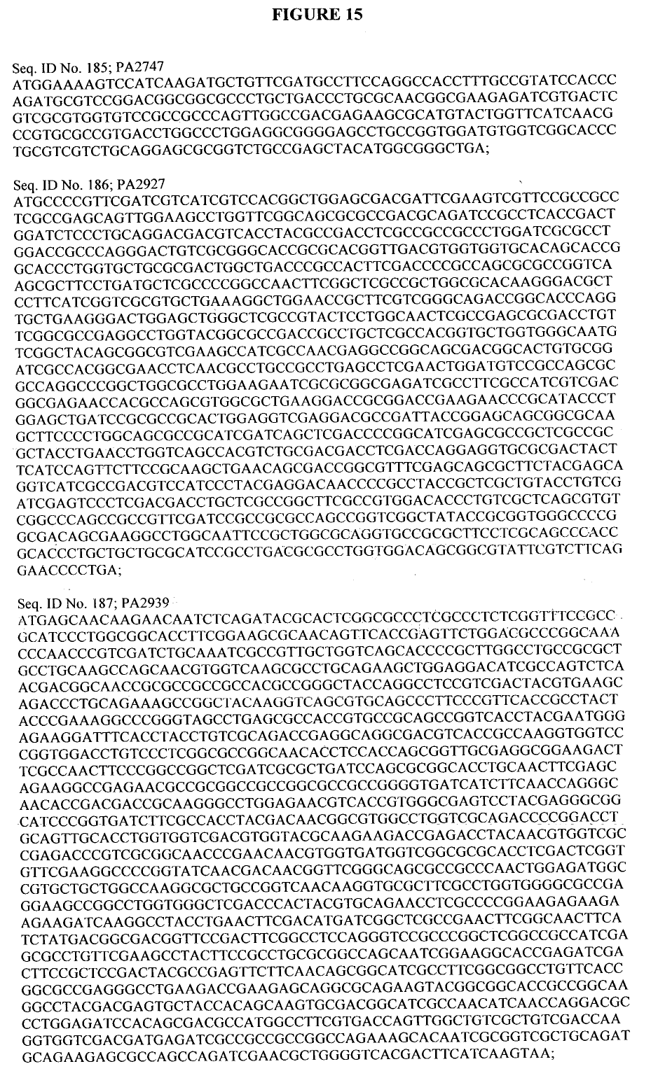

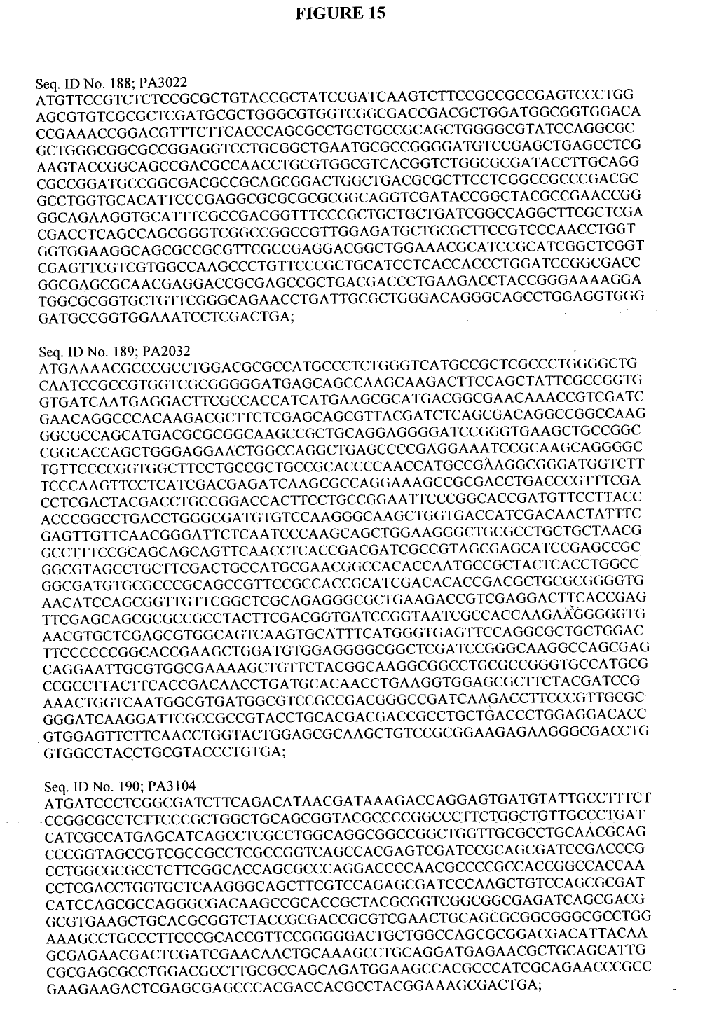

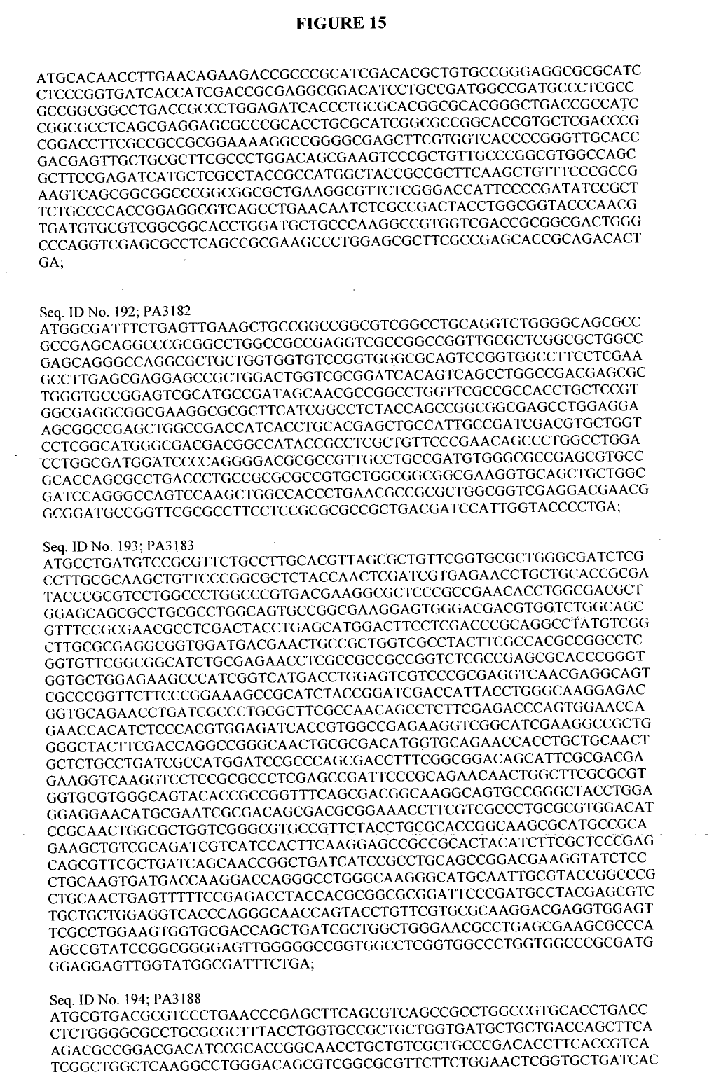

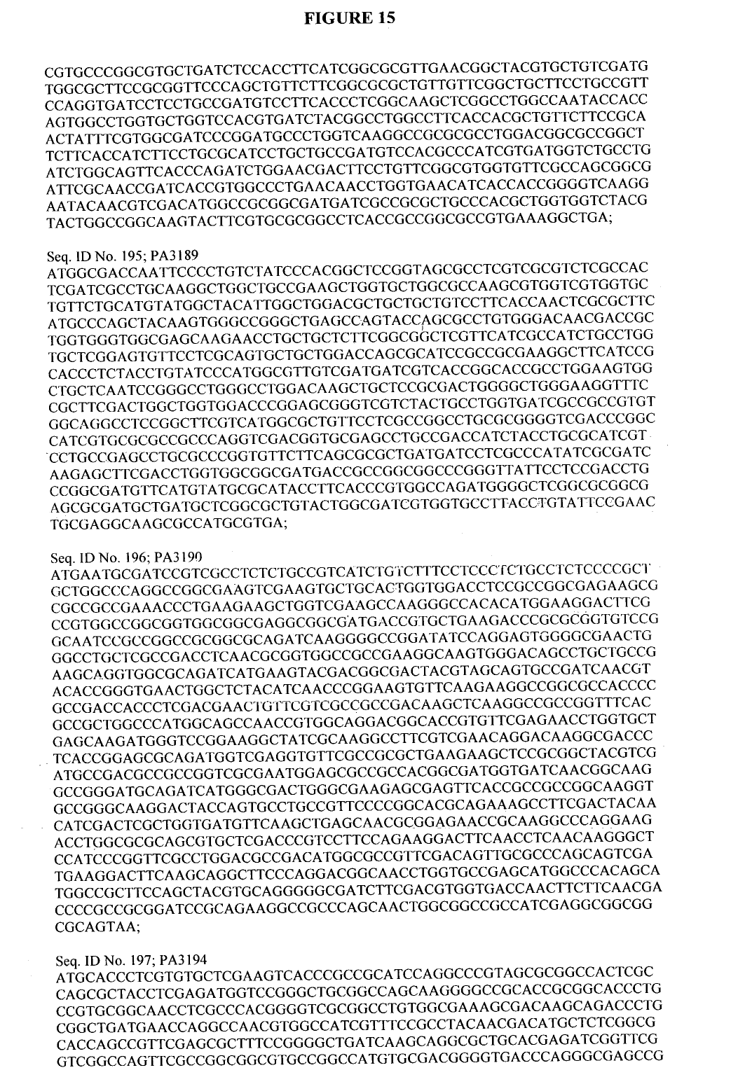

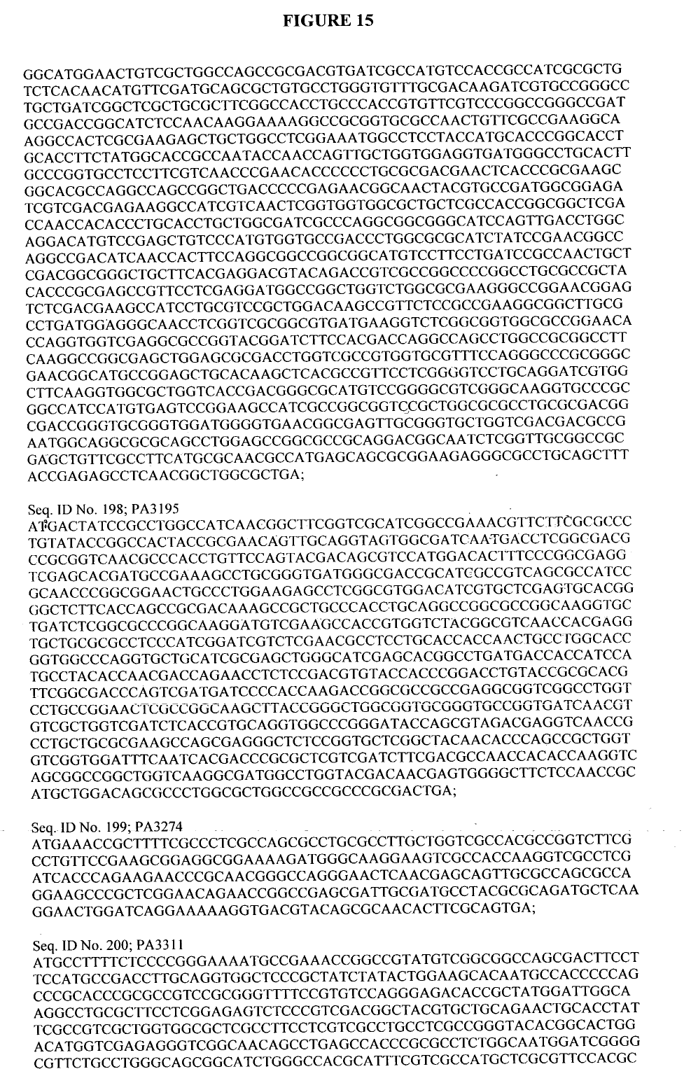

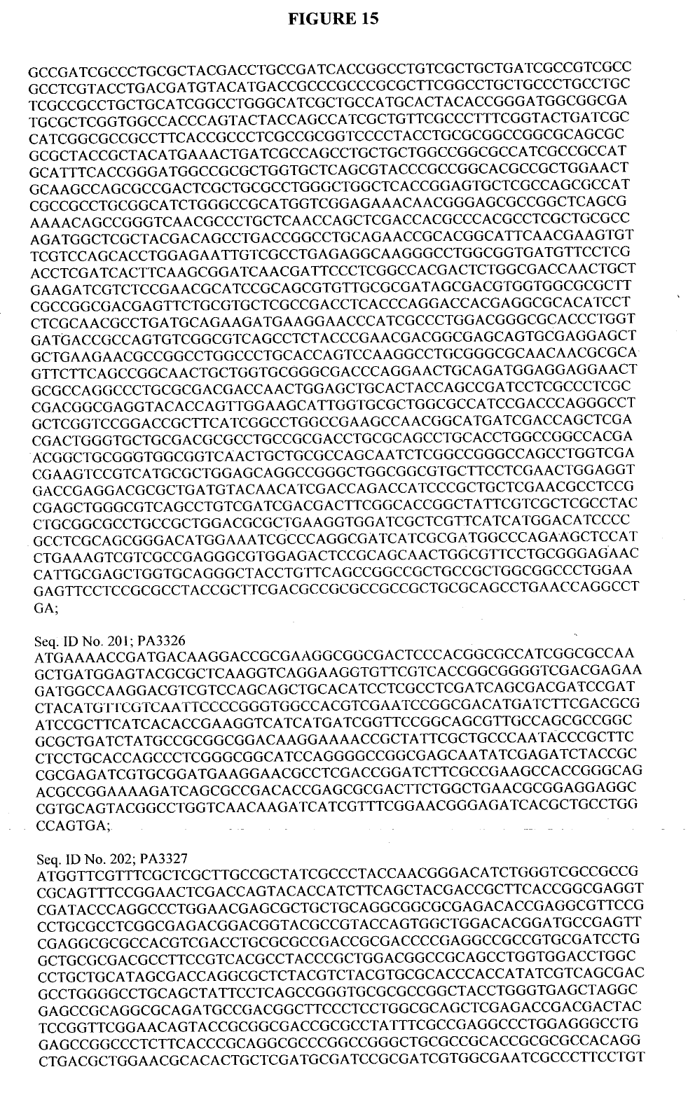

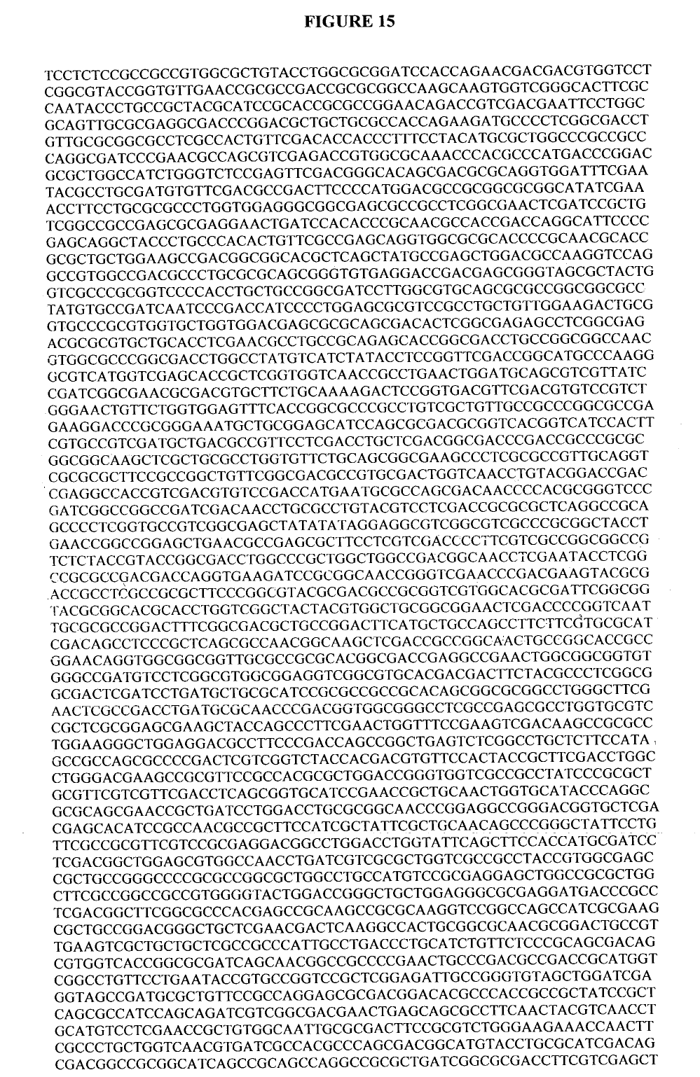

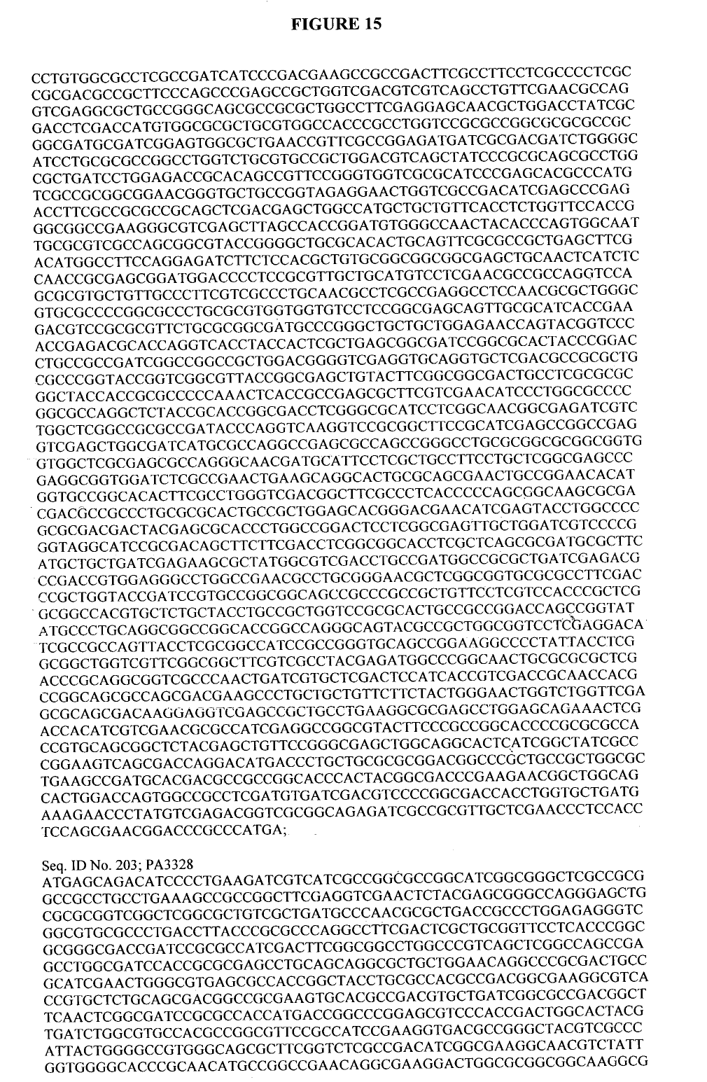

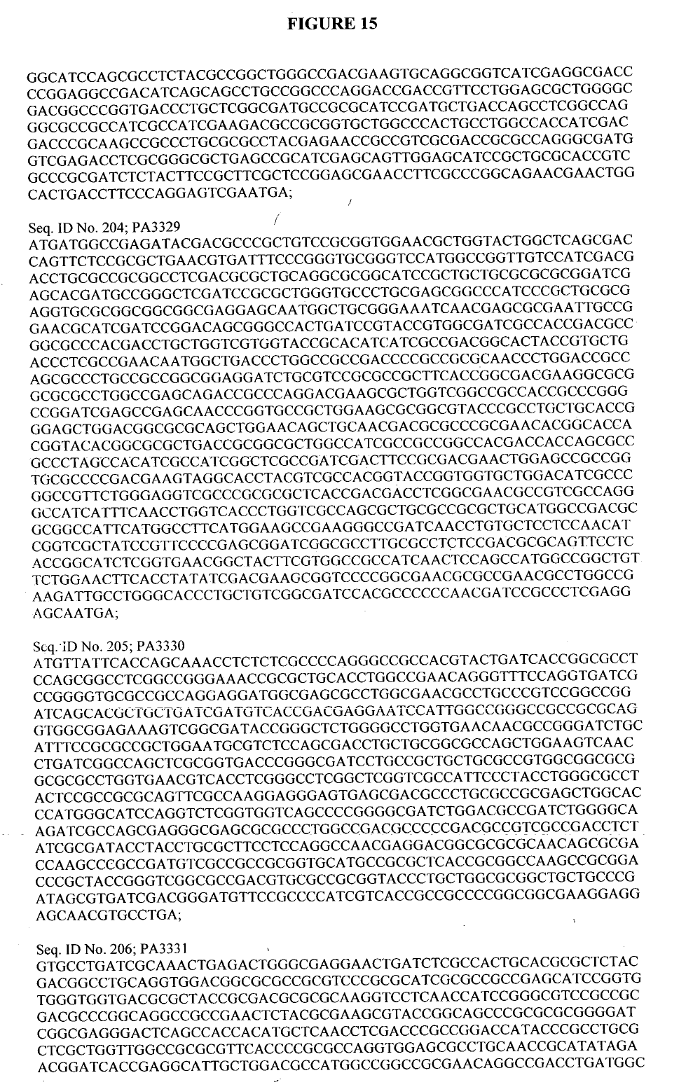

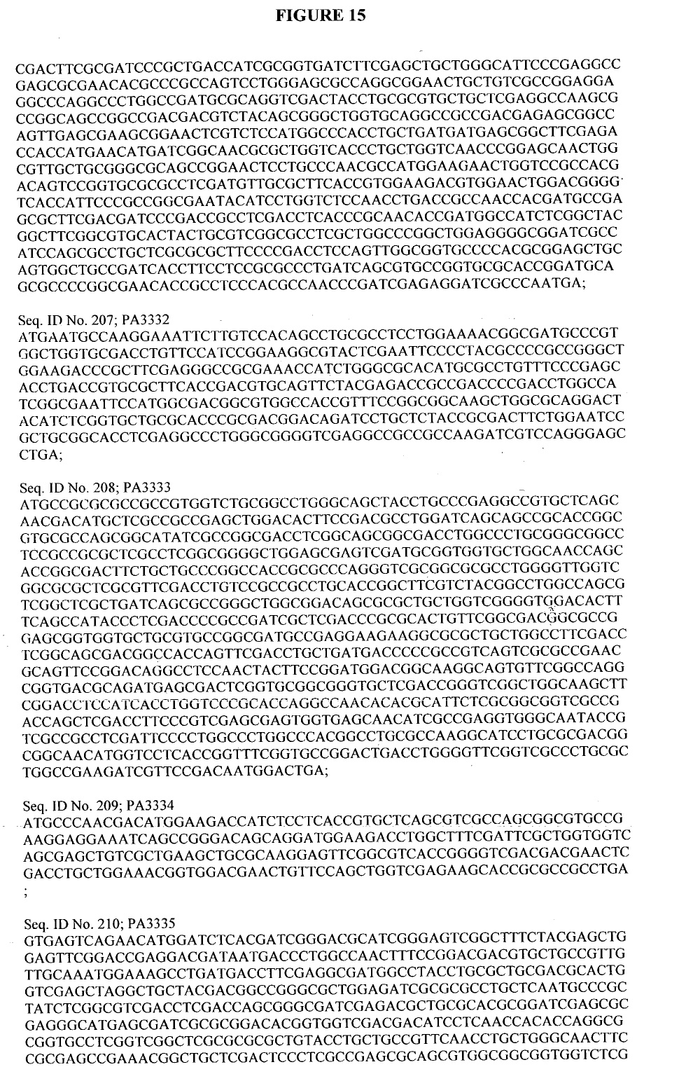

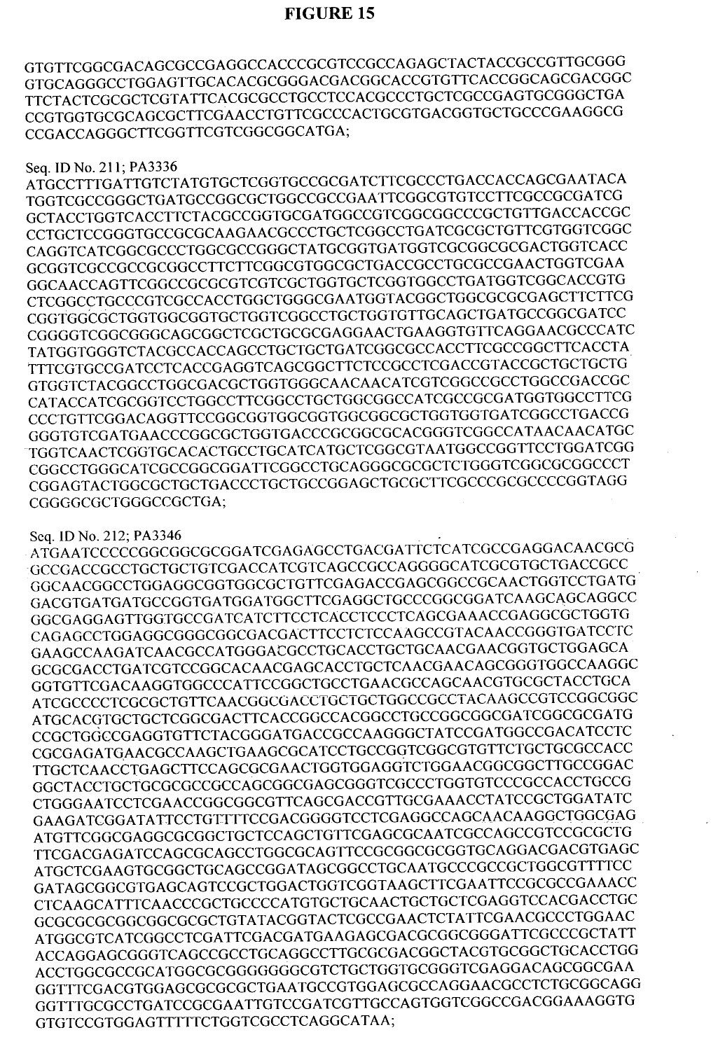

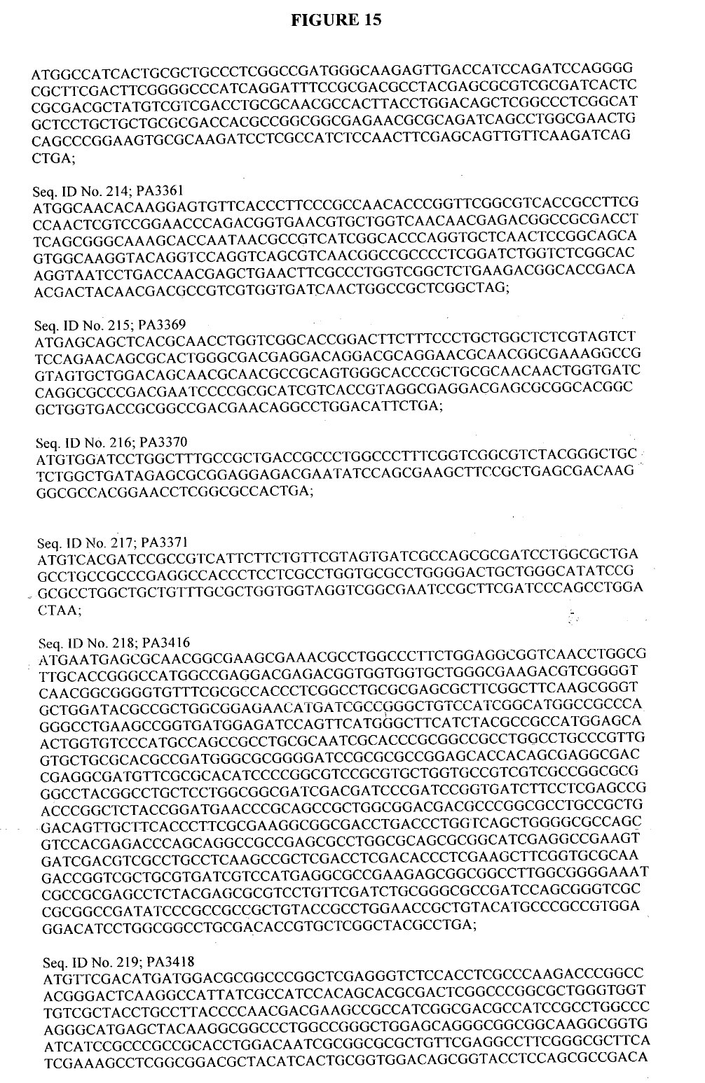

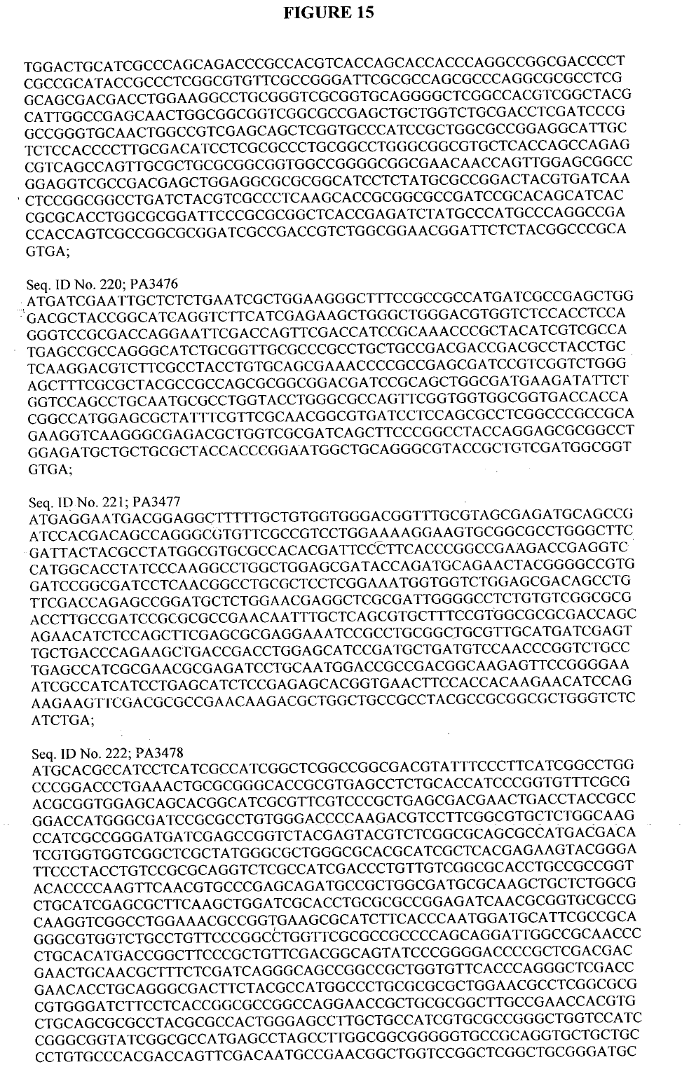

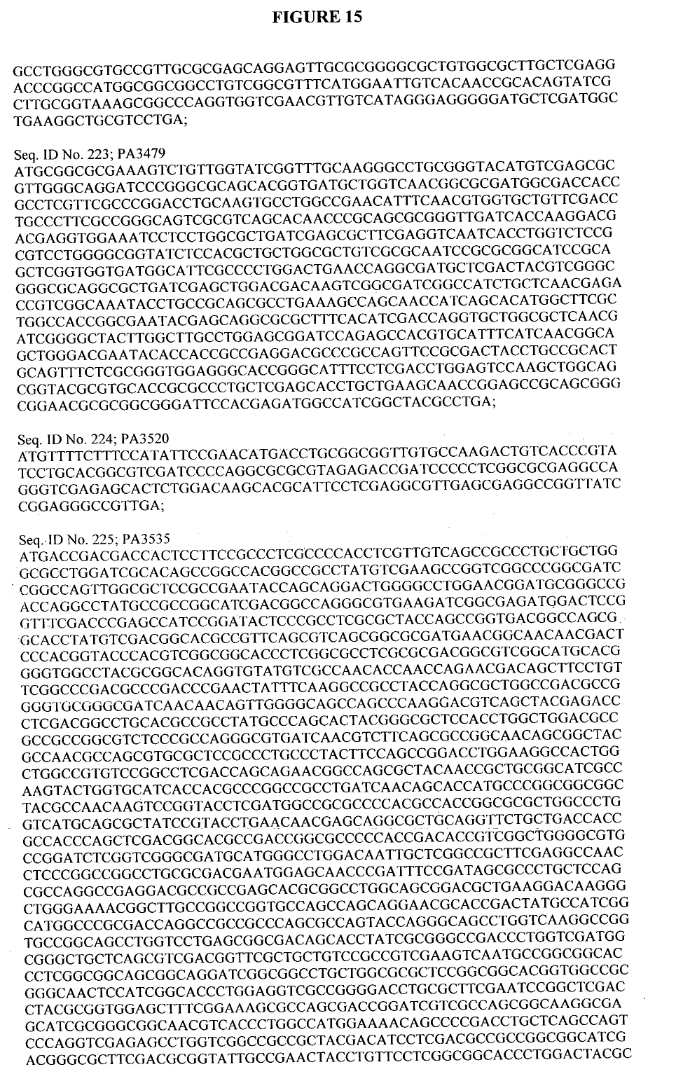

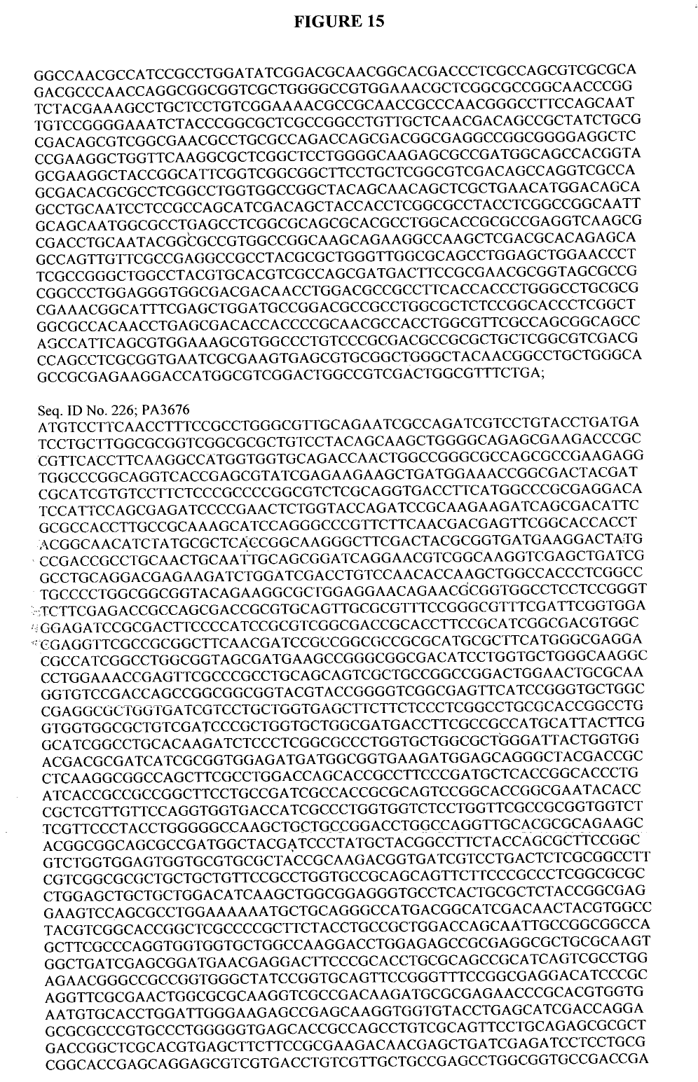

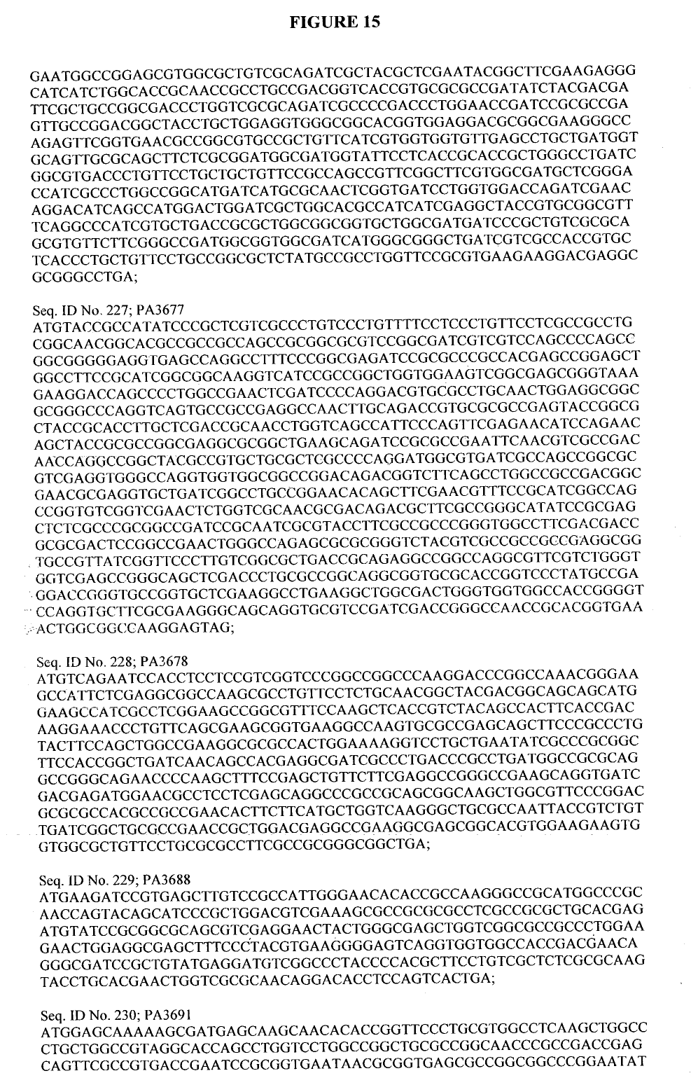

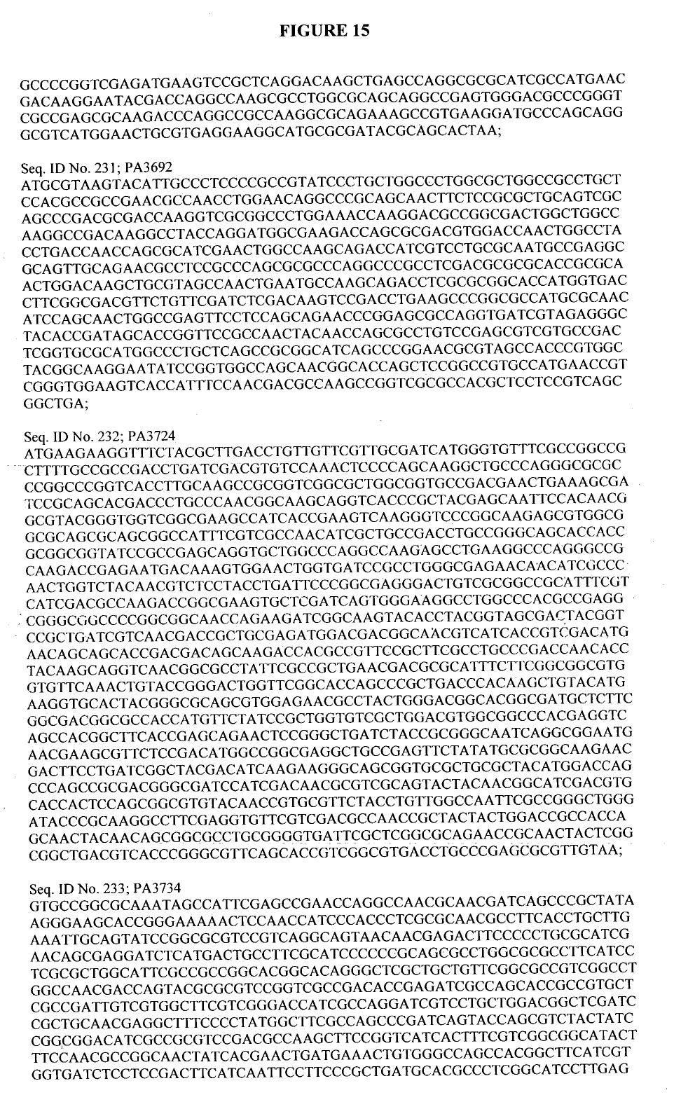

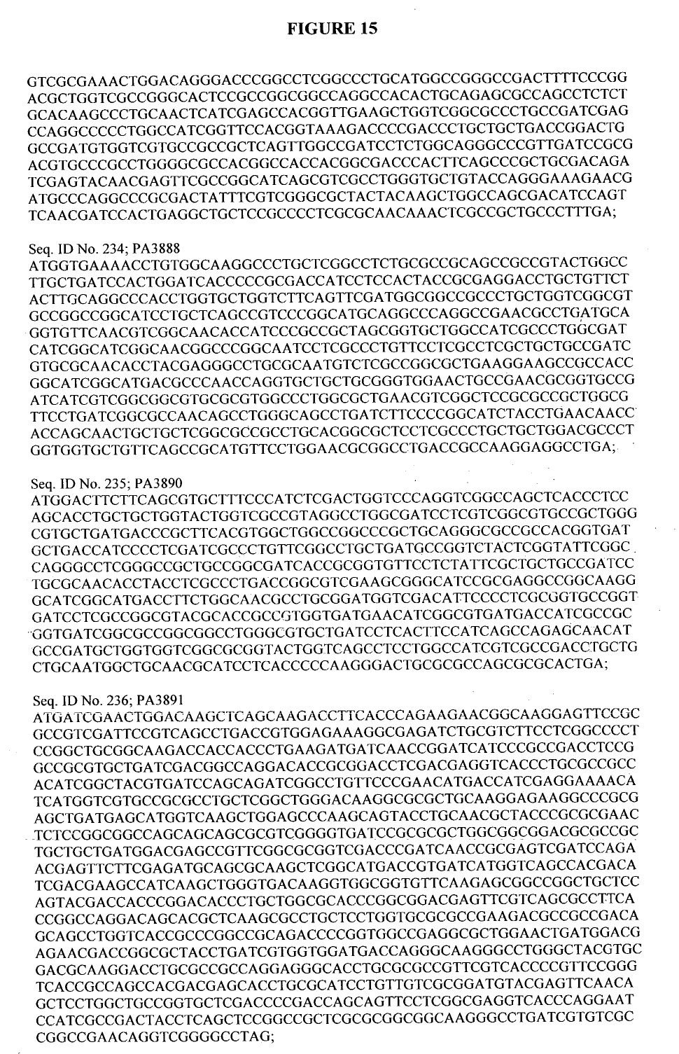

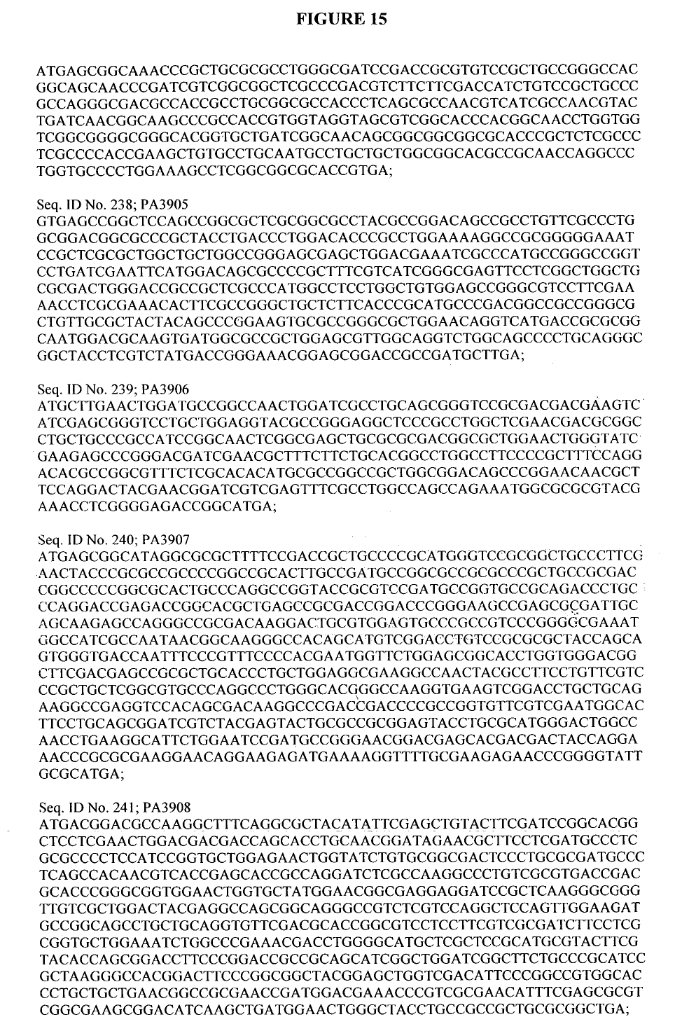

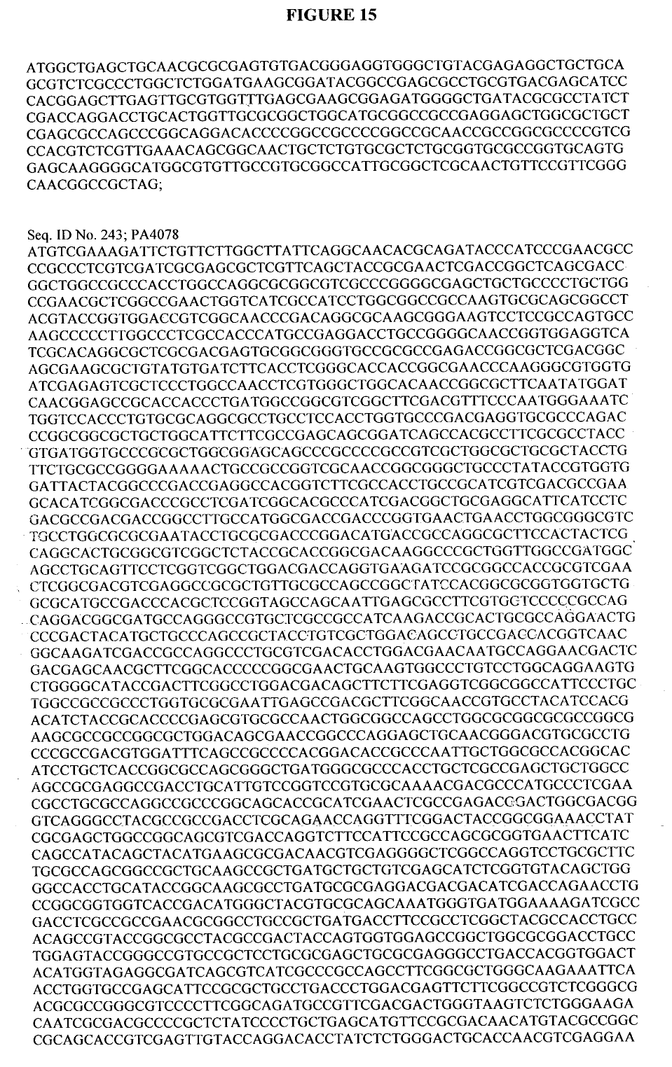

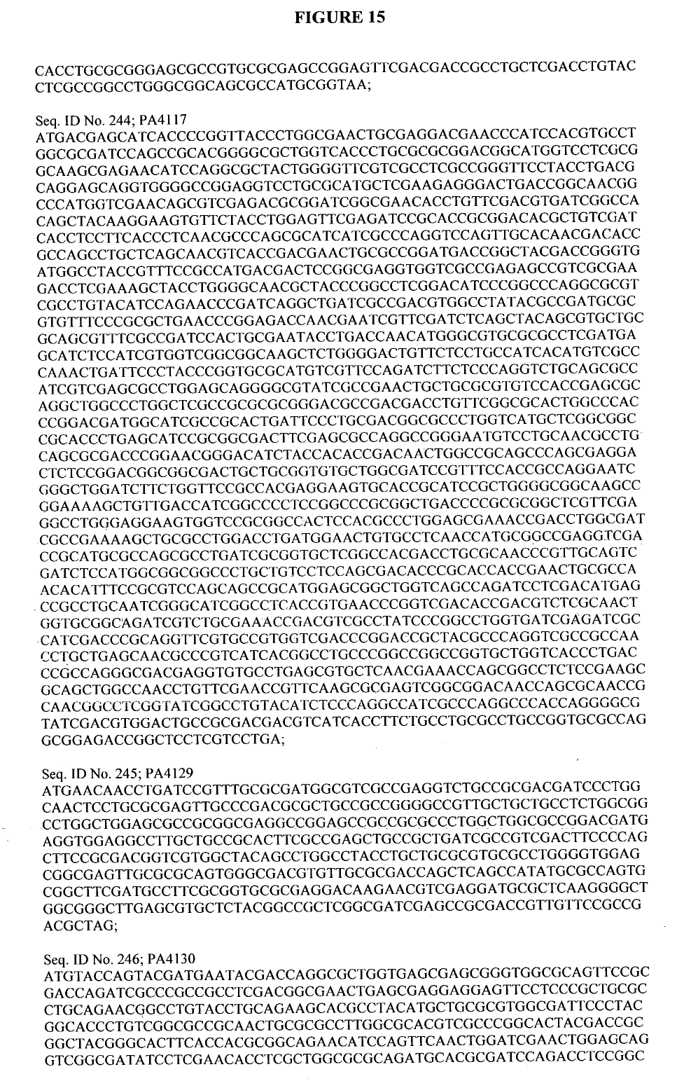

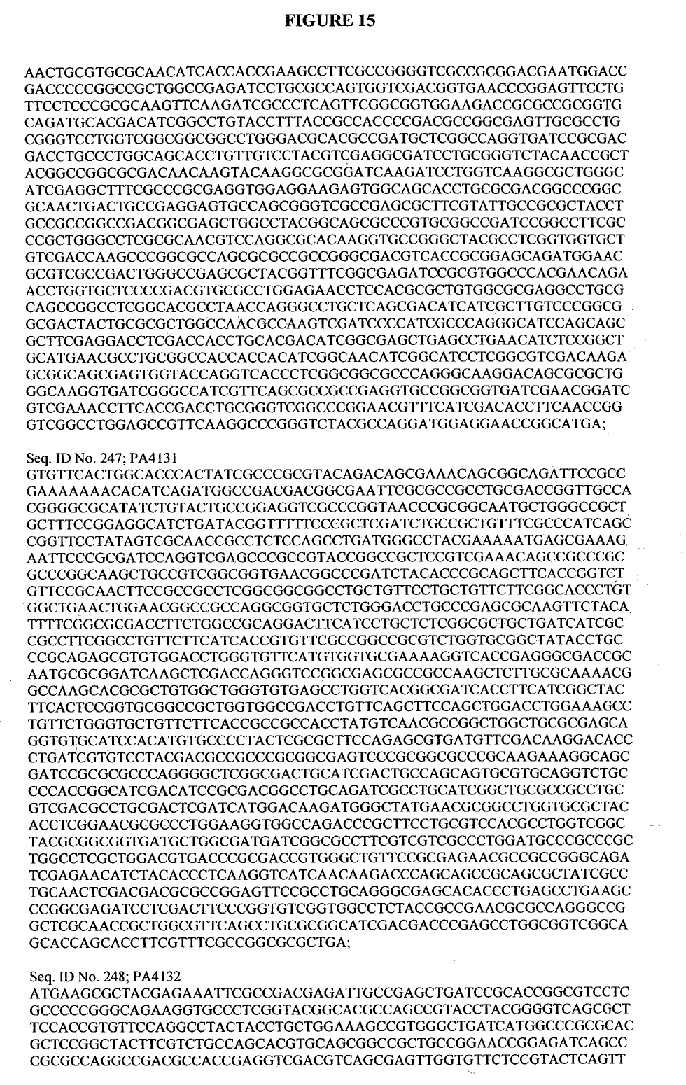

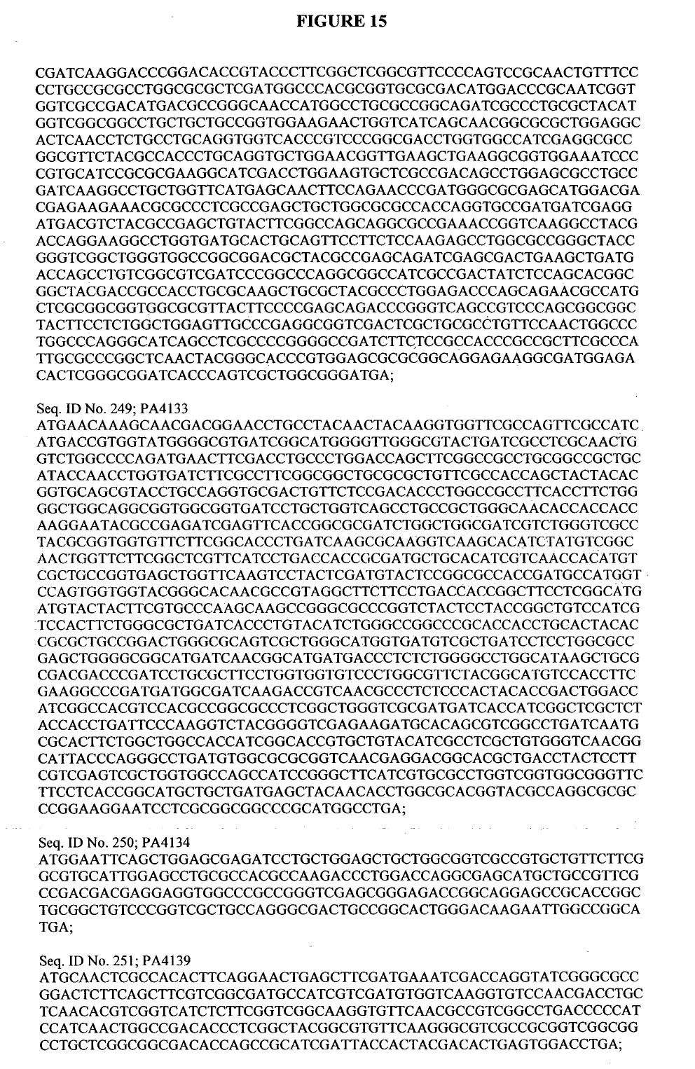

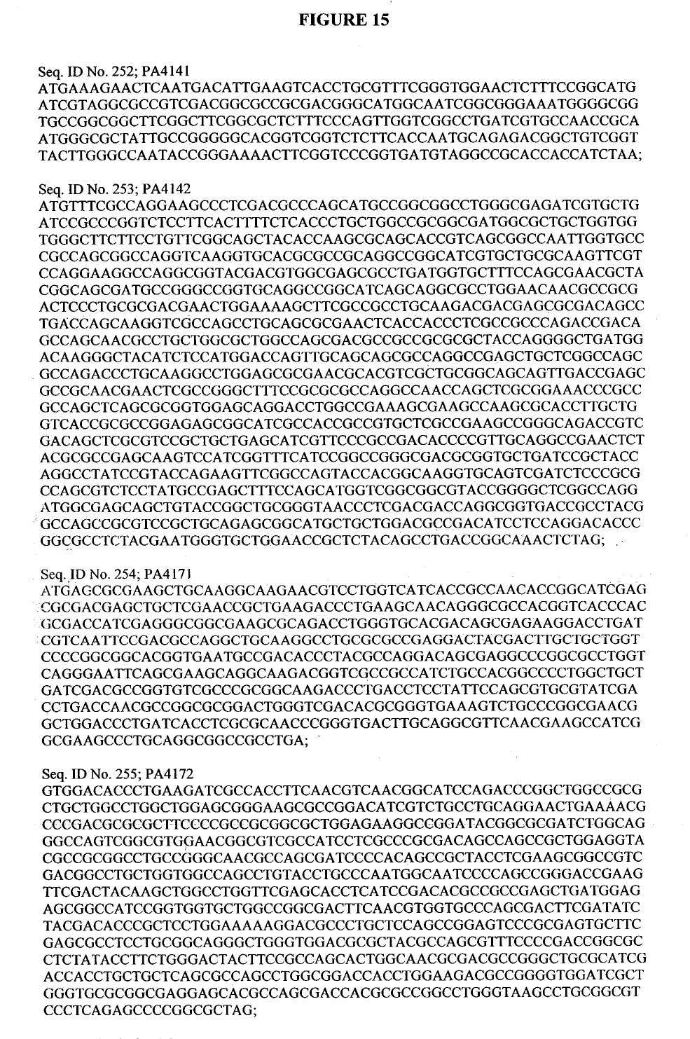

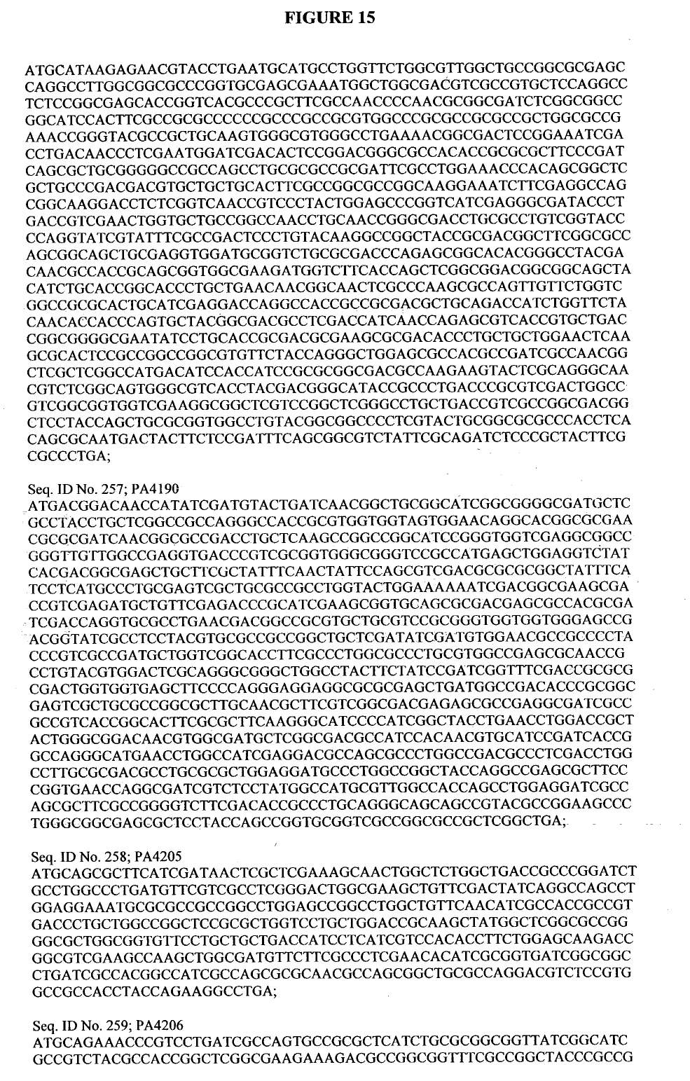

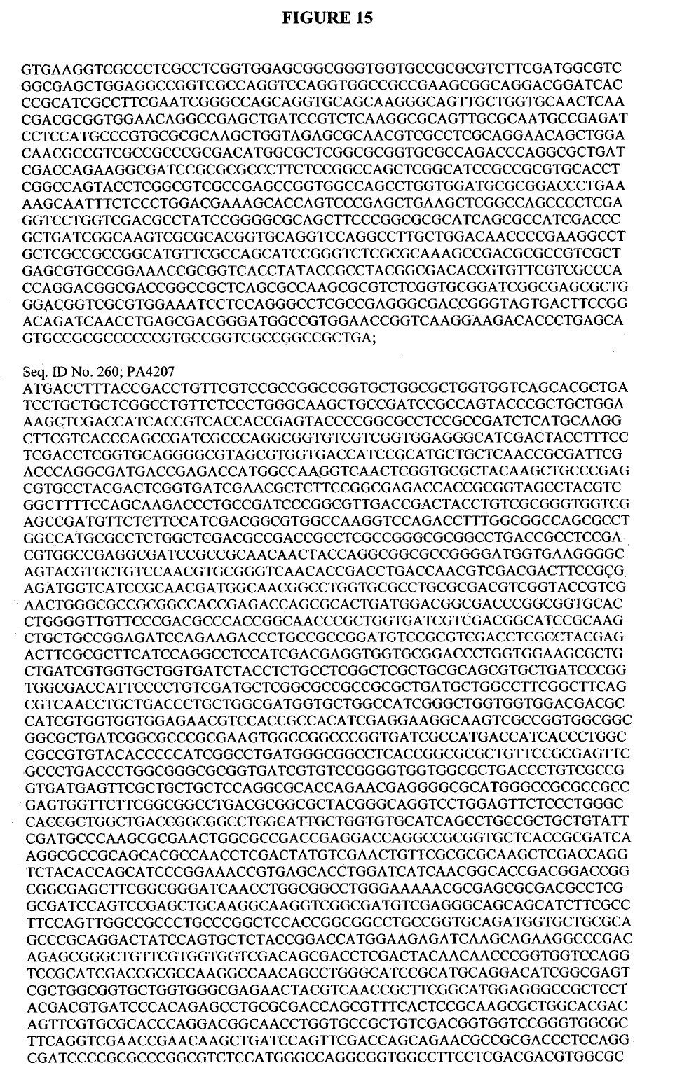

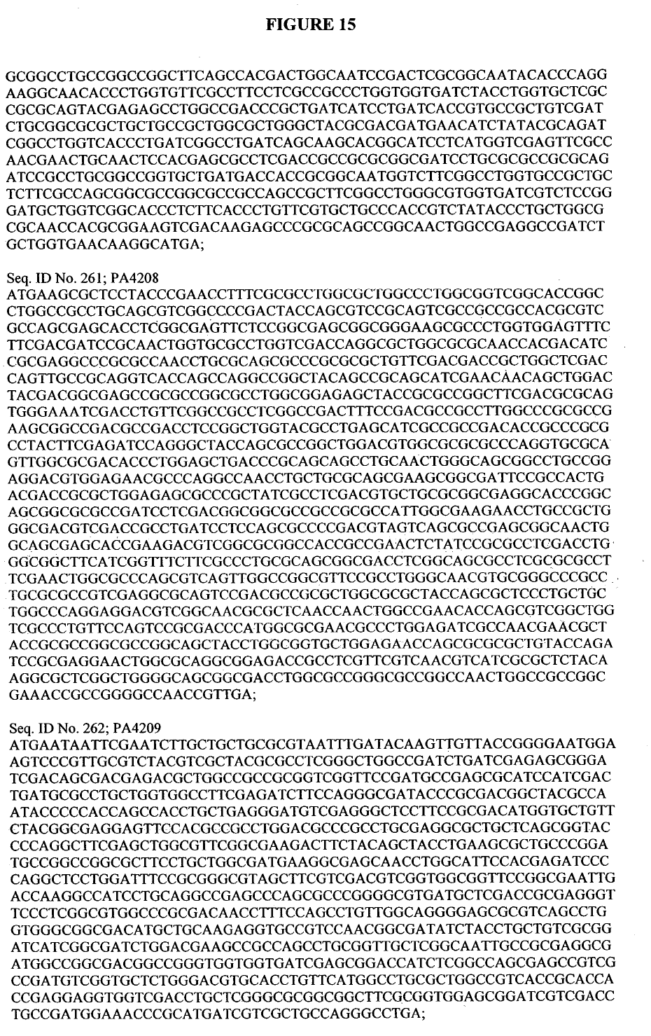

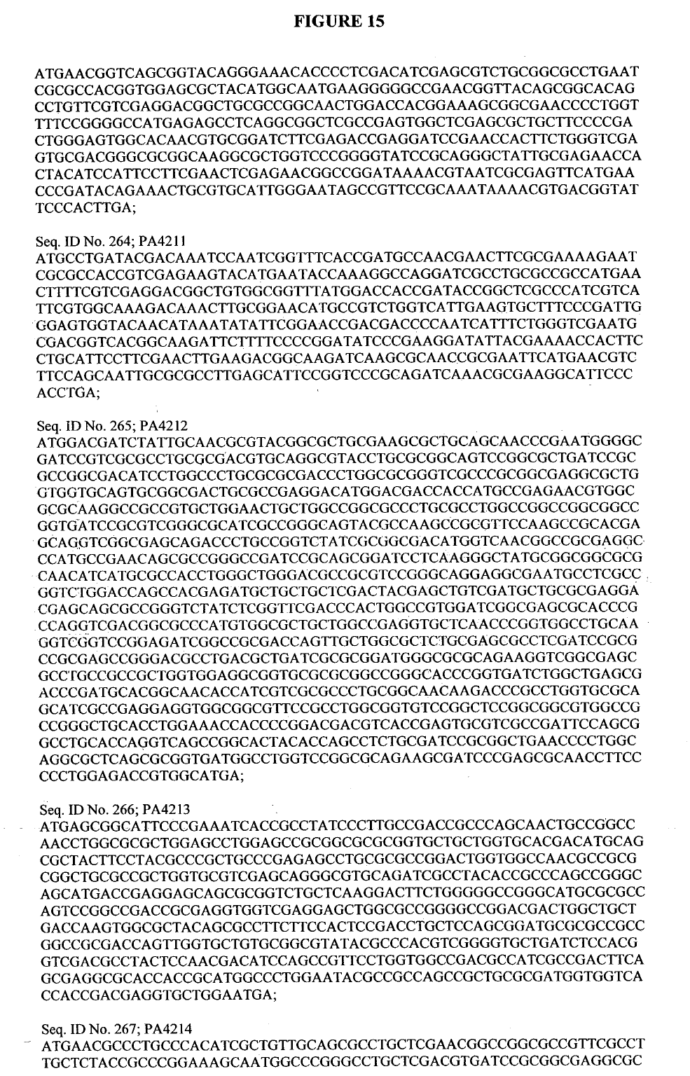

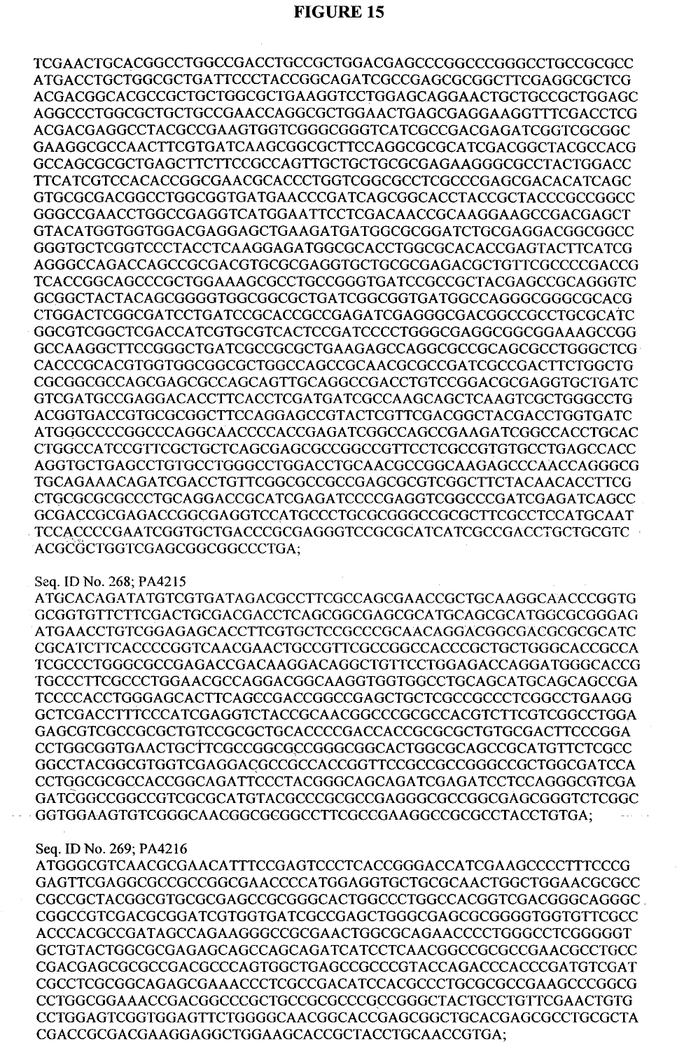

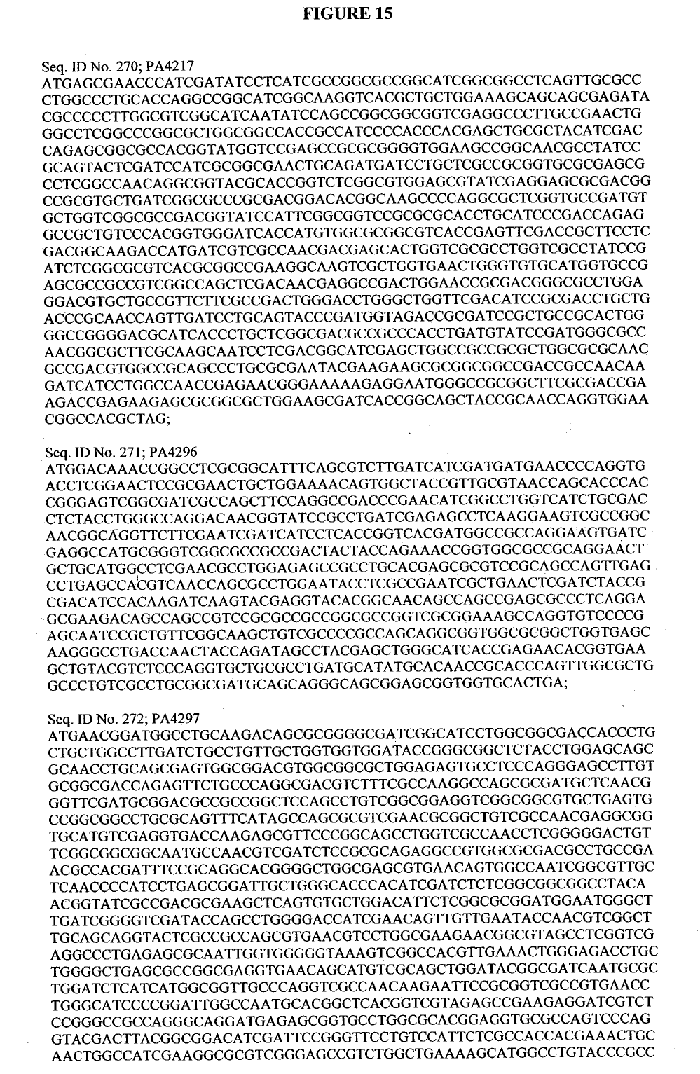

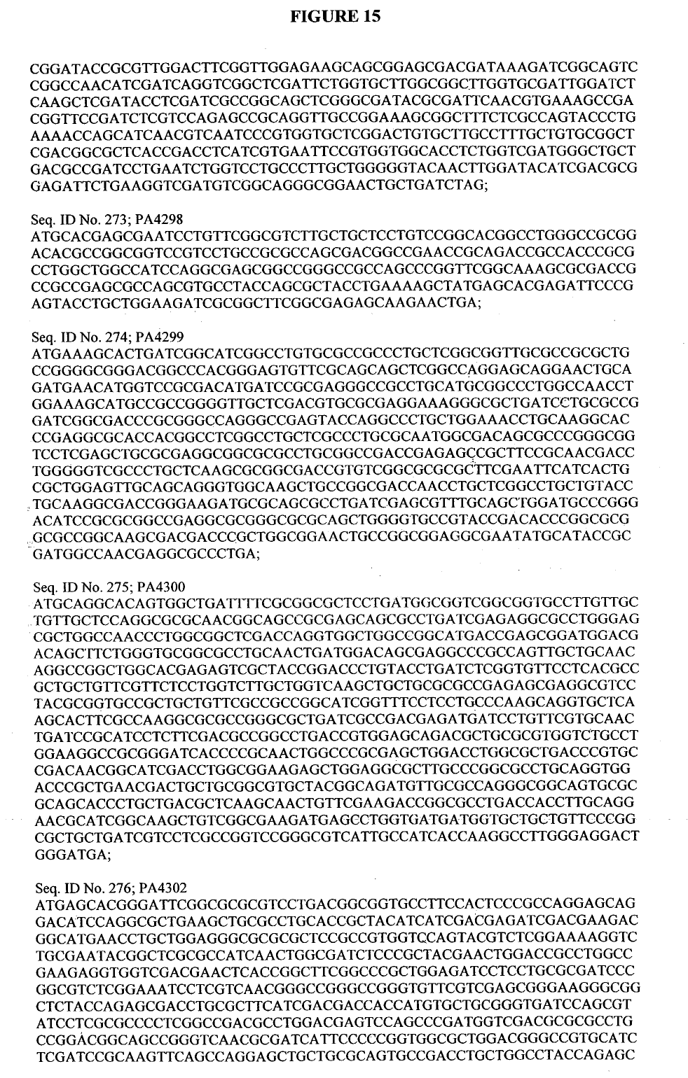

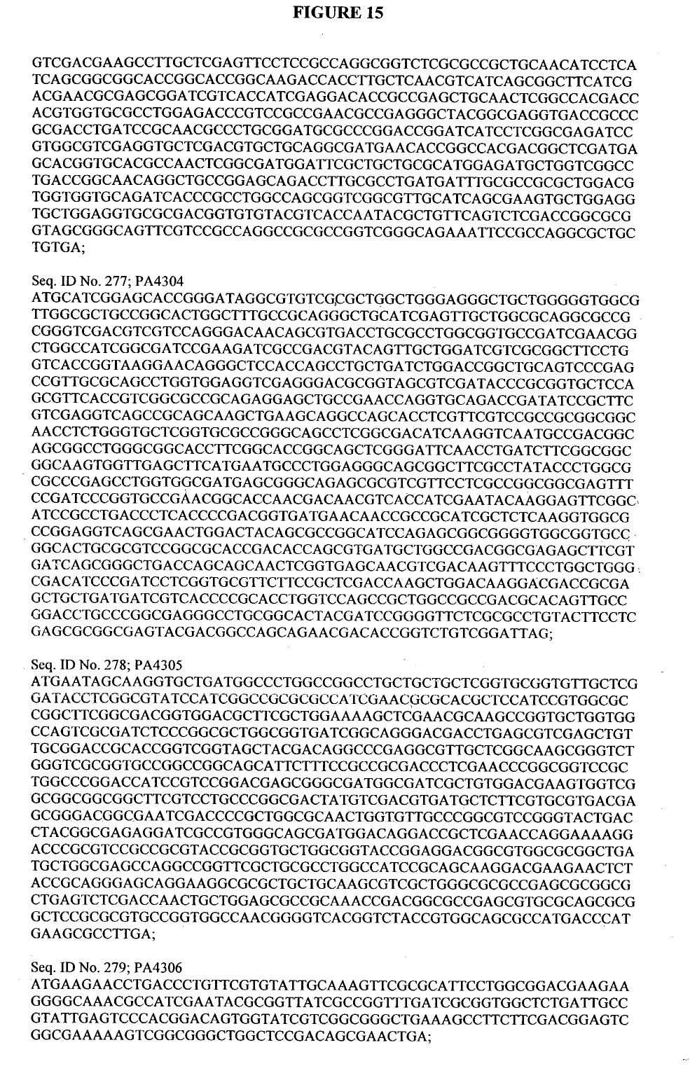

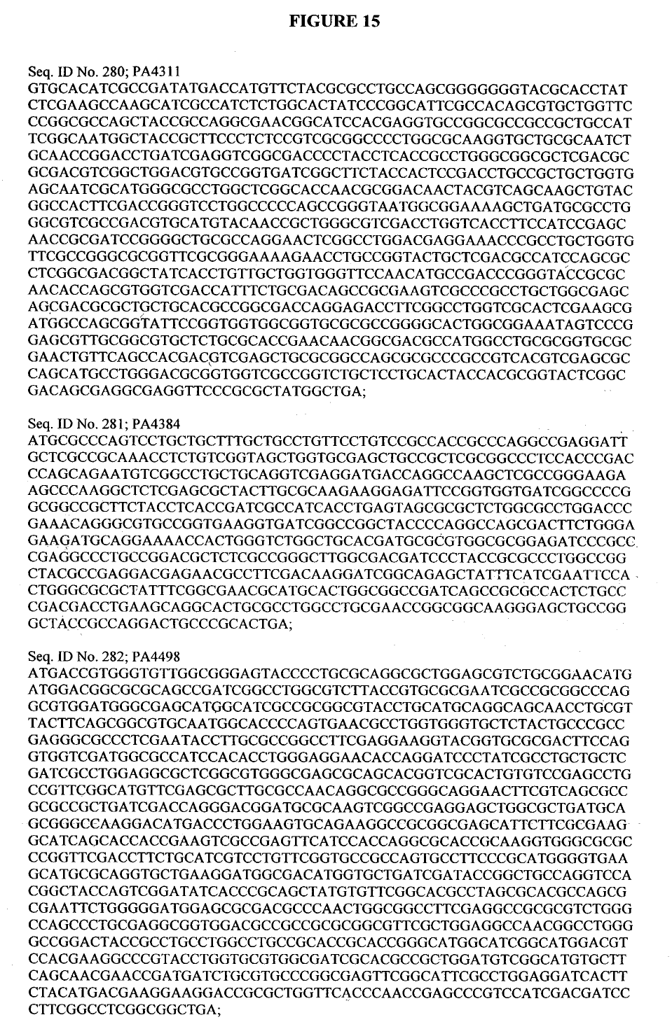

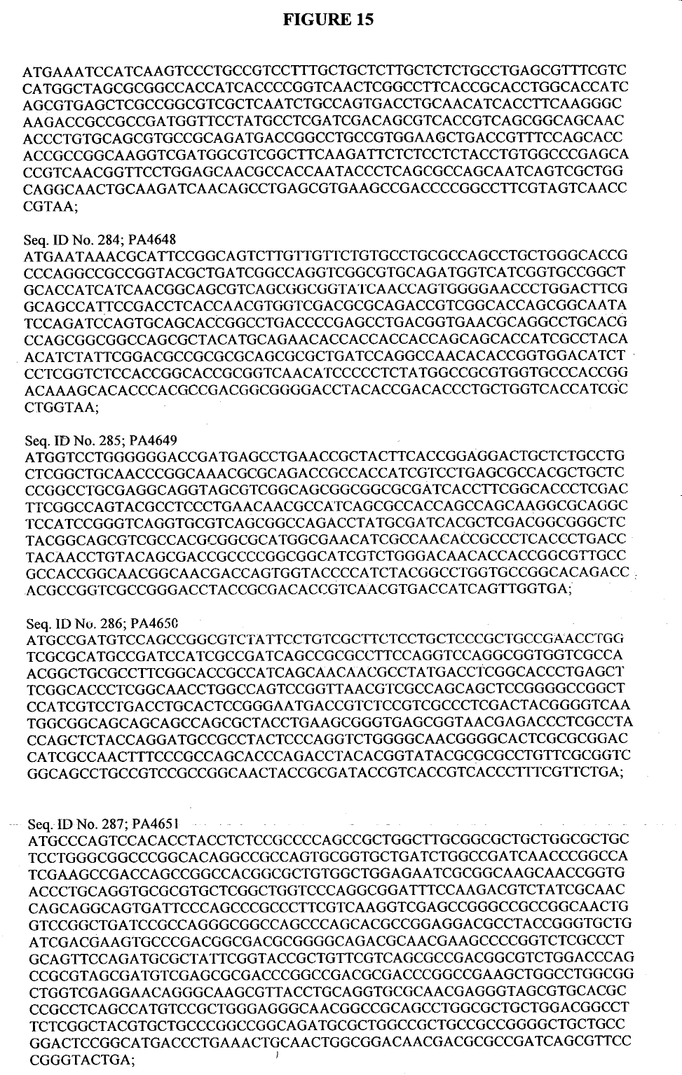

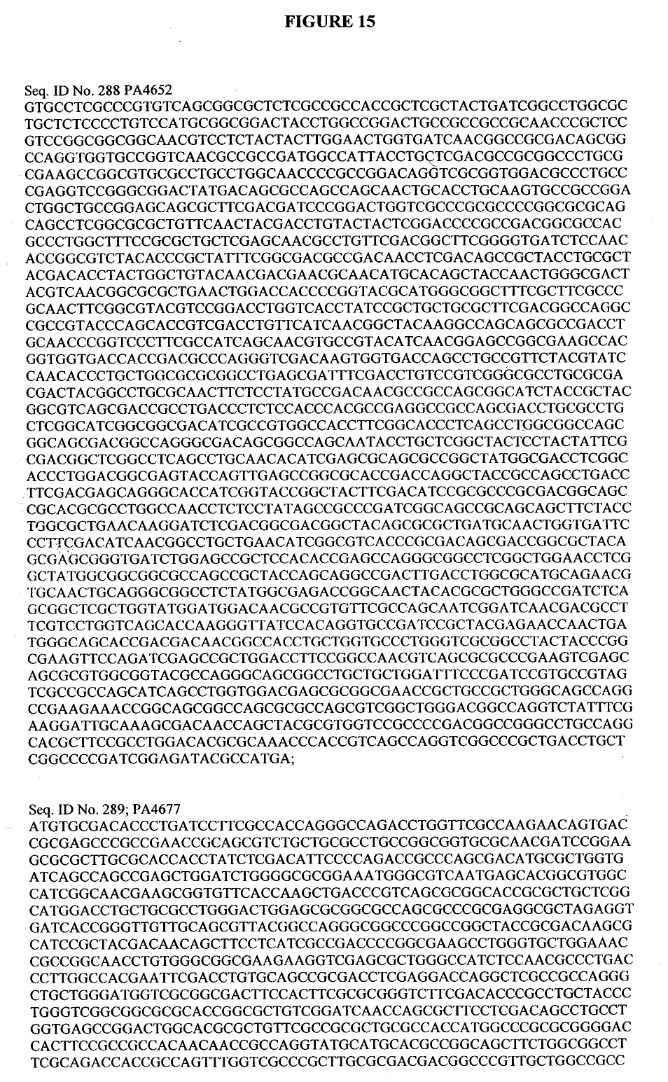

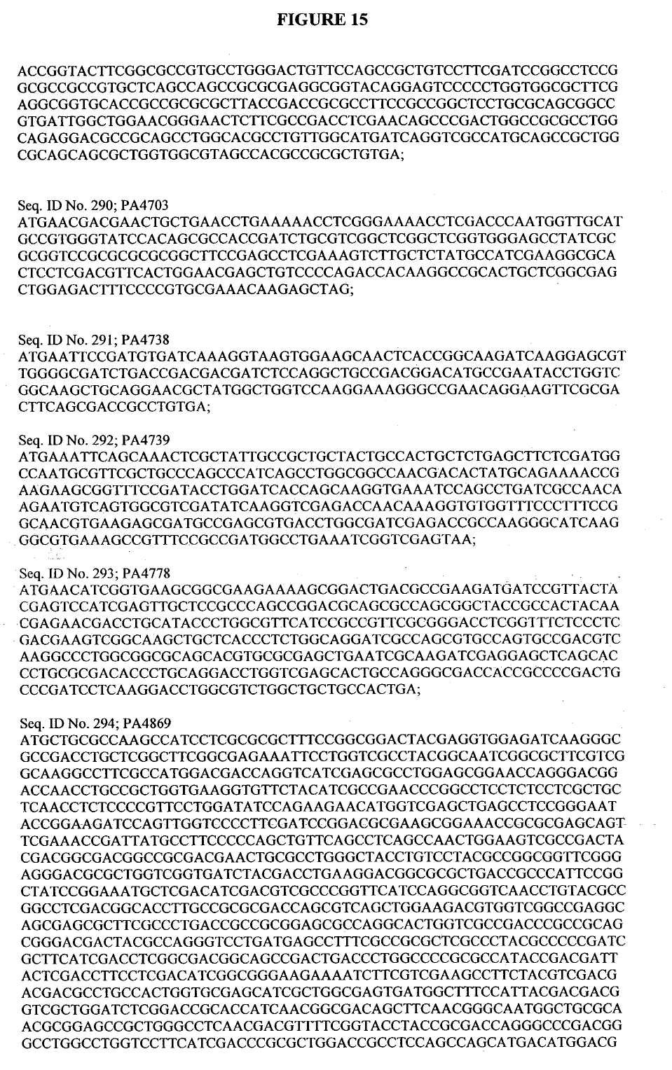

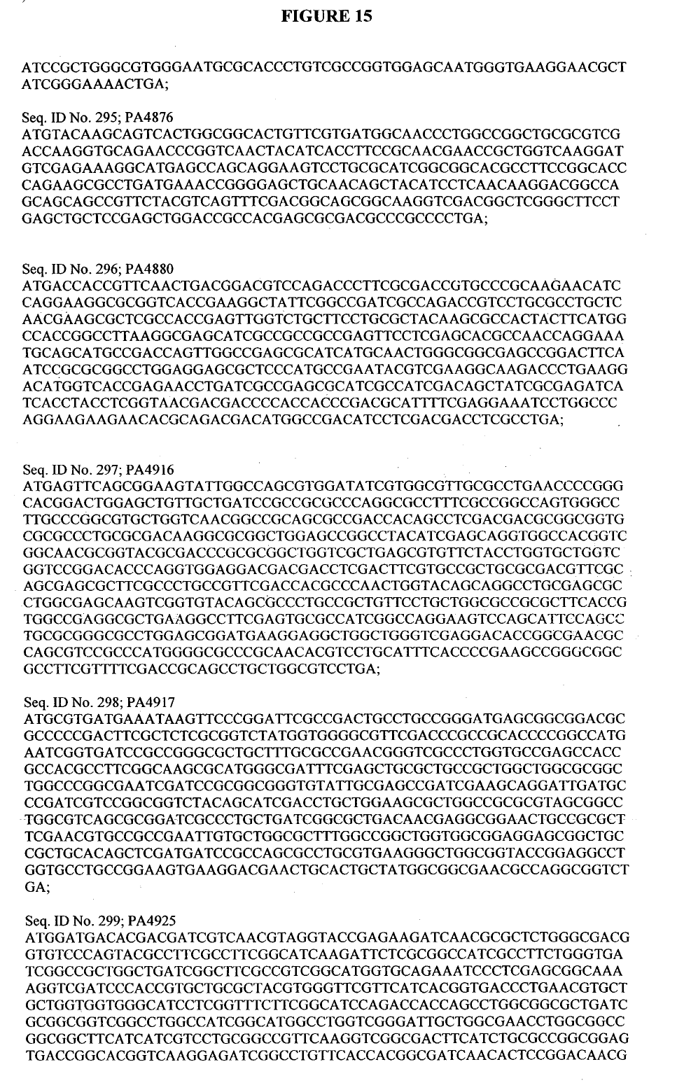

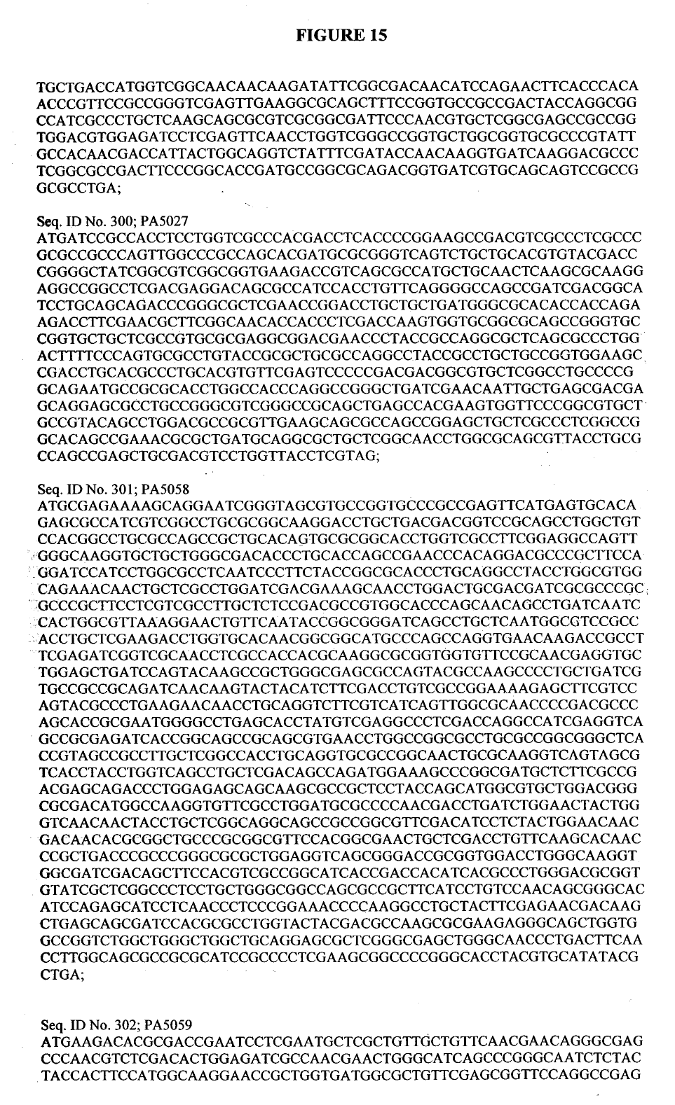

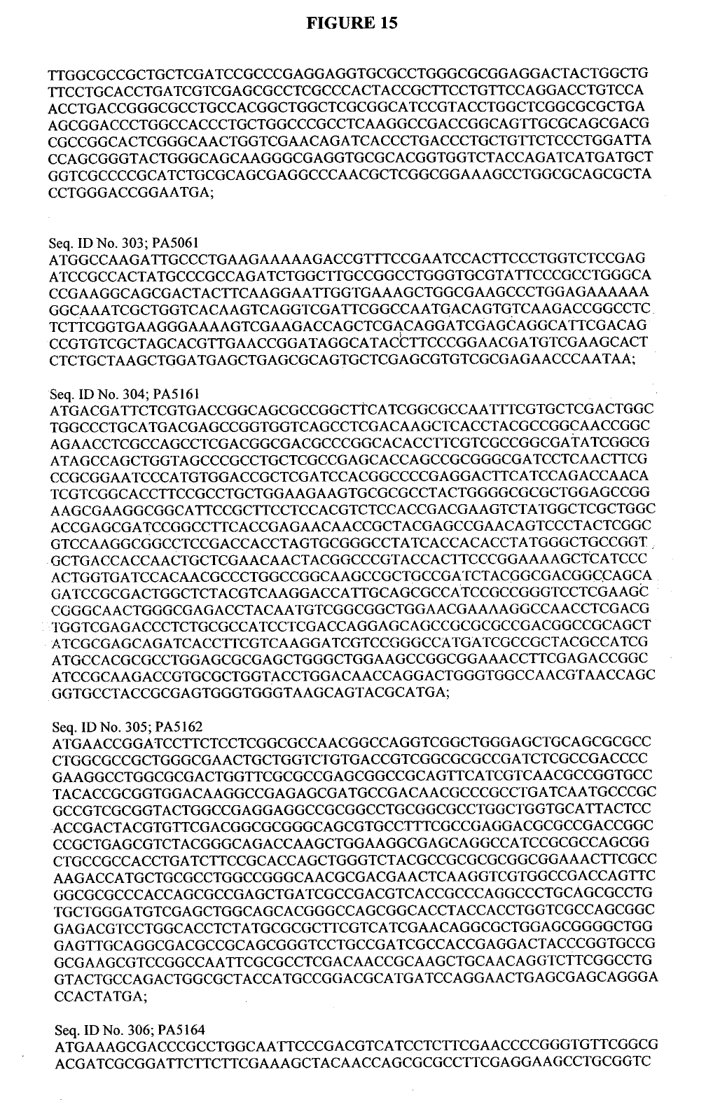

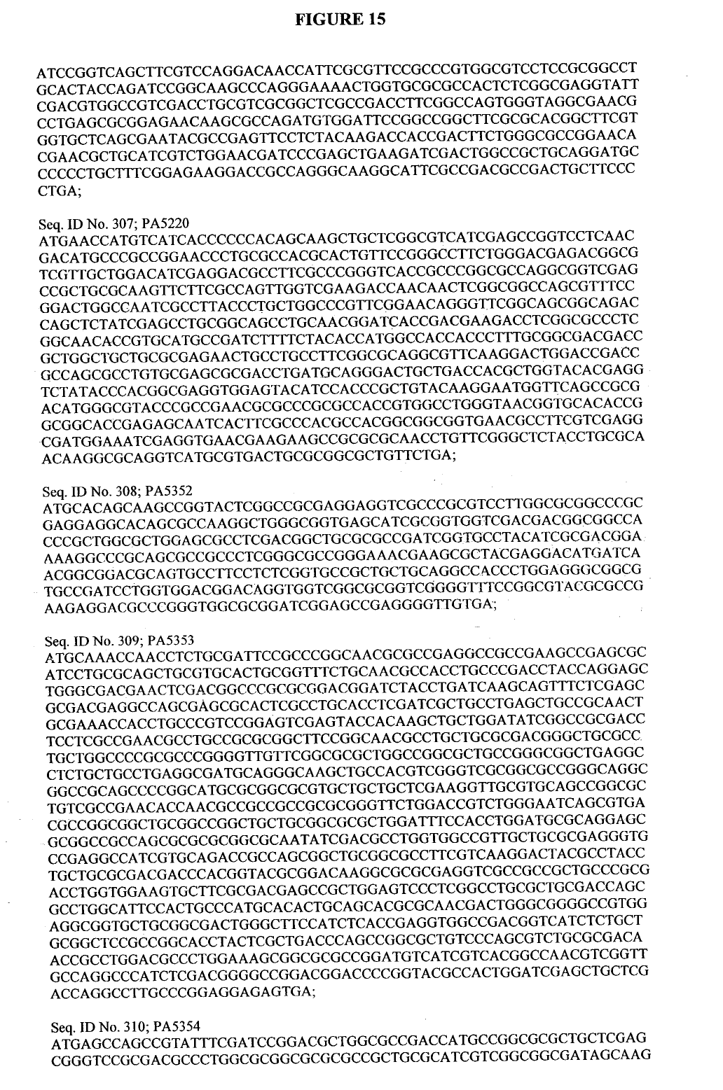

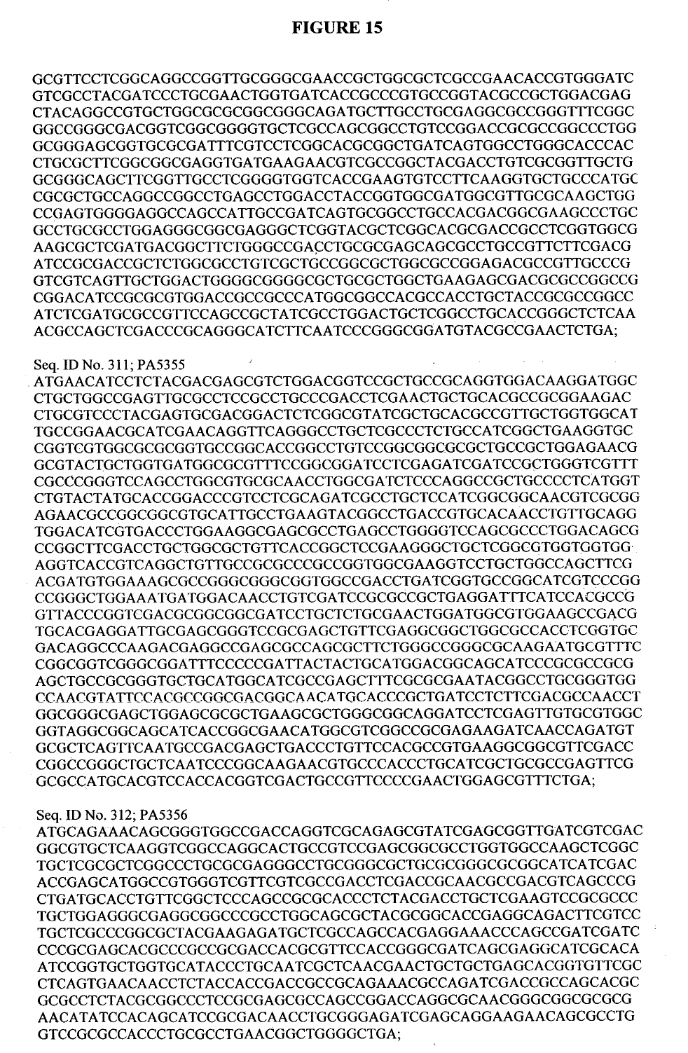

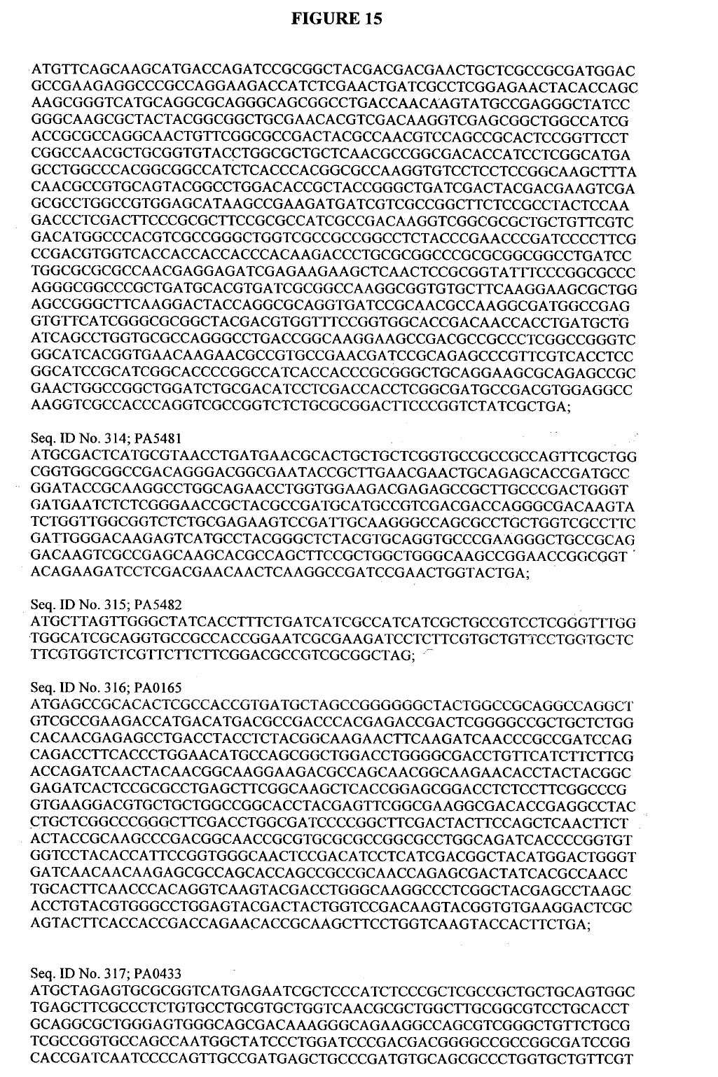

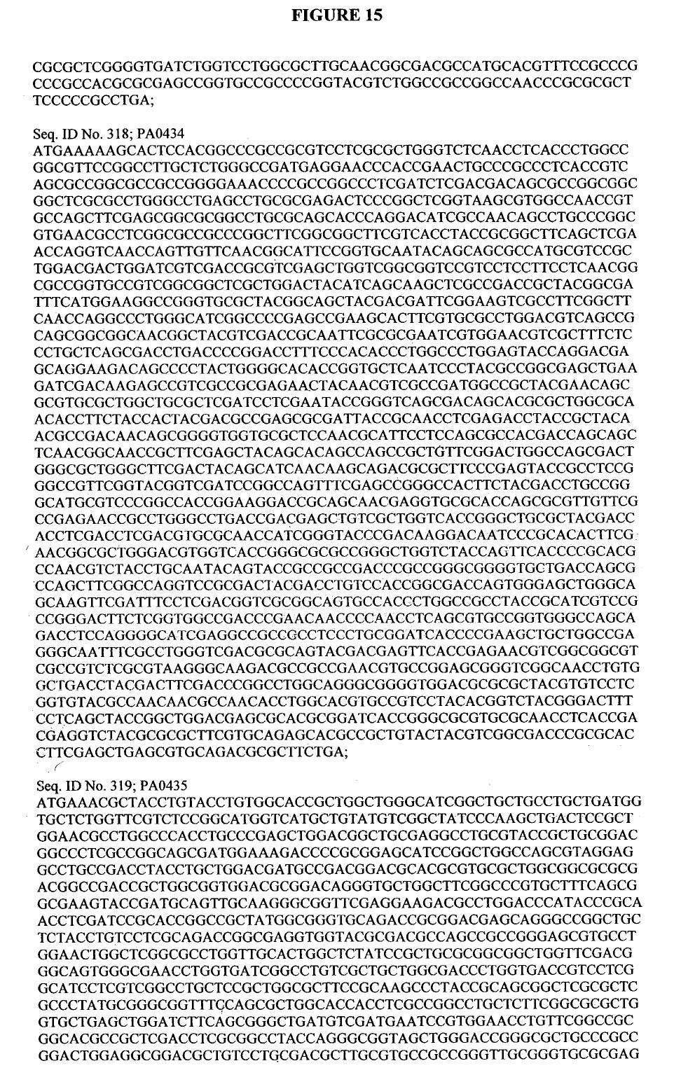

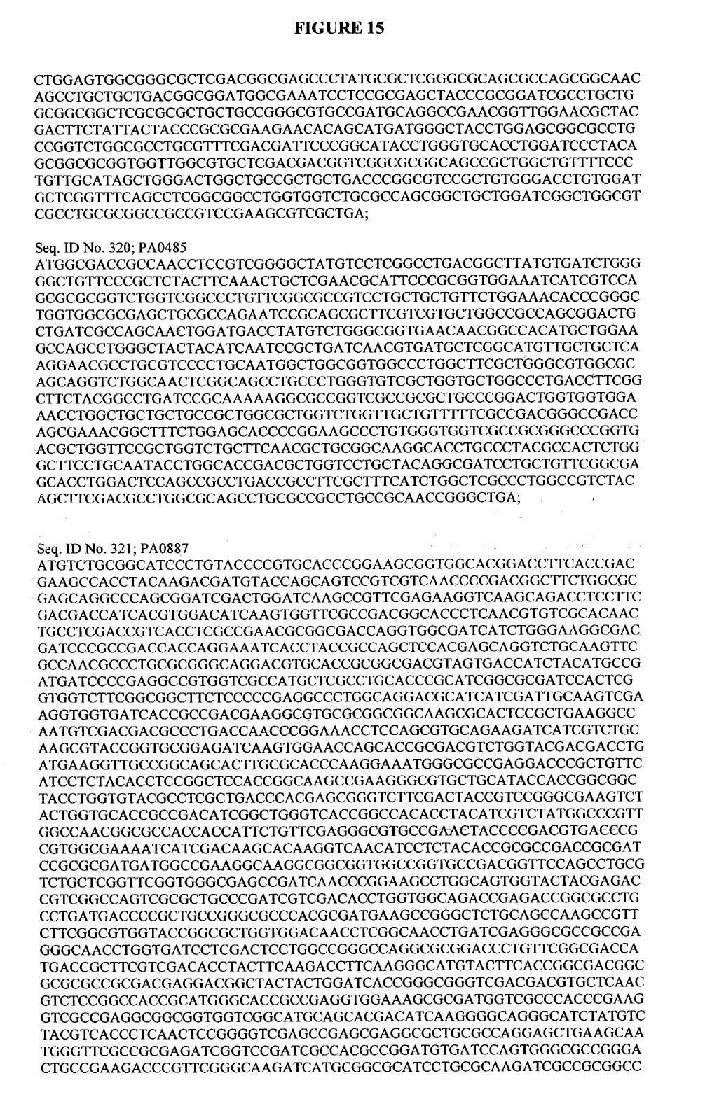

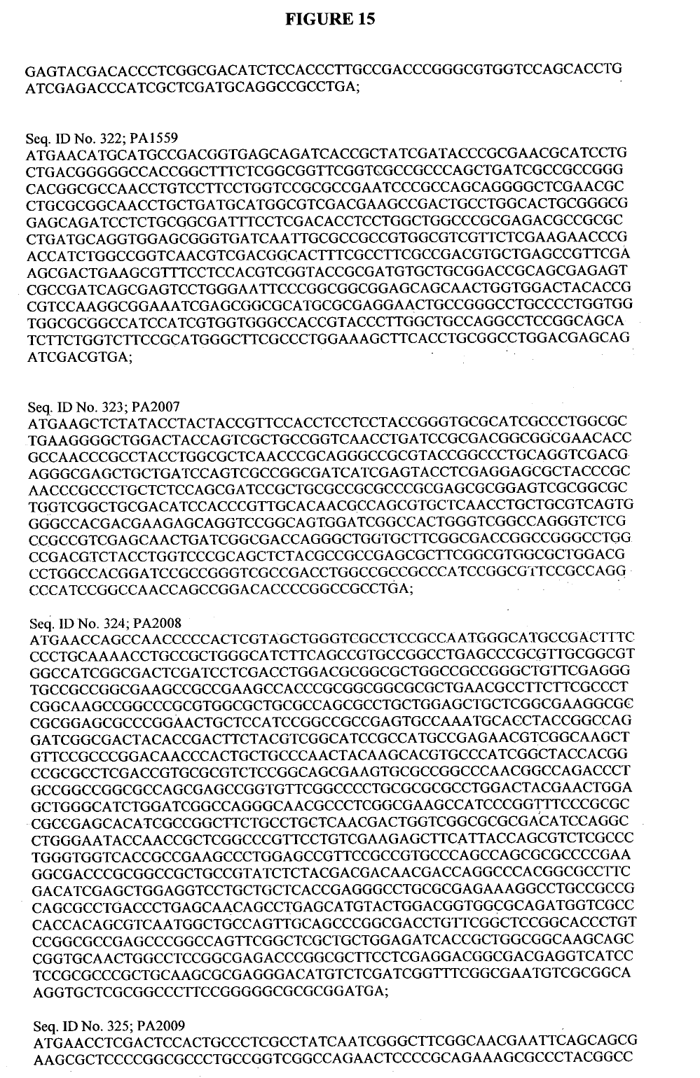

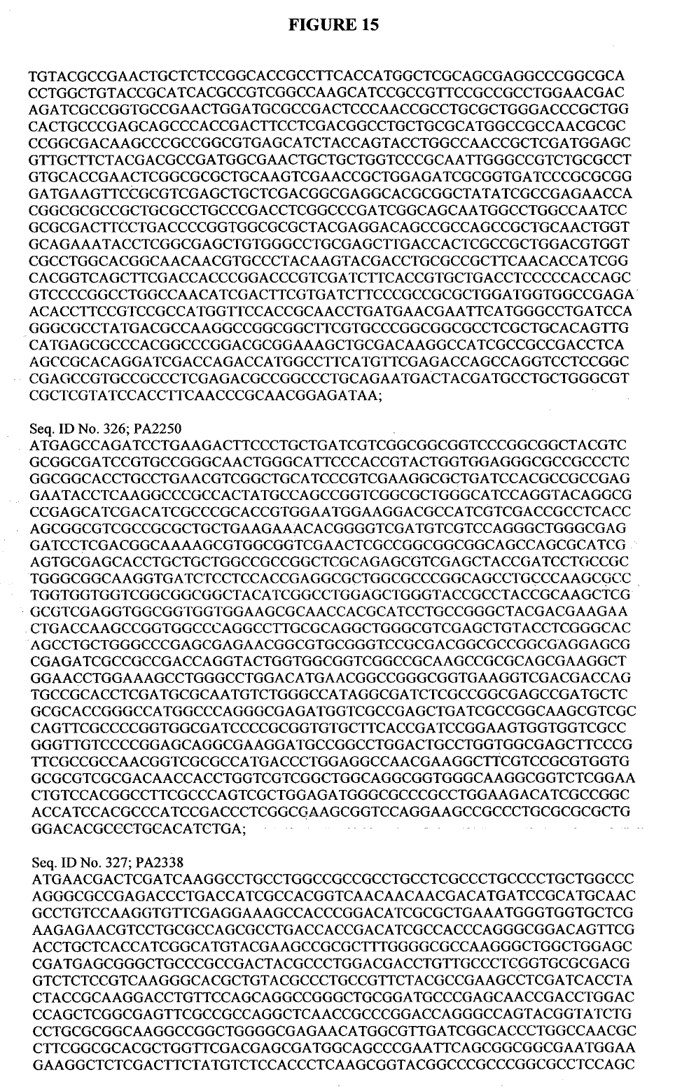

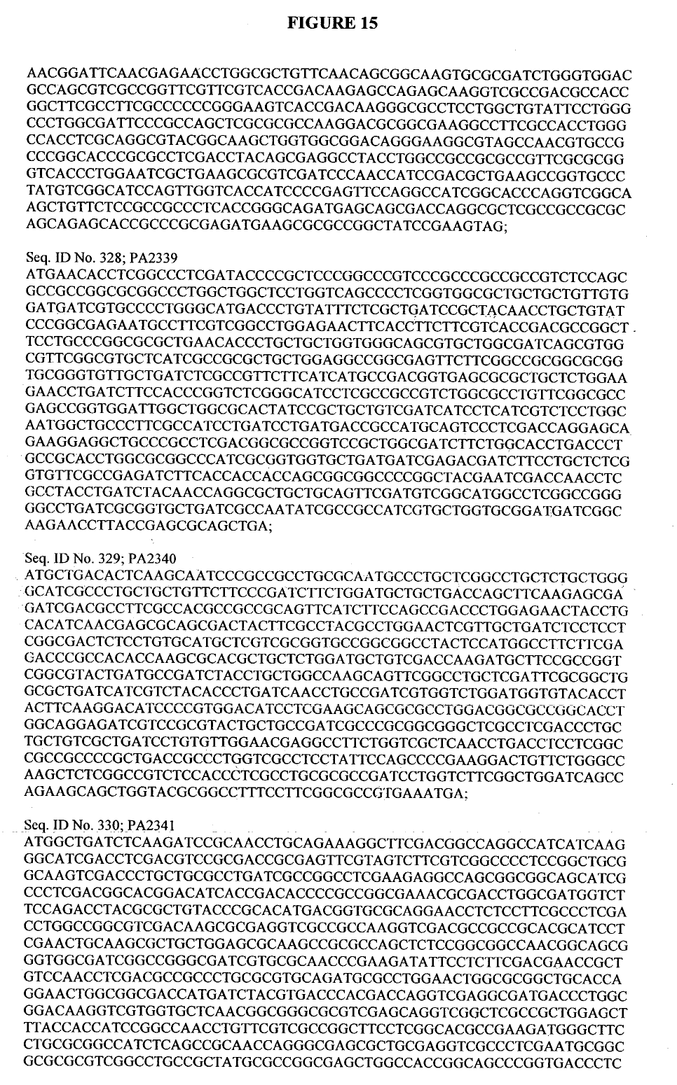

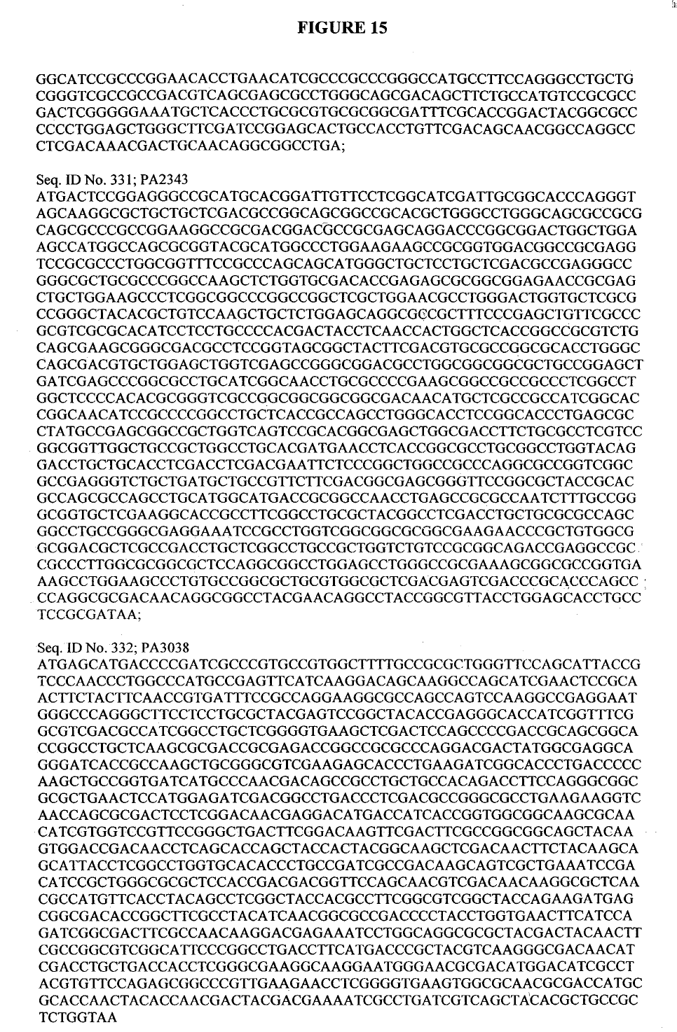

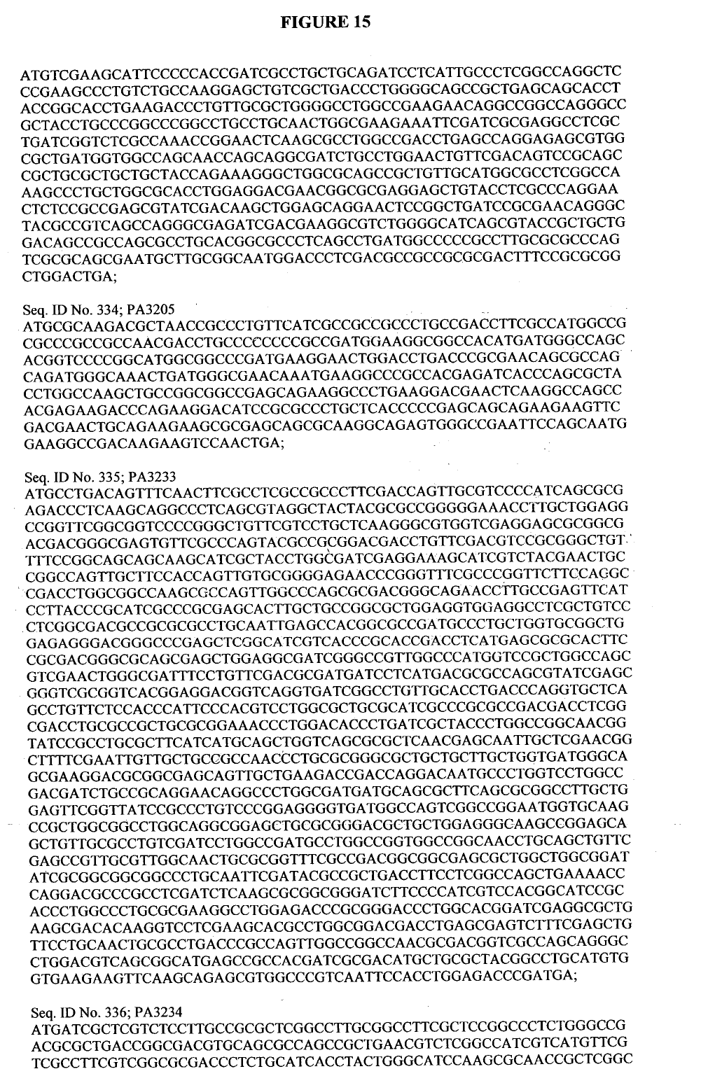

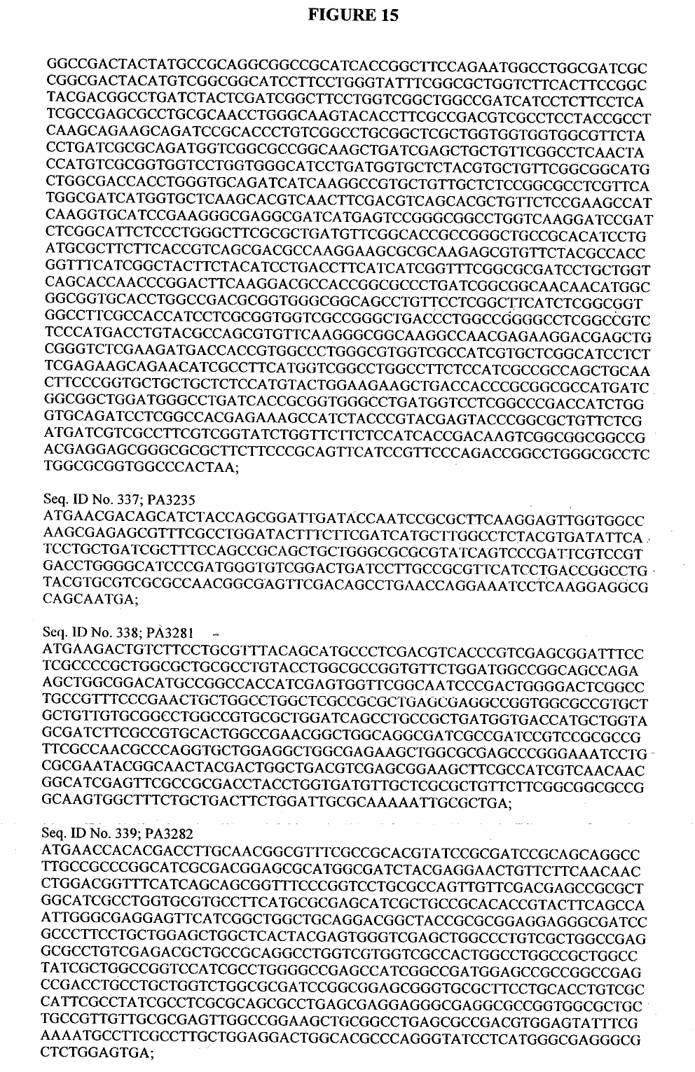

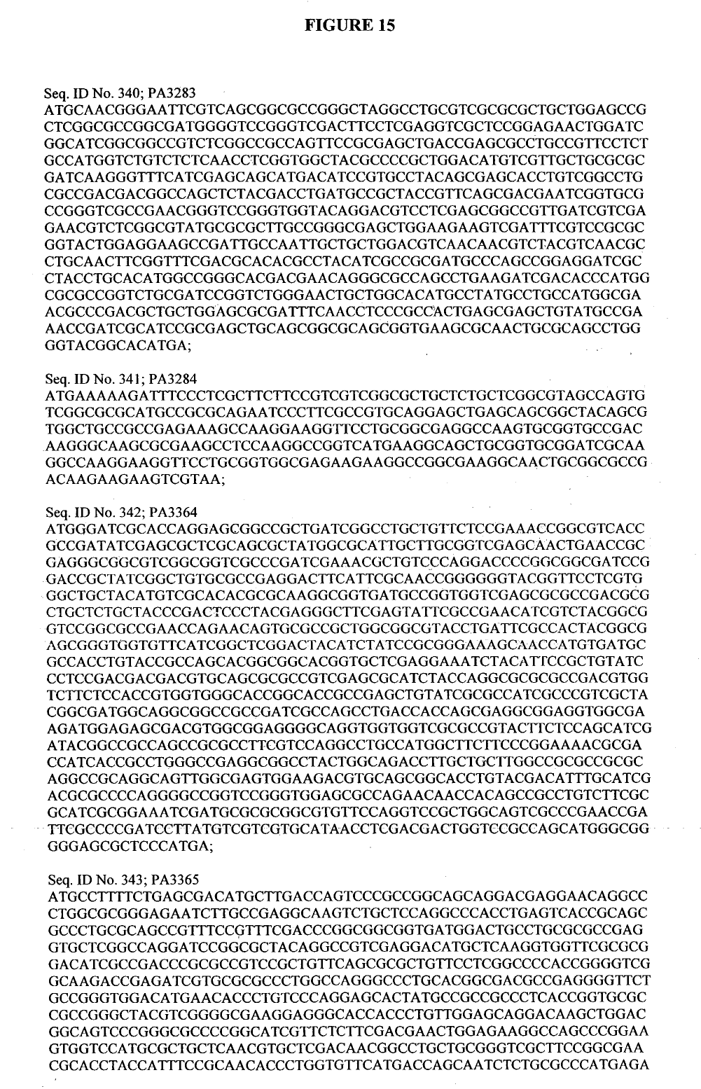

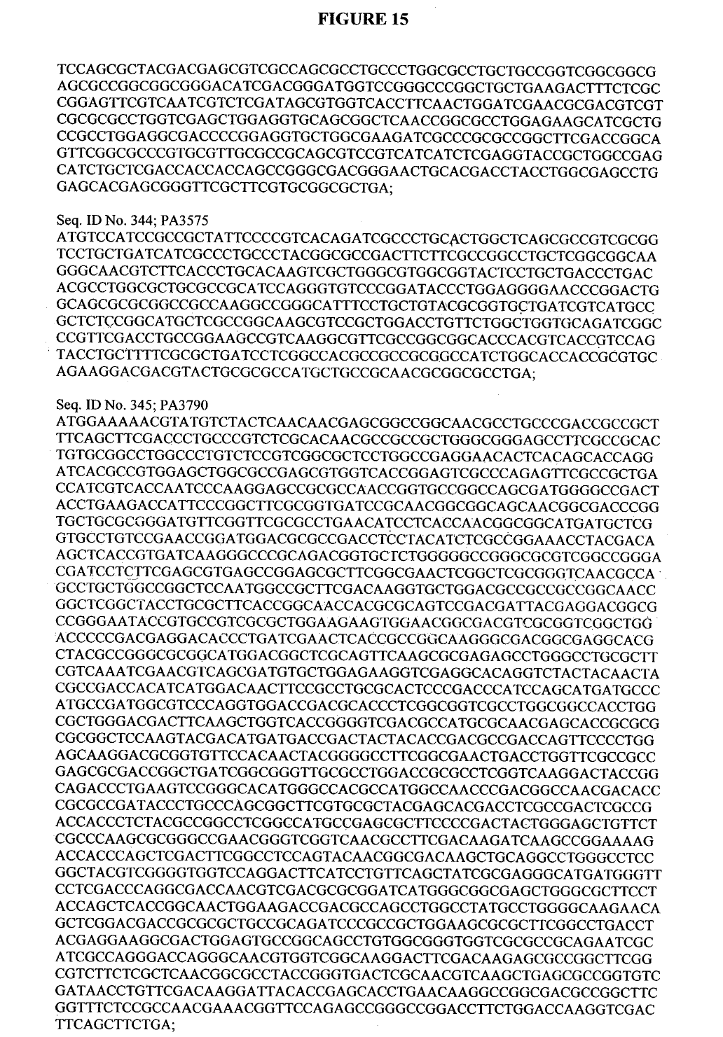

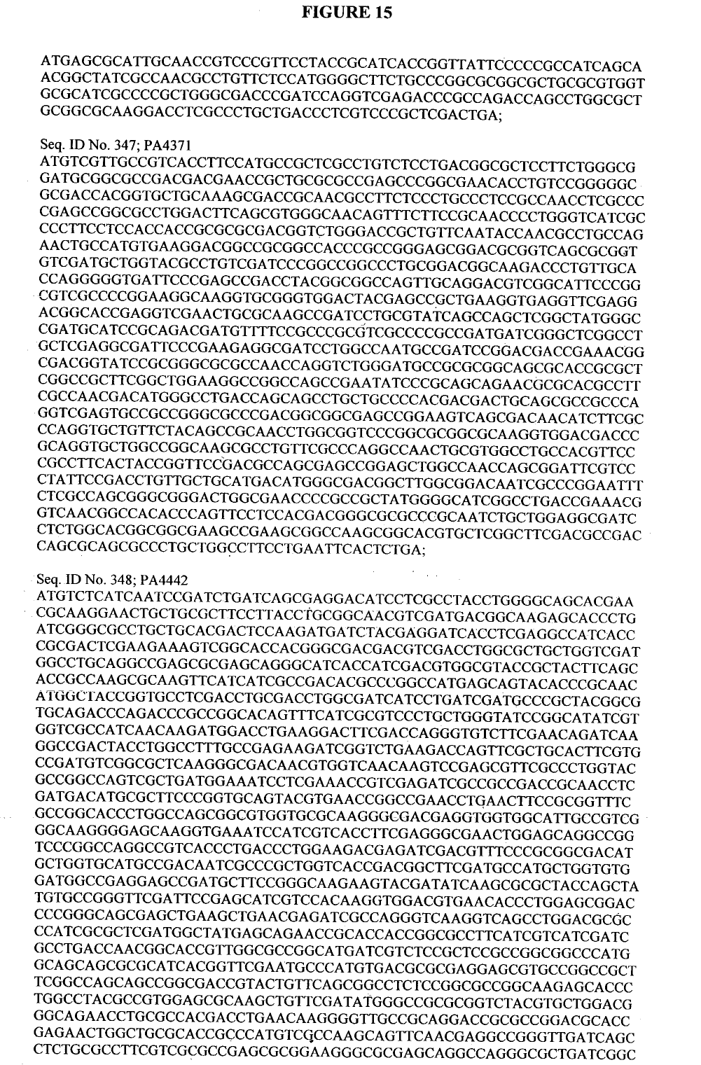

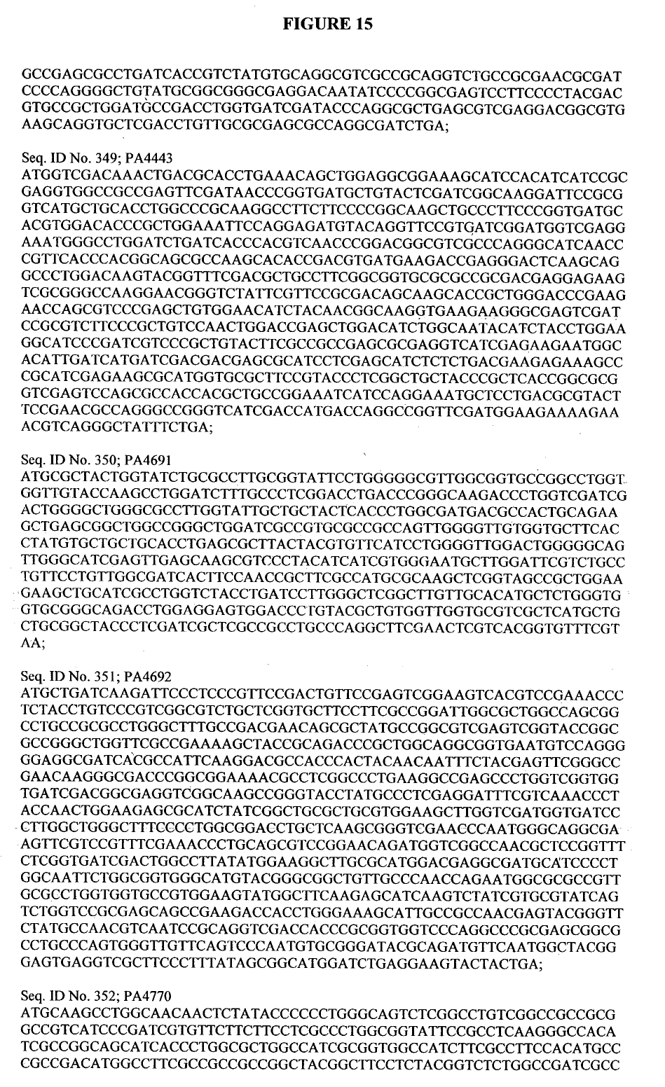

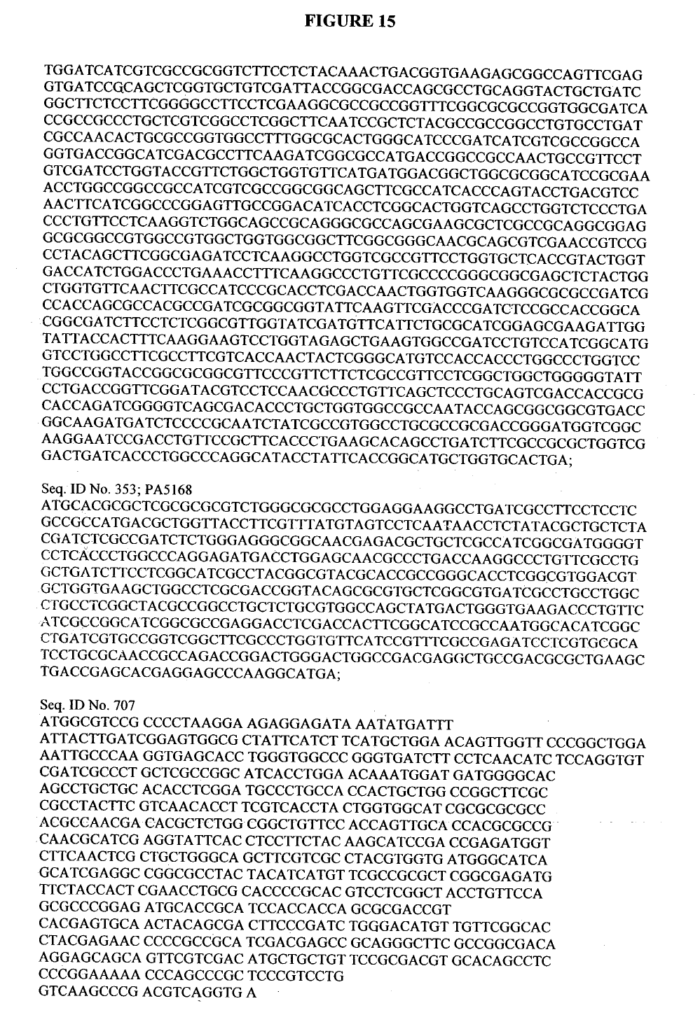

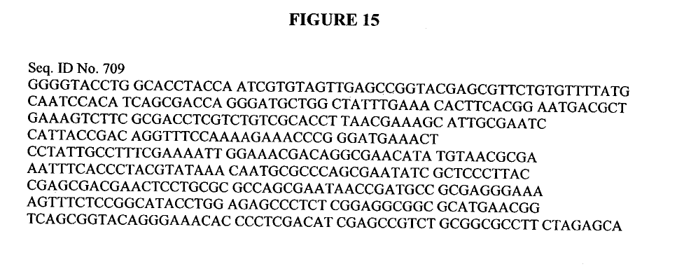

Each of the quorum sensing genes identified herein are listed in Tables 5 and 6, FIG. 15, and in the Sequence Listing. SEQ ID NOs:1-353 correspond to the quorum sensing controlled genes identified herein. SEQ ID NOs:354-706 correspond to the polypeptides encoded by SEQ ID NOs:1-353. The SEQ ID NOs listed in the Sequence Listing, FIG. 15, and referred to herein correspond to “PA” identification numbers. Using these identification numbers, the nucleotide and amino acid sequences of all of the genes and polypeptides listed in Tables 5 and 6, FIG. 15, and the Sequence Listing can be accessed through the Pseudomonas Genome Project.

Definitions

Before further description of the invention, certain terms employed in the specification, examples and appended claims are, for convenience, collected here.

The term “analog” as in “homoserine lactone analog” is intended to encompass compounds that are chemically and/or electronically similar but have different atoms, such as isosteres and isologs. An analog includes a compound with a structure similar to that of another compound but differing from it in respect to certain components or structural makeup. The term analog is also intended to encompass stereoisomers.

The language “autoinducer compounds” is art-recognized and is intended to include molecules, e.g., proteins which freely diffuse across cell membranes and which activate transcription of various factors which affect bacterial viability. Such compounds can affect virulence. and biofilm development. Autoinducer compounds can be acylated homoserine lactones. They can be other compounds similar to those listed in Table 1. Homoserine autoinducer compounds are produced in vivo by the interaction of a homoserine lactone substrate and an acylated acyl carrier protein in a reaction catalyzed by an autoinducer synthase molecule. In isolated form, autoinducer compounds can be obtained from naturally occurring proteins by purifying cellular extracts, or they can be chemically synthesized or recombinantly produced. The language “autoinducer synthase molecule” is intended to include molecules, e.g. proteins, which catalyze or facilitate the synthesis of autoinducer compounds, e.g in the quorum sensing system of bacteria. It is also intended to include active portions of the autoinducer synthase protein contained in the protein or in fragments or portions of the protein (e.g., a biologically active fragment). The language “active portions” is intended to include the portion of the autoinducer synthase protein which contains the homoserine lactone binding site.

Table 1 contains a list of exemplary autoinducer synthase proteins of the quorum sensing systems of various gram-negative bacteria.

TABLE 1
Summary of N-acyl homoserine lactone based regulatory systems

| Bacterial | | Regulatory | Target |
|---|---|---|---|
| species | Signal molecules^{a} | Proteins^{b} | function(s) |
| Vibrio fischeri | N-3-(oxohexanoyl)- | LuxI/LuxR | luxlCDABEG, |
| | homoserine lactone | | luxR |
| | (VAI-1) | | luminescence |
| | N-(octanoyl)-L- | AinS/AinR^{c} | luxlCDABEG,? |
| | homoserine lactone | | |
| | (VAI-2) | | |
| Vibrio harveyi | N-β- | LuxM/LuxN- | luxlCDABEG, |
| | (hydroxybutyryl)- | LuxO-LuxR^{d} | luminescence |
| | homoserine lactone | | and |
| | (HAI-1) | | polyhydroxy- |
| | HAI-2 | Lux?/LuxPQ- | butyrate |
| | | LuxO-LuxR^{d} | synthesis |
| | | | luxCDABEG |
| Pseudomonas | N-3- | LasI/LasR | lasB, lasA, |
| aeruginosa | (oxododecanyoyl)- | | aprA, toxA, |
| | L-homoserine | | virulence |
| | lactone | RhII/RhIR | factors |
| | (PAI-I) | | rhlAB, |
| | N-(butyryl)-L- | | rhamnolipid |
| | homoserine lactone | | synthesis, |
| | (PAI-2) | | virulence |
| | | | factors |
| Pseudomonas | (PRAI)^{e} | PhzI/PhzR | phz, phenazine |
| aeureofaciens | | | biosynthesis |
| Agroacterium | N-3-(oxooctanoyl)- | Tral/TraR-TraM | tra gens, |
| tumefaciens | L-homoserine | | traR, Ti |
| | lactone (AAI) | | plasmid |
| | | | conjugal |
| | | | transfer |
| Erwinia | VAI-1^{f} | Expl/ExpR | pel, pec, pep, |
| carotovora | | | exoenzyme |
| subsp. | | | synthesis |
| carotovora | | | |
| SCRI193 | | | |
| Erwinia | VAI-1^{f} | CarI/CarR | cap, |
| carotovora | | | carbapenem |
| subsp. | | | antibiotic |
| carotovora | | | synthesis |
| SCC3193 | | | |
| Erwinia | VAI-1^{f} | HsII/? | pel, pec, pep, |
| carotovora | | | exoenzyme |
| subsp. | | | synthesis |
| carotovora 71 | | | |
| Erwinia | VAI-1^{f} | Esal/EsaR | wts genes, |
| stewartii | | | exopoly- |
| | | | saccharide |
| | | | synthesis, |
| | | | virulence |
| | | | factors |
| Rhizobium | N-(3R-hydroxy- | ?/RhiR | rhiABC, |
| leguminosarum | 7-cis- | | rhizosphere |
| | tetradecanoyl-L- | | genes and |
| | homoserine | | stationary |
| | lactone, small | | phase |
| | bacteriocin, (RLAI) | | |
| Enterobacter | VAI-1^{f} | EagI/EagR | function |
| agglomerans | | | unclear |
| Yersenia | VAI-1^{f} | YenI/YenR | function |
| enterocolitica | | | unclear |
| Serratia | N-butanoyl-L- | Swrl/? | swarming |
| liquifaciens | homoserine lacton | | motility |
| | (SAI-1) | | |
| | N-hexanoyl- | Swrt/? | swarming |
| | L-homoserine | | motility |
| | lacton (SAI-2) | | |
| Aeromonas | (AHAI)^{e} | AhyI/AhyR | function |
| hydrophila | | | unclear |
| Escherichia | | ?/SdiA | ftsQAZ, cell |
| coli/?^{g} | | | division |

Autoinducer synthase molecules can be obtained from naturally occurring sources, e.g., by purifying cellular extracts, can be chemically synthesized or can be recombinantly produced. Recombinantly produced autoinducer synthase molecules can have the amino acid sequence of a naturally occurring form of the autoinducer synthase protein. They can also have a similar amino acid sequence which includes mutations such as substitutions and deletions (including truncation) of a naturally occurring form of the protein. Autoinducer synthase molecules can also include molecules which are structurally similar to the structures of naturally occurring autoinducer synthase proteins, e.g., biologically active variants.

TraI, LuxI, RhlI are the homoserine lactone autoinducer synthases of Agrobacterium tumefaceins, Vibrio fischeri, and Pseudomonas aeruginosa, respectively. The term “RhlI” is intended to include proteins which catalyze the synthesis of the homoserine lactone autoinducer of the RhlI quorum sensing system of P. aeruginosa, butyryl homoserine lactone.

The term “biofilm” is intended to include biological films that develop and persist at interfaces in aqueous environments. Biofilms are composed of microorganisms embedded in an organic gelatinous structure composed of one or more matrix polymers which are secreted by the resident microorganisms. The language “biofilm development” or “biofilm formation” is intended to include the formation, growth, and modification of the bacterial colonies contained with the biofilm structures as well as the synthesis and maintenance of the exopolysaccharide matrix of the biofilm structures.

The term “biofouling” includes the undesirable formation and/or accumulation of biofilms on surfaces. For example, biofilms may form in industrial settings and lead to material degradation, product contamination, mechanical blockage, and impedance of heat transfer in water-processing systems. Biofouling also includes to biological contamination of water distribution systems, e.g., due to growth on surfaces such as, for example, filtration devices. Biofouling further includes biofilm formation, for example, within food or on food processing devices, on medical devices, (e.g., catheters) or on the outside of vessels, e.g., boats or ships.

The term “biofilm-associated disease or disorder” includes diseases, disorders or conditions which are characterized or caused by the presence or potential presence of a biofilm, e.g., a bacterial biofilm. Biofilm-associated diseases or disorders include infection of the subject by one or more bacteria, e.g., Pseudomonas aeruginosa, Bacillus subtilis, Candida albicans, Staphylococcus aureus, Staphylococcus epidermidis, Enterococcus faecalis, Helicobacter pylori, Escherichia coli, Salmonella typhimurium, Legionella pneumophila, or other gram-negative or gram positive bacteria. Examples of biofilm-associated diseases or disorders include diseases or disorders caused by, for example, bacteria (e.g., gram-positive and/or gram-negative bacteria), fungi, viruses and parasites. Examples of biofilm-associated diseases or disorders include, but are not limited to, cystic fibrosis, AIDS, middle ear infections, osteomyelitis, acne, dental cavities, prostatitis, abscesses, bacteremia, contamination of peritoneal dialysis fluid, endocarditis, pneumonia, meningitis, cellulitis, pharyngitis, otitis media, sinusitis, scarlet fever, arthritis, urinary tract infection, laryngotracheitis, erysipeloid, gas gangrene, tetanus, typhoid fever, acute gastroenteritis, bronchitis, epiglottitis, plague, sepsis, chancroid, wound and bum infection, cholera, glanders, periodontitis, genital infections, empyema, granuloma inguinale, Legionnaire's disease, paratyphoid, bacillary dysentary, brucellosis, diphtheria, pertussis, botulism, toxic shock syndrome, mastitis, rheumatic fever, eye infections, including contact lens infections, periodontal infections, catheter- or medical device-associated infections, and plaque. Other biofilm-associated diseases or disorders include swine erysipelas, peritonitis, abortion, encephalitis, anthrax, nocardiosis, pericarditis, mycetoma, peptic ulcer, melioidosis, Haverhill fever, tularemia, Moko disease, galls (such as crown, cane and leaf), hairy root, bacterial rot, bacterial blight, bacterial brown spot, bacterial wilt, bacterial fin rot, dropsy, columnaris disease, pasteurellosis, furunculosis, enteric redmouth disease, vibriosis of fish, and fouling of medical devices.

The term “modulator”, as in “modulator of biofilm formation” is intended to encompass compounds capable of inducing and/or potentiating, as well as inhibiting and/or preventing quorum sensing controlled gene expression or quorum sensing controlled polypeptide activity. A “quorum sensing controlled nucleic acid modulator” or a “quorum sensing controlled gene modulator” is any compound which is capable of inducing and/or potentiating, as well as inhibiting and/or preventing quorum sensing controlled gene expression. A “quorum sensing controlled polypeptide” modulator is any compound which is capable of inducing and/or potentiating, as well as inhibiting and/or preventing quorum sensing controlled polypeptide expression or activity. A modulator of biofilm formation may act to modulate either signal generation, signal reception (e.g., the binding of a signal molecule to a receptor or target molecule), signal transmission (e.g., signal transduction via effector molecules to generate an appropriate biological response), biofilm formation or development, or antibiotic resistance.

Modulators may be purchased, chemically synthesized or recombinantly produced. Modulators can be obtained from a library of diverse compounds based on a desired activity, or alternatively they can be selected from a screening assay, such as a screening assay described herein. Examples of modulators include antibodies, polypeptides or fragments thereof, small molecules, nucleic acids or fragments thereof, or ribozymes. Small molecules include, but are not limited to, peptides, peptidomimetics, amino acids, amino acid analogs, polynucleotides, polynucleotide analogs, nucleotides, nucleotide analogs, organic or inorganic compounds (i.e.,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule compounds depends upon a number of factors within the knowledge of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the quorum sensing controlled molecule of the invention.

The term “compound” as used herein (e.g., as in “test compound,” or “modulator compound”) is intended to include both exogenously added test compounds and peptides endogenously expressed from a peptide library. Test compounds may be purchased, chemically synthesized or recombinantly produced. Test compounds can be obtained from a library of diverse compounds based on a desired activity, or alternatively they can be selected from a random screening procedure. In one embodiment, an indicator cell (e.g., a cell which responds to quorum sensing signals by generating a detectable signal) also produces the test compound which is being screened. For instance, the indicator cell can produce, e.g., a test polypeptide, a test nucleic acid and/or a test carbohydrate, which is screened for its ability to modulate quorum sensing signaling. In such embodiments, a culture of such reagent cells will collectively provide a library of potential modulator molecules and those members of the library which either stimulate or inhibit quorum sensing signaling can be selected and identified. In another embodiment, a test compound is produced by a second cell which is co-incubated with the indicator cell.

The terms “derived from” or “derivative”, as used interchangeably herein, are intended to mean that a sequence is identical to or modified from another sequence, e.g., a naturally occurring sequence. Derivatives within the scope of the invention include polynucleotide derivatives. Polynucleotide or nucleic acid derivatives differ from the sequences described herein (e.g., SEQ ID Nos.:1-353) or known in nucleotide sequence. For example, a polynucleotide derivative may be characterized by one or more nucleotide substitutions, insertions, or deletions, as compared to a reference sequence. A nucleotide sequence comprising a quorum sensing controlled genetic locus that is derived from the genome of P. aeruginosa, e.g., SEQ ID Nos.:1-353, includes sequences that have been modified by various changes such as insertions, deletions and substitutions, and which retain the property of being regulated in response to a quorum sensing signaling event. Such sequences may comprise a quorum sensing controlled regulatory element and/or a quorum sensing controlled gene. The complete genome of P. aeruginosa has been elucidated (Stover, et al. (2000) Nature 406:947-948). The nucleotide sequence of the P. aeruginosa genome and the encoded polypeptide sequences are available at online at the P. aeruginosa Genome Project website.

Polypeptide or protein derivatives of the invention include polypeptide or protein sequences that differ from the sequences described or known in amino acid sequence, or in ways that do not involve sequence, or both, and still preserve the activity of the polypeptide or protein. Derivatives in amino acid sequence are produced when one or more amino acids is substituted with a different natural amino acid, an amino acid derivative or non-native amino acid. In certain embodiments protein derivatives include naturally occurring polypeptides or proteins, or biologically active fragments thereof, whose sequences differ from the wild type sequence by one or more conservative amino acid substitutions, which typically have minimal influence on the secondary structure and hydrophobic nature of the protein or peptide. Derivatives may also have sequences which differ by one or more non-conservative amino acid substitutions, deletions or insertions which do not abolish the biological activity of the polypeptide or protein.

Conservative substitutions (substituents) typically include the substitution of one amino acid for another with similar characteristics (e.g., charge, size, shape, and other biological properties) such as substitutions within the following groups: valine, glycine; glycine, alanine; valine, isoleucine; aspartic acid, glutamic acid; asparagine, glutamine; serine, threonine; lysine, arginine; and phenylalanine, tyrosine. The non-polar (hydrophobic) amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid.

In other embodiments, derivatives with amino acid substitutions which are less conservative may also result in desired derivatives, e.g., by causing changes in charge, conformation and other biological properties. Such substitutions would include, for example, substitution of hydrophilic residue for a hydrophobic residue, substitution of a cysteine or proline for another residue, substitution of a residue having a small side chain for a residue having a bulky side chain or substitution of a residue having a net positive charge for a residue having a net negative charge. When the result of a given substitution cannot be predicted with certainty, the derivatives may be readily assayed according to the methods disclosed herein to determine the presence or absence of the desired characteristics. The polypeptides and proteins of this invention may also be modified by various changes such as insertions, deletions and substitutions, either conservative or nonconservative where such changes might provide for certain advantages in their use.

As used herein, the term “genetic locus” includes a position on a chromosome, or within a genome, which is associated with a particular gene or genetic sequences having a particular characteristic. For example, in one embodiment, a quorum sensing controlled genetic locus includes nucleic acid sequences which comprise an open reading frame (ORF) of a quorum sensing controlled gene. In another embodiment, a quorum sensing controlled genetic locus includes nucleic acid sequences which comprise transcriptional regulatory sequences that are responsive to quorum sensing signaling (e.g., a quorum sensing controlled regulatory element). Examples of quorum sensing controlled genetic loci of P. aeruginosa are described herein as SEQ ID NOs.:1-38.

The term “modulator”, as in “modulator of quorum sensing signaling” “quorum sensing controlled gene modulator” or a “quorum sensing controlled polypeptide modulator” is intended to encompass, in its various grammatical forms, induction and/or potentiation, as well as inhibition and/or downregulation of quorum sensing signaling and/or quorum sensing controlled gene and/or polypeptide expression. As used herein, the term “modulator of quorum sensing signaling” “quorum sensing controlled gene modulator” or a “quorum sensing controlled polypeptide modulator” includes a compound or agent that is capable of modulating or regulating at least one quorum sensing controlled gene or quorum sensing controlled genetic locus, e.g., a quorum sensing controlled genetic locus in P. aeruginosa, or the expression of a quorum sensing controlled polypeptide, as described herein. A modulator of quorum sensing signaling may act to modulate either signal generation (e.g., the synthesis of a quorum sensing signal molecule), signal reception (e.g., the binding of a signal molecule to a receptor or target molecule), or signal transmission (e.g., signal transduction via effector molecules to generate an appropriate biological response). In one embodiment, a method of the present invention encompasses the modulation of the transcription of an indicator gene in response to an autoinducer molecule. In another embodiment, a method of the present invention encompasses the modulation of the transcription of an indicator gene, preferably an quorum sensing controlled indicator gene, by a test compound.

The term “operatively linked” or “operably linked” is intended to mean that molecules are functionally coupled to each other in that the change of activity or state of one molecule is affected by the activity or state of the other molecule. In one embodiment, nucleotide sequences are “operatively linked” when the regulatory sequence functionally relates to the DNA sequence encoding the polypeptide or protein of interest. For example, a nucleotide sequence comprising a transcriptional regulatory element(s) (e.g., a promoter) is operably linked to a DNA sequence encoding the protein or polypeptide of interest if the promoter nucleotide sequence controls the transcription of the DNA sequence encoding the protein of interest. In addition, two nucleotide sequences are operatively linked if they are coordinately regulated and/or transcribed. Typically, two polypeptides that are operatively linked are covalently attached through peptide bonds.

The term “quorum sensing signaling” or “quorum sensing” is intended to include the generation of a cellular signal in response to cell density. In one embodiment, quorum sensing signaling mediates the coordinated expression of specific genes. A “quorum sensing controlled gene” is any gene, the expression of which is regulated in a cell density dependent fashion. In a preferred embodiment, the expression of a quorum sensing controlled gene is modulated by a quorum sensing signal molecule, e.g., an autoinducer molecule (e.g., a homoserine lactone molecule). The term “quorum sensing signal molecule” is intended to include a molecule that transduces a quorum sensing signal and mediates the cellular response to cell density. In a preferred embodiment the quorum sensing signal molecule is a freely diffusible autoinducer molecule, e.g., a homoserine lactone molecule or analog thereof. In one embodiment, a quorum sensing controlled gene encodes a virulence factor. In another embodiment, a quorum sensing controlled gene encodes a protein or polypeptide that, either directly or indirectly, inhibits and/or antagonizes a bacterial host defense mechanism. In yet another embodiment, a quorum sensing controlled gene encodes a protein or polypeptide that regulates biofilm formation.

The term “regulatory sequences” is intended to include the DNA sequences that control the transcription of an adjacent gene. Gene regulatory sequences include, but are not limited to, promoter sequences that are found in the 5′ region of a gene proximal to the transcription start site which bind RNA polymerase to initiate transcription. Gene regulatory sequences also include enhancer sequences which can function in either orientation and in any location with respect to a promoter, to modulate the utilization of a promoter, and other expression control elements (e.g., polyadenylation signals). Such regulatory sequences are described, for example, in Goeddel (1990) Methods Enzymol. 185:3-7. Transcriptional control elements include, but are not limited to, promoters, enhancers, and repressor and activator binding sites. The gene regulatory sequences of the present invention contain binding sites for transcriptional regulatory proteins. In one embodiment, a regulatory sequence includes a sequence that mediates quorum sensing controlled gene expression, e.g., a las box. In a preferred embodiment, gene regulatory sequences comprise sequences derived from the Pseudomonas aeruginosa genome which modulate quorum sensing controlled gene expression e.g., SEQ ID NOs.:708 and 709. In another preferred embodiment, gene regulatory sequences comprise sequences (e.g., a genetic locus) derived from the Pseudomonas aeruginosa genome which modulate the expression of quorum sensing controlled genes, e.g., SEQ ID NOs.:1-353.

The term “reporter gene” or “indicator gene” generically refers to an expressible (e.g., able to be transcribed and (optionally) translated) DNA sequence which is expressed in response to the activity of a transcriptional regulatory protein. Indicator genes include unmodified endogenous genes of the host cell, modified endogenous genes, or a reporter gene of a heterologous construct, e.g., as part of a reporter gene construct. In a preferred embodiment, the level of expression of an indicator gene produces a detectable signal.

Reporter gene constructs are prepared by operatively linking an indicator gene with at least one transcriptional regulatory element. If only one transcriptional regulatory element is included, it is advantageously a regulatable promoter. In a preferred embodiment at least one of the selected transcriptional regulatory elements is directly or indirectly regulated by quorum sensing signals, whereby quorum sensing controlled gene expression can be monitored via transcription and/or translation of the reporter genes.

Many reporter genes and transcriptional regulatory elements are known to those of skill in the art and others may be identified or synthesized by methods known to those of skill in the art. Reporter genes include any gene that expresses a detectable gene product, which may be RNA or protein. Preferred reporter genes are those that are readily detectable. In one embodiment, an indicator gene of the present invention is comprised in the nucleic acid molecule in the form of a fusion gene (e.g., operatively linked) with a nucleotide sequence that includes regulatory sequences (e.g., quorum sensing transcriptional regulatory elements, e.g., a las box) derived from the Pseudomonas aeruginosa genome (e.g., SEQ ID NOs:708, 709, or 710). In another embodiment, an indicator gene of the present invention is operatively linked to quorum sensing transcriptional regulatory sequences that regulate a quorum sensing controlled genetic locus derived from the Pseudomonas aeruginosa genome, e.g., a genetic locus comprising a nucleotide sequence set forth as SEQ ID NOs.: 1-353. In yet another embodiment, an indicator gene of the present invention is operatively linked to a nucleotide sequence comprising a quorum sensing controlled genetic locus derived from the Pseudomonas aeruginosa genome (e.g., SEQ ID NOs.:1-353, 707, 708, 709, or 710). In certain embodiments of the invention, an indicator gene (e.g., a promoterless indicator gene) is contained in a transposable element.

The term “detecting a change in the detectable signal” is intended to include the detection of alterations in gene transcription of an indicator or reporter gene induced upon modulation of quorum sensing signaling. In certain embodiments, the reporter gene may provide a selection method such that cells in which the transcriptional regulatory protein activates transcription have a growth advantage. For example the reporter could enhance cell viability, relieve a cell nutritional requirement, and/or provide resistance to a drug. In other embodiments, the detection of an alteration in a signal produced by an indicator gene encompass assaying general, global changes to the cell such as changes in second messenger generation.

The amount of transcription from the reporter gene may be measured using any method known to those of skill in the art. For example, specific mRNA expression may be detected using Northern blots, or a specific protein product may be identified by a characteristic stain or an intrinsic activity. In preferred embodiments, the gene product of the reporter is detected by an intrinsic activity associated with that product. For instance, the reporter gene may encode a gene product that, by enzymatic activity, gives rise to a detection signal based on color, fluorescence, or luminescence.

The amount of regulation of the indicator gene, e.g., expression of a reporter gene, is then compared to the amount of expression in a control cell. For example, the amount of transcription of an indicator gene may be compared between a cell in the absence of a test modulator molecule and an identical cell in the presence of a test modulator molecule.

As used interchangeably herein, the terms “transposon” and “transposable element” are intended to include a piece of DNA that can insert into and cut itself out of, genomic DNA of a particular host species. Transposons include mobile genetic elements (MGEs) containing insertion sequences and additional genetic sequences unrelated to insertion functions (for example, sequences encoding a reporter gene). Insertion sequence elements include sequences that are between 0.7 and 1.8 kb in size with termini approximately 10 to 40 base pairs in length with perfect or nearly perfect repeats. As used herein, a transposable element is operatively linked to the nucleotide sequence into which it is inserted. Transposable elements are well known in the art.

The present invention discloses a method for identifying modulators of quorum sensing signaling in bacteria, e.g., Pseudomonas aeruginosa. As described herein, the method of the invention comprises providing a cell which comprises a quorum sensing controlled gene, wherein the cell is responsive to a quorum sensing signal molecule such that a detectable signal is generated. A cell which responds to a quorum sensing signal molecule by generating a detectable signal is referred to herein as an “indicator cell” or a “reporter cell”. In a preferred embodiment of the invention, the cell is a P. aeruginosa bacterial cell. In another preferred embodiment, the cell is from a mutant strain of P. aeruginosa which comprises a reporter gene operatively linked to a regulatory sequence of a quorum sensing controlled gene, wherein said mutant strain is responsive to a quorum sensing signal molecule, such that a detectable signal is generated. In yet another preferred embodiment, the cell is a mutant strain of P. aeruginosa which comprises a promoterless reporter gene inserted in the chromosome at a quorum sensing controlled genetic locus, e.g., a genetic locus comprising a nucleotide sequence set forth as SEQ ID NOs.:1-353, wherein said mutant strain is responsive to a quorum sensing signal molecule such that a detectable signal is generated by the reporter gene. In a preferred embodiment, the reporter gene is contained in a transposable element. In a further preferred embodiment, the cell is from a strain of P. aeruginosa in which lasi and rhlI are inactivated, such that the cell does not express the lasi and RhlI autoinducer synthases which are involved in the generation of quorum sensing signal molecules. A compound is identified as a modulator of quorum sensing signaling in bacteria by contacting the cell with a quorum sensing signal molecule in the presence and absence of a test compound and detecting a change in the detectable signal.

Quorum sensing signal molecules that are useful in the methods of the present invention include autoinducer compounds such as homoserine lactones, and analogs thereof (see Table 1). In certain embodiments, the quorum sensing signal molecule is either 3-oxo-C12-homoserine lactone or C4-HSL. In one embodiment, the cell does not express the quorum sensing signal molecule. For example, the cell may comprise a mutant strain of Pseudomonas aeruginosa wherein lasI and rhlI are inactivated. Therefore, the cell is contacted with an exogenous quorum sensing signal molecule, e.g., a recombinant or synthetic molecule. In another embodiment, the quorum sensing signal molecule is produced by a second cell (e.g., a prokaryotic or eukaryotic cell), which is co-incubated with the indicator cell. For example, an indicator cell which does not express a quorum sensing signal molecule can be co-incubated with a wild type strain of Pseudomonas aeruginosa which produces a quorum sensing signal molecule. Alternatively, the indicator strain which does not express a quorum sensing signal molecule is co-incubated with a second cell which has been transformed, or otherwise altered, such that it is able to express a quorum sensing signal molecule. In yet another embodiment, the quorum sensing signal molecule is expressed by the indicator strain.

Similarly, the test compound can be exogenously added to an indicator strain, produced by a second cell which is co-incubated with the indicator strain, or expressed by the indicator strain. Exemplary compounds which can be screened for activity include, but are not limited to, peptides, nucleic acids, carbohydrates, small organic molecules, and natural product extract libraries.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:45).

Examples of methods for the synthesis of molecular libraries can be found in the art, for example, in: DeWitt et al. (1993) Proc. Natl. Acad Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g. Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. USA 87:6378-6382); (Felici (1991) J. Mol. Biol. 222:301-310); (Ladner supra.).

In certain embodiments of the instant invention, the compounds tested are in the form of peptides from a peptide library. The peptide library may take the form of a cell culture, in which essentially each cell expresses one, and usually only one, peptide of the library. While the diversity of the library is maximized if each cell produces a peptide of a different sequence, it is usually prudent to construct the library so there is some redundancy. Depending on size, the combinatorial peptides of the library can be expressed as is, or can be incorporated into larger fusion proteins. The fusion protein can provide, for example, stability against degradation or denaturation. In an exemplary embodiment of a library for intracellular expression, e.g., for use in conjunction with intracellular target receptors, the polypeptide library is expressed as thioredoxin fusion proteins (see, for example, U.S. Pat. Nos. 5,270,181 and 5,292,646; and PCT publication WO94/02502). The combinatorial peptide can be attached on the terminus of the thioredoxin protein, or, for short peptide libraries, inserted into the so-called active loop.

In one embodiment of the instant invention the cell further comprises a means for generating the detectable signal. For example, the cell may comprise a reporter gene, the transcription of which is regulated by a quorum sensing signal molecule. In a preferred embodiment, the reporter gene is operatively linked to a regulatory sequence of a quorum sensing controlled gene, e.g. a nucleotide sequence comprising at least one quorum sensing controlled regulatory element, e.g., a las-rhl box. In another embodiment, the reporter gene is operatively linked to a quorum sensing controlled genetic locus, e.g., a quorum sensing controlled gene, such that transcription of the indicator gene is responsive to quorum sensing signals. For example, in a preferred embodiment, a promoterless reporter gene is inserted into a quorum sensing controlled genetic locus derived from the genome of P. aeruginosa. Such quorum sensing controlled genetic loci, as described herein, include the loci in the P. aeruginosa genome which comprise the nucleotide sequences set forth as SEQ ID NOs.: 1-38. In another preferred embodiment, the promoterless reporter gene is contained in a transposable element that is inserted into a quorum sensing controlled genetic locus in the P. aeruginosa genome.

Examples of reporter genes include, but are not limited to, CAT chloramphenicol acetyl transferase) (Alton and Vapnek (1979), Nature 282: 864-869), and other enzyme detection systems, such as beta-galactosidase (lacZ), firefly luciferase (de Wet et al. (1987), Mol. Cell. Biol. 7:725-737); bacterial luciferase (Engebrecht and Silverman (1984), PNAS 1: 4154-4158; Baldwin et al. (1984), Biochemistry 23: 3663-3667); alkaline phosphatase (Toh et al. (1989) Eur. J. Biochem. 182: 231-238, Hall et al. (1983) J. Mol. Appl. Gen. 2: 101), human placental secreted alkaline phosphatase (Cullen and Malim (1992) Methods in Enzymol. 216:362-368), and horseradish peroxidase. In one preferred embodiment, the indicator gene is lacZ. In another preferred embodiment, the indicator gene is green fluorescent protein (U.S. Pat. No. 5,491,084; WO96/23898) or a variant thereof. A preferred variant is GFPmut2. Other reporter genes include ADE1, ADE2, ADE3, ADE4, ADE5, ADE7, ADE8, ASP3, ARG1, ARG3, ARG4, ARG5, ARG6, ARG8, ARO2, ARO7, BAR1, CAT, CHO1, CYS3, GAL1, GAL7, GAL10, HIS1, HIS3, HIS4, HIS5, HOM3, HOM6, ILV1, ILV2, ILV5, INO1, INO2, INO4, LEU1, LEU2, LEU4, LYS2, MAL, MEL, MET2, MET3, MET4, MET8, MET9, MET14, MET16, MET19, OLE1, PHO5, PRO1, PRO3, THR1, THR4, TRP1, TRP2, TRP3, TRP4, TRP5, URA1, URA2, URA3, URA4, URA5 and URA10.

In accordance with the methods of the invention, compounds which modulate quorum sensing signaling can be selected and identified. The ability of compounds to modulate quorum sensing signaling can be detected by up or down-regulation of the detection signal provided by the indicator gene. Any difference, e.g., a statistically significant difference, in the amount of transcription indicates that the test compound has in some manner altered the activity of quorum sensing signaling.

A modulator of quorum sensing signaling may act by inhibiting an enzyme involved in the synthesis of a quorum sensing signal molecule, by inhibiting reception of the quorum sensing signal molecule by the cell, or by scavenging the quorum sensing signal molecule. The term “scavenging” is meant to include the sequestration, chemical modification, or inactivation of a quorum sensing signal molecule such that it is no longer able to regulate quorum sensing gene control. After identifying certain test compounds as potential modulators of quorum sensing signaling, the practitioner of the subject assay will continue to test the efficacy and specificity of the selected compounds both in vitro and in vivo, e.g., in an assay for bacterial viability and/or pathogenecity.

In another aspect, the present invention discloses a method for identifying a quorum sensing controlled gene in bacteria, e.g., Pseudomonas aeruginosa. The method comprises providing a cell which is responsive to a quorum sensing signal molecule such that expression of a quorum sensing controlled gene is modulated, and wherein modulation of the expression of the quorum sensing controlled gene generates a detectable signal. The cell is contacted with a quorum sensing signal molecule and a change in the signal is detected to thereby identify a quorum sensing signaling controlled gene.

In one embodiment, the cell further comprises a means for generating the detectable signal, e.g., a reporter gene. For example, the cell may comprise a promoterless reporter gene that is operatively linked to a quorum sensing controlled genetic locus such that modulation of the expression of the quorum sensing controlled locus concurrently modulates transcription of the reporter gene. The position of the quorum sensing controlled genetic locus is then mapped based on the position of the reporter gene.

In a preferred embodiment of the invention, the cell is a P. aeruginosa bacterial cell. In another preferred embodiment, the cell is a mutant strain of P. aeruginosa which comprises a promoterless reporter gene inserted in the chromosome at a quorum sensing controlled genetic locus, e.g., a genetic locus comprising a nucleotide sequence set forth as SEQ ID NOs.:1-353, wherein said mutant strain is responsive to a quorum sensing signal molecule such that a detectable signal is generated by the reporter gene. In a preferred embodiment, the reporter gene is contained in a transposable element. In a further preferred embodiment, the cell is from a strain of P. aeruginosa in which lasI and rhlI are inactivated, such that the cell does not express the lasI and rhlI autoinducer synthases which are involved in the generation of quorum sensing signal molecules.

It is also to be understood that genomic sequences from a mutant bacterial strain (e.g., P. aeruginosa) in which a promoterless reporter gene (e.g., a reporter gene contained in a transposable element) has been inserted at a quorum sensing controlled locus, can be assayed in a heterologous cell that is responsive to a quorum sensing signal molecule such that quorum sensing signal transduction occurs. For example, the genomic DNA of a strain of P. aeruginosa subjected to transposon mutagenesis, as described herein, can be engineered into a library, and transferred to another cell capable of quorum sensing signaling (e.g., a different species of gram negative bacteria), and assayed to identify a quorum sensing controlled gene.

In one embodiment, the cell is contacted with an exogenous quorum sensing signal molecule, e.g., a recombinant or synthetic molecule, as described herein. In another embodiment, the quorum sensing signal molecule is produced by a second cell (e.g., a prokaryotic or eukaryotic cell), which is co-incubated with the indicator cell. For example, an indicator cell which does not express a quorum sensing signal molecule can be co-incubated with a wild type strain of Pseudomonas aeruginosa which produces a quorum sensing signal molecule. Alternatively, the indicator strain which does not express a quorum sensing signal molecule is co-incubated with a second cell which has been transformed, or otherwise altered, such that it is able to express a quorum sensing signal molecule. In yet another embodiment, the quorum sensing signal molecule is expressed by the indicator strain.

Another aspect of the invention provides a mutant strain of Pseudomonas aeruginosa comprising a promoterless reporter gene inserted in a chromosome at a genetic locus comprising a nucleotide sequence set forth as SEQ ID NOs:1-353, e.g., a quorum sensing controlled genetic locus. In one embodiment the reporter gene is contained in a transposable element. In another embodiment, the reporter gene is lacZ or GFP, or a variant thereof, e.g., GFPmut2. In yet another embodiment, lasi and rhlI are inactivated in the mutant strain of P. aeruginosa. The above-described cells are useful in the methods of the instant invention, as the cells are responsive to a quorum sensing signal molecule such that a detectable signal is generated by the reporter gene. These cells are also useful for studying the function of polypeptides encoded by the quorum sensing controlled loci comprising the nucleotide sequences set forth as SEQ ID NOs.:1-353.

Yet another aspect of the invention provides isolated nucleic acid molecules comprising a nucleotide sequence comprising a quorum sensing controlled genetic locus derived from the genome of Pseudomonas aeruginosa operatively linked to a reporter gene. In one embodiment, a reporter gene is operatively linked to a regulatory sequence derived from the genome of P. aeruginosa, wherein the regulatory sequence regulates a quorum sensing controlled genetic locus comprising a nucleotide sequence set forth as SEQ ID NO:1-353. In a preferred embodiment such regulatory sequences comprise at least one binding site for a quorum sensing controlled transcriptional regulatory factor (e.g., a transcriptional activator or repressor molecule) such that transcription of the reporter gene is responsive to a quorum sensing signal molecule and/or a modulator of quorum sensing signaling. In another embodiment, a reporter gene is operatively linked to a quorum sensing controlled genetic locus derived from the genome of P. aeruginosa, wherein the genetic locus comprises a nucleotide sequence set forth as SEQ ID NO:1-353. In yet another embodiment, a reporter gene is operatively linked to a nucleotide sequence which has at least 80%, and more preferably at least 85%, 90% or 95% identity to quorum sensing controlled genetic locus derived from the genome of P. aeruginosa, wherein the genetic locus comprises a nucleotide sequence set forth as SEQ ID NO:1-353. In a further embodiment, a reporter gene is operatively linked to a nucleotide sequence which hybridizes under stringent conditions to quorum sensing controlled genetic locus derived from the genome of P. aeruginosa, wherein the genetic locus comprises a nucleotide sequence set forth as SEQ ID NO:1-353.

The term “isolated nucleic acid molecule” includes nucleic acid molecules which are separated from other nucleic acid molecules which are present in the natural source of the nucleic acid. For example, with regard to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. As used interchangeably herein, the terms “nucleic acid molecule” and “polynucleotide” are intended to include DNA molecules (e.g., cDNA or genomic DNA) and RNA molecules (e.g., mRNA) and analogs of the DNA or RNA generated using nucleotide analogs. The nicleic acid molecule can be single-stranded or double-stranded, but preferably is double-stranded DNA. The term “DNA” refers to deoxyribonucleic acid whether single- or double-stranded. As used herein, the terms “gene” and “recombinant gene” refer to nucleic acid molecules which include an open reading frame encoding a protein, preferably a quorum sensing controlled protein, and can further include non-coding regulatory sequences, and introns.

The present invention includes polynucleotides capable of hybridizing under stringent conditions, preferably highly stringent conditions, to the polynucleotides described herein (e.g., a quorum sensing controlled genetic locus, e.g., SEQ ID NOs.:1-353). As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4, and 6. Additional stringent conditions can be found in Molecular Cloning: A Laboratory Manual, Sambrook et al., Cold Spring Harbor Press, Cold Spring Harbor, N.Y. (1989), chapters 7, 9, and 11. A preferred, non-limiting example of stringent hybridization conditions includes hybridization in 4×sodium chloride/sodium citrate (SSC), at about 65-70° C. (or alternatively hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or alternatively hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(% G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nlcleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stiingent hybridization conditions is hybridization in 0.25-0.5M NaH₂PO₄, 7% SDS at about 65° C., followed by one or more washes at 0.02M NaH₂PO₄, 1% SDS at 65° C. (see e.g., Church and Gilbert (1984) Proc. Natl. Acad. Sci. USA 81:1991-1995), or alternatively 0.2×SSC, 1% SDS.

The invention further encompasses nucleic acid molecules that differ from the quorum sensing controlled genetic loci described herein, e.g., the nucleotide sequences shown in SEQ ID NO:1-353. Accordingly, the invention also includes variants, e.g., allelic variants, of the disclosed polynucleotides or proteins encoded by the polynucleotides disclosed herein; that is naturally occurring and non-naturally occurring alternative forms of the isolated polynucleotide which may also encode proteins which are identical, homologous or related to that encoded by the polynucleotides of the invention.

Nucleic acid variants can be naturally occurring, such as allelic variants (same locus), homologues (different locus), and orthologues (different organism) or can be non naturally occurring. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product). Allelic variants result, for example, from DNA sequence polymorphisms within a population (e.g., a bacterial population) that lead to changes in the nucleic acid sequences of quorum sensing controlled genetic loci.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90% or 95% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available on the Internet at the Accelrys website), using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available on the Internet at the Accelrys website), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (Comput. Appl. Biosci., 4:11-17 (1988) which has been incorporated into the ALIGN program (version 2.0) (available at the ALIGN™ website), using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See the National Center for Biotechnology website. Additionally, the “Clustal” method (Higgins and Sharp, Gene, 73:237-44, 1988) and “Megalign” program (Clewley and Arnold, Methods Mol. Biol, 70:119-29, 1997) can be used to align sequences and determine similarity, identity, or homology.

Accordingly, the present invention also discloses recombinant vector constructs and recombinant host cells transformed with said constructs.

The term “vector” or “recombinant vector” is intended to include any plasmid, phage DNA, or other DNA sequence which is able to replicate autonomously in a host cell. As used herein, the term “vector” refers to a nucleic acid molecule capable of transporting another nucleic acid to which it has been linked. A vector may be characterized by one or a small number of restriction endonuclease sites at which such DNA sequences may be cut in a determinable fashion without the loss of an essential biological function of the vector, and into which a DNA fragment may be spliced in order to bring about its replication and cloning. A vector may further contain a marker suitable for use in the identification of cells transformed with the vector. Recombinant vectors may be generated to enhance the expression of a gene which has been cloned into it, after transformation into a host. The cloned gene is usually placed under the control of (i.e., operably linked to) certain control sequences or regulatory sequences, which may be either constitutive or inducible.

One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. Expression systems for both prokaryotic and eukaryotic cells are described in, for example, chapters 16 and 17 of Sambrook, J. et al. Molecular Cloning: A Laboratory Manual. 2^{nd}, ed., Cold Spring Harbor Laboratory, Cold Spring Harbor Laboratory Press, Cold Spring Harbor, N.Y., 1989.

In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include ψCrip,ψCre, ψ2 and ψAm. The genome of adenovirus can be manipulated such that it encodes and expresses a transcriptional regulatory protein but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are well known to those skilled in the art. Alternatively, an adeno-associated virus vector such as that described in Tratschin et al. ((1985) Mol. Cell. Biol. 5:3251-3260) can be used.

In general, it may be desirable that an expression vector be capable of replication in the host cell. Heterologous DNA may be integrated into the host genome, and thereafter is replicated as a part of the chromosomal DNA, or it may be DNA which replicates autonomously, as in the case of a plasmid. In the latter case, the vector will include an origin of replication which is functional in the host. In the case of an integrating vector, the vector may include sequences which facilitate integration, e.g., sequences homologous to host sequences, or encoding integrases.

Appropriate cloning and expression vectors for use with bacterial, fungal, yeast, and mammalian cellular hosts are known in the art, and are described in, for example, Powels et al. (Cloning Vectors: A Laboratory Manual, Elsevier, N.Y., 1985). Mammalian expression vectors may comprise non-transcribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a poly-adenylation site, splice donor and acceptor sites, and transcriptional termination sequences.

The vectors of the subject invention may be transformed into an appropriate cellular host for use in the methods of the invention.

As used interchangeably herein, a “cell” or a “host cell” includes any cultivatable cell that can be modified by the introduction of heterologous DNA. As used herein, “heterologous DNA”, a “heterologous gene” or “heterologous polynucleotide sequence” is defined in relation to the cell or organism harboring such a nucleic acid or gene. A heterologous DNA sequence includes a sequence that is not naturally found in the host cell or organism, e.g., a sequence which is native to a cell type or species of organism other than the host cell or organism. Heterologous DNA also includes mutated endogenous genetic sequences, for example, as such sequences are not naturally found in the host cell or organism. Preferably, a host cell is one in which a quorum sensing signal molecule, e.g., an autoinducer molecule, initiates a quorum sensing signaling response which includes the regulation of target quorum sensing controlled genetic sequences. The choice of an appropriate host cell will also be influenced by the choice of detection signal. For example, reporter constructs, as described herein, can provide a selectable or screenable trait upon activation or inhibition of gene transcription in response to a quorum sensing signaling event; in order to achieve optimal selection or screening, the host cell phenotype will be considered.

A host cell of the present invention includes prokaryotic cells and eukaryotic cells. Prokaryotes include gram negative or gram positive organisms, for example, E. Coli or Bacilli. Suitable prokaryotic host cells for transformation include, for example, E. coli, Bacillus subtilis, Salmonella typhimurium, and various other species within the genera Pseudomonas, Streptomyces, and Staphylococcus. In a preferred embodiment, a host cell of the invention is a mutant strain of P. aeruginosa in which lasI and rhlI are inactivated.

Eukaryotic cells include, but are not limited to, yeast cells, plant cells, fungal cells, insect cells (e.g., baculovirus), mammalian cells, and cells of parasitic organisms, e.g., trypanosomes. Mammalian host cell culture systems include established cell lines such as COS cells, L cells, 3T3 cells, Chinese hamster ovary (CHO) cells, embryonic stem cells, and HeLa cells. Other suitable host cells are known to those skilled in the art.

DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2^{nd}, ed., Cold Spring Harbor Laboratory, Cold Spring Harbor Laboratory Press, Cold Spring Harbor, N.Y., 1989), and other laboratory manuals.

Host cells comprising an isolated nucleic acid molecule of the invention (e.g., a quorum sensing controlled genetic locus operatively linked to a reporter gene) can be used in the methods of the instant invention to identify a modulator of quorum sensing signaling in bacteria.

Another aspect of the invention pertains to the use of isolated quorum sensing controlled polypeptides, and biologically active portions thereof, as well as the use of polypeptide fragments suitable for use as immunogens to raise anti-quorum sensing controlled antibodies. In one embodiment, native quorum sensing controlled polypeotides can be isolated from cells sources by an appropriate purification scheme using standard protein purification techniques. In another embodiment, quorum sensing controlled polypeptides are produced by recombinant DNA techniques. Alternative to recombinant expression, a quorum sensing controlled polypeptide can be synthesized chemically using standard peptide synthesis techniques.

An “isolated” or “purified” protein or biologically active portion thereof is substantially free of cellular material or other contaminating proteins from the cell or tissue source from which the quorum sensing controlled polypeptide is derived, or substantially free from chemical precursors or other chemicals when chemically synthesized. The language “substantially free of cellular material” includes preparations of quorum sensing controlled polypeptide in which the protein is separated from cellular components of the cells from which it is isolated or recombinantly produced. In one embodiment, the language “substantially free of cellular material” includes preparations of quorum sensing controlled polypeptide having less than about 30% (by dry weight) of non- quorum sensing controlled polypeptide (also referred to herein as a “contaminating protein”), more preferably less than about 20% of non-quorum sensing controlled polypeptide, still more preferably less than about 10% of non-quorum sensing controlled polypeptide, and most preferably less than about 5% non-quorum sensing controlled polypeptide. When the quorum sensing controlled polypeptide or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of quorum sensing controlled polypeptide in which the protein is separated from chemical precursors or other chemicals which are involved in the synthesis of the protein. In one embodiment, the language “substantially free of chemical precursors or other chemicals” includes preparations of quorum sensing controlled polypeptide having less than about 30% (by dry weight) of chemical precursors or non-quorum sensing controlled chemicals, more preferably less than about 20% chemical precursors or non-quorum sensing controlled chemicals, still more preferably less than about 10% chemical precursors or non-quorum sensing controlled chemicals, and most preferably less than about 5% chemical precursors or non-quorum sensing controlled chemicals.

As used herein, a “biologically active portion” of a quorum sensing controlled polypeptide includes a fragment of a quorum sensing controlled polypeptide which participates in an interaction between a quorum sensing controlled molecule and a non-quorum sensing controlled molecule. Biologically active portions of a quorum sensing controlled polypeptide include peptides comprising amino acid sequences sufficiently identical to or derived from the amino acid sequence of the quorum sensing controlled polypeptide, e.g., the amino acid sequence shown in SEQ ID NOs:354-706, which include less amino acids than the full length quorum sensing controlled polypeptides, and exhibit at least one activity of a quorum sensing controlled polypeptide. Typically, biologically active portions comprise a domain or motif with at least one activity of the quorum sensing controlled polypeptide, e.g., modulating signal generation, signal reception, biofilm formation, biofilm development, or antibiotic resistance. A biologically active portion of a quorum sensing controlled polypeptide can be a polypeptide which is, for example, 10, 15, 20, 25, 30, 35, 40, 45, 50, 75, 100, 125, 150, 175, 200 or more amino acids in length. Biologically active portions of a quorum sensing controlled polypeptide can be used as targets for developing compounds which modulate biofilm formation.

In a preferred embodiment, the quorum sensing controlled polypeptide has an amino acid sequence shown in SEQ ID NOs:354-706. In other embodiments, the quorum sensing controlled polypeptide is substantially identical to SEQ ID NOs:354-706, and retains the functional activity of the protein of SEQ ID NOs:354-706, yet differs in amino acid sequence due to natural allelic variation or mutagenesis, as described in detail herein. Accordingly, in another embodiment, the quorum sensing controlled polypeptide is a protein which comprises an amino acid sequence at least about 50%, 55%, 60%, 65%, 70%, 75%, 80%, 85%, 90%, 91%, 92%, 93%, 94%, 95%, 96%, 97%, 98%, 99% or more identical to any one of SEQ ID NOs:354-706.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the quorum sensing controlled amino acid sequence of SEQ ID NOs:354-706 having 419 amino acid residues, at least 120, preferably at least 160, more preferably at least 201, even more preferably at least 241, and even more preferably at least 281 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at the Genetics Computer Group website), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at the Genetics Computer Group website), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to quorum sensing controlled nucleic acid molecules of the invention. BLAST protein searches can be performed with the XBLAST program, score=100, wordlength=3 to obtain amino acid sequences homologous to quorum sensing controlled protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See the National Center for Biotechnology Information website.

The invention also provides quorum sensing controlled chimeric or fusion proteins. As used herein, a quorum sensing controlled “chimeric protein” or “fusion protein” comprises a quorum sensing controlled polypeptide operatively linked to a non-quorum sensing controlled polypeptide. A “quorum sensing controlled polypeptide” refers to a polypeptide having an amino acid sequence corresponding to quorum sensing controlled, whereas a “non-quorum sensing controlled polypeptide” refers to a polypeptide having an amino acid sequence corresponding to a protein which is not substantially homologous to a quorum sensing controlled polypeptide, e.g., a protein which is different from a quorum sensing controlled polypeptide and which is derived from the same or a different organism. Within a quorum sensing controlled fusion protein the quorum sensing controlled polypeptide can correspond to all or a portion of a quorum sensing controlled polypeptide. In a preferred embodiment, a quorum sensing controlled fusion protein comprises at least one biologically active portion of a quorum sensing controlled polypeptide. In another preferred embodiment, a quorum sensing controlled fusion protein comprises at least two biologically active portions of a quorum sensing controlled polypeptide. Within the fusion protein, the term “operatively linked” is intended to indicate that the quorum sensing controlled polypeptide and the non-quorum sensing controlled polypeptide are fused in-frame to each other. The non-quorum sensing controlled polypeptide can be fused to the N-terminus or C-terminus of the quorum sensing controlled polypeptide.

For example, in one embodiment, the fusion protein is a GST-quorum sensing controlled fusion protein in which the quorum sensing controlled sequences are fused to the C-terminus of the GST sequences. Such fusion proteins can facilitate the purification of recombinant quorum sensing controlled polypeptides.

In another embodiment, the fusion protein is a quorum sensing controlled polypeptide containing a heterologous signal sequence at its N-terminus. In certain host cells (e.g., mammalian host cells), expression and/or secretion of quorum sensing controlled polypeptide can be increased through use of a heterologous signal sequence.

The quorum sensing controlled fusion proteins of the invention can be incorporated into pharmaceutical compositions and administered to a subject in vivo. The quorum sensing controlled fusion proteins can be used to affect the bioavailability of a quorum sensing controlled substrate. Use of quorum sensing controlled fusion proteins may be useful therapeutically for the treatment of biofilm-associated diseases or disorders.

Moreover, the quorum sensing controlled -fusion proteins of the invention can be used as immunogens to produce anti-quorum sensing controlled antibodies in a subject, to purify quorum sensing controlled ligands and in screening assays to identify molecules which inhibit the interaction of quorum sensing controlled polypeptides with a quorum sensing controlled polypeptide substrate.

Preferably, a quorum sensing controlled chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypepide). A quorum sensing controlled molecule-encoding nucleic acid can be cloned into such an expression vector such that the fusion moiety is linked in-frame to the quorum sensing controlled polypeptide.

The present invention also pertains to the use of variants of the quorum sensing controlled polypeptides which function as either quorum sensing controlled polypeptide agonists (mimetics) or as quorum sensing controlled polypeptide antagonists. Variants of the quorum sensing controlled polypeptides can be generated by mutagenesis, e.g., discrete point mutation or truncation of a quorum sensing controlled polypeptide. An agonist of the quorum sensing controlled polypeptides can retain substantially the same, or a subset, of the biological activities of the naturally occurring form of a quorum sensing controlled polypeptide. An antagonist of a quorum sensing controlled polypeptide can inhibit one or more of the activities of the naturally occurring form of the quorum sensing controlled polypeptide by, for example, competitively modulating a quorum sensing controlled polypeptide-mediated activity of a quorum sensing controlled polypeptide. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the quorum sensing controlled polypeptide.

In one embodiment, variants of a quorum sensing controlled polypeptide which function as either quorum sensing controlled molecule agonists (mimetics) or as quorum sensing controlled molecule antagonists can be identified by screening combinatorial libraries of mutants, e.g., truncation mutants, of a quorum sensing controlled polypeptide for quorum sensing controlled polypeptide agonist or antagonist activity. In one embodiment, a variegated library of quorum sensing controlled molecule variants is generated by combinatorial mutagenesis at the nucleic acid level and is encoded by a variegated gene library. A variegated library of quorum sensing controlled molecule variants can be produced by, for example, enzymatically ligating a mixture of synthetic oligonucleotides into gene sequences such that a degenerate set of potential quorum sensing controlled sequences is expressible as individual polypeptides, or alternatively, as a set of larger fusion proteins (e.g., for phage display) containing the set of quorum sensing controlled gene sequences therein. There are a variety of methods which can be used to produce libraries of potential quorum sensing controlled variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential quorum sensing controlled gene sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a quorum sensing controlled polypeptide coding sequence can be used to generate a variegated population of quorum sensing controlled fragments for screening and subsequent selection of variants of a quorum sensing controlled polypeptide. In one embodiment, a library of coding sequence fragments can be generated by treating a double stranded PCR fragment of a quorum sensing controlled coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the quorum sensing controlled polypeptide.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of quorum sensing controlled polypeptides. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify quorum sensing controlled variants (Arkin and Yourvan (1992) Proc. Natl. Acad. Sci. USA 89:7811-7815; Delgrave et al. (1993) Protein Engineering 6(3): 327-331).

In one embodiment, cell based assays can be exploited to analyze a variegated quorum sensing controlled molecule library. For example, a library of expression vectors can be transfected into a cell line which ordinarily responds to a quorum sensing controlled molecule ligand in a particular quorum sensing controlled ligand-dependent manner. The transfected cells are then contacted with a quorum sensing controlled molecule ligand and the effect of expression of the mutant on, e.g., modulation of biofilm formation or modulation of antibiotic resistance can be detected. Plasmid DNA can then be recovered from the cells which score for inhibition, or alternatively, potentiation of signaling by the quorum sensing controlled molecule ligand, and the individual clones further characterized.

An isolated quorum sensing controlled polypeptide, or a portion or fragment thereof, can be used as an immunogen to generate antibodies that bind quorum sensing controlled polypeptide using standard techniques for polyclonal and monoclonal antibody preparation. A full-length quorum sensing controlled polypeptide can be used or, alternatively, the invention provides antigenic peptide fragments of quorum sensing controlled polypeptides for use as immunogens. The antigenic peptide of quorum sensing controlled polypeptide comprises at least 8 amino acid residues of the amino acid sequence shown in SEQ ID NOS:354-706 and encompasses an epitope of quorum sensing controlled such that an antibody raised against the peptide forms a specific immune complex with quorum sensing controlled polypeptide. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of quorum sensing controlled polypeptides that are located on the surface of the protein, e.g., hydrophilic regions, as well as regions with high antigenicity.

A quorum sensing controlled polypeptide immunogen typically is used to prepare antibodies by immunizing a suitable subject, (e.g., rabbit, goat, mouse or other mammal) with the immunogen. An appropriate immunogenic preparation can contain, for example, a recombinantly expressed quorum sensing controlled polypeptide or a chemically synthesized quorum sensing controlled polypeptide. The preparation can further include an adjuvant, such as Freund's complete or incomplete adjuvant, or similar immunostimulatory agent. Immunization of a suitable subject with an immunogenic quorum sensing controlled preparation induces a polyclonal anti-quorum sensing controlled antibody response.

Accordingly, another aspect of the invention pertains to the use of anti-quorum sensing controlled antibodies. The term “antibody” as used herein refers to immunoglobulin molecules and immunologically active portions of immunoglobulin molecules, i.e., molecules that contain an antigen binding site which specifically binds (immunoreacts with) an antigen, such as a quorum sensing controlled polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind quorum sensing controlled polypeptides. The term “monoclonal antibody” or “monoclonal antibody composition”, as used herein, refers to a population of antibody molecules that contain only one species of an antigen quorum sensing controlled polypeptide binding site capable of immunoreacting with a particular epitope of quorum sensing controlled polypeptide. A monoclonal antibody composition thus typically displays a single binding affinity for a particular quorum sensing controlled polypeptide with which it immunoreacts.

Polyclonal anti-quorum sensing controlled antibodies can be prepared as described above by immunizing a suitable subject with a quorum sensing controlled immunogen. The anti-quorum sensing controlled antibody titer in the immunized subject can be monitored over time by standard techniques, such as with an enzyme linked immunosorbent assay (ELISA) using immobilized quorum sensing controlled polypeptides. If desired, the antibody molecules directed against quorum sensing controlled polypeptides can be isolated from the mammal (e.g., from the blood) and further purified by well known techniques, such as protein A chromatography to obtain the IgG fraction. At an appropriate time after immunization, e.g., when the anti-quorum sensing controlled antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem.255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med, 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a quorum sensing controlled immunogen as described above, and the culture supernatants of the resulting hybridoma cells are screened to identify a hybridoma producing a monoclonal antibody that binds quorum sensing controlled polypeptides.

Any of the many well known protocols used for fusing lymphocytes and immortalized cell lines can be applied for the purpose of generating an anti-quorum sensing controlled monoclonal antibody (see, e.g., G. Galfre et al. (1977) Nature 266:55052; Gefter et al Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind quorum sensing controlled polypeptides, e.g., using a standard ELISA assay.

Alternative to preparing monoclonal antibody-secreting hybridomas, a monoclonal anti-quorum sensing controlled antibody can be identified and isolated by screening a recombinant combinatorial immunoglobulin library (e.g., an antibody phage display library) with quorum sensing controlled polypeptides to thereby isolate immunoglobulin library members that bind quorum sensing controlled polypeptides. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. Nature (1990) 348:552-554.

Additionally, recombinant anti-quorum sensing controlled antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-quorum sensing controlled antibody (e.g., monoclonal antibody) can be used to isolate quorum sensing controlled polypeptides by standard techniques, such as affinity chromatography or immunoprecipitation. An anti-quorum sensing controlled antibody can facilitate the purification of natural quorum sensing controlled polypeptides from cells and of recombinantly produced quorum sensing controlled expressed in host cells. Moreover, an anti-quorum sensing controlled antibody can be used to detect quorum sensing controlled polypeptide (e.g., in a cellular lysate or cell supernatant) in order to evaluate the abundance and pattern of expression of the quorum sensing controlled polypeptide. Anti-quorum sensing controlled antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Methods of Treatment of Subjects Suffering from Biofilm-Associated Disease or Disorders

The present invention provides for both prophylactic and therapeutic methods of treating a subject, e.g., a human, at risk of (or susceptible to) a biofilm-associated disease or disorder.

A. Prophylactic Methods

In one aspect, the invention provides a method for preventing in a subject, a biofilm-associated disease or disorder by administering to the subject an agent which modulates quorum sensing controlled gene expression or quorum sensing controlled polypeptide activity (e.g., a modulator identified by a screening assay described herein). Subjects at risk for a biofilm-associated disease or disorder can be identified by, for example, any or a combination of the diagnostic or prognostic assays described herein. Administration of a prophylactic agent can occur prior to the manifestation of symptoms characteristic of aberrant quorum sensing controlled gene expression or polypeptide activity, such that a biofilm-associated disease or disorder is prevented or, alternatively, delayed in its progression. Depending on the type of biofilm-associated aberrancy, for example, a quorum sensing controlled molecule agonist or quorum sensing controlled molecule antagonist agent can be used for treating the subject. The appropriate agent can be determined based on screening assays described herein.

B. Therapeutic Methods

Another aspect of the invention pertains to methods for treating a subject suffering from a biofilm-associated disease or disorder. These methods involve administering to a subject a quorum sensing controlled nucleic acid modulator or a quorum sensing controlled polypeptide modulator (e.g., a modulator identified by a screening assay described herein), or a combination of such modulators.

The agents or compounds which modulate biofilm formation can be administered to a subject using pharmaceutical compositions suitable for such administration. Such compositions typically comprise the agent (e.g., nucleic acid molecule, protein, or antibody) and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition used in the therapeutic methods of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the agent that modulates biofilm formation in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the agents that modulate biofilm formation are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the agent that modulates biofilm formation and the particular therapeutic effect to be achieved, and the limitations inherent in the art of compounding such an agent for the treatment of subjects.

Toxicity and therapeutic efficacy of such agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and can be expressed as the ratio LD50/ED50. Agents which exhibit large therapeutic indices are preferred. While agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage for use in humans. The dosage of such biofilm modulating agents lies preferably within a range of circulating concentrations that include the ED50 with little or no toxicity. The dosage may vary within this range depending upon the dosage form employed and the route of administration utilized. For any agent used in the therapeutic methods of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in-the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, nitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil. melphalan, carmustine (BSNU) and lomustine (CCNU), cyclothosphamide, busulfan, libromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules used in the methods of the invention can be inserted into vectors and used as gene therapy vectors. Gene therapy vectors can be delivered to a subject by, for example, intravenous injection, local administration (see U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

Incorporation by Reference

The contents of all references, patents and published patent applications cited throughout this application, as well as the figures and the sequence listing, are incorporated herein by reference.

### EXEMPLIFICATION

The invention is further illustrated by the following examples which should not be construed as limiting.

### Example 1

Identification of Quorum Sending Genes of P. aeruginosa

Materials and Methods

Bacterial Strains, Plasmids, and Media. The bacterial strains and plasmids used in this example are listed in Table 2.

E. coli and P. aeruginosa were routinely grown in Luria-Bertani (LB) broth or LB agar (Sambrook, et al. (1989) Molecular Cloning: a Laboratory Manual. (Cold Spring Harbor Laboratory, Cold Spring Harbor, N.Y.)), supplemented with antimicrobial agents when necessary. The antimicrobial agents were used at the following concentrations: HgCl₂, 15 μg/ml in agar and 7.5 μg/ml in broth; nalidixic acid 20 μg/ml; carbenicillin, 300 μg/ml; tetracycline, 50 μg/ml for P. aeruginosa and 20 μg/ml for E. coli; and gentamicin, 100 μg/ml for P. aeruginosa and 15 μg/ml for E. coli. Synthetic acyl-HSL concentrations were 2 μM for 3OC₁₂-HSL and 5 μM for C₄-HSL, and 5-bromo-4-chloro-3-indolyl-β-D-galactopyranoside (X-Gal) was used at 50 μg/ml.

DNA Manipulations and Plasmid Constructions. DNA treatment with modifying enzymes and restriction endonucleases, ligation of DNA fragments with T4 ligase, and transformation of E. coli were performed according to standard methods (Ausubel, F. et al. (1997) Short Protocols in Molecular Biology. (John Wiley & Sons, Inc., New York, N.Y.)). Plasmid isolation was performed using QIAprep spin miniprep kits (Qiagen Inc.) and DNA fragments were excised and purified from agarose gels using GeneClean spin kits (Bio101 Corp.). DNA was sequenced at the University of Iowa DNA core facility by using standard automated sequencing technology.

To construct pMW10, the pBR322 tetA(C) gene-containing ClaI-NotI DNA fragment in pJPP4 was replaced with a tetA(B)-containing BstB 1-NotI fragment from Tn10. It was necessary to use tetA(B) rather than tetA(C) to inactivate lasI because the tetA(C) gene from pBR322 was a hot spot for Tn5::B22 mutagenesis (Berg, D. E. et al. (1983) Genetics 105, 813-828).

To construct pMW300 a 1.6-kb SmaI fragment from pGMΩ1 that contained the aacC1 gene (encoding gentamicin acetyltransferase-3-1) was cloned into EagI digested pTL61T, which had been polished with T4 polymerase. The resulting plasmid pTL61T-GMΩ1 was digested with SmaI and MscI to release a 6.5-kb lacZ-aacC1 fragment. A

TABLE 2
Bacterial strains and plasmids

| | | Source |
|---|---|---|
| Strain or plasmid | Relevant characteristics | (reference) |
| Strains | | |
| P. aeruginosa | Parental strain | (1) |
| PAO1 | | |
| P. aeruginosa | Δrhll::Tn501 derivative of PAO1, Hg^{r} | (2) |
| PDO100 | | |
| P. aeruginosa | Δlasl, Δrhll derivative of PDO100, Hg^{r}, | This study |
| PAO-MW1 | Tc^{r} | |
| P. aeruginosa | lasB::lacZ chromosomal insertion in | This study |
| PAO-MW10 | PAO-MW1 | |
| E. coli DH5α | F⁻ φ80ΔlacZ, ΔM15, Δ(lacZYA-argF) | (3) |
| | U169, endAl, recAl, hsdR17, deoR, | |
| | gyrA96, thi-1 relAl, supE44 | |
| E. coli HB101 | F⁻ mcrB, mrr hsdS20, recA13, leuB6, | (3) |
| | ara-14 proA2, lacY1, galK2, xyl-5, | |
| | mtl-l, rpsL20 (Sm^{r}), supE44 | |
| E. coli SY327 λpir | (λpir), A(lac pro), argE(Am), rif, nlA, | (4) |
| | recA56 | |
| E. coli S17-1 | thi, pro, hsdR, recA, RP4-2 (Tet::Mu) | (5) |
| | (Km::Tn7) | |
| Plasmids | | |
| pJPP4 | oriR6K, mobRP4, Δlasl, Tc^{r}, Ap^{r} | (6) |
| pTL61T | lacZ transcriptional fusion vector, Ap^{r} | (7) |
| pGMΩ1 | Contains aacl flanked by transcriptional | (8) |
| | and translational stops, Gm^{r} | |
| pTL61T-GMΩ1 | pTL61T with aacl gene from pGMΩ1 | This study |
| | upstream of lacZ, Ap^{r}, Gm^{r} | |
| pMW100 | pJPP4 with 2.7-kb tetA(B) from Tn10 | This study |
| | in place of the pBR322 tetA(C), Tc^{r}, | |
| | Ap^{r} | |
| pRK2013 | ori (ColE1), tra⁺, (RK2)Km^{r} | (9) |
| pSUP102 | pACYC184 carrying mobRP4, Cm^{r}, Tc^{r} | (10) |
| pSUP102-lasB | pSUP102 carrying lasB on a 3.1-kb | This study |
| | P. aeruginosa chromosomal DNA | |
| | fragment, Cm^{r}, Tc^{r} | |
| pMW300 | pSUP102-lasB containing lacZ-aacl | This study |
| | from pTL61T-GMΩ1 (lasB-lacZ | |
| | transcriptional fusion knockout | |
| | plasmid), Cm^{r}, Gm^{r} | |
| pTn5-B22 | pSUP102 with Tn5-B22 (‘lacZ), Gm^{r} | (28) |

Abbreviations for antibiotics are as follows: kanamycin, Km; gentamicin, Gm; ampicillin, Ap; tetracycline, Tc; streptomycin, Sm.

3.1-kb P. aeruginosa PAO1 chromosomal DNA fragment containing the lasB gene was amplified by PCR using the Expand™ Long Template PCR System (Boehringer Mannheim). This fragment was cloned into BamHI-digested pSUP102. The resulting plasmid, pSUP 102-lasB was digested with NotI, polished with T4 polymerase and ligated with the 6.5-kb lacZ-aacC1 fragment from pTL61T-GMΩ1 to generate pMW300. The promoterless lacZ gene in pMW300 is 549 nucleotides form the start of the lasB ORF, it is flanked by 1.5 kb upstream and 1.6 kb downstream P. aeruginosa DNA, and it contains the p15A ori, which does not support replication in P. aeruginosa.

Construction of P. aeruginosa Mutants. A lasI, rhlI mutant strain of P. aeruginosa PAO-MW1 was generated by insertional mutagenesis of lasI in the rhlI deletion mutant, PDO100. For insertional mutagenesis, the lasI::tetA(B) plasmid, pMW100 was mobilized from E. coli SY327 λpir into PDO100 by triparental mating with the help of E. coli HB101 containing pRK2013. Because pMW100 has a λpir-dependent origin of replication, it cannot replicate in P. aeruginosa. A tetracycline-resistant, carbenicillin-sensitive exconjugant was selected, which was shown by a Southern blot analysis to contain lasI:tetA but not lasI or pMW100. To confirm the inactivation of the chromosomal lasI in this strain, PAO-MW1, the amount of 3OC₁₂-HSL in the fluid from a stationary phase culture (optical density at 600 nm, 5) was assessed by a standard bioassay (Pearson, J. P. et al. (1994) PNAS, 91, 197-201). No detectable 3-OC₁₂-HSL (<5 nM) was found.

A mutant strain, P. aeruginosa PAO-MW10. which contains a lacZ reporter in the chromosomal lasB gene was constructed by introduction of pMW300 into PAO-MW1 by triparental mating as described above. Exconjugants resistant to gentamicin and sensitive to chloramphenicol were selected as potential recombinants. Southern blotting of chromosomal DNA with lasB and lacZ probes indicated that the pMW300 lasB-lacZ insertion had replaced the wt lasB gene.

Southern Blotting. Chromosomal DNA was prepared using the QIAMP tissue kit (Qiagen Inc.). Approximately 2 μg of chromosomal DNA was digested with restriction endonucleases, separated on a 0.7% agarose gel, and transferred to a nylon membrane according to standard methods (Ausubel, F. et al. (1997) Short Protocols in Molecular Biology. (John Wiley & Sons, Inc., New York, N.Y.). DNA probes were generated using digoxigenin-11-dUTP by random primed DNA labeling or PCR. The Southern blots were visualized using the Genius# system as outlined by the manufacturer (Boehringer Mannheim).

Tn5 Mutagenesis. Tn5::B22, which carries a promoterless lacZ gene, was used to mutagenize P. aeruginosa PAO-MW1 (Simon, R. et al. (1989) Gene 80, 161-169). Equal volumes of a late logarithmic phase culture of E. coli S17-1 carrying pTn5::B22 grown at 30° C. with shaking and a late logarithmic phase culture of P. aeruginosa PAO-MW1 grown at 42° C. without shaking were mixed. The mixture was centrifuged at 6000×g for 10 minutes at room temperature, suspended in LB (5% of the original volume), and spread onto LB plates (100 μl per plate). After 16 to 24 hours at 30° C., the cells on each plate were suspended in 500 μl LB and 100 μl volumes were spread onto LB agar plates containing HgCl₂, gentamicin, tetracycline and nalidixic acid. The nalidixic acid prevents growth of E. coli but not P. aeruginosa. After 48 to 72 hours at 30° C., 20 colonies were selected from each mating and grown on LB selection agar plates containing X-gal. Ten of the 20 were picked for further study. The colonies picked showed a range in the intensity of the blue color on the X-gal plates. In this way, the selection of siblings in a mating were minimized. A Southern blot using a probe to lacZ was performed on 20 randomly chosen transconjugants indicated that the Tn5 insertion in each was in a unique location.

The Screen for qsc Fusions. A microtiter dish assay was used to identify mutants showing acyl-HSL-dependent β-galactosidase expression (quorum sensing-controlled or qsc mutants). Each transconjugant was grown in four separate wells containing LB broth without added autoinducer, with added 3OC₁₂-HSL, C₄-HSL, or both 3OC₁₂-HSL and C₄-HSL for 12-16 hours at 37° C. Inocula were 10 μl of an overnight culture and final culture volumes were 70 μl. The β-galactosidase activity of cells in each microtiter dish well was measured in microtiter dishes with a luminescence assay (Tropix) Luminescence was measured with a Lucy I microtiter dish luminometer (Anthos).

Patterns of Acyl-HSL Induction of β-galactosidase Activity in qsc Mutants. The pattern of β-galactosidase expression was examined in response to acyl-HSLs in each of 47 qsc mutants identified in the initial screen. Each mutant was grown in 1 ml of MOPS (50 mM, pH 7.0) buffered LB broth containing one, the other, both, or neither acyl-HSL signal in an 18 mm culture tube at 37° C. with shaking. A mid-logarithmic phase culture was used as an inoculum and initial optical densities (ODs) at 600 nm were 0.1. Growth was monitored as OD at 600 nm and β-galactosidase activity was measured in 0.1 ml samples taken at 0, 2, 5, and 9 hours after inoculation.

DNA Sequencing and Sequence Analysis. To identify DNA sequences flanking Tn5::B22 insertions, arbitrary PCR was performed with primers and conditions as described (Caetano-Annoles, G. (1993) PCR Methods Appl. 3, 85-92; O'Toole, G. A. et al. (1998) Mol. Microbiol. 28, 449-461). Tn5 flanking sequences that could not be identified using arbitrary PCR were cloned. For cloning, 3 μg of chromosomal DNA was digested with EcoRI and ligated with EcoRI-digested, phosphatase treated pBR322. E. coli DH5α was transformed by electroporation with the ligation mixtures and plasmids from gentamicin resistant colonies were used for sequencing Tn5-flanking DNA.

DNA sequences flanking Tn5-B22 insertions were located on the P. aeruginosa PAO1 chromosome by searching the chromosomal database at the P. aeruginosa Genome Project web site (www.pseudomonas.com). The ORFs containing the insertions are those described at the web site. Functional coupling from the Argonne National Labs (http://wit.mcs.anl.gov/WIT2), sequence analysis, and expression patterns of the qsc mutants were used to identify potential operons (Overbeek, R. et al. (1999) PNAS 96, 2896-2901).

Results

Identification of Pseudomonas aeruginosa qsc Genes. Seven thousand Tn5::B22 mutants of P. aeruginosa PAO-MW1 were screened. Tn5::B22 contains a promoterless lacZ. P. aeruginosa PAO-MW1 is a lasI rhlI mutant that does not make acyl-HSL signals. Thus, transcription of the Tn5::B22 lacZ in a qsc gene was expected to respond to an acyl-HSL signal. The screen involved growth of each mutant in a complex medium in a microtiter dish well with no added acyl-HSL, 3OC₁₂-HSL, C₄-HSL, or both 3OC₁₂-HSL and C₄-HSL. After 12-16 hours, β-galactosidase activity in each culture was measured. Two hundred-seventy mutants showed greater than 2 fold stimulation of β-galactosidase activity in response to either or both acyl-HSL. Of these, 70 showed a greater than 5-fold stimulation of β-galactosidase activity in response to either or both acyl-HSL, and were studied further. Each mutant was grown with shaking in culture tubes and 47 showed a reproducible greater than 5-fold stimulation of β-galactosidase activity in response to either or both of the acyl-HSL signals. These were considered to have Tn5::B22 insertions in qsc genes. It was shown by a Southern blot analysis with a lacZ probe that each mutant contained a single Tn5::B22 insertion.

This collection of 47 mutants is not believed to represent the entire set of quorum sensing regulated genes in P. aeruginosa. The threshold of greater than 5-fold induction may be too stringent, enough mutants may not have been screened to be assure that insertions in all of the genes in the chromosome have been tested, and there may be conditions other than those which were employed that would have revealed other genes which were not detected in the present screen. Nevertheless, a set of 47 insertions in genes have been identified that show significant activation in response to acyl-HSL (qsc genes).

Responses of qsc Mutants to Acyl-HSL Signals. For cultures of each of the 47 qsc mutants, β-galactosidase activity was measured at different times after addition of acyl-HSL signals. The basal levels of β-galactosidase varied depending on the mutant. The responses to the acyl-HSL signals could be grouped into 4 general classes based on which of the two signals was required for activation of lacZ, and whether the response to the signal(s) occurred immediately or was delayed until stationary phase. A response was considered immediate if there was a 5-fold or greater response within 2 hours of acyl-HSL addition (the optical densities(ODs) of the cultures ranged from 0.5-0.7 at 2 hours). A response was considered delayed or late if there was <5-fold induction at 2 hours but greater than 5-fold induction of β-galactosidase at 5 hours or later (ODs of 2 or greater). In some strains activation of lacZ required 3OC₁₂-HSL, others required both 3OC₁₂-HSL and C₄-HSL for full activation of lacZ. A number of strains responded to either signal alone but showed a much greater response with both 3OC₁₂-HSL and C₄-HSL. None of the mutants responded well to C₄-HSL alone (Table 3). This was expected because expression of RhlR, which is required for a response to C₄-HSL is dependent on 3OC₁₂-HSL (Pesci, E. C. et al. (1997) J. Bacteriol. 179, 3127-3132). Therefore at least some of the insertions exhibiting a response to both acyl-HSLs may be responding to the rhl system, which requires activation by the las system.

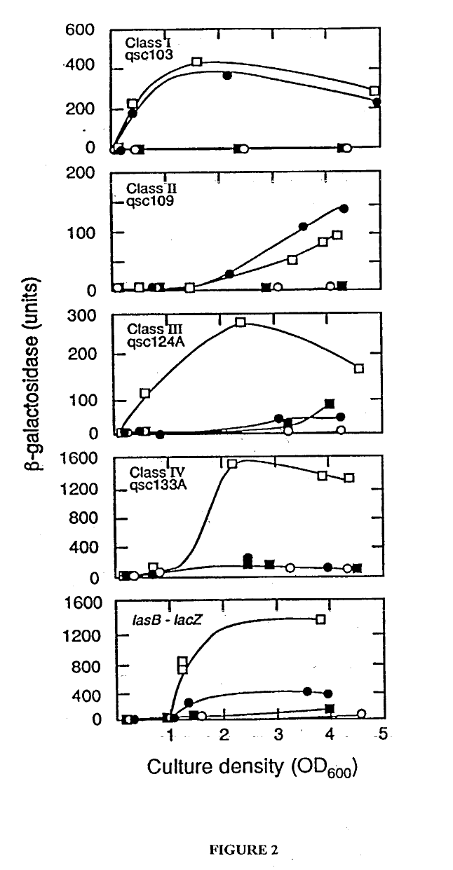

Class I mutants responded to 3OC₁₂-HSL immediately, Class II responded to 3OC₁₂-HSL late, Class III respond best to both signals early, and Class IV to both signals late. There were 9 Class I, 11 Class II, 18 Class III, and 9 Class IV mutants. FIG. 2 shows responses of representative members of each class to acyl-HSLs. Generally, most early genes (Class I and III genes) showed a much greater induction than most late genes (Class II and IV). Many of the Class III mutants showed some response to either signal alone but showed a greater response in the presence of both signals (Table 3 and FIG. 2).

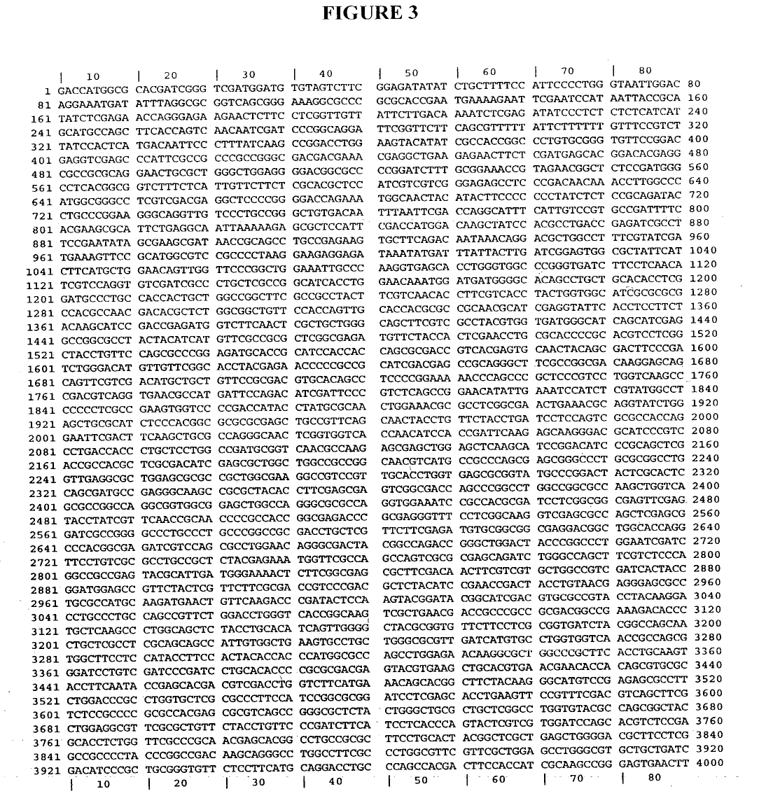

Identity and Analysis of qsc Genes. The Tn5-B22-marked qsc genes were identified by coupling arbitrary PCR or transposon cloning with DNA sequencing. The sequences were located in the P. aeruginosa PAO1 chromosome by searching the Pseudomonas aeruginosa Genome Project web site (www.pseudomonas.com). To confirm the locations of the Tn5-B22 insertions in each qsc mutant, a Southern blot analysis was performed with Tn5-B22 as a probe. The sizes of Tn5-B22 restriction fragments were in agreement with those predicted based on the P. aeruginosa genomic DNA sequence (data not shown). The 47 qsc mutations mapped in or adjacent to 39 different open reading frames (ORFs). For example FIG. 3 depicts the nucleic acid sequence of the quorum sensing controlled locus on the P. aeruginosa chromosome mapped in the P. aeruginosa mutant strain qsc 102.

TABLE 3
Quorum sensing-controlled genes in Pseudomonas aeruginosa

| | | Signal response^{b} | | | Genomic |
|---|---|---|---|---|---|
| Classification | Identity^{a} | 3OC₁₂-HSL | C₄-HSL | Both | Position^{e} |
| Class I | | | | | |
| qsc100 | Peptide synthetase | 65 | 3 | 69 | 5801998 |
| qsc101 | No match | 145 | 1 | 184 | 7730 |
| qsc102 | No match | 350 | 1 | 400 | 5547 |
| qsc103 | No match | 85 | 1 | 95 | 3961920 |
| qsc104 | Polyamine binding protein | 7 | 2 | 8 | 5402505 |
| qsc105 | FAD-binding protein | 40 | 1 | 42 | 5410045 |
| qsc106A&B | No match | 9 | 1 | 10 | 2870317 |
| qsc107 | No match | 44 | 2 | 50 | 5799641 |
| Class II | | | | | |
| qsc108 | ORF 5 | 13 | 1 | 7 | 5617382 |
| qsc109 | Bacitracin synthetase 3 | 13 | 1 | 8 | 5651872 |
| qsc110A&B | Pyoverdine synthetase D | 10 | 1 | 7 | 5661697 |
| qsc111 | Pyoverdine synthetase D | 11 | 1 | 7 | 5666282 |
| qsc112A&B | Aculeacin A acylase | 15 | 1 | 12 | 5701004 |
| qsc113 | Trransmembrane protein | 5 | 1 | 5 | 3771157 |
| qsc114^{c} | No match | 9 | 1 | 7 | 5209051 |
| qsc115^{d} | No match | 4 | 1 | 5 | 1941557 |
| qsc116 | No match | 5 | 1 | 5 | 1138940 |
| Class III | | | | | |
| qsc117^{d} | ACP-like protein | 22 | 22 | 186 | 41430 |
| qsc118 | RhlI | 38 | 14 | 70 | 4447967 |
| qsc119 | RhlB | 9 | 7 | 100 | 4446918 |
| qsc120 | Chloramphenicol resistance | 3 | 7 | 24 | 4592102 |
| qsc121 | 3-Oxoacyl ACP synthase | 13 | 27 | 105 | 4594988 |
| qsc122A&B | Cytochrome p450 | 2 | 10 | 90 | 4593538 |
| qsc123 | 9-Cis retinol dehydrogenase | 14 | 28 | 96 | 4597340 |
| qsc124A&B | Pyoverdine synthetase D | 35 | 50 | 148 | 4598281 |
| qsc125 | Zeaxanthin synthesis | 20 | 65 | 140 | 4600099 |
| qsc126 | Pristanimycin I synthase 3 & 4 | 3 | 5 | 24 | 4603518 |
| qsc127^{c} | No match | 5 | 2 | 15 | 4608787 |
| qsc128 | Hydrogen cyanide synthase HcnB | 19 | 12 | 42 | 5924799 |
| qsc129A&B | Cellulose binding protein p40 | 15 | 1 | 100 | 1141723 |
| qsc130 | glc operon transcriptional activator | 5 | 1 | 14 | 2313744 |
| qsc131 | PhzC | 50 | 168 | 742 | 1110 |
| Class IV | | | | | |
| qsc132A&B | Unknown (B. pertusis) | 1 | 1 | 40 | 3616599 |
| qsc133A&B | AcrB | 1 | 1 | 9 | 3628342 |
| qsc134 | Saframycin Mx1 synthetase A | 6 | 1 | 28 | 3781254 |
| qsc135 | Cytochrome C precursor | 3 | 1 | 6 | 4942182 |
| qsc136^{c} | No match | 7 | 3 | 45 | 851491 |
| qsc137 | Asparagine synthetase | 1 | 1 | 10 | 2007007 |
| qsc138 | No match | 3 | 5 | 32 | 2459178 |

^{a}The bold letters indicate matches were to known P. aeruginosa genes.
^{b}The signal response is given as β-galactosidase activity in cells grown in the presence of the indicated signal(s) divided by the β-galactosidase activity of cells grown in the absence of added signals. Maximum responses are indicated.
^{c}The lacZ insertions in these strains are in the opposite orientation of the ORFs described in the P. aeruginasa Genome Project web site. The insertions are which in locations with no reported identity are been indicated.
^{d}Insertions do not lie in but are near the putative ORFs indicated. In qsc117 the insertion is 129 bp downstream of the ACP ORF and interrupts a potential rho-independent transcription terminator. The qsc115 insertion is 60 bp upstream of the ORF listed in Materials and Methods.
^{e}Genomic position as identified using sequence information described in the P. aeruginosa Genome Project web site (Jul. 15, 1999 release).

The genomic sequences comprising the ORFs in Table 3 are described in the Pseudomonas aeruginosa Genome Sequencing Project web site, as detailed in Table 4.

Only 2 genes were identified that already were known to be controlled by quorum sensing, rhlI and rhlB. Several other genes potentially involved in processes known to be regulated by quorum sensing were also identified including phzC (phenazine synthesis), a putative cyanide synthesis gene (related to the Pseudomonas fluorescens hcnB), and ORF 5 (pyoverdine synthesis) (Latifi, A. et al. (1995) Mol. Microbiol. 17, 333-344; Cunliffe, H. E. et al. (1995) J. Bacteriol. 177, 2744-2750). Interestingly, lasB was not identified by the assay, yet the LasI-LasR quorum sensing system was originally described as regulating lasB (Gambello, M. J. et al. (1991) J. Bacteriol. 173, 3000-3009). A lasB-lacZ chromosomal fusion in P. aeruginosa PAO -MW1 was constructed, so that regulation of lasB by quorum sensing could be compared to the genes identified by the assay. The lasB-lacZ fusion only responded slightly to 3OC₁₂-HSL (3-fold stimulation). The full response (12-13-fold over background) required both C₄-HSL and 3OC₁₂-HSL., and the response was late (FIG. 2). Thus, lasB shows the characteristics of a Class IV gene.

Some of the qsc mutants had obvious phenotypes. Unlike the parent, on LB agar, colonies of the Class II mutants qsc108, 109, 110A and B, and 111 were not fluorescent. Because pyoverdine is a fluorescent pigment, and because the qsc110 and 111 mutations were in genes coding for pyoverdine synthetase-like proteins, it was suspected that these mutations define a region involved in pyoverdine synthesis. The insertion in qsc131 is in phzC which is required for pyocyanin synthesis. Although the parent strain produced a blue pigment in LB broth, qsc131 did not. The two qsc132 mutants also did not produce detectable levels of pyocyanin but did produce a water-soluble red pigment.

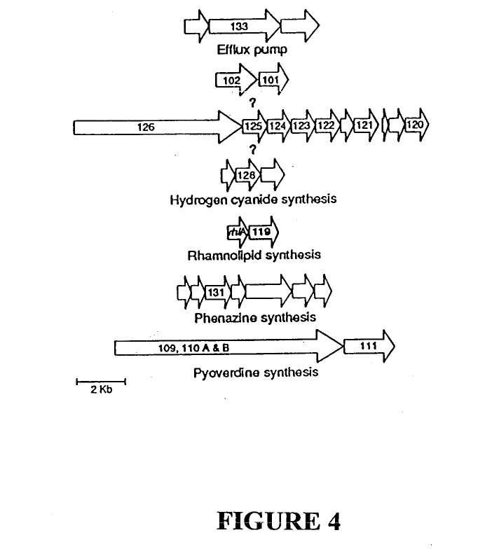

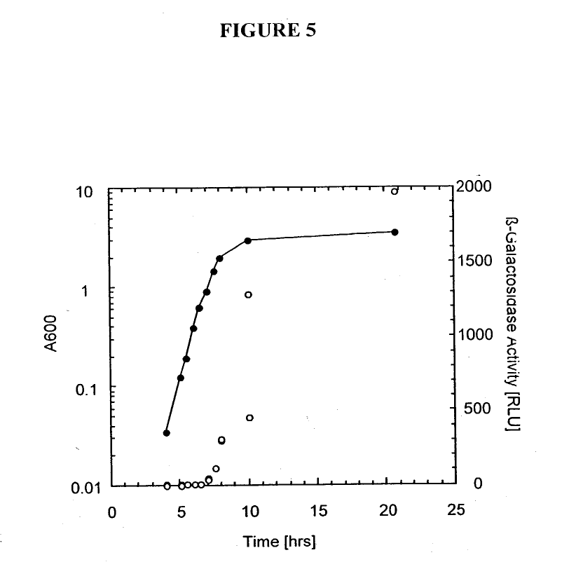

Functional coupling and sequence analysis were used to identify 7 putative qsc operons, one of which is the previously described rhlAB operon (FIG. 4). Functional coupling will not organize genes encoding polypeptides without known relatives into operons, and organization of genes in an operon was disallowed in cases where there was greater than 250 bp of intervening sequence between two adjacent ORFs. The

TABLE 4
ORFs of quorum sensing-controlled genes in Pseudomonas aeruginosa

| | | | Open | |
|---|---|---|---|---|
| | Insertion | Insertion | Reading Frame | |
| | Jul. 15, 1999 | Dec. 15, 1999 | Dec. 15, 1999 | |
| QSC | release | release | release | Orientation |
| 131 | 1110 | 4715256 | 4714774-4715991 | Forward |
| 102 | 5547 | 2067716 | 2066736-2068517 | Reverse |
| 101 | 7730 | 2065297 | 2064803-2065495 | Reverse |
| 117 | 41430 | 2031833 | 2031245-2031655 | Forward |
| 136 | 851491 | 1221771 | 1221374-1221961 | Reverse |
| 116 | 1138940 | 934322 | 934191-935210 | Reverse |
| 129 | 1141723 | 931539 | 930603-931772 | Reverse |
| 115 | 1941557 | 131753 | 131583-131792 | Reverse |
| 137 | 2007007 | 66507 | 66264-68135 | Forward |
| 130 | 2313744 | 6023975 | 6023787-6024542 | Forward |
| 138 | 2459178 | 5878418 | 5877776-5878597 | Forward |
| 106 | 2870317 | 5467402 | 5466520-5467887 | Forward |
| 132 | 3616599 | 4721118 | 4720249-4721457 | Forward |
| 133 | 3628342 | 4709375 | 4707483-4710572 | Forward |
| 113 | 3771157 | 4566558 | 4565369-4567903 | Reverse |
| 134 | 3781254 | 4556461 | 4555202-4558177 | Forward |
| 103 | 3961920 | 4375793 | 4375589-4376680 | Forward |
| 119 | 4446918 | 3890793 | 3890724-3892004 | Reverse |
| 118 | 4447967 | 3889744 | 3889088-3889738 | Reverse |
| 120 | 4592102 | 3745609 | 3744850-3746016 | Forward |
| 121 | 4594988 | 3742723 | 3742643-3743635 | Forward |
| 122 | 4595538 | 3742173 | 3740961-3742217 | Forward |
| 123 | 4597340 | 3740171 | 3740054-3740968 | Forward |
| 124 | 4598281 | 3739430 | 3738724-3740052 | Forward |
| 125 | 4600099 | 3737612 | 3737561-3738727 | Forward |
| 126 | 4603518 | 3734193 | 3730455-3737564 | Forward |
| 127 | 4608787 | 3728924 | | Reverse |
| 135 | 4942182 | 3395532 | 3395274-3396677 | Reverse |
| 114 | 5209051 | 3128663 | 3127731-3129116 | Forward |
| 104 | 5402505 | 2935208 | 2934490-2935593 | Forward |
| 105 | 5410045 | 2927668 | 2926722-2927972 | Reverse |
| 108 | 5617382 | 2720329 | 2718890-2720643 | Reverse |
| 109 | 5651872 | 2678258 | 2671678-2679012 | Reverse |
| 110 | 5661697 | 2676014 | 2671678-2679012 | Reverse |
| 111 | 5666282 | 2671429 | 2669119-2671674 | Reverse |
| 112 | 5701004 | 2636707 | 2636467-2638800 | Reverse |
| 107 | 5799641 | 2538070 | 2532619-2539008 | Reverse |
| 100 | 5801998 | 253S711 | 2532619-2539008 | Reverse |
| 128 | 5924799 | 2412909 | 2412807-2414201 | Forward |

qsc101 and 102 genes are an example of a putative operon that was not identified by functional coupling (FIG. 4). These two ORFs did not show significant similarities with other polypeptides. Nevertheless, they are transcribed in the same direction, closely juxtaposed, qsc101 and 102 are both Class I genes, and there is a las box-like element upstream of these ORFs. Expression of the qsc102 insertion is controlled by an upstream ORF (SEQ ID NO:707) which comprises the sequences between postions 2068711 to 267911 of the P. aeruginosa genome (Dec. 15, 1999 release) which in turn is preceded by a las box regulatory element (SEQ ID NO:708) which comprises the sequences between positions 2068965 to 2068946 of the P. aeruginosa genome (Dec. 15, 1999 release). The las box is a palindromic sequence found upstream of and involved in LasR-dependent activation of lasB (Rust, L. et al., (1996) J. Bacteriol. 178, 1134-1140).

The qsc133A and B insertions are in a putative 3-gene operon with similarity to acrAB-tolC from E. coli and the mex-opr family of efflux pump operons in P. aeruginosa, one of which (mexAB-oprN) has been shown to aid 3OC₁₂-HSL efflux (Kohler, T., et a/. (1997) Mol. Microbiol. 23, 345-354; Poole, K, et al. (1993) J. Bacteriol. 175, 7363-7372; Poole, K. et al. (1996) Mol. Microbiol. 21, 713-724; Evans, K., et al. (1998) J. Bacteriol. 180, 5443-5447; Pearson, J. P. et al. (1999) J. Bacteriol. 181, 1203-1210). The qsc133 mutations are within a gene encoding a MexF homolog. The qsc133 mutants show typical Class IV regulation. Expression of lacZ is late and dependent on the presence of both acyl-HSL signals (Table 3 and FIG. 2). No las box-like sequences upstream of this suspected efflux pump operon were identified.

A third possible operon identified by functional coupling is about 8 kb and contains 10 genes. Eight strains with insertions in 6 of the 10 genes were obtained, all of which are Class III mutants (Table 3). A las box-like sequence was identified upstream of the first gene of this operon. The function of these 10 genes is unknown but the similarities shown in Table 2 suggest that they may encode functions for synthesis and resistance to an antibiotic-like compound.

The qsc128 mutation is within a gene coding for a polypeptide that shows similarity to the P. fluorescens hcnB product and appears to be in a 3-gene operon (Table 3, FIG. 4). By analogy to the P. fluorescens hcn operon, this operon is likely required for the production of the secondary metabolite, hydrogen cyanide. Previous investigations have shown that hydrogen cyanide production is reduced in P. aeruginosa rhl quorum sensing mutants. Consistent with this, qsc128 is a Class III mutant (Table 2). Full induction required both acyl-HSL signals, however, some induction of lacZ resulted from the addition of either signal alone (Table 3). A las box-like sequence was identified in the region upstream of the translational start codon of the first gene in this operon. This las-type box may facilitate an interaction with either LasR or RhlR.

The phz operon, required for phenazine biosynthesis, has been described in other pseudomonads and the insertion in strain qsc131 is located in a gene encoding a phzC homolog. Analysis of the sequence around this phzC homolog revealed an entire phenazine biosynthesis operon, phzA-G (Georgakopoulos, D. G. et al. (1994) Appl. Environ. Microbiol. 60, 2931-2938; Mavrodi, D. V. et al. (1998) J. Bacteriol. 180, 2541-2548). As discussed above, qsc131 does not produce the blue phenazine pigment pyocyanin. PhzC is part of an operon of several genes including PhzABCDEFG, and transcription of this operon is controlled by the promoter region (SEQ ID NO:709) in front of the first gene in the operon, PhzA. The phz operon in P. aeruginosa also contains a las-box like sequence upstream of the first gene of the operon. The PhzA promoter region (SEQ ID NO:709) has been cloned into a plasmid, transcriptionally fused to lacZ. The resulting plasmid (pMW303G) was transformed into PAO1 and used as a reporter strain. The resultant bacterial strain generates a quorum sensing signal and responds to it by increased β-galactosidase activity. As shown in FIG. 5, this strain displayed a high level of induction between early and late growth, thus providing a dynamic range for detecting modulation (e.g., inhibition) of quorum sensing signaling. Accordingly this strain may be useful for a single strain assay for identifying for inhibitors of quorum sensing singaling, as described herein.

The final putative operon consists of 2 or 3 genes, qsc 109-111, which appear to be involved in pyoverdine synthesis. These ORFs were not identified in the P. aeruginosa genome project web site but were identified and shown to be functionally coupled with the Argonne National Laboratory web site.

For three of the qsc insertions, the lacZ gene was in an orientation opposite to the ORF described in the Genome Project web site (qsc114, 127, and 136).

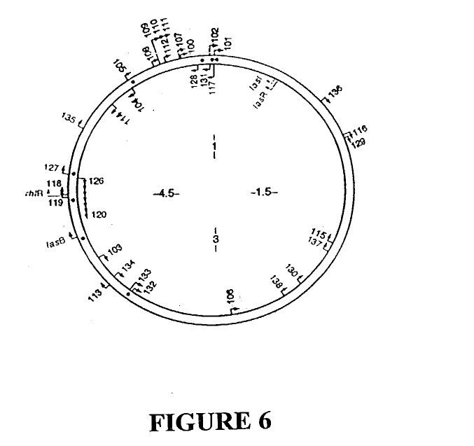

Locations of qsc Genes on the P. aeruginosa Chromosome. The qsc genes were mapped to sites on the P. aeruginosa chromosome (FIG. 6). In addition lasB, lasR and lasI, and rhlR were placed on this map. The distribution of currently identified qsc genes is patchy. For example, 16 of the 39 qsc genes representing 3 of the classes mapped to a 600-kb region of the 6 megabase chromosome. A 140-kb island of 12 Class III genes, 8 transcribed in one direction and 4 transcribed in the other direction (including the rhl genes) formed another cluster on the chromosome.

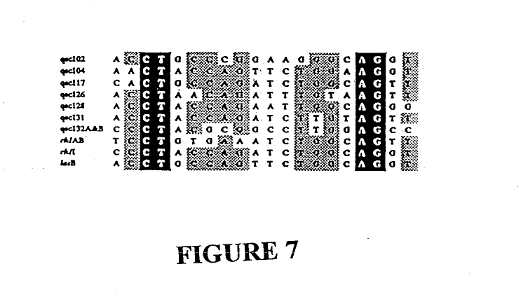

Identification of las Box-Like Sequences that Could be Involved in qsc Gene Control. As discussed above, the las box is a palindromic sequence found upstream of and involved in LasR-dependent activation of lasB (Rust, L. et al. (1996) J. Bacteriol. 178, 1134-1140). The las box shows similarity to the lux box, which is the promoter element required for quorum control of the V. fischeri luminescence genes (Devine, J. et al. (1989) PNAS 86, 5688-5692). Elements similar to a las box were identified by searching upstream of qsc ORFs. A search was done for sequences with at least 50% identity to the las box found 42 bp upstream of the lasB transcriptional start site (Rust, L. et al. (1996) J. Bacteriol. 178, 1134-1140). las box-like sequences were identified which are suspected to be involved in the regulation of 14 of the 39 qsc genes listed in Table 1 (FIG. 7). Because there is little information on the transcription starts of most of the genes identified in the screening assay, some relevant las boxes may have been missed and some of the identified sequences may not be in relevant positions.

Discussion

By screening a library of lacZ promoter probes introduced into P. aeruginosa PAO1 by transposon mutagenesis, 39 quorum sensing controlled (qsc) genes were identified. Most of these genes were not identified as quorum sensing-controlled previously. Mutations were not found in every gene in putative qsc operons (FIG. 4). Mutants that showed only a small degree of acyl-HSL-dependent lacZ induction in the initial screen were not studied.

Several mutants, for example qsc 101 and 102 showed an immediate and relatively large response to 3OC₁₂-HSL (Class I mutants, Table 3). The qsc101 and 102 genes code for proteins with no matches in the databases. Several mutants showed a relatively large and immediate response when both signals were supplied in the growth medium. Examples are qsc119 (rhlB), 121-125, and 129A and B. The qsc mutant showing the largest response was qsc131. The level of β-galactosidase activity when this mutant was grown in the presence of both signals was greater than 700 times that in the absence of the signals (Table 3). The qsc131 mutation is in phzC, which is a phenazine biosynthesis gene, and the qsc131 mutant did not produce the blue phenazine pigment pyocyanin at detectable levels. Many of the mutants that responded best to both signals early (Class III mutants) showed a small response when exposed to one or the other signal. The reasons for the small response to either signal are unclear at present but the data suggest that these genes may be subject to signal cross talk, or they may show a response to either LasR or RhlR. One reason they may respond to both signals better than they respond to C₄-HSL alone is that 3OC₁₂-HSL and LasR are required to activate RhlR, the transcription factor required for a response to C₄-HSL (Latifi, A. et al. (1996) Mol. Microbiol. 21, 1137-1146; Pesci, E. C. et al. (1997) J. Bacteriol. 179, 3127-3132). There were two mutant classes that showed a delayed response to the signals; Class II mutants which required only 3OC₁₂-HSL, and Class IV mutants, which required both signals for full induction. These mutants showed between 5 and 45-fold activation of gene expression (Table 3). There are a number of possible explanations for a delayed response to signal addition. It is possible that some of these genes are stationary phase genes. It is also possible that some are iron repressed. For example, it is known that the synthesis of pyoverdine is regulated by iron and the Class II, delayed response, qsc108-111 mutations are in genes involved in pyoverdine synthesis (Cunliffe, H. E. et al. (1995) J. Bacteriol. 177, 2744-2750; Rombel, I. et al. (1995) Mol. Gen. Genet. 246, 519-528). It is also possible that some of these genes are not regulated by quorum sensing, directly. The acyl-HSL signals might control other factors that influence expression of any of the genes that have been identified and this possibility seems most likely with the late genes in Classes II and IV. Indirect regulation may not be common for late genes. This is known because the lasB-lacZ chromosomal insertion which was generated by site-specific mutation was in Class IV, and it is known from other investigations that lasB responds to LasR and 3OC₁₂-HSL, directly (Passador, L. et al. (1993) Science 260, 1127-1130; Rust, L. et al. (1996) J. Bacteriol. 178, 1134-1140). The two classes of late qsc genes may be comprised of several subclasses.

Las boxes are genetic elements which may be involved in the regulation of qsc genes. Although sequences with characteristics similar to las boxes were identified, (FIG. 7), the locations of these sequences have not provided insights about the differences in the patterns of gene expression among the four classes of genes. It is possible that when the promoter regions of the qsc genes are studied that particular motifs in the regulatory DNA of different classes of genes will be revealed.

Many of the qsc genes appear to be organized in two patches or islands on the P. aeruginosa chromosome (FIG. 7). Because LasR mutants are defective in virulence it is tempting to speculate that these gene clusters may represent pathogenicity islands. The rhlI-rhlR quorum sensing modulation occurs on one of the qsc islands, but none of the qsc genes are tightly linked to the lasR-lasI modulon. Genes representing each of the 4 classes occur over the length of the chromosome and on both DNA strands. This is consistent with the view that quorum sensing is a global regulatory system in P. aeruginosa. Of interest there is a third LuxR family member in P. aeruginosa. This gene is adjacent to and divergently oriented from qsc103.

### Example 2

Identification of Additional Quorum Sensing Controlled Genes Using Transcriptome Analysis

Quorum sensing is critical for virulence of P. aeruginosa and for the development of mature biofilms. The methodology disclosed herein for identification of quorum sensing controlled genes provides a manageable group of genes to test for function in virulence and biofilm development. This Example describes the identification of quorum sensing controlled genes using transcriptome analysis that utilizes P. aeruginosa GeneChips™ (Affymetrix™). Experiments were carried out as described below.

Materials and Methods

Bacterial strains and growth conditions. The P. aeruginosa strains used were PAO-MW1 (rhlI::Tn501, lasI::tetA) as well as PAO lasR rhlR (ΔlasR::Tc^{R}, ΔrhlR::Gm^{R}) and the isogenic PAO1 parent. Bacteria were grown in buffered Luria-Bertani (LB) broth which contained the following components per liter: 10 g Typtone (Difco), 5 g yeast extract (Difco), 5 g NaCl and 50 mM 3-(N-Morpholino)propanesulfonic acid, pH 7.0. Synthetic acyl-HSLs (Aurora Biosciences™) were added to PAO-MW1 cultures at final concentrations of 2 μM for 3OC12-HSL and 10 μM for C4-HSL as indicated. To inoculate the cultures used for transcript profiling, cells grown to mid-logarithmic phase were added to 100 ml of pre-warmed medium in 500 ml culture flasks. The initial optical densities (OD₆₀₀) were 0.05 for PAO-MW1 and 0.01 for PAO1 and PAO lasR rhlR. Cultures were incubated at 37° C. in a rotating shaker at 250 rpm. Growth was monitored as OD₆₀₀.

Expression profiling experiments. For studies with the signal generation mutant, RNA was isolated from cultures at the following optical densities: 0.2, 0.4, 0.8, 1.4, 2.0, 3.0, and 4.0. For studies of the signal receptor mutants cells, RNA was isolated from cultures at densities of 0.05, 0.1, 0.2, 0.4, 0.8, 1.4, 2.0, 3.0, and 4.0. Between 1×10⁹ and 2×10⁹ cells were mixed with RNA Protect Bacteria Reagent™ (Qiagen™) and treated according to the manufacturer's mechanical disruption and lysis protocol. RNA was purified using RNeasy™ mini columns (Qiagen™) including the described on-column DNAse I digestion. In addition, the eluted RNA was treated for 1 hour at 37° C. with DNAse I (0.1 unit per μg of RNA). DNAse I was removed by using DNA-Free™ (Ambion™) or by RNeasy column purification. Further sample preparation and processing of the P. aeruginosa GeneChip™ genome arrays were done as described by the manufacturers, with minor modifications.

For cDNA synthesis, 12 μg of purified RNA were used, semi-random hexamer primers with an average G+C content of 75%, and Superscript II™ reverse transcriptase (LifeTechnologies™). Control RNAs from yeast, Arabidopsis, and Bacillus subtilis genes were added to the reaction to monitor assay performance. Probes for these transcripts are tiled on the GeneChip™. RNA was removed from the PCR reactions by alkaline hydrolysis. The cDNA synthesis products were purified, fragmented by brief incubation with DNAse I, and the 3'termini of the fragmentation products were labeled with biotin-ddUTP as described by the GeneChip™ manufacturer (Affymetrix™). Fragmented and labeled cDNA was hybridized to the array by overnight incubation at 50° C. Washing, staining, and scanning of Genechips™ were performed in an Affymetrix Fluidic Station™.

Analysis of GeneChip Experiments.

The Affymetrix Microarray Software Suite™ (MAS, Version 5.0) was used to determine transcript levels and whether there were differences in transcript levels when different samples were compared. Affymetrix™ global scaling was used to normalize data from different arrays. A scale factor is derived from the mean signal od all the probe sets on an array and a user-defined target signal. The signal from each individual probe set is multiplied by this scale factor. For any given array between 18 and 28% of the mRNAs were called “absent” by MAS, indicating that the corresponding genes were not expressed above background levels. Furthermore, average changes in control transcript intensities were less than twofold between any comparison of array data. This indicates that the efficiency of cDNA synthesis and labeling was similar from sample to sample.

For comparison analyses, the log₂ ratio between absolute transcript signals obtained from a given pair of arrays was calculated in MAS version 5.0. A statistical algorithm of the software also assigned a “change call” for each transcript pair, indicating whether the level of transcript was significantly increased, decreased, or not changed compared to the baseline sample. Baseline samples were those derived from cultures of PAO-MW1 without added acyl-HSL and of PAO lasR rhlR. Graphical analysis of the signal log ratios from each experiment (any pair of arrays) revealed a normal distribution with a mean very close to zero (no change). Among those transcripts with a significant increase or decrease compared to the baseline in one or more samples, those that showed a ≧2.5-fold change were sorted for further analysis.

For cluster analyses and transcript pattern analyses, GeneSpring software (Silicon Genetics™, Redwood City, Calif.) was used. The foldchange values for each gene were normalized independently by assigning the half-maximal value for that gene to 1 and representing all other values as a ratio of that value. This scaling procedure allowed direct visual comparison of gene expression patters within an experiment, as well as between experiments. GeneSpring™ was also used for functional classification according to the procedure of the P. aeruginosa Genome Project (see the P. aeruginosa Genome Project website).

Identification of las-rhl Box-Like Sequences.

A 20-bp consensus sequence “ACCTGCCAGATCTGGCAGGT” (SEQ ID NO:710) was derived from the following previously identified las-rhl box-like sequences in quorum sensing controlled genes: PA1869 (qsc117), PA1896 (qsc102), hcnA, lasB, lasI, and phzA, as well as PA2592 (qsc104), PA3327 (qsc126), PA4217 (qsc132), rhlA, and rhlI. To search the entire P. aeruginosa genome for sequences similar to this consensus, a computer program was developed based on that previously described to search for LexA binding sites. The scoring matrix of the program is based on an heterology index (HI), which determines the degree of divergence of any 20 nucleotide sequence from the consensus las-rhl box. Sequences in a region 400 bp upstream to 50 bp downstream of annotated translational start sites were considered as potential las-rhl boxes if they showed an HI score below 13.

Results

Genes Induced by Addition of Acyl-HSL Signals to the P. aeruginosa Signal Generation Mutant—a Validation of the GeneChip™ Analysis.

Thirty-nine (39) P. aeruginosa loci were previously identified as being quorum sensing controlled by studying the effects of acyl-HSLs on chromosomal lacZ insertions in a quorum-sensing signal generation mutant (MW1, a lasI, rhlI mutant) (see U.S. patent application Ser. No. 09/653,730 and Whiteley, et al. (1999) PNAS 96:13904-13909, the entire contents of each of which are expressly incorporated herein by reference).

In order to validate the GeneChip™ approach, this signal-generation mutant was grown with or without addition of 3OC12-HSL and C4-HSL under conditions identical to those described in Whiteley, et al. (1999) supra, and asked whether the genes identified previously would respond to signal addition in a transcriptome analysis. Most genes in the P. aeruginosa genome showed no significant response. Among 638 genes that showed a maximal response to acyl-HSL addition of at least 2.5-fold induction, 29 of the 35 previously described qsc genes (Whiteley, et al. (1999) supra) were identified (Table 5). The 6 remaining genes all exhibited relatively low induction levels in the previous study (Whiteley, et al. (1999) supra). Four of the 6, PA2385 (qsc112), PA2401 (qsc111), PA2402 (qsc109&110), and PA2426 (qsc108), showed a significant response to signal addition, but the response was <2.5-fold. Two, PA0051 (qsc137), and PA4084 (qsc113), showed no response. Taken together, these results showed agreement with the previous study (Whiteley, et al. (1999) supra).

The Quorum-Activated Regulon.

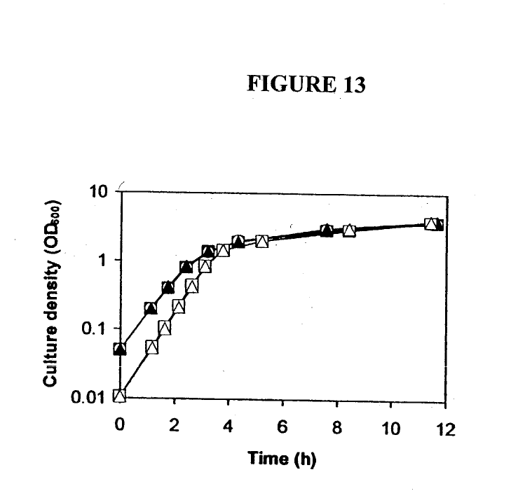

To identify a larger group of genes in the quorum sensing regulon of P. aeruginosa the results of the experiment described above were used and an additional independent experiment was performed in which transcripts in a quorum sensing signal receptor double mutant was compared to the parent strain. This is an independent method to assess whether genes are controlled by quorum sensing. It was reasoned that genes showing differential regulation with both approaches, addition of signals to a signal generation mutant, and a parent compared to a signal receptor mutant, were likely influenced by quorum sensing. The wild-type P. aeruginosa, the signal receptor mutant, and the signal generation mutant grown with or without added acyl-HSL signals showed similar growth patterns under the conditions of the experiments (FIG. 13).

As mentioned above, 638 genes were identified that were induced or repressed by addition of the acyl-HSLs signals to the signal generation mutant. 810 genes were identified that were induced or repressed in the parent as compared to the signal receptor mutant. In all there was an overlapping set of 411 genes. Visual inspection of the expression patterns of individual genes led to the exclusion of 58 genes. These genes either showed expression levels close to the background or inconsistent regulatory patterns when comparing the two experimental approaches. An interesting example of an inconsistent regulatory pattern was observed with a few genes identified and classified as late 3OC12-HSL-dependent in Whiteley, et al. (1999) supra, and in Example 1. These genes, PA2401, 2402 and 2385 (qsc109-110, 111, and 112) showed the predicted regulatory pattern in the transcriptome analysis of the signal generation mutant (although they showed low response levels of 2.0, 2.1, and 2.3, respectively), but they showed quorum-controlled repression when comparing the parent to the signal receptor mutant. In all, 315 genes have been identified which are quorum activated. These genes and information regarding their expression are listed in Table 5, below.

There is no obvious chromosomal clustering of the genes identified. The final set of quorum-induced genes represents about 5% of the genome. This is remarkably close to the previous prediction that somewhere around 2-4% of the genome would be quorum induced. However, the identified genes are quite likely a subset of the quorum regulon. For example, one standard growth condition was used for all of the experiments; it is not unreasonable to believe that other genes in the regulon might be revealed by altering the growth medium or culture conditions. In these experiments about 20-30% of the transcripts were at undetectable levels; some of these might be quorum controlled or expressed at higher levels under different conditions. As discussed above, the date set was also filtered, and we do not consider the genes that survived the filter to represent an exhaustive compilation of quorum-induced genes. Rather it is a conservative estimate of quorum-induced genes. Among the genes listed in Table 5, the most prevalent categories consisted of genes known or predicted to be involved in the production of secreted products, and genes of unknown function.

Quorum-Repressed Genes.

38 quorum-repressed genes were identified (Table 6). These genes showed lower transcript levels in late logarithmic and stationary phase in the wild-type compared to the receptor mutant and in the signal-generation mutant in the presence of signals as compared to the mutant grown without added signal. All of the repressed genes responded as well or nearly as well to 3OC12-HSL alone as they did to both 3OC12-HSL and C4-HSL together. These genes are expressed at low levels throughout growth of the parent strain. They are derepressed only in the mutants and only during late logarithmic and stationary phase. Among the quorum-repressed genes with known or predicted functions, those involved with carbohydrate utilization or nutrient transport appeared to be the most abundant (Table 6).

Operons and las-rhl Box-Like Sequences.

It would be expected that all of the genes in an operon should show similar quorum control. It was observed that strings of genes appeared (Tables 5 and 6). These strings often represent known or suspected operons, and the genes within a given string show similar quorum responses (signal responses and timing of induction). For example, the hcn genes (PA2193-2195) are known to exist in an operon. Consistent with this, the transcriptome analysis indicated these genes were co-induced by quorum sensing. PA2365 to PA2372 represents a string of quorum-controlled genes with unknown function. These genes may represent an operon. However, many of the quorum-controlled genes are not adjacent to other quorum-controlled genes listed in Tables 5 and 6. To assess whether these genes may also be organized in operons (neighboring genes would have been eliminated if they showed induction just under the 2.5-fold threshold), and to confirm the notion that strings of adjacent quorum-controlled genes are in operons, a more systematic analysis has been undertaken. Operon organization was only allowed if every gene within a gene cluster was in the same orientation, if every gene was activated or every gene was repressed, if there were <250 bp between two adjacent open reading frames (ORFs), and if the absolute transcript profiles of the candidate genes in the parent P. aeruginosa showed patterns similar to each other (correlation coefficient ≧0.95 using the GeneSpring standard correlation algorithm). By using these criteria, 87 possible operons were identified, 71 of which were activated and 16 of which were repressed (FIG. 15). More than sixty additional genes showing coregulation with the genes listed in Tables 5 and 6 were identified by this analysis.

A computer algorithm was used to search for las-rhl boxes in regions upstream of quorum-regulated genes. By using stringent criteria (an HI score of <10), 55 of all P. aeruginosa genes contain a box in their upstream regulatory region. Twenty-five (45%) of these genes are quorum controlled, and 15 represent the first gene in a predicted operon (identified in Table 5 and in FIG. 14). At lower stringency (an HI score of <13), 185 genes were identified with las-rhl like sequences. Forty-eight (26%) of these genes are quorum-controlled, and 19 represent the first gene in a predicted operon. Only one las-rhl box-like sequence was found upstream of a quorum-repressed gene. Potential boxes were not identified for all of the quorum-activated genes. Therefore, some of the genes may be controlled indirectly by quorum sensing.

Signal Specificity.

Whiteley, et al. ((1999) PNAS 96:13904-13909) and Example 1, above, classified genes into categories based on their response to the signals: those that responded equally well to 3OC12-HSL and to both 3OC12-HSL and C4-HSL together vs. those that responded best only when both 3OC12-HSL and C4-HSL were present. The GeneChip™ data set forth herein suggests that the responses are on a continuum with some genes responding no better to both signals than they do to 3OC12-HSL alone, and other genes showing progressively greater responses to both signals as compared to 3OC12-HSL alone. For example, PA2423 responded no better to both signals then it did to 3OC12-HSL alone, PA0122 responded well to 3OC12-HSL alone but showed about 3-times the response with both signals, and PA2069 did not respond at all to 3OC12-HSL alone but showed a large response in the presence of both signals (Table 5). This suggests that some genes respond to 3OC12-HSL specifically, others respond with varying specificities to either signal, and some respond to C4-HSL specifically. The genes encoding anthranilate dioxygenase, antABC (PA2512-2514), represented an exceptional case. They were strongly repressed in the presence of 3OC12-HSL alone, but activated in the presence of both signals.

Timing of Quorum-Controlled Gene Activation.

The timing of quorum sensing-controlled gene induction in the wild-type strain was elucidated by examining the GeneChip™ data to obtain a broader understanding of the influence of acyl-HSL signal addition on control of the quorum regulon. The patterns of quorum-controlled gene expression were remarkably similar in the parent and in the signal-generation mutant grown in the presence of 3OC12-HSL and C4-HSL. A small number of transcripts showed their greatest induction early in growth. Other genes exhibited increased expression early in growth but did not reach maximum levels until later in growth. Most transcripts were induced at culture densities between 0.8 and 2.0. Some transcripts only showed increased abundance relative to the baseline at culture densities above 2.0 (stationary phase). Thus the transcriptome analysis suggests that the timing of quorum-controlled gene induction is on a continuum, although most genes in the regulon appeared to be activated during the transition from logarithmic to stationary phase (optical densities between 0.8 and 2.0). The timing of induction for most genes was not affected by exogenous addition of 3OC12-HSL and C4-HSL.

While not intending to be bound by theory, it appears that even in the parent strain at the earliest sampling (optical density, 0.05), there were sufficient acyl-HSL levels for induction of the early genes and that some other factor was limiting expression of transcripts that were triggered to accumulate later in growth. Another factor which might account for the acyl-HSL-independent triggering of quorum gene induction is that the acyl-HSL receptors are limiting in early logarithmic phase and that the abundance of these factors increases during culture growth. It is hypothesized that in early logarithmic phase, the most active quorum-controlled promoters bind the transcription factors and effectively titrate them away from other quorum-controlled promoters. As the level of LasR increases, additional quorum-controlled genes should show expression. A prediction of this hypothesis is that lasR should show increased transcript abundance as a culture grows. Thus, the GeneChip™ data was interrogated with respect to lasR. Starting at an optical density of approximately 0.8, the level of lasr transcript increased markedly, consistent with previous results obtained with reporter fusions. The increase was observed in the wild-type strain and in the signal-generation mutant with or without added signal. Thus the increase is independent of quorum sensing. This result is consistent with but does not constitute proof of the model for timing of quorum-controlled gene expression described above.

TABLE 5
Quorum-activated genes.

| | | Maximum repression (fold)^{c} | | |
|---|---|---|---|---|
| | | lasl⁻ rhll⁻ mutant | | Wt vs. |
| Gene no.^{a} | Description^{b} | 30C12-HSL | C4 + 3OC12-HSL | lasR⁻ rhlR⁻ |
| PA0007 | hypothetical protein | 4.4 | 5.7 (2.0) | 13.5 (1.4) |
| PA0026 | hypothetical protein^{d} | 4.4 | 4.4 (1.4) | 5.9 (0.2) |
| PA0027 | hypothetical protein | 3.8 | 4.9 (0.8) | 5.7 (0.2) |
| PA0028 | hypothetical protein | 5.8 | 7.5 (1.4) | 8.2 (1.4) |
| PA0050 | hypothetical protein | 2.8 | 2.5 (2.0) | 3.0 (1.4) |
| PA0052 | hypothetical protein^{d} | 4.7 | 8.3 (1.4) | 22.2 (2.0) |
| PA0059 | osmC, osmotically inducible protein OsmC | 2.5 | 6.7 (2.0) | 8.9 (2.0) |
| PA0105 | coxB, cytochrome c oxidase, subunit II | 3.4 | 4.0 (2.0) | 2.6 (2.0) |
| PA0106 | coxA, cytochrome c oxidase, subunit I | 4.2 | 4.8 (1.4) | 3.3 (1.4) |
| PA0107 | conserved hypothetical protein | 4.1 | 4.8 (2.0) | 4.9 (2.0) |
| PA0108 | colII, cytochrome c oxidase, subunit III | 3.0 | 3.6 (2.0) | 2.8 (2.0) |
| PA0109 | qsc115, hypothetical protein | 2.1 | 3.5 (1.4) | 4.1 (1.4) |
| PA0122 | conserved hypothetical protein^{e} | 12.8 | 36.0 (1.4) | 50.9 (1.4) |
| PA0132 | beta-alanine--pyruvate transaminase | 1.6 | 3.1 (1.4) | 4.1 (2.0) |
| PA0143 | probable nucleoside hydrolase | 4.7 | 4.7 (0.4) | 5.4 (0.1) |
| PA0144 | hypothetical protein | 1.5 | 19.3 (2.0) | 28.4 (2.0) |
| PA0158 | probable RND efflux transporter | 2.6 | 2.6 (2.0) | 2.6 (2.0) |
| PA0175 | probable chemotaxis protein methyltransferase | 2.0 | 2.6 (3.0) | 4.6 (1.4) |
| PA0176 | probable chemotaxis transducer | 2.1 | 2.6 (3.0) | 3.9 (1.4) |
| PA0179 | probable two-component response regulator | 2.7 | 2.8 (1.4) | 3.7 (1.4) |
| PA0198 | exbBl, transport protein ExbB | 7.3 | 10.3 (4.0) | 3.7 (4.0) |
| PA0263 | hcpC, secreted protein Hcp | 1.7 | 8.9 (1.4) | 9.4 (1.4) |
| PA0355 | pfpl, protease PfpI | 2.3 | 4.8 (2.0) | 8.1 (2.0) |
| PA0364 | probable oxidoreductase | 2.9 | 3.1 (2.0) | 3.0 (2.0) |
| PA0365 | hypothetical protein | 2.0 | 2.5 (2.0) | 2.7 (2.0) |
| PA0366 | probable aldehyde dehydrogenase | 2.4 | 2.8 (2.0) | 2.5 (3.0) |
| PA0534 | conserved hypothetical protein | 1.5 | 2.9 (4.0) | 9.8 (2.0) |
| PA0567 | conserved hypothetical protein | 6.9 | 15.0 (2.0) | 10.7 (2.0) |
| PA0572 | hypothetical protein | 19.3 | 22.3 (0.4) | 18.6 (0.05) |
| PA0586 | conserved hypothetical protein | 2.1 | 2.6 (1.4) | 4.6 (1.4) |
| PA0852 | qsc129, cpbD, chitin-binding protein CbpD precursor^{d} | 11.4 | 42.8 (0.4) | 94.4 (0.1) |
| PA0855 | qsc116, hypothetical protein | 2.4 | 2.5 (0.8) | 3.0 (0.8) |
| PA0996 | probable coenzyme A ligase^{e} | 218.3 | 89.9 (0.8) | 42.2 (0.2) |
| PA0997 | hypothetical protein | 108.4 | 95.7 (0.8) | 195.4 (0.05) |
| PA0998 | hypothetical protein | 67.6 | 39.9 (0.4) | 195.4 (0.2) |
| PA0999 | fabH1,3-oxoacyl-[acyl-carrier-protein] synthase III | 37.3 | 25.3 (0.8) | 45.3 (0.2) |
| PA1000 | hypothetical protein | 22.2 | 12.4 (0.8) | 44.0 (0.2) |
| PA1001 | phnA, anthranilate synthase component I | 38.6 | 23.4 (0.8) | 286.0 (0.2) |
| PA1002 | phnB, anthranilate synthase component II | 17.4 | 12.9 (1.4) | 28.4 (0.8) |
| PA1003 | probable transcriptional regulator | 8.1 | 6.6 (0.2) | 77.7 (0.05) |
| PA1130 | hypothetical protein | 2.4 | 9.4 (1.4) | 16.1 (1.4) |
| PA1131 | probable MFS transporter^{d} | 1.7 | 5.0 (2.0) | 7.9 (1.4) |
| PA1173 | napB, cytochrome c-type protein NapB precursor | 2.3 | 2.8 (2.0) | 4.1 (1.4) |
| PA1175 | napD, NapD protein of periplasmic nitrate reductase | 2.6 | 2.4 (2.0) | 3.8 (1.4) |
| PA1176 | napF, ferredoxin protein NapF | 2.5 | 2.5 (2.0) | 5.8 (1.4) |
| PA1177 | napE, periplasmic nitrate reductase protein NapE | 2.9 | 3.6 (1.4) | 3.6 (1.4) |
| PA1215 | hypothetical protein | NC | 18.0 (1.4) | 55.3 (1.4) |
| PA1216 | hypothetical protein | 4.7 | 15.3 (0.8) | 121.1 (0.8) |
| PA1217 | probable 2-isopropylmalate synthase | 2.9 | 41.1 (1.4) | 382.7 (1.4) |
| PA1218 | hypothetical protein | NC | 6.9 (1.4) | 156.5 (1.4) |
| PA1221 | hypothetical protein^{e} | NC | 3.1 (3.0) | 10.9 (1.4) |
| PA1245 | hypothetical protein^{e} | 8.6 | 10.3 (0.8) | 11.3 (0.2) |
| PA1246 | aprD, alkaline protease secretion protein AprD | 8.6 | 9.8 (1.4) | 6.6 (0.8) |
| PA1247 | aprE, alkaline protease secretion protein AprE | 6.2 | 6.4 (1.4) | 9.1 (1.4) |
| PA1248 | aprF, alkaline protease secretion protein AprF | 7.2 | 7.6 (1.4) | 5.2 (1.4) |
| PA1249 | aprA, alkaline metalloproteinase precursor | 24.8 | 27.1 (1.4) | 22.3 (1.4) |
| PA1250 | aprI, alkaline proteinase inhibitor AprI^{d} | 20.1 | 20.0 (0.2) | 23.6 (0.05) |
| PA1289 | hypothetical protein | 2.9 | 5.7 (1.4) | 2.6 (1.4) |
| PA1317 | cyoA, cytochrome o ubiquinol oxidase subunit II | 2.5 | 4.7 (4.0) | 14.5 (2.0) |
| PA1318 | cyoB, cytochrome o ubiquinol oxidase subunit I | NC | 3.9 (1.4) | 16.0 (2.0) |
| PA1319 | cyoC, cytochrome o ubiquinol oxidase subunit III | 2.0 | 4.8 (4.0) | 7.9 (3.0) |
| PA1320 | cyoD, cytochrome o ubiquinol oxidase subunit IV | 42.2 | 70.5 (4.0) | 9.1 (3.0) |
| PA1323 | hypothetical protein | 2.8 | 6.1 (2.0) | 9.6 (2.0) |
| PA1324 | hypothetical protein | 2.4 | 5.3 (2.0) | 8.5 (2.0) |
| PA1404 | hypothetical protein | 2.0 | 2.7 (2.0) | 3.8 (2.0) |
| PA1431 | rsaL, regulatory protein RsaL^{d} | 352.1 | 340.1 (0.2) | 38.6 (0.8) |
| PA1432 | lasI, autoinducer synthesis protein LasI^{d} | NC⁴ | NC⁴ | 7.7 (0.8) |
| PA1656 | hypothetical proteine^{a} | 2.4 | 3.7 (1.4) | 5.7 (0.8) |
| PA1657 | conserved hypothetical protein | 5.9 | 15.2 (0.4) | 23.9 (0.8) |
| PA1658 | conserved hypothetical protein | 3.9 | 9.3 (0.8) | 17.4 (0.8) |
| PA1659 | hypothetical protein | 4.1 | 8.5 (0.8) | 17.0 (0.8) |
| PA1660 | hypothetical protein | 2.6 | 7.9 (0.8) | 15.8 (0.8) |
| PA1661 | hypothetical protein | 2.3 | 4.4 (1.4) | 4.4 (0.8) |
| PA1662 | probable ClpA/B-type protease | 2.9 | 6.6 (1.4) | 7.7 (0.8) |
| PA1663 | probable transcriptional regulator | 2.5 | 4.5 (0.8) | 9.1 (0.8) |
| PA1664 | hypothetical protein | 5.9 | 16.2 (0.4) | 22.3 (0.8) |
| PA1665 | hypothetical protein | 20.8 | 54.9 (1.4) | 27.5 (0.8) |
| PA1666 | hypothetical protein | 2.9 | 11.8 (0.8) | 37.5 (0.8) |
| PA1667 | hypothetical protein | 3.1 | 7.6 (0.8) | 11.8 (0.8) |
| PA1668 | hypothetical protein | 2.8 | 4.6 (0.8) | 6.3 (0.8) |
| PA1669 | hypothetical protein | 2.2 | 3.8 (1.4) | 16.6 (0.8) |
| PA1670 | stpl, serine/threonine phosphoprot. phosphatase Stpl | NC | 2.8 (1.4) | 3.6 (0.8) |
| PA1745 | hypothetical protein | 2.1 | 2.6 (2.0) | 2.8 (1.4) |
| PA1784 | hypothetical proteine^{e} | 14.2 | 14.8 (1.4) | 17.9 (1.4) |
| PA1869 | qsc117, probable acyl carrier protein^{e} | 7.8 | 40.8 (0.2) | 337.8 (0.2) |
| PA1870 | hypothetical protein | NC | 3.2 (2.0) | 6.3 (2.0) |
| PA1871 | lasA, LasA protease precursor^{d} | 47.5 | 88.0 (0.8) | 130.7 (1.4) |
| PA1881 | hypothetical protein | 2.4 | 2.6 (2.0) | 2.8 (1.4) |
| PA1888 | hypothetical protein | 2.7 | 2.3 (2.0) | 4.3 (1.4) |
| PA1891 | hypothetical protein | 3.3 | 4.3 (2.0) | 6.5 (0.8) |
| PA1893 | hypothetical protein | 15.9 | 12.7 (0.4) | 2.7 (2.0) |
| PA1894 | qsc101, hypothetical protein | 58.9 | 58.5 (0.8) | 5.0 (1.4) |
| PA1895 | hypothetical protein | 35.5 | 31.3 (0.8) | 4.2 (1.4) |
| PA1896 | hypothetical protein | 41.4 | 48.5 (1.4) | 3.1 (1.4) |
| PA1897 | qsc102, hypothetical protein^{e} | 132.5 | 129.8 (0.4) | 8.5 (0.8) |
| PA1914 | conserved hypothetical protein | 41.9 | 194.0 (2.0) | 704.3 (2.0) |
| PA1921 | hypothetical protein | NC | 14.1 (2.0) | 12.8 (2.0) |
| PA1930 | probable chemotaxis transducer | 2.2 | 2.9 (2.0) | 3.8 (1.4) |
| PA1939 | hypothetical protein | 2.6 | 3.2 (1.4) | 2.9 (2.0) |
| PA2030 | hypothetical protein | 3.3 | 4.2 (2.0) | 13.5 (2.0) |
| PA2031 | hypothetical protein | 4.5 | 6.5 (0.4) | 11.7 (1.4) |
| PA2066 | hypothetical protein | 1.9 | 3.7 (2.0) | 11.6 (1.4) |
| PA2067 | probable hydrolase | 1.8 | 5.0 (2.0) | 18.8 (1.4) |
| PA2068 | probable MFS transporter | NC | 16.0 (1.4) | 152.2 (1.4) |
| PA2069 | probable carbamoyl transferase^{e} | NC | 44.6 (1.4) | 112.2 (0.2) |
| PA2076 | probable transcriptional regulator^{d} | 3.7 | 4.3 (0.2) | 4.3 (0.2) |
| PA2080 | hypothetical protein | 3.5 | 4.0 (0.2) | 4.0 (0.2) |
| PA2081 | hypothetical protein | 3.3 | 4.3 (0.2) | 3.6 (0.2) |
| PA2134 | hypothetical protein | 3.1 | 5.4 (3.0) | 7.9 (2.0) |
| PA2142 | probable short-chain dehydrogenase | NC | 3.6 (3.0) | 19.4 (2.0) |
| PA2143 | hypothetical protein | 20.7 | 38.6 (2.0) | 50.6 (2.0) |
| PA2144 | glgP, glycogen phosphorylase | 2.5 | 4.7 (3.0) | 15.1 (2.0) |
| PA2146 | conserved hypothetical protein | 2.7 | 4.8 (3.0) | 11.4 (2.0) |
| PA2147 | katE, catalase HPII | 3.5 | 7.1 (2.0) | 34.5 (2.0) |
| PA2148 | conserved hypothetical protein | NC | 3.1 (2.0) | 3.4 (2.0) |
| PA2151 | conserved hypothetical protein | 2.6 | 37.5 (2.0) | 33.8 (2.0) |
| PA2152 | probable trehalose synthase | 2.1 | 5.3 (2.0) | 6.1 (2.0) |
| PA2153 | glgB, 1,4-alpha-glucan branching enzyme | 2.1 | 5.6 (2.0) | 16.3 (2.0) |
| PA2156 | conserved hypothetical protein | 2.3 | 4.8 (3.0) | 16.6 (2.0) |
| PA2157 | hypothetical protein | 2.1 | 2.8 (3.0) | 2.9 (3.0) |
| PA2158 | probable alcohol dehydrogenase (Zn-dependent) | 6.7 | 14.9 (2.0) | 26.0 (2.0) |
| PA2159 | conserved hypothetical protein | 4.1 | 5.9 (2.0) | 10.1 (2.0) |
| PA2160 | probable glycosyl hydrolase^{d} | 2.3 | 4.4 (3.0) | 5.8 (2.0) |
| PA2161 | hypothetical protein^{d} | 4.4 | 6.3 (2.0) | 10.4 (2.0) |
| PA2163 | hypothetical protein | 2.2 | 6.9 (2.0) | 31.1 (2.0) |
| PA2164 | probable glycosyl hydrolase | 2.5 | 4.7 (2.0) | 6.5 (2.0) |
| PA2165 | probable glycogen synthase | 3.2 | 5.7 (2.0) | 6.3 (2.0) |
| PA2166 | hypothetical protein | 3.1 | 9.1 (2.0) | 16.6 (2.0) |
| PA2167 | hypothetical protein | 2.3 | 2.6 (2.0) | 4.7 (2.0) |
| PA2169 | hypothetical protein | 2.8 | 5.7 (2.0) | 5.1 (2.0) |
| PA2170 | hypothetical protein | 3.6 | 6.9 (2.0) | 13.3 (2.0) |
| PA2171 | hypothetical protein | 5.2 | 9.1 (2.0) | 21.6 (2.0) |
| PA2172 | hypothetical protein | 3.8 | 7.7 (2.0) | 12.1 (2.0) |
| PA2173 | hypothetical protein | 3.5 | 6.5 (2.0) | 17.1 (2.0) |
| PA2176 | hypothetical protein | 1.4 | 5.3 (2.0) | 26.5 (2.0) |
| PA2180 | hypothetical protein | 1.9 | 2.6 (3.0) | 2.7 (3.0) |
| PA2190 | conserved hypothetical protein | 3.4 | 4.5 (2.0) | 7.5 (2.0) |
| PA2192 | conserved hypothetical protein | NC | 10.3 (2.0) | 8.4 (2.0) |
| PA2193 | hcnA, hydrogen cyanide synthase HcnA^{e} | 139.1 | 187.4 (0.2) | 88.0 (0.2) |
| PA2194 | qsc 128, hcnB, hydrogen cyanide synthase HcnB | 36.8 | 50.9 (0.2) | 58.5 (0.8) |
| PA2195 | hcnC, hydrogen cyanide synthase HcnC | 15.5 | 29.7 (0.4) | 46.2 (0.8) |
| PA2274 | hypothetical protein | NC | 3.4 (3.0) | 10.5 (2.0) |
| PA2300 | chiC, chitinase^{e} | 1.7 | 13.5 (1.4) | 103.3 (1.4) |
| PA2302 | qsc100, probable non-ribosomal peptide synthetase | 5.2 | 7.9 (0.8) | 126.2 (1.4) |
| PA2303 | qsc107, hypothetical protein | 24.8 | 28.1 (0.4) | 129.8 (0.2) |
| PA2304 | hypothetical protein | 8.4 | 12.1 (0.8) | 28.8 (0.8) |
| PA2305 | probable non-ribosomal peptide synthetase | 52.3 | 50.9 (0.2) | 69.6 (0.2) |
| PA2327 | probable permease of ABC transporter | 5.9 | 8.9 (4.0) | 6.9 (4.0) |
| PA2328 | hypothetical protein | 6.8 | 9.1 (2.0) | 7.5 (3.0) |
| PA2329 | probable component of ABC transporter | 7.8 | 9.9 (1.4) | 18.0 (3.0) |
| PA2330 | hypothetical protein | 7.9 | 10.6 (0.8) | 14.9 (2.0) |
| PA2331 | hypothetical protein | 8.3 | 19.3 (1.4) | 20.4 (1.4) |
| PA2345 | conserved hypothetical protein^{e} | 2.2 | 3.2 (2.0) | 2.6 (2.0) |
| PA2365 | conserved hypothetical protein | 4.7 | 5.4 (1.4) | 5.9 (1.4) |
| PA2366 | conserved hypothetical protein | 4.3 | 5.2 (1.4) | 6.9 (1.4) |
| PA2367 | hypothetical protein | 4.8 | 5.1 (1.4) | 6.4 (1.4) |
| PA2368 | hypothetical protein | 3.5 | 3.4 (1.4) | 7.5 (1.4) |
| PA2370 | hypothetical protein | 2.9 | 3.6 (3.0) | 3.5 (1.4) |
| PA2371 | probable ClpA/B-type protease | 2.4 | 2.6 (3.0) | 5.0 (1.4) |
| PA2372 | hypothetical protein | 3.2 | 2.7 (2.0) | 3.7 (1.4) |
| PA2414 | L-sorbosone dehydrogenase | 3.1 | 4.9 (2.0) | 20.7 (0.2) |
| PA2415 | hypothetical protein | 3.5 | 5.6 (2.0) | 13.6 (2.0) |
| PA2423 | hypothetical protein | 10.8 | 10.5 (0.4) | 12.6 (0.2) |
| PA2433 | hypothetical protein | 2.8 | 5.9 (2.0) | 10.9 (2.0) |
| PA2442 | gcvT2, glycine cleavage system protein T2 | 2.0 | 2.6 (3.0) | 3.1 (3.0) |
| PA2444 | glyA2, serine hydroxymethyltransferase | 9.1 | 12.4 (3.0) | 10.0 (3.0) |
| PA2445 | gcvP2, glycine cleavage system protein P2 | 6.6 | 7.5 (4.0) | 10.8 (3.0) |
| PA2446 | gcvH2, glycine cleavage system protein H2 | 11.8 | 17.0 (4.0) | 17.5 (3.0) |
| PA2448 | hypothetical protein | NC | 4.1 (3.0) | 11.8 (1.4) |
| PA2512 | antA, anthranilate dioxygenase large subunit | −604.7 | 42.5 (2.0) | 27.3 (3.0) |
| PA2513 | antB, anthranilate dioxygenase small subunit | −95.7 | 14.4 (2.0) | 12.9 (3.0) |
| PA2514 | antC, anthranilate dioxygenase reductase | −66.7 | 9.3 (2.0) | 3.8 (4.0) |
| PA2564 | hypothetical protein | 2.9 | 7.8 (2.0) | 21.1 (1.4) |
| PA2565 | hypothetical protein | 3.1 | 6.6 (2.0) | 14.2 (2.0) |
| PA2566 | conserved hypothetical protein^{e} | 6.5 | 12.7 (2.0) | 21.0 (1.4) |
| PA2570 | palL, PA-I galactophilic lectin^{d} | NC | 26.2 (1.4) | 195.4 (1.4) |
| PA2572 | probable two-component response regulator | 2.3 | 2.8 (1.4) | 3.3 (1.4) |
| PA2573 | probable chemotaxis transducer | 2.3 | 4.1 (1.4) | 3.9 (1.4) |
| PA2587 | qsc105, probable FAD-dependent monooxygenase | 12.3 | 11.8 (0.2) | 15.0 (0.1) |
| PA2588 | probable transcriptional regulator | 15.1 | 22.2 (0.2) | 46.2 (0.8) |
| PA2591 | probable transcriptional regulator^{e} | 21.3 | 24.8 (0.2) | 42.2 (0.2) |
| PA2592 | qsc104, probable spermidine/putrescine-binding protein^{e} | 5.6 | 8.7 (0.4) | 14.5 (0.8) |
| PA2593 | hypothetical protein | NC | 4.6 (2.0) | 29.4 (0.8) |
| PA2717 | cpo, chloroperoxidase precursor | 2.4 | 2.6 (2.0) | 3.4 (1.4) |
| PA2747 | hypothetical protein | 3.6 | 7.2 (2.0) | 10.6 (2.0) |
| PA2927 | hypothetical protein | 2.6 | 3.4 (2.0) | 13.5 (1.4) |
| PA2939 | probable aminopeptidase | 37.8 | 41.6 (1.4) | 26.5 (1.4) |
| PA3022 | hypothetical protein | 3.5 | 4.7 (2.0) | 4.3 (2.0) |
| PA3032 | qsc135, cytochrome c | 2.3 | 3.3 (2.0) | 9.3 (2.0) |
| PA3104 | xcpP, secretion protein XcpP | 3.1 | 3.2 (2.0) | 4.7 (1.4) |
| PA3181 | 2-keto-3-deoxy-6-phosphogluconate aldolase | 1.6 | 2.9 (3.0) | 3.2 (3.0) |
| PA3182 | conserved hypothetical protein | 1.7 | 3.2 (3.0) | 5.0 (3.0) |
| PA3183 | zwf, glucose-6-phosphate 1-dehydrogenase | 2.0 | 3.7 (3.0) | 4.0 (3.0) |
| PA3188 | probable permease of ABC sugar transporter | 2.9 | 4.2 (2.0) | 6.8 (3.0) |
| PA3189 | probable permease of ABC sugar transporter | 2.0 | 2.5 (3.0) | 3.0 (3.0) |
| PA3190 | probable component of ABC sugar transporter | 2.7 | 3.4 (2.0) | 4.1 (3.0) |
| PA3194 | edd, phosphogluconate dehydratase | 2.0 | 3.2 (3.0) | 2.9 (3.0) |
| PA3195 | gapA, glyceraldehyde 3-phosphate dehydrogenase | 3.1 | 5.0 (3.0) | 5.4 (3.0) |
| PA3274 | hypothetical protein^{d} | 1.9 | 4.3 (2.0) | 10.1 (2.0) |
| PA3311 | conserved hypothetical protein | 3.6 | 3.6 (2.0) | 6.0 (1.4) |
| PA3326 | probable Clp-family ATP-dependent protease^{e} | 6.6 | 19.7 (0.4) | 19.3 (0.8) |
| PA3327 | qsc126, probable non-ribosomal peptide synthetase^{e} | NC | 6.8 (0.8) | 19.6 (0.8) |
| PA3328 | qsc125, probable FAD-dependent monooxygenase | NC | 16.8 (0.4) | 46.9 (0.8) |
| PA3329 | qsc124, hypothetical protein | NC | 247.3 (0.4) | 310.8 (0.8) |
| PA3330 | qsc123, probable short chain dehydrogenase | NC | 124.5 (0.4) | 117.4 (0.8) |
| PA3331 | qsc122, cytochrome P450 | 3.5 | 38.9 (0.4) | 61.8 (0.8) |
| PA3332 | conserved hypothetical protein | 2.3 | 35.0 (0.8) | 40.8 (1.4) |
| PA3333 | qsc121, fabH2, 3-oxoacyl-[acyl-carrier-protein] synthase III | NC | 32.4 (0.4) | 64.4 (0.8) |
| PA3334 | probable acyl carrier protein | 1.8 | 49.2 (0.4) | 68.6 (0.8) |
| PA3335 | hypothetical protein | NC | 9.6 (0.4) | 29.4 (1.4) |
| PA3336 | qsc120, probable MFS transporter | NC | 21.6 (1.4) | 23.8 (0.8) |
| PA3346 | probable two-component response regulator | 2.7 | 2.8 (2.0) | 4.7 (2.0) |
| PA3347 | hypothetical protein^{c} | 2.3 | 2.8 (2.0) | 4.3 (1.4) |
| PA3361 | hypothetical protein | 10.0 | 13.4 (1.4) | 68.1 (1.4) |
| PA3369 | hypothetical protein | 1.9 | 3.3 (2.0) | 4.8 (2.0) |
| PA3370 | hypothetical protein | 1.7 | 3.5 (2.0) | 5.6 (2.0) |
| PA3371 | hypothetical protein | 1.7 | 3.6 (2.0) | 6.0 (2.0) |
| PA3416 | probable pyruvate dehydrogenase component | 2.5 | 3.2 (2.0) | 4.1 (1.4) |
| PA3418 | ldh, leucine dehydrogenase | 2.6 | 3.7 (1.4) | 5.0 (1.4) |
| PA3476 | qsc118, rhlI, autoinducer synthesis protein RhlI^{e} | NC⁴ | NC⁴ | 33.6 (0.05) |
| PA3477 | rhlR, transcriptional regulator RhlR | 8.5 | 9.6 (0.4) | 130.7 (0.05) |
| PA3478 | qsc119, rhlB, rhamnosyltransferase chain B | 5.3 | 88.6 (0.8) | 121.9 (1.4) |
| PA3479 | qsc119, rhlA, rhamnosyltransferase chain A^{e} | 10.1 | 120.3 (0.8) | 203.7 (0.8) |
| PA3520 | hypothetical protein^{d} | 2.2 | 12.6 (1.4) | 32.2 (1.4) |
| PA3535 | probable serine protease | 7.5 | 8.1 (0.4) | 5.9 (0.8) |
| PA3676 | probable RND efflux transporter | 3.9 | 1.9 (2.0) | 5.8 (1.4) |
| PA3677 | probable RND efflux protein precursor | 3.6 | 3.8 (2.0) | 8.3 (1.4) |
| PA3678 | probable transcriptional regulator | 2.9 | 1.6 (2.0) | 3.5 (1.4) |
| PA3688 | hypothetical protein | 3.0 | 5.4 (0.2) | 3.5 (1.4) |
| PA3691 | hypothetical protein | 2.4 | 4.5 (2.0) | 6.3 (2.0) |
| PA3692 | probable outer membrane protein | 3.0 | 5.8 (2.0) | 6.9 (2.0) |
| PA3724 | lasB, elastase LasB^{e} | 113.8 | 176.1 (0.8) | 242.2 (0.8) |
| PA3734 | hypothetical protein | NC | 4.1 (3.0) | 15.5 (2.0) |
| PA3888 | probable permease of ABC transporter | NC | 3.2 (2.0) | 3.9 (2.0) |
| PA3890 | probable permease of ABC transporter | 1.8 | 4.1 (2.0) | 4.7 (2.0) |
| PA3891 | probable component of ABC transporter | 2.1 | 5.0 (2.0) | 8.2 (2.0) |
| PA3904 | hypothetical protein | 49.2 | 41.6 (0.2) | 45.9 (0.05) |
| PA3905 | hypothetical protein | 36.8 | 58.5 (0.2) | 87.4 (0.05) |
| PA3906 | hypothetical protein | 141.0 | 134.4 (0.2) | 70.5 (0.05) |
| PA3907 | qsc103, hypothetical protein | 19.6 | 18.9 (0.2) | 58.1 (0.05) |
| PA3908 | hypothetical protein | 10.2 | 10.9 (0.2) | 54.9 (0.05) |
| PA3986 | hypothetical protein | 2.7 | 3.3 (1.4) | 2.8 (2.0) |
| PA4078 | qsc134, probable nonribosomal peptide synthetase^{d} | 3.2 | 4.6 (2.0) | 19.8 (2.0) |
| PA4117 | probable bacteriophytochrome | 5.3 | 5.6 (1.4) | 4.3 (1.4) |
| PA4129 | hypothetical protein | 25.1 | 30.7 (0.8) | 14.6 (0.8) |
| PA4130 | probable sulfite or nitrite reductase | 23.3 | 26.7 (0.8) | 10.7 (0.8) |
| PA4131 | probable iron-sulfur protein | 23.9 | 30.1 (0.8) | 21.0 (0.8) |
| PA4132 | conserved hypothetical protein | 13.7 | 14.5 (0.8) | 6.4 (0.8) |
| PA4133 | cytochrome c oxidase subunit (cbb3-type) | 104.0 | 102.5 (0.8) | 37.3 (0.8) |
| PA4134 | hypothetical protein | 43.1 | 46.9 (0.8) | 20.7 (0.8) |
| PA4139 | hypothetical protein | 3.1 | 2.9 (2.0) | 3.9 (2.0) |
| PA4141 | hypothetical protein | 2.6 | 26.0 (0.4) | 73.0 (1.4) |
| PA4142 | probable secretion protein | NC | 5.2 (2.0) | 15.7 (1.4) |
| PA4171 | probable protease | 3.5 | 4.6 (2.0) | 5.1 (2.0) |
| PA4172 | probable nuclease | 2.0 | 3.4 (2.0) | 13.9 (2.0) |
| PA4175 | probable endoproteinase Arg-C precursor | 11.1 | 15.0 (2.0) | 23.3 (1.4) |
| PA4190 | probable FAD-dependent monooxygenase | 3.0 | 2.5 (1.4) | 4.0 (0.2) |
| PA4205 | hypothetical protein | 1.9 | 8.7 (3.0) | 55.7 (2.0) |
| PA4206 | probable RND efflux protein precursor | 1.7 | 6.3 (3.0) | 30.1 (2.0) |
| PA4207 | qsc133, probable RND efflux transporter | NC | 2.5 (3.0) | 16.8 (2.0) |
| PA4208 | probable outer membrane efflux protein | 1.6 | 3.1 (3.0) | 19.4 (2.0) |
| PA4209 | probable O-methyltransferase^{e} | 4.6 | 11.2 (1.4) | 27.1 (1.4) |
| PA4210 | probable phenazine biosynthesis protein^{e,g} | NC | 59 (1.4) | 71 (1.4) |
| PA4211 | probable phenazine biosynthesis protein^{d} | 10 | 69 (0.8) | 220 (0.8) |
| PA4212 | qsc131, phenazine biosynthesis protein PhzC^{d} | 2.2 | 15 (1.4) | 77 (1.4) |
| PA4213 | phenazine biosynthesis protein PhzD | 3.7 | 36 (1.4) | 210 (1.4) |
| PA4214 | phenazine biosynthesis protein PhzE | 2.5 | 18 (1.4) | 59 (1.4) |
| PA4215 | probable phenazine biosynthesis protein | 3.1 | 24 (1.4) | 110 (1.4) |
| PA4216 | probable pyridoxamine 5-phosphate oxidase | 3.0 | 21 (1.4) | 56 (1.4) |
| PA4217 | qsc132, probable FAD-dependent monooxygenase | 4.4 | 27.5 (1.4) | 40.5 (1.4) |
| PA4296 | probable two-component response regulator | 2.4 | 3.6 (1.4) | 5.6 (1.4) |
| PA4297 | hypothetical protein | 2.4 | 3.3 (2.0) | 11.6 (2.0) |
| PA4298 | hypothetical protein | 2.3 | 4.7 (2.0) | 8.7 (2.0) |
| PA4299 | hypothetical protein | 2.1 | 3.6 (2.0) | 7.0 (2.0) |
| PA4300 | hypothetical protein | 2.0 | 3.5 (2.0) | 7.8 (2.0) |
| PA4302 | probable type II secretion system protein | 3.2 | 6.1 (2.0) | 7.4 (2.0) |
| PA4304 | probable type II secretion system protein | 2.2 | 3.1 (2.0) | 6.1 (2.0) |
| PA4305 | hypothetical protein | 2.1 | 2.7 (2.0) | 5.9 (2.0) |
| PA4306 | hypothetical protein | 10.1 | 15.8 (1.4) | 38.3 (1.4) |
| PA4311 | conserved hypothetical protein | 2.5 | 3.2 (2.0) | 2.6 (2.0) |
| PA4384 | hypothetical protein | NC | 2.7 (3.0) | 4.0 (3.0) |
| PA4498 | probable metallopeptidase | 1.6 | 4.5 (3.0) | 9.1 (3.0) |
| PA4590 | pra, protein activator | 9.3 | 13.5 (1.4) | 12.8 (0.8) |
| PA4648 | hypothetical protein | 3.4 | 7.7 (2.0) | 16.9 (1.4) |
| PA4649 | hypothetical protein | NC | 3.2 (2.0) | 7.4 (1.4) |
| PA4650 | hypothetical protein | NC | 3.3 (2.0) | 8.8 (2.0) |
| PA4651 | probable pili assembly chaperone^{d} | NC | 4.6 (2.0) | 14.6 (2.0) |
| PA4652 | hypothetical protein | 6.0 | 12.9 (2.0) | 9.6 (2.0) |
| PA4677 | hypothetical protein | 16.4 | 13.1 (0.2) | 36.0 (0.1) |
| PA4703 | hypothetical protein | 3.1 | 4.3 (2.0) | 3.5 (1.4) |
| PA4738 | conserved hypothetical protein | 3.8 | 9.1 (2.0) | 11.2 (2.0) |
| PA4739 | conserved hypothetical protein | 4.2 | 9.4 (2.0) | 14.0 (2.0) |
| PA4778 | probable transcriptional regulator | 5.4 | 4.9 (0.4) | 8.6 (0.1) |
| PA4869 | qsc106, hypothetical protein^{d} | 5.0 | 5.7 (0.4) | 3.8 (0.1) |
| PA4876 | osmE, osmotically inducible lipoprotein OsmE | 2.3 | 3.6 (2.0) | 4.9 (2.0) |
| PA4880 | probable bacterioferritin | 2.2 | 4.6 (2.0) | 5.8 (2.0) |
| PA4916 | hypothetical protein | 1.5 | 4.3 (4.0) | 6.1 (2.0) |
| PA4917 | hypothetical protein^{d} | 1.4 | 5.8 (2.0) | 7.7 (2.0) |
| PA4925 | conserved hypothetical protein | 3.8 | 3.7 (2.0) | 5.7 (1.4) |
| PA5027 | hypothetical protein^{e} | 1.5 | 2.8 (2.0) | 3.2 (3.0) |
| PA5058 | phaC2, poly (3-hydroxyalkanoic acid) synthase 2^{e} | 4.5 | 4.7 (1.4) | 9.2 (1.4) |
| PA5059 | probable transcriptional regulator | 4.4 | 5.9 (2.0) | 9.3 (1.4) |
| PA5061 | conserved hypothetical protein | 1.7 | 2.5 (4.0) | 2.6 (4.0) |
| PA5161 | rmlB, dTDP-D-glucose 4,6-dehydratase | NC | 2.9 (4.0) | 5.9 (3.0) |
| PA5162 | rmlD, dTDP-4-dehydrorhamnose reductase | NC | 2.5 (4.0) | 4.9 (3.0) |
| PA5164 | rmlC, dTDP-4-dehydrorhamnose 3,5-epimerase | NC | 2.6 (3.0) | 5.6 (2.0) |
| PA5220 | qsc138, hypothetical protein | 2.8 | 18.1 (0.8) | 26.2 (1.4) |
| PA5352 | conserved hypothetical protein | 2.0 | 2.8 (1.4) | 2.9 (1.4) |
| PA5353 | glcF, glycolate oxidase subunit GlcF | 1.9 | 3.4 (1.4) | 3.5 (1.4) |
| PA5354 | glcE, glycolate oxidase subunit GlcE | 2.0 | 2.6 (1.4) | 3.2 (1.4) |
| PA5355 | glcD, glycolate oxidase subunit GlcD | 2.1 | 3.6 (1.4) | 3.8 (1.4) |
| PA5356 | qsc130, glcC, transcriptional regulator GlcC | 2.4 | 4.1 (1.4) | 2.8 (1.4) |
| PA5415 | glyAl, serine hydroxymethyltransferase | 2.6 | 2.8 (3.0) | 5.0 (3.0) |
| PA5481 | hypothetical protein | 4.1 | 10.6 (2.0) | 15.2 (1.4) |
| PA5482 | hypothetical protein | 5.4 | 15.0 (2.0) | 17.8 (1.4) |

^{a}Gene Identification Number from the Pseudomonas genome project (www.pseudomonas.com).
^{b}Boldface type indicates genes or gene products previously reported to be controlled by quorum sensing. RND, resistance nodulation-cell division; FAD, flavin adenine dinucleotide.
^{c}Maximum changes in gene expression (rounded to two significant figures) in the signal generation mutant in the presence of the signal(s) indicated compared with the absence of signal and in wild-type P. aeruginosa strain compared with the receptor mutant. The values in the parentheses are the OD₆₀₀ at which the earliest change of ≧2.5 was observed (for the signal generation mutant, both time courses were considered). NC, no change.
^{d}There is a las-rhl box-like sequence with an HI of ≧10 and <13.
^{e}There is a las-rhl box-like sequence with an HI of <10.
^{f}The transcript levels for lasI and rhlI were close to background in the signal generation mutant due to the disruption of both loci by insertional mutagenesis.
^{g}The GeneChip ™ probes for PA4210 to PA4216 are identical to those for PA1899 to PA1905. Although the sequences for the genes in these two clusters are almost identical, the region upstream of PA4210 contains a las-rhl box-like sequence, but the region upsteam of PA1899 does not.

TABLE 6
Quorum-repressed genes.¹

| | | Maximum repression (fold)^{c} | | |
|---|---|---|---|---|
| | | lasI⁻ rhlI⁻ mutant | | Wt vs. |
| Gene no.^{a} | Description^{b} | 3OC12-HSL | C4 + 3OC12-HSL | lasR⁻ rhlR⁻ |
| PA0165 | hypothetical protein | −2.7 | −2.9 (2.0) | −4.8 (2.0) |
| PA0433 | hypothetical protein | −6.8 | −19.7 (2.0) | −8.9 (1.4) |
| PA0434 | hypothetical protein | −7.7 | −8.5 (2.0) | −5.6 (2.0) |
| PA0435 | hypothetical protein | −9.4 | −25.5 (2.0) | −33.8 (2.0) |
| PA0485 | conserved hypothetical protein^{c} | −1.7 | −3.4 (1.4) | −3.0 (3.0) |
| PA0887 | acsA, acetyl-coenzyme A synthetase | −3.3 | −4.2 (2.0) | −3.6 (3.0) |
| PA1559 | hypothetical protein | −2.4 | −3.5 (2.0) | −3.2 (1.4) |
| PA2007 | maiA, maleylacetoacetate isomerase | −3.2 | −1.4 (4.0) | −3.2 (3.0) |
| PA2008 | fahA, fumarylacetoacetase | −3.7 | −1.5 (4.0) | −2.6 (3.0) |
| PA2009 | hmgA, homogentisate 1,2-dioxygenase | −4.0 | −1.5 (4.0) | −2.7 (3.0) |
| PA2250 | lpdV, lipoamide dehydrogenase-Val | −3.1 | −1.8 (4.0) | −2.6 (3.0) |
| PA2338 | probable component of ABC maltose transporter | −5.0 | −3.2 (3.0) | −4.2 (3.0) |
| PA2339 | probable maltose/mannitol transport protein - | 1.9 | −6.8 (3.0) | −4.1 (3.0) |
| PA2340 | probable maltose/mannitol transport protein - | 3.4 | −2.0 (3.0) | −3.7 (3.0) |
| PA2341 | probable component of ABC maltose transporter | −3.1 | −2.0 (3.0) | −4.2 (3.0) |
| PA2343 | mtlY, xylulose kinase | −1.7 | −4.0 (3.0) | −3.2 (4.0) |
| PA3038 | probable porin | −2.3 | −3.5 (2.0) | −4.4 (3.0) |
| PA3174 | probable transcriptional regulator | −2.1 | −3.5 (4.0) | −6.5 (3.0) |
| PA3205 | hypothetical protein | −1.3 | −3.1 (4.0) | −3.1 (4.0) |
| PA3233 | hypothetical protein | −2.2 | −2.7 (3.0) | −5.1 (3.0) |
| PA3234 | probable sodium: solute symporter | −4.5 | −3.4 (2.0) | −7.0 (3.0) |
| PA3235 | conserved hypothetical protein | −3.9 | −4.2 (3.0) | −6.6 (3.0) |
| PA3281 | hypothetical protein | −5.7 | −6.4 (1.4) | −24.9 (1.4) |
| PA3282 | hypothetical protein | −8.5 | −8.8 (1.4) | −21.3 (1.4) |
| PA3283 | conserved hypothetical protein | −9.0 | −8.8 (1.4) | −27.7 (1.4) |
| PA3284 | hypothetical protein | −7.1 | −10.4 (2.0) | −24.3 (1.4) |
| PA3364 | amiC, aliphatic amidase expression-regulating protein | −2.7 | −1.8 (4.0) | −2.7 (1.4) |
| PA3365 | probable chaperone | −3.0 | −1.7 (4.0) | −4.0 (1.4) |
| PA3575 | hypothetical protein | −1.6 | −2.7 (1.4) | −3.3 (2.0) |
| PA3790 | oprC, outer membrane protein OprC | −2.7 | −3.7 (2.0) | −4.6 (2.0) |
| PA4359 | conserved hypothetical protein | −1.4 | −2.7 (2.0) | −2.8 (1.4) |
| PA4371 | hypothetical protein | −1.9 | −4.1 (2.0) | −2.8 (1.4) |
| PA4442 | cysN, ATP sulfurylase GTP-binding subunit | −2.8 | −3.4 (3.0) | −7.6 (2.0) |
| PA4443 | cysD, ATP sulfurylase small subunit | −3.1 | −3.4 (3.0) | −6.5 (2.0) |
| PA4691 | hypothetical protein | −2.5 | −2.8 (2.0) | −2.9 (2.0) |
| PA4692 | conserved hypothetical protein | −3.8 | −3.4 (2.0) | −5.0 (1.4) |
| PA4770 | lldP, L-lactate permease | −1.8 | −3.7 (2.0) | −5.0 (2.0) |
| PA5168 | probable dicarboxylate transporter | −2.7 | −1.9 (4.0) | −5.8 (2.0) |

^{a}Gene Identification Number from the Pseudomonas genome project (www.pseudomonas.com).
^{b}Maximum changes in gene expression (rounded to two significant figures) in the signal generation mutant in the presence of the signal(s) indicated compared with the absence of signal and in wild-type P. aeruginosa strain compared with the receptor mutant. The values in the parentheses are the OD₆₀₀ at which the earliest change of ≧2.5 was observed (for the signal generation mutant, both time courses were considered). NC, no change.
^{c}There is a las-rhl box-like sequence with an HI of ≧10 and <13.

### Example 3

Screening Assay for Quorum Sending Inhibiting Compounds

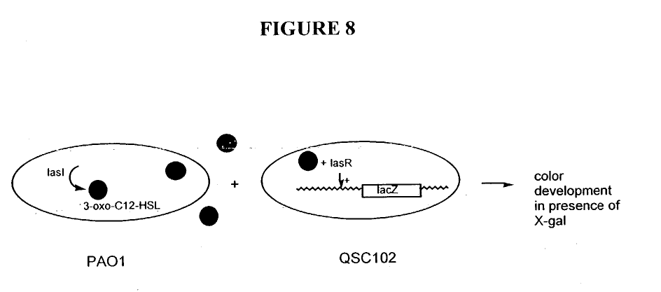

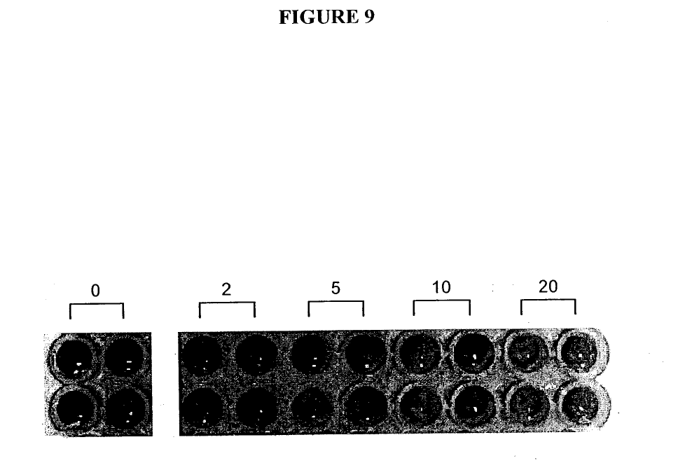

In this example, the screening assay used two strains of P. aeruginosa: a wild type P. aeruginosa (PAO1) and QSC102, from Example 1 (see FIG. 8). This assay will detect inhibition of all aspects of quorum sensing signaling, e.g., signal generation and signal reception.

Procedural Overview

Microtiter plates are prepared by adding 200 μL Luria Broth (“LB”) agar, containing 0.008% 5-bromo-4-chloro-3-indolyl-β-D-galactose (X-gal) to each well. Overnight cultures of PAO1 and QSC102 are subcultured in LB to a starting absorbance at 600 nm (“A600”) of 0.05 and grown at 37° C. to an A600 of 1.0. PAO1 is diluted 2.5×10⁵-fold in LB and 5 μL of this is applied to the surface of the LB agar in each well. Plates are then dried in a laminar flow hood for 60 minutes. A tenfold dilution of QSC102 in LB is used to inoculate each well using a replicator. Plates are then sealed and incubated at 37° C. for 40 hours. Growth and color development are evaluated visually and the data is recorded with a camera.

The test compound was present in a microtiter well and overlaid with LB agar and 5-bromo-4-chloro-3-indolyl-β-D-galactose (X-gal). Both strains were spotted on the agar in each well. PAO1 emitted the acyl-HSL signal (3-oxo-C12-HSL), to which QSC102 responded by turning blue QSC102 expressed β-galactosidase only in response to the LasI signal (3-oxo-C12-HSL); the lacZ fusion in QSC102 did not respond to the RhlI signal (C4-HSL). Hence, the assay was selective for inhibitors of the Las system. Inhibition of signaling was evaluated qualitatively by the absence or weakening of the blue color development.

The assay was used to test 6 product analogs, two of which showed an inhibitory effect: butyrolactone and acetyl-butyrolactone. Although bacterial growth was not inhibited, the color development was reduced. Color reduction correlated directly with test compound concentration, although relatively high concentrations (˜20 mM) were required to suppress color development completely).

### Example 4

Development of A P. aeruginosa Strain for a High Throughput Screening Asaay

A. Construction of Reporter Strain-Chromosomal Insertion of Reporter

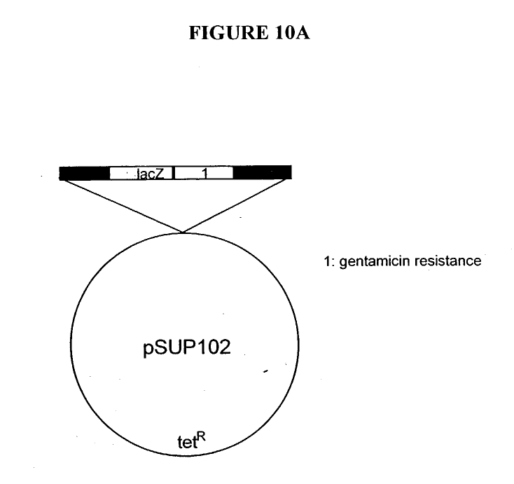

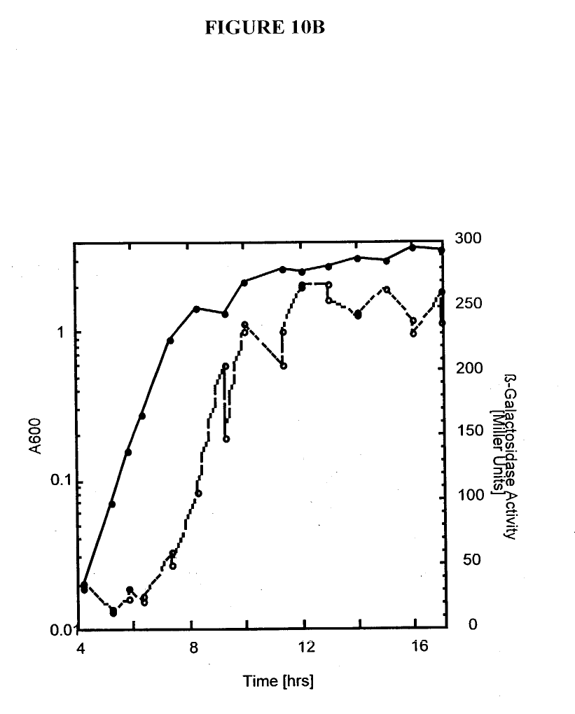

A strain for use in high-throughput screening was constructed by inserting the lacZ transcriptional fusion, linked gentamicin resistance marker, and about 2 kb of flanking DNA from strain QSC102 into a mobilizable plasmid (such as PSUP102) as depicted in FIG. 10A. Plasmid pSUP 102 confers tetracycline resistance and does not replicate in P. aeruginosa (Simon, R. et al. (1986) Meth. Enzym. 118:640-659). The pSUP102-derivative was then transferred into PAO1 by bi- or triparental mating, selecting for gentamicin resistance (Suh, S. J. et al. (1999) J Bacteriol. 181(13):3890-7). Gentamicin resistant isolates were screened for tetracycline sensitivity (i.e., a double cross-over event has resulted in a chromosomal insertion). Southern blotting was used to confirm the nature of the recombination event and to rule out candidates with more than one insertion. The resultant bacterial strain generates the signal (3-oxo-C12-HSL) and responds to it by increased β-galactosidase activity. A similar strategy is used to create a reporter strain that expresses gfp instead of lacZ. The initial GFP variant is the stable and bright variant GFPmut2 (Cormack, B. P. et al. (1996) Gene. 173(1):33-38).

Procedural Overview of Assay

A culture of PAQ1 reporter strain (carrying the reporter gene lacZ transcriptionally fused to the regulatory sequence of qsc 102 in the wildtype background, PAO1) was grown in LB, 100 μg/ml gentamicin overnight, such that the A600 was around 0.1. The culture was washed in LB twice and used to subculture at a 1:1000 dilution in LB. The subculture was grown in the presence or absence of test compound. Growth was monitored at A600 and expression of β-galactosidase activity is measured according to the Miller assay (Miller, J. A. (1976) in Experiments in Molecular Genetics pp 352-355, Cold Spring Harbor Lab. Press, Plainview, N.Y.).

The reporter strain was tested by growing it in microtiter plates in the presence and absence of known inhibitors of bacterial signaling. Examples of known inhibitors are: acetyl-butyrolactone, butyrolactone, and methylthioadenosine, a product of the synthase reaction that was shown to be inhibitory to the RhlI synthase (Parsek, M. R. et al. (1999) Proc. Natl. Acad. Sci. USA. 96:4360-4365). Initial characterization of the assay entailed following the optical density (cell growth) in individual sample wells and measuring induction levels at different time points. FIG. 10B shows the induction of β-galactosidase as PAQ1 reaches high density, wherein cell growth is measured at 600 nm (closed circles) and expression of β-galactosidase is measured in Miller units (open circles). For GFP fusions, the fluorescence of the culture is determined after excitation at 488 nm.

B. Construction of Reporter Strain-Reporter on a Plasmid

The PAO1/pMW303G strain is constructed as described in Example 1 above.

Procedural Overview of the Assay

An overnight culture of PAO1/pMW303G was diluted to an A600 of 0.1 in LB, 300 μg/ml carbenicillin. Of this, 50 μL were added to microtiter plate wells and grown at 37° C., shaking at 250 rpm, in the presence or absence of test compounds. Culture growth was monitored directly in the microtiter plate at 620 nm. Expression of the reporter gene, β-galactosidase was measured with the Galacton substrate by Tropix as follows. 12A 20 μL aliquot of the culture was added to 70 μL of 1:100 diluted Galacton substrate (Tropix, PE Biosystems, Bedford, Mass.) and incubated in the dark at room temperature for 60 minutes. The reaction was stopped and light emission was triggered by the addition of 100 μL Accelerator II (Tropix, PE Biosystems, Bedford, Mass.), and luminescence was read with plate reader (SpectrofluorPlus, Tecan). Timepoints were taken at 5, 8 and 12 minutes.

In either embodiment of the assay (chromosomal insertion of reporter, or reporter on a plasmid), a satisfactory assay shows normal cell growth but reduced β-galactosidase activity or gfp expression in the presence of a known signaling inhibitor. Possible problems associated with the use of fluorescence in whole-cell systems are interference by turbidity as cell density increases and the production of pyocyanin and pyoverdine, fluorescent molecules that are excreted by wild type P. aeruginosa. However, interference due to endogenous fluorescent pigments may be reduced by using mutants that lack these pigments (Byng, G. S. et al. (1979) J Bacteriol. 138(3):846-52).

### Example 5

Screening Assay to Determine Inhibition of the Signal Synthase

An assay was developed to measure inhibition of RhlI activity, based on a previously published enzyme assay for RhlI (Parsek, M. R. et al. (1999) Proc. Natl. Acad. Sci. USA. 96:4360-4365). It was shown that the substrates for RhlI are S-adenosylmethionine (SAM) and butanoyl-acyl carrier protein (C4-ACP). It is proposed that RhlI can be used as a model enzyme to study inhibition of acyl-HSL synthases. This is based on the observation that TraI from Agrobacterium tumefaciens (Moré, M. I. et al. (1996) Science. 272(5268): 1655-8) and LuxI from Vibrio fischeri (Schaefer, A. L. et al. (1996) Proc Natl Acad Sci USA. 93(18):9505-9), two homologs of RhlI and LasI, that also utilize SAM and the respective acylated-acyl carrier protein as their substrates.

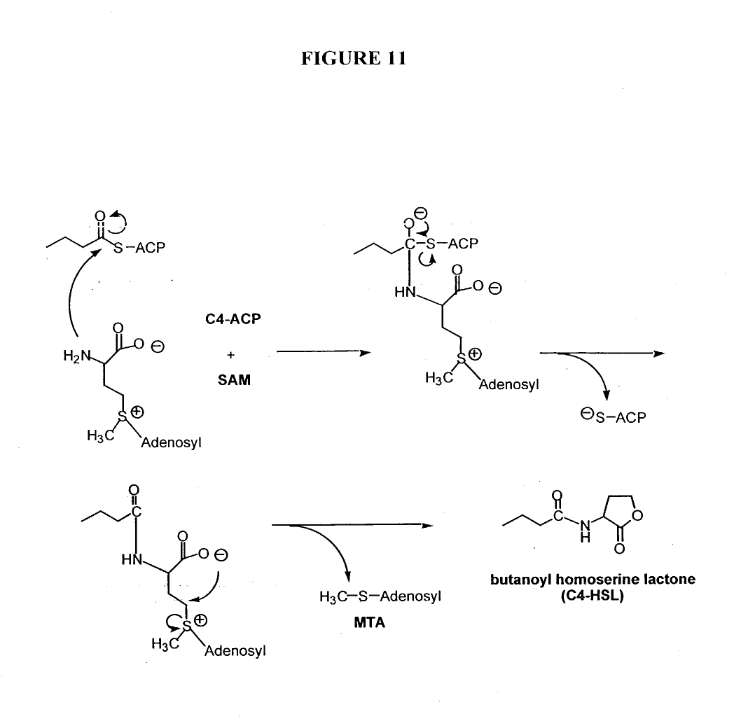

RhlI activity assay. Studies of autoinducer synthases have been hampered by the low solubility of the enzyme. It is only in the past year that the first rigorous characterization of an autoinducer synthase was published (Parsek, M. R. et al. (1999) Proc. Natl. Acad. Sci. USA. 96:4360-4365). This study was performed on RhlI, which had been slightly overproduced in a LasI minus strain of P. aeruginosa, thereby avoiding previously encountered problems of solubility. The reaction mechanism deduced for RhlI is summarized in FIG. 11. The substrates for the synthase are butanoyl-acyl carrier protein (C4-ACP) and S-adenosylmethionine (SAM). The amino-group of SAM attacks the thioester of C4-ACP to form a peptide bond between butanoic acid and SAM. The first product, acyl carrier protein (ACP) is released. Next, the SAM-moiety undergoes internal ring closure to form a homoserine lactone (HSL). Methylthioadenosine (MTA) and butanoyl-HSL (C4-HSL) are released.

The enzyme assay reaction mixture contains 60 μM ¹⁴C-labeled SAM and 40 μM C4-ACP in a final volume of 100 μL (buffer: 2 mM dithiothreitol, 200 mM NaCl, 20 mM Tris-HCL, pH 7.8). The reaction is started with the addition of 70 ng RhlI, incubated at 37° C. and quenched after 10 min by addition of 4 μL of 1 M HCl. Product formation is quantitated by extracting the reaction mixtures with 100 μL ethyl acetate and scintillation counting the radiolabeled C4-HSL, which partitions into the organic phase. (SAM remains in the aqueous phase.)

Other variations on the assay include detection of the non-acylated ACP (i.e., ACP with a free thiol group). Non-acylated ACP can be detected through the use of a thiol reagent such as dithionitrobenzoic acid (DTNB), which releases a highly colored thiolate (ε₄₁₂=13 600 cm⁻¹ M⁻¹) upon reaction with thiol groups (Ellman, G. L. (1959) Arch. Biochem. Biophys. 82:70-77). Another variation of this assay uses an even more sensitive reagent, 4,4′-dithiobipyridyl which has a ε₃₂₄=20 000 cm⁻¹ M⁻¹ (Jamin, M. et al. (1991) Biochem J. 280(Pt 2):499-506). Use of DTNB eliminates the need for radioactivity and allows for a continuous assay.

Another variation on the assay includes using a substitute for the substrate C4-ACP. It has already been found that RhlI turns over butanoyl-CoA in lieu of C4-ACP (Parsek, M. R. et al. (1999) Proc. Natl. Acad. Sci. USA. 96:4360-4365). The K_{M} for the CoA substrate is 230 μM, compared to 6 μM for C4-ACP, but Vₘₐₓ is only one order of magnitude slower. N-Acetylcysteamine represents a truncated moiety of CoA and acylated N-acetylcysteamines often function as substrate analogs for CoA-dependent enzymes (Bayer et al. (1995) Arch Microbiol. 163(4):310-2; Singh, N. et al. (1985) Biochem Biophys Res Commun. 131(2):786-92; Whitty, A. (1995) Biochemistry. 34(37):11678-89). It will be determined whether butandyl-N-acetylcysteamine is turned over by RhlI. If so, an assay will be developed for the release of free thiol groups with a thiol reagent such as DTNB. Butanoyl-N-acetylcysteamine is readily synthesized from the commercially available precursors butyrylchloride and N-acetylcysteamine.

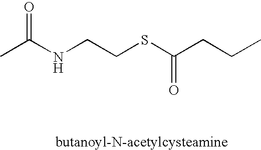

LasI activity assay. In analogy with RhlI, TraI, and LuxI, proposed substrates for LasI are SAM and 3-oxo-C12-ACP. In this assay, compounds are tested for inhibiting the activity of LasI. This assay is based on observations that bacterial strains incubated with ¹⁴C-labeled methionine produce radiolabeled acylated-HSLs, which can be isolated from the culture supernatant and identified by their retention times (in comparison to known standards) when eluted over a high pressure liquid chromatography (HPLC) reversed phase column. A synthase-inhibitor assay has been set up using this methodology.

A Pseudomonas strain that expresses lasI but not rhlI, such as PDO100, is grown in the presence and absence of the test compound (Brint, J. M. et al. (1995) J Bacteriol. 177(24):7155-63). Cells are pulsed for 10-30 minutes with ¹⁴C-labeled methionine (available from American Radiochemicals) and pelleted by centrifugation. The supernatant liquid is extracted with ethyl acetate and the products separated by HPLC. If the test compound inhibits LasI synthase, the amount of 3-oxo-C12-HSL produced will be significantly reduced when compared to the control.

An in vitro assay for LasI activity similar to the radiometric assay used to study RhlI will be developed. The substrates for this assay are ¹⁴C-labeled SAM (available Amersham Pharmacia) and 3-oxo-C12-ACP (similar methodology in Moré, M. I. et al. (1996) Science. 272(5268):1655-8). LasI activity is monitored by the appearance of radiolabeled 3-oxo-C12-HSL, after extraction into ethyl acetate and scintillation counting. Initially, crude extracts of LasI overexpressed in E. coli sérve as the source of enzyme. Once a satisfactory assay is in place, a purification protocol will be developed to obtain LasI in a soluble and active form. The purification may involve expression at low levels (low plasmid copy number, weak promoter, low growth temperature) in a P. aeruginosa rhlI mutant. Purification will follow standard techniques such as ammonium sulfate precipitation, anion exchange chromatography, cation exchange chromatography and size-exclusion chromatography.

### Example 6

In Vivo Assays to Determine Inhibition of Signal Binding

In vivo assays were also used to determine whether a test compound inhibits signal reception by LasR.

One assay used the P. aeruginosa strain QSC102 (Table 3), which responds to the presence of exogenous 3-oxo-C12-HSL by inducing β-galactosidase activity up to 400-fold (Example 1). Cells were grown in the presence of a minimal concentration of 3-oxo-C12-HSL and in the presence and absence of the test compound. If the test compound interferes with signal reception, β-galactosidase activity is reduced. Interference can be a result of any of several mechanisms. The simplest is, if the test compound prevents the 3-oxo-C12-HSL from binding to LasR. Alternatively, the test compound may prevent LasR from binding to DNA or interacting productively with RNA polymerase.

A further in vivo assay is used to determine whether a test compound inhibits binding of 3-oxo-C12-HSL to LasR. This assay is based on an observation originally made with LuxR of Vibrio fischeri. Namely, the autoinducer binds to Escherichia coli cells in which LuxR is produced, provided that LuxR is co-expressed with Hsp60 (Adar et al. (1993) J Biolumin Chemilumin. 8(5):261-6). This finding was used to develop a competition-assay for binding of inhibitors to LuxR (Schaefer, A. L. et al. (1996) J Bacteriol. 178(10):2897-901) and LasR (Passador, L. et al. (1996) J Bacteriol. 178(20):5995-6000). Briefly, cultures of E. coli harboring expression plasmids for Hsp60 and LasR (or LuxR) are induced for several hours, at which time an aliquot of cells is added to tritiated signal molecule, alone or in combination with a potential inhibitor. After 10-15 minutes, cells are pelleted by centrifugation, washed, and the amount of radioactivity bound to the cells is determined by scintillation counting.

Plasmids for expression of LasR (pKDT37) (Passador, L. et al. (1996) J Bacteriol. 178(20):5995-6000) and Hsp60 (pGroESL) have been made. A simple method for preparing ¹⁴C-labeled 3-oxo-C12-HSL has been developed. E. coli cells expressing lasI excrete ¹⁴C-labeled 3-oxo-C12-HSL into the medium when incubated in the presence of ¹⁴C-labeled methionine. The ¹⁴C-labeled 3-oxo-C12-HSL can be recovered by extraction into ethyl acetate and purified by HPLC. The correct product is identified by its radioactivity and by the correct HPLC retention time compared to an unlabeled standard.

### Example 7

Assay for Inhibition of Biofilms

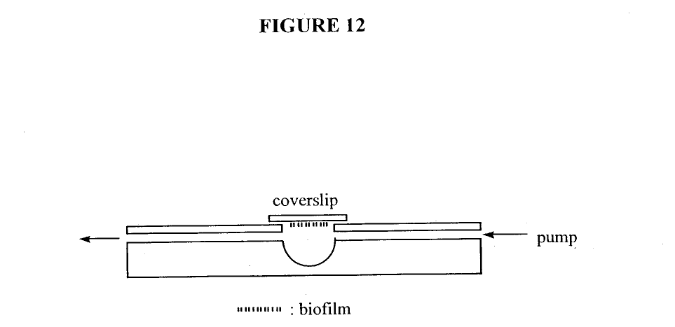

This assay tests whether compounds useful for inhibiting quorum sensing also inhibit or modulate the formation or growth of biofilms. The LasI/LasR signaling system was found to regulate not only the expression of virulence factors, but also the development of mature biofilms (Davies, D. G. et al. (1998) Science. 280(5361):295-8). This was demonstrated by using a simple flow-through system, as shown in FIG. 12, that allows fresh medium to be pumped through a small chamber in a Plexiglas body.

Cultures of P. aeruginosa expressing green fluorescent protein (GFP) were grown in a chamber that was sealed with a coverslip and flushed with fresh medium. Surface attachment and biofilm maturation were determined by examining the coverslip by epifluorescence and confocal microscopy. Both wild type PAO1 and a rhlI mutant strain were able to attach to the surface and form the mushroom-shaped structure characteristic of a biofilm. However, a lasI mutant that cannot synthesize the signal molecule 3-oxo-C12-HSL was only able to attach to the surface. It did not encase itself in an extracellular matrix or form any kind of three-dimensional structure. It also remained susceptible to 0.2% sodium dodecyl sulfate, which was used to mimic the susceptibility to a biocide. When the 3-oxo-C12-HSL signal was added back to the lasI mutant cells, the wild type phenotype was restored. The cells formed biofilms and remained resistant to sodium dodecyl sulfate.

Accordingly, the bioreactor depicted in FIG. 12 is inoculated with wild type P. aeruginosa PAO1 that expresses GFP. Test compounds (signaling inhibitors) are added to the flow-through medium to determine whether they prevent formation of the three-dimensional structures typical of a bacterial biofilm. Biofilm formation is monitored using a confocal microscope.

### References

1. Holloway, B. W., Krishnapillai, V. & Morgan, A. F. (1979) Microbiol. Rev. 43, 73-102.

2. Brint, J. M. & Ohman, D. E. (1995) J. Bacteriol. 177, 7155-7163.

3. Sambrook, J., Fritsch, E. F. & Maniatis, T. (1989) Molecular cloning: a laboratory manual (Cold Spring Harbor Laboratory Press, Cold Spring Harbor, N.Y.).

4. Miller, V. L. & Mekalonos, J. J. (1988) J. Bacteriol. 170, 2575-2583.

5. Simon, R., Priefer, U. & Puhler, A. (1983) Bio-Technology 1, 37-45.

6. Pearson, J. P., Pesci, E. C. & Iglewski, B. H. (1997) J. Bacteriol. 179, 5756-5767.

7. Linn, T. & St Pierre, R. (1990) J. Bacteriol. 172, 1077-1084.

8. Schweizer, H. P. (1993) Biotechniques 15, 831-833.

9. Figurski, D. H. & Helinski, D. R. (1979) Proc. Natl. Acad. Sci. USA 76, 1648-1652.

10. Simon, R., O'Connell, M., Labes, M. & Puhler, A. (1986) in Methods in Enzymology, Vol. 118, pp. 640-659.

11. Albus, A. M., E. C. Pesci, L. J. Runyen-Janecky, S. E. West, and B. H. Iglewski. 1997. Vfr controls quorum sensing in Pseudomonas aeruginosa. J. Bacteriol. 179:3928-35.

12. Brint, J. M., and D. E. Ohman. 1995. Synthesis of multiple exoproducts in Pseudomonas aeruginosa is under the control of RhlR-RhlI, another set of regulators in strain PAO1 with homology to the autoinducer-responsive LuxR-LuxI family. J. Bacteriol. 177:7155-63.

13. Chapon-Herve, V., M. Akrim, A. Latifi, P. Williams, A. Lazdunski, and M. Bally. 1997. Regulation of the xcp secretion pathway by multiple quorum-sensing modulons in Pseudomonas aeruginosa. Mol. Microbiol. 24:1169-78.

14. Chugani, S. A., M. Whiteley, K. M. Lee, D. D. Argenio, C. Manoil, and E. P. Greenberg. 2001. QscR, a modulator of quorum-sensing signal synthesis and virulence in Pseudomonas aeruginosa. Proc. Natl. Acad. Sci. USA 98:2752-2757.

15. de Kievit, T., P. C. Seed, J. Nezezon, L. Passador, and B. H. Iglewski. 1999. RsaL, a novel repressor of virulence gene expression in Pseudomonas aeruginosa. J. Bacteriol. 181:2175-84.

16. Diggle, S. P., K. Winzer, A. Lazdunski, P. Williams, and M. Camara. 2002. Advancing the quorum in Pseudomonas aeruginosa: MvaT and the regulation of N-acylhomoserine lactone production and virulence gene expression. J. Bacteriol. 184:2576-2586.

17. Fernandez De Henestrosa, A. R., T. Ogi, S. Aoyagi, D. Chafin, J. J. Hayes, H. Ohmori, and R. Woodgate. 2000. Identification of additional genes belonging to the LexA regulon in Escherichia coli. Mol. Microbiol. 35:1560-72.

18. Fuqua, C., M. R. Parsek, and E. P. Greenberg. 2001. Regulation of gene expression by cell-to-cell communication: acyl-homoserine lactone quorum sensing. Annu. Rev. Genet. 35:439-68.

19. Gambello, M. J., and B. H. Iglewski. 1991. Cloning and characterization of the Pseudomonas aeruginosa lasR gene, a transcriptional activator of elastase expression. J. Bacteriol. 173:3000-9.

20. Gambello, M. J., S. Kaye, and B. H. Iglewski. 1993. LasR of Pseudomonas aeruginosa is a transcriptional activator of the alkaline protease gene (apr) and an enhancer of exotoxin A expression. Infect. Immun. 61:1180-4.

21. Glessner, A., R. S. Smith, B. H. Iglewski, and J. B. Robinson. 1999. Roles of Pseudomonas aeruginosa las and rhl quorum-sensing systems in control of twitching motility. J. Bacteriol. 181:1623-9.

22. Hassett, D. J., J. F. Ma, J. G. Elkins, T. R. McDermott, U. A. Ochsner, S. E. West, C. T. Huang, J. Fredericks, S. Burnett, P. S. Stewart, G. McFeters, L. Passador, and B. H. Iglewski. 1999. Quorum sensing in Pseudomonas aeruginosa controls expression of catalase and superoxide dismutase genes and mediates biofilm susceptibility to hydrogen peroxide. Mol. Microbiol. 34:1082-93.

23. Latifi, A., M. Foglino, K. Tanaka, P. Williams, and A. Lazdunski. 1996. A hierarchical quorum-sensing cascade in Pseudomonas aeruginosa links the transcriptional activators LasR and RhIR (VsmR) to expression of the stationary-phase sigma factor RpoS. Mol. Microbiol. 21:1137-46.

24. Latifi, A., M. K. Winson, M. Foglino, B. W. Bycroft, G. S. Stewart, A. Lazdunski, and P. Williams. 1995. Multiple homologues of LuxR and LuxI control expression of virulence determinants and secondary metabolites through quorum sensing in Pseudomonas aeruginosa PAO1. Mol. Microbiol. 17:333-43.

25. Lyczak, J. B., C. L. Cannon, and G. B. Pier. 2000. Establishment of Pseudomonas aeruginosa infection: lessons from a versatile opportunist. Microbes Infect. 2:1051-1060.

26. Ochsner, U. A., and J. Reiser. 1995. Autoinducer-mediated regulation of rhamnolipid biosurfactant synthesis in Pseudomonas aeruginosa. Proc. Natl. Acad. Sci. USA 92:6424-8.

27. Parsek, M. R., and E. P. Greenberg. 2000. Acyl-homoserine lactone quorum sensing in gram-negative bacteria: a signaling mechanism involved in associations with higher organisms. Proc. Natl. Acad. Sci. USA 97:8789-8793.

28. Passador, L., J. M. Cook, M. J. Gambello, L. Rust, and B. H. Iglewski. 1993. Expression of Pseudomonas aeruginosa virulence genes requires cell-to-cell communication. Science 260:1127-30.

29. Pearson, J. P., E. C. Pesci, and B. H. Iglewski. 1997. Roles of Pseudomonas aeruginosa las and rhl quorum-sensing systems in control of elastase and rhamnolipid biosynthesis genes. J. Bacteriol. 179:5756-67.

30. Pesci, E. C., J. P. Pearson, P. C. Seed, and B. H. Iglewski. 1997. Regulation of las and rhl quorum sensing in Pseudomonas aeruginosa. J. Bacteriol. 179:3127-32.

31. Pessi, G., and D. Haas. 2000. Transcriptional control of the hydrogen cyanide biosynthetic genes hcnABC by the anaerobic regulator ANR and the quorum-sensing regulators LasR and RhlR in Pseudomonas aeruginosa. J. Bacteriol. 182:6940-9.

32. Pessi, G., F. Williams, Z. Hindle, K. Heurlier, M. T. Holden, M. Camara, D. Haas, and P. Williams. 2001. The global posttranscriptional regulator RsmA modulates production of virulence determinants and N-acylhomoserine lactones in Pseudomonas aeruginosa. J. Bacteriol. 183:6676-83.

33. Rahim, R., U. A. Ochsner, C. Olvera, M. Graninger, P. Messner, J. S. Lam, and G. Soberon-Chavez. 2001. Cloning and functional characterization of the Pseudomonas aeruginosa rhlC gene that encodes rhamnosyltransferase 2, an enzyme responsible for di-rhamnolipid biosynthesis. Mol. Microbiol. 40:708-18.

34. Reimmann, C., M. Beyeler, A. Latifi, H. Winteler, M. Foglino, A. Lazdunski, and D. Haas. 1997. The global activator GacA of Pseudomonas aeruginosa PAO positively controls the production of the autoinducer N-butyryl-homoserine lactone and the formation of the virulence factors pyocyanin, cyanide, and lipase. Mol. Microbiol. 24:309-19.

35. Stintzi, A., K. Evans, J. M. Meyer, and K. Poole. 1998. Quorum-sensing and siderophore biosynthesis in Pseudomonas aeruginosa: lasR/lasI mutants exhibit reduced pyoverdine biosynthesis. FEMS Microbiol. Lett. 166:341-5.

36. Stover, C. K., X. Q. Pham, A. L. Erwin, S. D. Mizoguchi, P. Warrener, M. J. Hickey, F. S. Brinkman, W. O. Hufnagle, D. J. Kowalik, M. Lagrou, R. L. Garber, L. Goltry, E. Tolentino, S. Westbrock-Wadman, Y. Yuan, L. L. Brody, S. N. Coulter, K. R. Folger, A. Kas, K. Larbig, R. Lim, K. Smith, D. Spencer, G. K. Wong, Z. Wu, and I. T. Paulsen. 2000. Complete genome sequence of Pseudomonas aeruginosa PAO1, an opportunistic pathogen. Nature 406:947-948.

37. Toder, D. S., M. J. Gambello, and B. H. Iglewski. 1991. Pseudomonas aeruginosa LasA: a second elastase under the transcriptional control of lasR. Mol. Microbiol. 5:2003-10.

38. van Delden, C., R. Comte, and A. M. Bally. 2001. Stringent response activates quorum sensing and modulates cell density-dependent gene expression in Pseudomonas aeruginosa. J. Bacteriol. 183:5376-84.

39. Whitehead, N. A., A. M. Barnard, H. Slater, N. J. Simpson, and G. P. Salmond. 2001. Quorum-sensing in Gram-negative bacteria. FEMS Microbiol. Rev. 25:365-404.

40. Whiteley, M., and E. P. Greenberg. 2001. Promoter specificity elements in Pseudomonas aeruginosa quorum-sensing-controlled genes. J. Bacteriol. 183:5529-34.

41. Whiteley, M., K. M. Lee, and E. P. Greenberg. 1999. Identification of genes controlled by quorum sensing in Pseudomonas aeruginosa. Proc. Natl. Acad. Sci. USA 96:13904-13909.

42. Whiteley, M., M. R. Parsek, and E. P. Greenberg. 2000. Regulation of quorum sensing by RpoS in Pseudomonas aeruginosa. J. Bacteriol. 182:4356-60.

43. Winson, M. K., M. Camara, A. Latifi, M. Foglino, S. R. Chhabra, M. Daykin, M. Bally, V. Chapon, G. P. Salmond, B. W. Bycroft, and et al. 1995. Multiple N-acyl-L-homoserine lactone signal molecules regulate production of virulence determinants and secondary metabolites in Pseudomonas aeruginosa. Proc. Natl. Acad. Sci. USA 92:9427-31.

34. Winzer, K., C. Falconer, N. C. Garber, S. P. Diggle, M. Camara, and P. Williams. 2000. The Pseudomonas aeruginosa lectins PA-IL and PA-IIL are controlled by quorum sensing and by RpoS. J. Bacteriol. 182:6401-11.

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

What is claimed is:

1. A method for identifying a modulator of quorum sensing signaling in bacteria, said method comprising:
providing a cell which comprises a quorum sensing controlled gene, wherein said cell is responsive to a quorum sensing signal molecule such that a detectable signal is generated;
contacting said cell with a quorum sensing signal molecule in the presence and absence of a test compound;
and detecting a change in the detectable signal to thereby identify said test compound as a modulator of quorum sensing signaling in bacteria.

2. The method of claim 1, wherein said quorum sensing controlled gene contains or is controlled by a las-rhl box sequence.

3. The method of claim 1, wherein said cell further comprises means for generating said detectable signal.

4. The method of claim 3, wherein said signal generation means comprises a reporter gene, and wherein said quorum sensing signal molecule causes transcription of said reporter gene, said transcription providing said detectable signal.

5. The method of claim 4, wherein said reporter gene is operatively linked to a regulatory sequence of said quorum sensing controlled gene.

6. The method of claim 5, wherein said reporter gene is selected from the group consisting of ADE1, ADE2, ADE3, ADE4, ADE5, ADE7, ADE8, ASP3, ARG1, ARG3, ARG4, ARG5, ARG6, ARG8, ARO2, ARO7, BAR1, CAT, CHO1, CYS3, GAL1, GAL7, GAL10, GFP, HIS1, HIS3, HIS4, HIS5, HOM3, HOM6, ILV1, ILV2, ILV5, INO1, INO2, INO4, lacZ, LEU1, LEU2, LEU4, luciferase, LYS2, MAL, MEL, MET2, MET3, MET4, MET8, MET9, MET14, MET16, MET19, OLE1, PHO5, PRO1, PRO3, THR1, THR4, TRP1, TRP2, TRP3, TRP4, TRP5, URA1, URA2, URA3, URA4, URA5 and URA10.

7. The method of claim 6, wherein said reporter gene is lacZ or GFP.

8. The method of claim 1, wherein said cell does not express said quorum sensing signal molecule.

9. The method of claim 8, wherein said quorum sensing signal molecule is produced by a second cell.

10. The method of claim 1, wherein said cell is a prokaryote or eukaryote.

11. The method of claim 10, wherein said cell is a bacterium.

12. The method of claim 9, wherein said second cell is a prokaryote or eukaryote.

13. The method of claim 12, wherein said second cell is a bacterium.

14. The method of claims 11 or 13, wherein said bacterium is a gram negative bacterium.

15. The method of claim 14, wherein said gram negative bacterium is Pseudomonas aeruginosa.

16. The method of claim 11, wherein said bacterium is a mutant strain of Pseudomonas aeruginosa which comprises a regulatory sequence of a quorum sensing controlled gene operatively linked to a reporter gene, wherein in said mutant strain, lasI and rhlI are inactivated.

17. The method of claim 13, wherein said second cell is wild type Pseudomonas aeruginosa.

18. The method of claim 1, wherein said quorum sensing controlled gene is endogenous to said cell.

19. The method of claim 11, wherein said quorum sensing controlled gene encodes a virulence factor.

20. The method of claim 11, wherein said quorum sensing controlled gene encodes a polypeptide which inhibits a bacterial host defense mechanism.

21. The method of claim 11, wherein said quorum sensing controlled gene encodes a polypeptide which regulates biofilm formation.

22. The method of claim 1, wherein said quorum sensing signal molecule is an autoinducer of said quorum sensing controlled gene.

23. The method of claim 22, wherein said autoinducer is a homoserine lactone.

24. The method of claim 23, wherein said test compound is a homoserine lactone analog.

25. The method of claim 1, wherein said modulator inhibits an enzyme involved in the synthesis by said bacterium of said quorum sensing signal molecule.

26. The method of claim 1, wherein said modulator inhibits reception of said quorum sensing signal molecule by said bacterium.

27. The method of claim 1, wherein said modulator scavenges said quorum sensing signal molecule.

28. A method for identifying a modulator of quorum sensing signaling in Pseudomonas aeruginosa, said method comprising:
providing a wild type strain of Pseudomonas aeruginosa which produces a quorum sensing signal molecule;
providing a mutant strain of Pseudomonas aeruginosa which comprises a reporter gene operatively linked to a regulatory sequence of a quorum sensing controlled gene, wherein said mutant strain is responsive to said quorum sensing signal molecule produced by said wild type strain, such that a detectable signal is generated;
contacting said mutant strain with said quorum sensing signal molecule and a test compound; and
detecting a change in the detectable signal to thereby identify said test compound as a modulator of quorum sensing signaling in Pseudomonas aeruginosa.

29. The method of claim 28, wherein in said mutant strain, lasI and rhlI are inactivated.

30. The method of claim 28, wherein said reporter gene is lacZ or GFP.

31. The method of claim 30, wherein said reporter gene is lacZ.

32. The method of claim 30, wherein said reporter gene is GFP.

33. The method of claim 32, wherein said reporter gene is a variant of GFP.

34. The method of claim 33, wherein said variant is GFPmut2.

35. The method of claim 28, wherein said mutant strain of Pseudomonas aeruginosa comprises a promoterless reporter gene inserted at a genetic locus in the chromosome of said Pseudomonas aeruginosa, wherein said locus comprises a nucleotide sequence of any of the nucleic acid molecules of Tables 5 and 6.

36. The method of claim 35, wherein said reporter gene is contained in a transposable element.

37. A mutant strain of Pseudomonas aeruginosa comprising a promoterless reporter gene inserted at a genetic locus in the chromosome of said Pseudomonas aeruginosa, wherein said locus comprises a nucleotide sequence of any of the nucleic acid molecules of Tables 5 and 6.

38. The mutant strain of claim 37, wherein said reporter gene is contained in a transposable element.

39. The mutant strain of claim 37, wherein lasI and rhlI are inactivated.

40. The mutant strain of claim 37, wherein said strain is responsive to a quorum sensing signal molecule such that a detectable signal is generated by said reporter gene.

41. The mutant strain of claim 37, wherein said reporter gene is lacZ or GFP.

42. The method of claim 41, wherein said reporter gene is a variant of GFP.

43. The method of claim 42, wherein said variant is GFPmut2.

44. A method for identifying a modulator of a quorum sensing signaling in Pseudomonas aeruginosa, said method comprising:
providing a wild type strain of Pseudomonas aeruginosa which produces a quorum sensing signal molecule;
providing a mutant strain of Pseudomonas aeruginosa which comprises a promoterless reporter gene inserted at a genetic locus in the chromosome of said Pseudomonas aeruginosa, wherein said locus comprises a nucleotide selected from any of the nucleic acid molecules of Tables 5 and 6; and wherein said mutant strain is responsive to said quorum sensing signal molecule produced by said wild type strain, such that a detectable signal is generated by said reporter gene;
contacting said mutant strain with said quorum sensing signal molecule and a test compound; and
detecting a change in the detectable signal to thereby identify said test compound as a modulator of quorum sensing signaling in Pseudomonas aeruginosa.

45. The method of claim 44, wherein said reporter gene is contained in a transposable element.

46. An isolated nucleic acid molecule comprising a nucleotide sequence, said nucleotide sequence comprising:
a regulatory sequence derived from the genome of Pseudomonas aeruginosa, wherein said regulatory sequence regulates a quorum sensing controlled genetic locus of the Pseudomonas aeruginosa chromosome, and wherein said locus comprises a nucleotide sequence selected from any of the nucleic acid molecules of Tables 5 and 6; and
a reporter gene operatively linked to said regulatory sequence.

47. An isolated nucleic acid molecule comprising a quorum sensing controlled genetic locus derived from the genome of Pseudomonas aeruginosa, wherein said locus comprises a nucleotide sequence selected any of the nucleic acid molecules of Tables 5 and 6, operatively linked to a reporter gene.

48. An isolated nucleic acid molecule comprising a polynucleotide having at least 80% identity to a quorum sensing controlled genetic locus derived from the genome of Pseudomonas aeruginosa, wherein said locus comprises a nucleotide sequence selected from any of the nucleic acid molecules of Tables 5 and 6, operatively linked to a reporter gene.

49. An isolated nucleic acid molecule comprising a polynucleotide that hybridizes under stringent conditions to the complement of a nucleotide sequence comprising a quorum sensing controlled genetic locus derived from the genome of Pseudomonas aeruginosa, wherein said locus comprises a nucleotide sequence selected from any of the nucleic acid molecules of Tables 5 and 6, operatively linked to a reporter gene.

50. The nucleic acid molecule of any one of claims 47, 48, 49, and 50, wherein said reporter gene is contained in a transposable element.

51. A vector comprising the isolated nucleic acid molecule of any one of claims 47, 48, 49, and 50.

52. A cell containing an isolated nucleic acid molecule of any one of claims 47, 48, 49, and 50.

53. A method for identifying a modulator of quorum sensing signaling in bacteria, said method comprising:
providing the cell of claim 52, wherein said cell is responsive to a quorum sensing signal molecule such that a detectable signal is generated;
contacting said cell with a quorum sensing, signal molecule in the presence and absence of a test compound;
and detecting a change in the detectable signal to thereby identify said test compound as a modulator of quorum sensing signaling in bacteria.

54. A compound which inhibits quorum sensing signaling in Pseudomonas aeruginosa, said compound having been identified by the method of claim 28.

55. The compound of claim 54, which inhibits quorum sensing signaling in Pseudomonas aeruginosa by inhibiting an enzyme involved in the synthesis of a quorum sensing signal molecule, by interfering with quorum sensing signal reception, or by scavenging the quorum sensing signal molecule.

56. A method for identifying a quorum sensing controlled gene in bacteria, said method comprising:
providing a cell which is responsive to a quorum sensing signal molecule such that expression of a quorum sensing controlled gene is modulated, and wherein modulation of the expression of said quorum sensing controlled gene generates a detectable signal;
contacting said cell with a quorum sensing signal molecule;
and detecting a change in the detectable signal to thereby identify a quorum sensing signaling controlled gene in bacteria.

57. The method of claim 56, wherein said cell further comprises means for generating said detectable signal.

58. The method of claim 57, wherein said signal generation means comprises a reporter gene, and wherein modulation of the expression of said quorum sensing controlled gene modulates transcription of said reporter gene, said transcription providing said detectable signal.

59. The method of claim 58, wherein said reporter gene is operatively linked to a regulatory sequence of said quorum sensing controlled gene.

60. The method of claim 58, wherein said reporter gene is operatively linked to said quorum sensing controlled gene.

61. The method of either of claims 59 and 60, wherein said reporter gene is contained in a transposable element.

62. The method of claim 58, wherein said reporter gene is selected from the group consisting of ADE1, ADE2, ADE3, ADE4, ADE5, ADE7, ADE8, ASP3, ARG1, ARG3, ARG4, ARG5, ARG6, ARG8, ARO2, ARO7, BAR1, CAT, CHO1, CYS3, GAL1, GAL7, GAL10, GFP, HIS1, HIS3, HIS4, HIS5, HOM3, HOM6, ILV1, ILV2, ILV5, INO1, INO2, INO4, lacZ, LEU1, LEU2, LEU4, luciferase, LYS2, MAL, MEL, MET2, MET3, MET4, MET8, MET9, MET14, MET16, MET19, OLE1, PHO5, PRO1, PRO3, THR1, THR4, TRP1, TRP2, TRP3, TRP4, TRP5, URA1, URA2, URA3, URA4, URA5 and URA10.

63. The method of claim 56, wherein said quorum sensing signal molecule is produced by a second cell.

64. The method of claim 63, wherein said second cell is a prokaryote or eukaryote.

65. The method of claim 64, wherein said second cell is a bacterium.

66. The method of claim 56, wherein said cell is a prokaryote or eukaryote.

67. The method of claim 66, wherein said cell is a bacterium.

68. The method of either of claims 65 and 67, wherein said bacterium is a gram negative bacterium.

69. The method of claim 68, wherein said gram negative bacterium is Pseudomonas aeruginosa.

70. The method of claim 67, wherein said bacterium is a mutant strain of Pseudomonas aeruginosa in which lasI and rhlI are inactivated.

71. The method of claim 65, wherein said second cell is wild type Pseudomonas aeruginosa.

72. The method of claim 56, wherein said quorum sensing signal molecule is an autoinducer of said quorum sensing controlled gene.

73. The method of claim 72, wherein said autoinducer is a homoserine lactone, or an analog thereof.

74. The method of claim 56, wherein said quorum sensing signal molecule induces the expression of said quorum sensing controlled gene.

75. A method of assessing whether a subject is afflicted with a biofilm-associated disease or disorder, the method comprising comparing:
a) the level of expression of a quorum sensing controlled gene in a sample derived from said subject, wherein the quorum sensing controlled gene is selected from any of the nucleotide sequences of Tables 5 and 6, with
b) the level of expression of the quorum sensing controlled gene in a control non-biofilm producing sample,
wherein differential expression of the quorum sensing controlled gene in the sample derived from said subject compared to the non-biofilm producing bacterial sample is an indication that the said subject is afflicted with a biofilm-associated disease or disorder, thereby assessing whether a subject is afflicted with a biofilm-associated disease or disorder.

76. The method of claim 75, wherein said subject is human.

77. The method of claim 75, wherein said subject is immunocompromised.

78. The method of claim 75, wherein said biofilm-associated disease or disorder is selected from the group consisting of cystic fibrosis, AIDS, middle ear infections, acne, periodontal disease, catheter-associated infections, and medical device-associated infections.

79. A method for treating a subject having a biofilm-associated disease or disorder comprising administering to the subject a therapeutically effective amount of a quorum sensing controlled nucleic acid modulator or quorum sensing controlled polypeptide modulator, thereby treating said subject having a biofilm-associated disease or disorder.

80. A method for modulating biofilm formation and development comprising contacting biofilm forming bacteria with an effective amount of a quorum sensing controlled gene modulator or a quorum sensing controlled polypeptide modulator, thereby modulating biofilm formation and development.

81. The method of claim 79 or 80, wherein the quorum sensing controlled polypeptide modulator is selected from the group consisting of a small molecule, an antibody specific for a quorum sensing controlled polypeptide, a quorum sensing controlled polypeptide, and a fragment of a quorum sensing controlled polypeptide.

82. The method of claim 79 or 80, wherein the quorum sensing controlled nucleic acid modulator is selected from the group consisting of a quorum sensing controlled nucleic acid molecule or protein, a fragment of a quorum sensing controlled nucleic acid molecule, an antisense quorum sensing controlled nucleic acid molecule, and a ribozyme.

83. The method of claim 79, wherein said quorum sensing controlled gene or quorum sensing controlled protein modulator is administered in a pharmaceutically acceptable formulation.

84. The method of claim 79 or 80, wherein said quorum sensing controlled polypeptide modulator comprises the amino acid sequence of any of the polypeptides of Tables 5 and 6, or a fragment thereof.

85. The method of claim 79 or 80, wherein said quorum sensing controlled nucleic acid modulator is administered using a gene therapy vector.

86 The method of claim 79 or 80, wherein said quorum sensing controlled nucleic acid modulator comprises the nucleotide sequence of any one of the nucleic acid molecules of Tables 5 and 6 or a fragment thereof.

87. The method of claim 79, wherein the subject is a mammal.

88. The method of claim 79, wherein the subject is human.

89. The method of claim 79, wherein said subject is immunocompromised.
